SEQUENCE LISTING (121 sequences)

SEQ ID NO: 1 - DNA, 4017 bp, synthetic construct
  source 1..4017: mol_type = other DNA
atgaagatttcgaaggtagatcatactaagagtgcggtgagtgtacaaactgcacagggacagcaaggtattctttataaggatccgtccacagaagagatgagcgtggaagatcgcgtcaccaaaagagcagatgctacgaaagcattatacgctgtgttcaatcaacctaaggataaacgaagcatttcaggggaagcaacaacggttgcatcttcttttaactatgtgatcaaagacctgaaaaaaagcaaatcacttaatgggaagttgagtgtggagagcctctatgaagctgtgggaaatgaacttaaaggaaaacatgcgagtgctgaagagatagatttggcaattacattattgctgaagaaaagtctgcgtagagattcctttatagaggcgttaaagctggtgctgggtaaagcgtataaaggagaaaagctaaatgaggaagataagagaataatcaaggacgatctgatagttcctttgataaaggattatgataaatcatctatcagagagcaggcagtggcctcgatcaaacatcagaaccttatcgcacagccggattccaagtctgatgacgcagtaatggttatatccaatatagccggggcttctgagagaagtacgaatgagaaagaagctttaaggcagtttatatctgaatatgcagtgctagatgacagtgttcgtcatgatatgcgtgttaaactccgtcgtctggtgatcctgtatttttatggcatggatgtagttccgacgggtgattttgacgaatgggaagatcatgtccagaggggaaagaccgctgatctgttcatagactttgcacctgtcggaggtaagacagatgctgatagattaaaggatgctattcggaaaatgaacattgaaagataccgatatagcgtagatgcgatagatcaggataatacagaactcttcttcgaagatatgatgatcaacaaattctttattcatcatattgaaaacgaagtagagcggatatataggaatactaagcctggtgatgagttcaagagaagcttaggctatatcagcgagcgtgtgtggaagggaattattaactatctgtgtattaagtatatagcaataggaaaggctgtatataattgcgctatggctgggttgggctctgatcagcccgatattaaattgggagtcatcgacagagtgtatgctgatggcatcagttcctttgattatgagattatcaaggcacaggaaacactccagagagaaacctctgtatacgtttcatttgcgattaatcatcttggggctgctactgttaatctgactgaaaaagagaccgattttcttacacttgataataaacagattaaggaactggcaaagacaggtgttctcaggaatattcttcagttttttggtggtaaatccgtatggaaaaatttcgagtttgctcctgaagggggtaccggcaatgaagaaattgtattgttatattaccttaaagacatcctgtatgcgatgagaaatgagaactttcatttttcaacagccagcattaatgatggctcatgggacactgatctgatcggcaggatgtttgcatacgactgtaccagagcaggggtgggacaaaagaacaaattctattccaacaaccttccaatgttctataaatcagaggacctggaacgagcattacatattttgtatgatcactatagcgagcgagcttcgcaggttccggcgtttaatacggtatttgtgagaaagaacttttctgaaatacttaaggggcagaatctgcctatgccaacttcagctgaagaatctcttaaatatcagaatgcgatctattatctctataaagagatttattataacgtatttctgagttcatcagaaagccgagattatttcattaaagcagtcaagtcacttaggtgggaaaactcgaatgaagagaatgccgtaaaagactttcaaaatcgaattaatgaactgaccggaaaatacagtttatcccagatttgtcagttgatcatgacagaatacaatcagcaaaacagcggaagcagaaaaaagaagactgctaaggatgaacagaataaaccggatattttcaagcactataagatgcttttgtataagagcattcgggaagcaatgcttaagtatgtggacgataaatcagaggactttggttttataaaaagtccggtattcggaaaggatgacaactgcattgcgttagaagaattcctccctgattatgaatcgacgcaaaatgcaaaactgatagaacgcgtgaagtcagatttcagacttcagaaatggtacattttgggaagactgctcaatcctaaacaggtaaatcagcttgccggatctatcaggagttatattcagtattcagatgatgtaaagagacgtgcaaaagaaaatggtaataagattcatgtatccacggaatcgtatccttatcagactgttttgagagtcattgatctgtgtgcgaaattgagtggacttaccaccaataacatagatgactattttgatggtagtggggattatctgtcataccttgctcgctttgtggagtatgatccgaatgatataccgaagatatatcatgatgaagctaatcccattcttaatcgcaacatcattatggctaaattatatggcgcaggagatgttattaccaatgcagtagaacatgtcaatacaagtatgatcagagatcttgaatcatatgagaagaaaacgttggggtatcgctcttctggagtatgtaaggacaaggatgaacaggaaacgctaaaaaaatatcaggaattaaagaaccgggtagagcttcgagagatagtggagtgttctgaaattataaatgaacttcagggacagctgatcaactggtgttatctgagagagagggatctgatgtatttccagctggggttccattatacctgtttgaagaattcatccgataaaccggagatgtatgtaaaagcgaaaaccgtagatggaactattgatggatttatcctgcaccagatcgccgcattatatacgaatggactaaagctgtattcttgcggtaaagctgtaagagatgataatagaaagataattcattatgacctgagcagtggaaaagaattaaagggcaatgataagagtgcagccgggaaaaagattactgactttatggggtatacgtcattagcgttaaacaggacagagaatgacatactccctatatccggtgatttttattatgccgggctggaactgtttgaaaacgtaaatgaacacgagaatatcataagcctcaggaattatatcgatcacttccactattatgcaaaacatgacagaagcatgatcgatatctacagcgaggtatttgacagattcttctcctatgacatgaaatatcgtaagaatgtgcctaatatgatgtacaacattctgctgtcgcattttgtgaaagcacaattcgtattcggatcaggaatgaaggagtccggagaaaagaccaagtcccaggccagatttgacttgaaggataaagccgggcttgagcccgaacaattgacatataaggttgcgaattctgagaaaccggtgcaactttctgctaaagacaaacaattcctgaagacggttgcattattgttgtattatccggaaaaaaagacgtttccggaggggatgtatgccgataccaggtttgtagaagggacatcatcaaataaaaggaataacaattcttcaggcaaccgacatggcaatggtaatcacaatgtaggaggacataacaaagggtacaatcagggcaggaaaaacggcaattggtctaaggacaaatccggagacagaaatgccggaaagaaacaaacaaataaaaaccggaaagatagtaccagcgtctataaagatgaaggattcagtaacagaataaatattccttctgagtattattctcagaaaccggggaaaaagtag

SEQ ID NO: 2 - DNA, 4149 bp, synthetic construct
  source 1..4149: mol_type = other DNA
atgaaattatcaaaaactggaaaaaacggctggcatcacagaaacggagtaaaggttaacaacagtaaacaggagggatttgtttacagtattcctcataatgatggtgagagtacagataagtttgttgaagacagaaagaaagattttaagagactttataaggtatttccttctgttgaaaaagccagaaacataagtgaagagattgctgcagtcatagataaaacgatcagaaataaaagaacagaaatatggaccggaaaaaatgattattctgaaatggcctgcagattcagaaatctgcttcagcgcgaatctatgttcagacagcctgtagaagtaaaaaccgctgaatatatggtttatggtcttttgcgatcaagcctgcgttctgaaaaaacggagaaggatcttatagattttttatgccatgtgaatgacaagtcctcatcagctggtgcaatatttatgcaggagctgaccagggattatatgggtgaaaagatcaattataagtcgataataaaccagaatctcgtaatccagcctgttaagacagaatccttaaaagataacattgatcaggaggatgttttactaactgtttccgaaaggaaaaatcctgaagactcggcaaagaattataaaagtattgaaaataaagcattacggagctttttattggaatatgccagccttgatgataataagcgtaaggatctgagaaaaaagctccgcaggatagttgtactctatttttatggaaagtccgaagcagacggcttaggagaaaactttgatgagtggaatgaccatgaatccaggcgtgcatgtgaggagaagttcattgaatttgatgagagtaccaaggataatttcagatccaaaacatctaagctgataagaaatgccaatatcacagcatacaggacttcaaaagaaatcattgaaaacaatcatgacgggctgtattttgccaatcctgattataatttcctttggttaaggcatttatccagggaggttgagcgtctgacatcaaatatcaatgctgataagacgtataagctgaataagggctatttaagtgaaaagtcctggaagggaataatcaattatcttagcataaagtatatagctataggcaaatctgtattcaatttcgcatcttccggagtagattctgatggcagtgatatacagattggcgaagtaaacagggagttccaaaacggaataagttcatttgaatatgagaggataaaagccgaagaaacacttcagagagaaagtgcggtaaaagttgcctttgctgcaagacatcttgcatcggctacgatgaatcttacaccggaagattcagatatgctcctgtttgataaggacaaaatgtctcagaatctaaaagatacaggcagggtactggctgatgtattgcagttttttggaggacagtctgtctggaaggattatattataaaagaaactgaaaagtattcaagtgaagaagaattcggaactgatttattatataatctgaaaaaatgcgtctatgccttaagaaatgacagtttccatttcaaaactttgaataataaggctgattgggatactgaccttgtagcaggattgtttgaaaaagactgtgaaaacatggtaggccttgataaagataagttttatgataataacctttacaagttctttaaacaggaagacctgaaaaaagtattagacaaactgtatgataaaatccatgacagggcatctcagatacctgctttcaatacagtgtttgtaagaaataatttcagcaggttcctgttgtctaaaggtatcacacgctcatttgcgtcagaggagcagggaagacagtttgcaagtgcagtatattatcttttcaaagaaatctattacaatgattttatccagtcaggaaatgctaaaacgctgttcctgaattatgttaattcaatcaaaatcgagaaagcaattaacagatacgggaaagaagaaactaaaagagaatgcaaaccggcagaagatttcaagacatatataacgttgtgccggaatatgagtttctctgagatatgtcaggcagtaatgactgagtataaccagcagaacaaccagtccagaaagaaaaaatcagcttttgatacagcaaaaaacaaagataaattcagacattatgtagatattttacatgaagggatcagggaagcatttgcagcatatattggtctcaatgatcagaaaaattatgatggtatatatggctttgtaaagtcatttaacagcagcgatgtgttcacagtcgaaaaggacaagtttattgaagggtacagatctgaacgtttccaaaatctgatcagtaaagtgagacagaatcctgaacttcaaaagtggtatattgtggctagacttctgaatcccaaacaggtaaatgaactttccggatcgataagaagctataagcagtatattgaagatgtatgccgcaggtcgatcgctgaaaaatgtcctgtaaggaaaaatgatggaaaagaggtaagcaaagcttcttttgataatgatgtaaaagaactgatgtccatcgattatatgggtgttgtagcgatattggagatatgtatacgtcttaacggaagattttcaacagtatgcgatgattattttaaggatggtgctgacggctatgctgaatatctggagcagtatcttgattatcaagatgaaaaaactaaggatgccggtgtaagtccttcaaccatgctgtcgatgttttcagaagaagtctcagcggataatactgataagaatcagggaatcatatatcatgacgggactaatcccattatgaacagaaatattctcttatcgaaactctatggtggagcaaattcggtaatacattctgtaaagaaggtggataaccgcctgatagcagattttattaaaagcggtaagcttatacaggaatacaataagagaggatactgtattaatgaggaggaacagaaaaatctgaagagatatcaggctcttaagaacagggttgagttccgagatatagtagaatatggcgaaatcctggatgaactgcaagggcaactgattaactggagttatttaagggaacgtgatctgatgtatttccagcttggtttccattatacgagccttcataattctgaaagaaagtttgaagggtacagatatataactaaagaagacggaagtgttatagagaatgcagtactgcaccagattctatcattatatattaacggaataccgttctattacagctacgccgatgtggaaggcagagaccggtttatttgctgtgcactaaagaagaaagaacctgttgatggtaccagcaacaagtttgaagataccggaacaaagatgagatatgtaggatattactgcaaagaaggtgataattatctgggtgaagggatatatcttgcaggtctggaactgtttgagaatattgctgaacatgataatatcatcaagcttagaaactacatagatcatttccattattatattgaagacgacaggtcgatgctggatgtttacagtgaagtttttgacagatatttttcatatgatattaaatatcagaagaatgttgttaatatgctgtataatgtccttttgagtcatttcgctaaagccggatttgaatttggcgaaggaataaaacagataggaagcagcaaaaagaacgaaatgccgttaaccaaaaagatggcgcgcattctgctgaaatcacttgaatcggatgattttacttataagattggtaatacatctgaagcaaagaatcaggaaacggtcgttcttcctgccagagatgatcttttccttgatgctcttggaaaagtacttaattgggatggcagtattactgatgaaaacgctttacagaaaacagagattattacaggaagaagtggcaattatctgaagaaatcaaggaacagtgataagaagaatgatggtaataaaagaagtgatataaaaaaatcattcaataaagataaaaacatacccgaaaagaaagaagcactgaccagtactccgtttgcaaacctgtttaataatttacatatggattttgattaa

SEQ ID NO: 3 - DNA, 3924 bp, synthetic construct
  source 1..3924: mol_type = other DNA
atgaagatatccaaggttaatcatactaagtcagccgttagtgtttctgaaggttctcccaagggaatactgtatgaggatccgaccaaaagcggaacgaaggatcttgaaactcgaatactagagcgtaacgaagctgcgaaattactgtataaccctataaatacatcgagaagcagaaagaaaacacataaaataatcaatagaagtcttagggctttcttcaatcgagtaaagaagaaaaccggtgggagtttctcttgggatgaattgaaaagagtatcgtatgattcttctttggatgcagagcgtgataagattactgattctgatatagattctgttgttgaagcctgtttgaagaagagtctttcaaccccggaatgtatagaggcggtaaagcagattacaagagttttgtgtggcaagaatacctcccatgatctggatgacaaacttatagggaaattgtcttccaaattacatgatgactactcgaaggaacgattgctgggtaacatcaagaagtcgattgaaaaccagaatatggtagttcaacccggacaggttgacggagaaagcatttttaaattaactggtgatgattctttgaaagagaatccggagaaggtttctttcgaaagattccttattagctatgccaatctcgacaagaaattccgtgattgtgaattgcgcaagttaagaaggctgatagttctatatttttatggtgagacagaagtagatacgacagatgattttgacgtttgggcagatcacaaaaagcagcgtaattttaaatggtttatttcagatatcgagtttattgctacatatgaaaaataccttaaagaattacagtttgaggatagaaggaatcatcatacaaagatatcggaacctgagttcagggagaagataagacaagaaaacataaacagatataggaattcaattgcagtaattaacaaatcaaatgaagtgtactttgatgatcctgtccttaacaaattctggatccatcatatagaaaattctgttgaaaaactgctgaaaagggtaaatcccgccgattcatttaagctaaatgttgcatatataggcgagaaagtctggaaagaggttattaactatctgagtattaaatatattgctgttggtaaggctgtctatcgttttgcagttgatgatatgacctatggcgtcataccggatctgtataagtcaggaataagctcatttgattatgaactcataaaagccgacgaatcgcttcaaagggatattgccgtttcggtagcatttgctgccaataatatggcacgagctactgttgtgctcgatgagaagagcagcgattttcttgttgagagttttgatctggaaaaatccattagaacagatgttgcgctcgatatggcgatactgcaatttttcgggggcaaatcgtcttggaaacagtgcgacgcattgaaagattgtaaatatatcgatcttctttatgacatgaagaaaatgctctattccattcgtaatatgagttttcattttatttcatctgaagaaggagataacggttataagactaacgggataatcccggcgatgttcaatcaagaaataacaacctatacgacgattcttaagagcaagttctattctaacaatctgcctgctttttataatgattcagatttggaaggagaatttaaacttctatataaaaattacgtagaaagagcatctcaagttccttcttttaatagtgttgttgtcagaaagagtcttccggattttgtaaaaagagatctgaagattaaaacagctctctcgggagatgatctcacaaagtggcatagcgctctatactatcttctcaaggaaatctattacaatctcttccttgcaagtgatgatgcaaagattttgtttttgaaagcagttgaaaataataagaattctaataatagcagtgtttctgataagaatgaccatcgaagagaagctggaattgattttgccgagcgaatagagagtataaaagatcatagtctttcagagatatgtcagatcattatgaccgagtataaccagcagaatcaaagcagaaaggttaagacggctcaagatgagaagaataagaaatctttattcattcactacaagatgttgcttaatctttgccttcggaatgcatttaagatgttccttgatcgcaacgagttctcattcttaaagagtattcataatcgcgaagttaagagttcctctgatgaatggaccgctgctttttgcgcggattggacatcaaatgcatatagcatgattcaagatgagattaataaaaatccttcgttgcaaagctggtatatactatcaagattcattacaacaaaacagcttaaccatcttagtggagatatcagacattttattcagtatgtagaggacgttaagaggcgagccaaagagacaggaaatgcatgtaaatacgatcttgataataaggtttgtatataccgtaaagtacttcaggttctggatttttgtaacaaaacaagcggaatagtatcttcagagattggtgactattttaaagatgatgatgaatatgcaaagtttgttagtaactatcttgattttggtggaacgacgaagctggaattgattgcgtttacaaaccagactgttggagatgatcagatcaatatatattgcaacgattcaaagccaatcctcaacaggaatattgtaatggcaaaactgtttgcccctacagatacgataagtaaggcgatagctgcaaatggaaaccgagtaactgtcgatgatattgaggagttctattctattaaaccgattgctcagaaatttctttctgatggtgatagtgttgccaagaaagaaaagaagcaacttatagaggaacttaagaaaacaaagcgatatcaagagatagttaatcgcattgagttccgcaatattgtagattatgcagagatgataaatgacttgcttggtcagttggtcagttggtcatatctgagggaaagagatctgttgtatttccagttgggtttccattatctttgtcttatcaatgattcttacaagccggataagtatagagtccttcgtgatggagagcggataataaacaatgccgcactatatcagatcgtttcgttgtattcctttaatgtagatacttttagagatgataatgataaagataaaggtaaaaaatataataatatatgtgagtacagcctgaatattggtttagatgaagaatggcaattctatactgccgggcttgaactattcgagactatcactgaacatgattctattaagaagtttagggattatattgatcacttccattactacacaaatcaggatagaagcattctggatatgtatagcgaggtctttgaccgattcttctcttatgatatgaaattccgtaaaaacactgtcgttatcttgcagaatatactgaaatcttatttagtaataatgccggttaaatttaattcgaagtataaatcaacagataacggtagtagtaaaatgcgtgccaatgttgacatgggtgaaaaaggcttaaagtcagaggtgtttacttataagtattctgacagctgtaaggtcatcttgcctgcaaggtcgattaattatcttaaggatgttgcatctatcctgtattatcctcataaaacgcctcgtgatgctgttgatatggaagattttaaaaagaactatgaggttgcacaaactttaaataaggcaaaagacaatcatcataagtctaaaaatgattataagaataatgatcgtcctaaagataattattctccgtttaagagccagtttgataaattgaaaaagaagggtattacatttgaagataactga

SEQ ID NO: 4 - DNA, 3960 bp, synthetic construct
  source 1..3960: mol_type = other DNA
atgaaaatatcaaaagtagatcatacaagaacggcagttggggtgaatgagaatggaccattgggaattgtttattcagatccatctcagaatgctgttcaaaatccagagattagagttaggactagaattaaaaaagcaaatatgctttatacggtttttggacctacgaatgatgagatggattctcaacgagagaatggaatagcaaaagaattcaataaaataatcaaaagatataataataaaatagatcccaagaggggagaaaaggaaactgataaaaagatatataaaatgaattctgatgaactgattaaagatattaaatcggtttttggcaattattctttaaatgaatcaacgagaaaagaaattgatgaagcgttaaatgtattaattaaacgttctcttagaaaaaaggaaactattgaatcattaaatctcttatttgaaaaaacaataaagggagaggagtttaaggcggaagagaaggataagattcaaaagtatgtggttgatcgtatagttgctgactattcaaagaatacgctatcaaaaaatactataaaatcaataaaaaatcaaaatcttgtggtccaacctcaaaataaaaatggcgaatttgtttttacacaggcaaaaaataggatgaatgggaaagtaaatcaaggaagtataaggatttcaaaagctcaggaaaaagatgctttaaatgattttcttgatggttttgcagtgcttgataaacaaatgagagataagcaactcatgaaaataagacggttagttgatttatacttttatgggattgatgaggttgtaaaggaagacttttcagtttgggaaagacatgaaaaaacaaaaggtaatgataaaaaaatcattcctttttctcgtactgatatatcgactttacaaataaaacgaggagattctgaggacgagaaaaaaaggaagaatagagaaaagaaaataattaaaaaatctgatagcgctaagttggacgatatgataagaaggtggaacatagatagattccgtgaatcgtttagtgctattgataaaagtgataacagtttgttttttgatgataaaaacatttctaaattctttattcatcatattgaaaatgaagtagaaaggttatttaattcagagagattggatgattataaaatgcatattggttatgttagcgagaaggtatggaagggaattattaattatctaagtataaagtatatatcaataggcaaatctgtttataattatgcaatggaggagttaaataattcatcaggagatgtaaatcttggtgtgatagatagtagatatttgacaggtattagttccttcgattatgaaaagataagtgctgaagaaactcttcaacgagagacagcagtatatgtttcatttgcttctagtaatttgtcgagagctgtttttaaagatggagtagattgtgacttaatgtcgactaagattatagataatcacgataagtttgatgaaagcaaagtaaaaaaaagagttctacagttttttggaggagaaagctcttgggatggttttggaaagacgtttttatctgaagaatataatgaattcgattttttggaagatctaaagacacttatttaccagatgagaaatgagagttttcattttaatactgaaaagaaaaatgtagatataaaaaatcctaaacttttttcagatatgtttgcttatgaatgcagtaaagcgtgtgtgtctgaaaaagataaattttattcgaataatttacctctcttctattcagaaaaacctcttgagaaagttctgaataaactatacaccaagtataatgatcgtaagtcgcaagttccgtcttttgagaaggtaatgaaaagaagtgaatttggaaaatatctcataaaatctggagttgccaccaactttaataaagaggatacagataaacttgagtcgggactatattacctgtataagcagatatattataatgatttcctagttaacgatatgattgcaaaggggatatttgtagataatataaacaataagaaactaagaagaaatgagaataataaagtaatcaaagctgataaagggcttgaagattttaagaaacgcttgaatgaaataaaaaattattctctttctgaaatatgccaaattataatgacagaatataatcagcaaaataatcaaaagaagaaatctcagaaaaatgaggagatattccagcattataaattgggactttattcataccttcgcgaggcgttaattatatatattaataataatagtgatatttatggttttataaaacaacctactattaaatctgaaggtaaaatgccgaatattaatgagtttctaccagattattcttcaagtcagtatgatgatttgatagcaaaggtctccgattcttttgaactgaaaaagtggtatgtaatgactagattcttaaatcctaaacagactaatcatttggttggagcgttaaggaattatatacagtatgtggaaagcataaaaagaagggcggaagaaacaggaaataaaatatatatagattgtcagattttagaatctgtaaaagatatcacaaaagtagtagatatgtgtaccagaatatgtggtaatacttctaatgaaatctctgattactttgatgataacgatgactatgcaggttatctagaacgttttttagactttgaatataaagaatctttgggctcgaaatcatcaatgcttggagcattctgtatgaccaaaattaatagtgaagagataaagatttaccatgatggaacaaatcctatacttaatcgaaatatcgtattatctaaactatatggagcaaatagtataatttcagaggctgttccaaaagttgatcaaaatatgataaaagaatactatattgtggctgataaaattaaagaatatcgaaagagtggcgattgtaaaaacattgatgagattaaacagcttaaagaataccaagaattaaagaatagggttgaatttagagatattgttgaatattcagaaatacttagtgagctgcaaggacagctcgttaattgggcatatcttagggaacgagatttgatgtatttccaattagggtttcattacgtatgtctcaagaatgatagtcaaaagccagaagcatataaaatgattgaggtgccttgcgttgatgggtcttctcgaatgataaatggtgctattctttatcagatagtcgcaatgtatacttatggaatgaatatatactatagggggcataaaaaagatgaggagtacaatgatagtgaaaatcgatgggaagccttcaatggttcaataggtgagagaattccaagatttgccttgtattctggatatatgattaagggagacaatgctaagtacaaactatcatataatatttatacatcagggttggaactatttgaagttcttgaagaacatggtaacatagttgattttaggaacgatatagaccattttaactactatcaaaagaaggatagaagtatgcttgattattatagtgaggcttttgatagattttttacatatgatatgaagtatcggaaaaatgttcctaatacattatctaatattttagcgtctcattttcttgttccttcttttgtgtttgggacttcttcaaaaaaagtggggaacaaaaactatattgaaaagaagtgtgctcacattagatttaatactaagaatccgttaaaaccaggcagttttacatatgtaatttcggaagataaacgtgtagtaggaccagcgagactgaaggggtatgtaaaaaatgtattgaatattttatattatccagaagtgcctgaaatggagcttttagattcctcttatatatttaaagaagaaaaaaagagaaaacttctcaaataa

SEQ ID NO: 5 - DNA, 4221 bp, synthetic construct
  source 1..4221: mol_type = other DNA
atgaagatttctaaagtaagaggaactcagggcaaaggaagtaaacttacaattaatgcaaaggcagcagttgtaattaacccaaccggtcaggagggtattctctatgatgatccgtcaagaatgggcgaatcaagaaaaaatgacaagcagagagaatcatacatcaaggatcgtattcgtgcctctcaaaaattgtattcaattttcaatagtaatcaaaaaatccccaaaaacaaaaaaactgaatctgaaaaagcaattgatatgattattgctggtttttcatcagaagatggcgccagttttcgtttaatgtttaaagatttcgctgaaattctggataaatatgcagaaaaaagttatgaaaacagaagaaatcatatagacgaatctcccgaattatcaaagcttggggtaaatataagtgacaatcagataaacgccctttctaatctattaagtgaagaaagtatagctataaaaataaaaaaaggaactgaatcagtaaaagataaggtaaaggttagcgagagggatattgattcggcaatatcaaattgcctaaagaaatgtatgtgcagggtaaaaacaaagaaagcgttaaaggctcttcttatgaaagtttttgatatcccatatactttagatggagacgtcaatattagaagagattttattgattatgcgatagaagattattgccgtattcgtgtaaaaaacagtgtctctgaatcaattaaaaagaataatatgccggttcagccgacgagttcggaaggtgttactgtttttcagatgccttctttgcaggaaacaaaaagtaccaagagtaaagaaagggaagcatttaatcatttcctgtcagaatatgcagatttagatgagaataagagaaagtcgttacgaataaaactccgaagacttaatgatttatatttttatggaaaagacgcaactatggcattggctgataacgaggatgtggacgtttgggaagaccatgcaaaacatggcgatattaaagagttatttataaaagttcaaaaaccacaaatcacaggtgacggaaaagcggataagctggctatgagtcagtatgaagataatattcgaactaaatatagagaagcaaatattacttgttacagaaaagcagtagaagaaatagataatgacaagagtctattctttgaagataatatgctcaatatgtttgtgcttcacagaattgagagtggtgtagaacgaatatattcgcatattaaagccaatgaggagtacaagcttcaaacgggatatgtaagtgaaaaagtatggaaagatctgattaactatatttccataaaatatattgctattggaaaagcggtgtataactatgccatggatgaacttgtcagtggagataagagtattgaaatgggcaagattaatgacaattatatttccggtataagttcctttgactatgagcttattaaggctgaggagatgcttcagagagaaactgctgtttatgttgcatttgcagcaagacatcttgctcatcagacagtcgacttagacgagaaaaattcagattttttactatttccggataagagtagaaaagataaagatggtaaaaatataaatgattttataaaagaaggtattaatcttcgttctactatattgcagtattttggtggggcatcttcatggagtgatttttcatttgaaaagtatatgacagatggtcgcgatgatgtagatctgcttaccgatttacagaaagcaatttattctatgcgaaatgacagttttcattatacatctaaaaaccataataatgatggttggaataaagaattaattggagcattgtttgaatatgaggctaatcggctgactataatacagaaagataaattttattcaaacaatttaccaatgttttatgatgaaagtaatttgaaggaattattatcatctctatacagtaaatctgtagaaagggcttctcaggttccttcatttaacagtgtatttgtcagaaaatcattcccaaaagtctgtacacaagacttatctattgatgtaaagacaatgaatgaagaggataaacttaagttttataatgctctttattttatgtttaaagagatttactacaatctttttcttaatgattcaaacgttttaaatcgttttattgatatttcgacaaaaacaaaaaagaacggaaagggagatgaaggtactcattattgggcagaaaaagatttcagacagaggattctctctataatagagagcagaaaaaattatactctttcacagatatgtcagttaattatgactgaatataatcagcagaatacaggcaatatgagacataagtctgctgataaaaacggtaaaaatcctgacagttatcagcattacaaaatgcttctattgtcatatcttggagaagcgtttgttgaatttgtaaaagaaaaatatgattttgtattcacaccggttaaaagggatttgatggataaagaggcatttttgcctgatttcgcaaagacagtcaatcctcttggcgatttaattgaaagagtgaaagaatccggtgtattgcaaaaatggtatatagtaggcagattcctcagtcccaaacaggctaatcagatgcttggttctttgcacagttataagcagtatgtgtgggatatatatcgaagagcagaagagactggtacgaaaattaataaacgtgtttcagaagataccatatcaggagttgccattagagatatagacagcgtgcttgatctttgtgtaaagatgtccggaactattacgaataatctgacagattatttcaaagacaaggaagaatatgcagcttatattaacgattttcttgattttgagtataaaaccggagattacaattgggctcttaaagacttttgtaaagaaataacggatgaagatgacaaagaaggtatttattatgacggggaaaatccgataataaatcgtaatattgttatatcaaaactgtatggcgaagcggaatttgtttcaaaaatcttcaaaagagtaaataaagaagatataaaagtatataaagaccttaagaaaaatatcgaaccatatcagaatatgggaacatttgaaacaaaagagcagcaggaaaatgttaaacgtttccaggaattaaagaatcatatagaattcagagatcttgttgattacagcgaaataacaaatgaacttcaggggcagcttgtaaactggatttatttgcgtgaacgagatttaatgtatttccagcttggattccattatttatgtttgaataataacagtgagaaaccggagctatataagaaaatagaattcaaggacgaaaaagtcattgataatgccgtactttatcaaatttgtgctatgtataccaacggtttgccattatactatagcagtaccaaaaatgcaaacataaaggaagtgagtgcaaaagcgggaacgagcacaaaagtagataaattctattcatctggaatcagagctaatggtgaaagttattcgagagactatacaacttatatggctggtttggagctattcgaaaatacaaaggaacatataaatattactatgtttagaaatgatattgagcatttcagatatttagtttcaaacacaagaagtatgttggatgtatacagtgaaatatttgatcggttctttacatatgatatgaaatacaggaaaaatataccaaacattttatataatattttattggcccattttgtgaatgttcaattcgatttttctacagggaaaaagaatattggaacaggggagaatatatatgaaaagaaatgcgcgaaaataaatatccaaaataatggtggaattgtatctgagaagtttacttataaattaaaggacgagaaaacgattgatttgcctgcaaggggacgaaggtacatggaaaccgtagcaaggttattatactatcctgaaacagttgatgaggagaaaatggtaaaagatttggtcattaaagataataagccatttggaaagaaacgaaataataagtatagtaacagaaaagaaggtgcttcggatagaaaaaaatatgaagagaataaagccaggaaaaaagataatagttttatgtccggaatggacggtgtagattggtctaaattaaattttaaataa

SEQ ID NO: 6 - DNA, 4026 bp, synthetic construct
  source 1..4026: mol_type = other DNA
atgaaaatatcaaaagttgatcacaccaggatggcggttgctaaaggtaatgaacttaggagagatgagatcagtggaatcctctataaggatccgacaaaggcagggagtataaactttgatgaacggttcaataaattgaatcaatcggcaaaaatcctgtatcacgtgttcaatggagttgttacaggaaacaaacattttattaatactgttaaaagggttaatgacaatttagacagggtattattcacaggtaggaacgatgaaagaaaatctatcacagatacagatgttgttctgagaaatgcggataggatcaatgcattcgataggatttcaacagacgagagaaaacagataattgatgagttattggagatccaactgagaaagggcttgagaaagggaaagaccggacttagagagatattgctgataggtgccggagtaaaaggcagaactgacaggaaacaggatatagctaagttccttgagatcttggatgaagatttcaataagacaaagcaggctaagaatataaagttgtccatagagaatcagggattggtagtagcgcctgtagaaaaaggagaggacaggatctttgatgtcagcggggttcagaaaggaaaaagcagcaaaaaagctcaggagaaagaagctctgtctgcatttctgtcagattatgctgatctggacaagagcgtcaggactgagtatcttcgtaagatcagaagactgataaatctatatttctacgtcaaaaacgatgacgatctgtcttcagcagaaattccggcagaagtgaatctggaaaaggactttgatatctggagagatcacgaacaaaaaaagggagaaaaaggagactttgttgactacccggacatacttttggcagatcgtgatgagaagaaaagaaacagtaaacaggtaaaaattgcagagaagcaattaagggagtcaatacgcgaaaataatataaaacggtatagatttagcataaagacaatcgaaaaagatgatggaacatacttctttgcagataagcagataagtgcattctggattcaccatatcgaaaatgcggttgaacgaatattaggatcgattaatgacaaaaaactgtacagattgcatttaggatatcttggagaaaaagtctggaaggatatacttaatttccttagcataaaatacatcgctgtgggtaaggcggtatttaattttgcaatggatgatctgcaggagaaggatagagatatcgaacccggcaagatatcagaaaaagcattaaatggattgacatcatttgattatgaacagataaaggctgatgaaatgctgcagagggaagtagctgtcaatgtggcattcgcagcaaataatctggccagggtaactgtagatattcctcaagatgaaaacaaggacaaagaggatatccttctttggaataagcaggacatacacaaatacaaaaagaagtctcagaaaggtattctgaagtctactctgcagttttttggaggcgcttcaacctgggatcttaaaatgtttgagaaggcatatccggaccagaaagaggattacgaagaagaatatctatatgacattatccggatcatttatgcactcaggaataagagctttcatttcaagacatatgatcagggtgacaggaattggaacagcaaactgatcggaatgatgatcgagcatgatgctgagaaagttgtttctgttgagagagaaaagttccattccaataatctgccgatgttttataaagacgctgatctagagaagatgttggatctcttatacagcgactatacaggacgagcatcgcaggttccggcatttaacactgttttggttcgaaagaatttcccggaatttcttaggaaagacatgggctataaggttcatttcagcaatcctgaggtagagaatcagtggcacagtgcggtgtattacctatataaagagatttattacaatctgtttttgagggataaagatgtaaagaatcttttttatacttcgttaaagaatataggcaatgaagtttcggacaaaaaacaaaagctggcttcggatgattttgcgtccagatgtaaagaaataaaggatagagacctttcggaaatctgtcagatgataatgacagaatataacgctcagaactccggcaatagaaaagttaaatctcagcgtatgatcgagaaaaataaggatattttcagacattataaaatgctgttgataaagactctatccggtgcttttgcactttacttaaagcaggaaaaattcgcatttatcggaaatgcggcaacgataccgtatgaaacaactgatgtgaaggaatttttgcctgaatggaaatccggaatgtatgcatcgcttgtagatgagataaaggagaatcttgatcttcaagaatggtatatcactgggcgattcctcaatggaaggatgcttaatcagttggcaggaagcctgcgttcatacatacagtatgcagaagacatagaacgtcgtgcagcagaaaataggaataagcttttctataagtctgacgaaaagattgagacatgtaaaaaggcagttagagtacttgatctctgcataaaaatttccacaagaatatctgcagagtttacagactattttgatagcgaagatgattatgcagattatcttgaaaattatcttagctatcaggatgatacgattaaggaattatccggatcttcgtatgctgcgcttgatcatttttgcaacaaagatgatctgaaatttgatatctatgtaaatgctggacagaagccgatcctgcagagaaatatcgtgatggcaaagcttttcggacctgatagtattttaccggaggttatggaaaaggtcacagaaagtgacatacgggaatactatgactatctgaaaaaagtatcaggttatcgcgtaaagggaaaatgcagtaccgtgaaagaacaggatgatctgctgaagtttcagagattgaaaaatgcagtagaattccgggatgttactgagtatgcagaggttatcaatgagcttttaggacagctgatcagctggtcatatcttagagagagggatctgctgtatttccagttgggattccattatatgtgtctgaaaaacaaatctttcaagccggcagaatatatggatattaagagaaagaatggtacaactatacataatgcgattttgtaccagatcgtatctatgtatattaatggattagatttctacagctgtgaaaaagataatgacaagctagaagtggcggcagcaggaaagggagtaggaagtaagatatcgctttttataaagtactcagagtatttgtataatgatccgtcatataagtatgagatctataatgcaggattagaagtttttgaaaacaatgatgagcatgataatattacggatcttagaaagtatgtagatcattttaagtattatgcgtcggatgattctgataaaaaaatgagcctgcttgatctttatagtgaattctttgatcgattctttacatatgatatgaagtatcagaagaatgtggtgaatgtgttagaaaacatccttttgaggcattttgtcattttctatccaaagtttggatcaggaacaaaagaggttggagtcaagaactgtaaaaaagaaaaagatagggctcagattgaaataagtgaacagagccttacttcggaagactttatgtttaagcttgatgacaaatcggaaggagaaccaaagaagtttccggcaagggatgaacgttatctccagacgatcgccaagttgctttattatcctaaaaaagatgttgatttgaacaaattcatgacaaaagaagaatcaatgaataaaaaagttcagttcaatagaaaaaaggaaacaaacaggagacaacagaataattcatcaagcggagcattatcttcatctatgggtgatttattaaagaacatcaaattgtaa

SEQ ID NO: 7 - DNA, 3909 bp, synthetic construct
  source 1..3909: mol_type = other DNA
atgaaaatatctaaagttaatcatgtgagaacaggaacgagaattaaagaaaacaatggtgaaggagtattatatgctaatccttcaaaacagacaaatgccgtaaaagatttatctaagcatatccaggatgtaaatcagaaagctcaaggattgtattctccattaaacccggtaaaatctcttattaatcctaaaatgccaaaagaaaagaaggatgagattaatggttcatacaaagcttttaagagcgttgttatcggtattgtaaaagaaaatgaaacaggaattccggattctgcttctgtaattaggactttatatgaaaaagctaaaaaaatagatctaaaagtttcggatgcatcttatttgtcttcgaagctgatagacaagtgcttaagaaaaagtctcgagtcaaaatctgagattgcaaaagaaatattaaaggcaattatttctacggataaaagtgcggttaattcgcttaatgccgaagaagtaaaggctttctttgaactggttcataaggattattataagaaagaacaactcaaagcaattgaaaagtctatagaaaataaagatgtcaaagttcaggtaaaaacaggacaaaatggcgaaaatcatcttgttctttcaaatgctgatagtgcgaaaaagcattattattttgattttgtaaaagaatttgccacaaaagacaaagctgaaagagaagaaatgattatcagatttcgtcagttgattattcttttttattcgggttcagagtcttataaactttcgattggctctgatgttggggcttggacttttggttcgtctcttcctgaagttacagccaatgtcgacgatgaaattgcttctttgattgcagaatataatgaaaatattgcgcgcaaaaacgatattcaaaaatcgattgatttgaaatccaatcaaatgaagaattataagtttaattctcctgaatataaaaaattagatgatcaggtttcaaaactaaaggatgaacagggagattgtaagcatgcaatatccgacgcaaaaagaaagattaaagctcttgttgagaatctgatatgcacaaaatatcgtgatgctgttaaggcagagggcttaactgattctgatatattctggataggatatattcagcaggttgctcaaaaacagtttagcaagaaggacgcatataacaattacagaatatcaactaaatacctgtatgaagttacatttaatgagtggatttcgttcatggcatcaaaatacattgatcttggaaaggcagtatatcattttgcgatgcctgactttagcgatattaaatcaggtaaggaagtccatgcgggaaaagtacaacccgcttttgaagatggaattacaagcttcgattatgaaagaattaaagccaaggaaacattggcaagagatttttcagtgtatgccacttattcatcaggcattttctccaatgctgttacagatagcgaatataggctaaaagatgaaaaagaagatgctttgttttataagcaggaagattgggagcaagcgcttttgcccaatgctaagaagaagcttcttatgtattttggcggtcaaacaaaatgggaggactctgaaattgaaaagttgtcggatctagagatgactaaagcatttcaggatatgataaacgtcatccgtaactcgaactatcattatgcaggaagtgtgttggaacccggtgagcaaagcgttaacattgcaaaaatgctttttgaaaaagagttttcccagcttggaagaataataagggaaaagtatttatccaataatgttcccgtatattacaacgttgaagatattaataagatgatgacttatctatatcagggtgaatccaagagagaagcacagattccatcatttggcaatgttcttaaaaagaaagaaatgcccggatttgtatctaagtatattcccggaaacttacttgctaagtttgattccgaaggtatggacaagttcagagcatctctttactttgtattgaaggaagcgtattattatggatttttgaatgagactaatcttaaagacagatttattatggcatttaaaaattccgaaaaagatgccaagaatcctgaagctattgaaaacttcaaagcacgaatcgctgatatggatgattcatgttcgtttggtgagatatgtcagattcttatgacagattacaatcagcaaaatcagggtgaatataaggtaaagtctcagataaaacaaaatcaagatgagaaagacaacaaaggtcataagtattctcatttcaaaatgcttttgtatgtaactctgcaaaaagcgtttattgattatatttttgaaaaacaggatatatatggctatattaaagctccgattttcaaaagtaatttctttgacggagatgaacctcaaaagtttgtagaatcatgggaagccaatctgtttggcgatgtaaagaaaacaacagaaacagactcgtactatttggcatggtatgtgctttctcatatgcttccggcaaagcaggttaatcaacttcaaggcggaataaagagttatattcaatttgtcacagatataaacagacgtgaaaagagtgtactcggaacagaaaaagataatagcttggtcaataatatagattattatcaaaatatacttaaggtccttgagtttgtaatgtgttttgttggaaaaacatccaatgttttgacagattactttgctgatgaagatgactatgcattgcatttgtattcctatgtcggattcgctggtaagaaagaggagaaaaccaattctactctttccggtttttgcagtaaatctataacaaaagcaggaaaagtattaacggacagaataggaatatatcatgatggaactaaccctattgtaaataataatgtcgttaaagcgctcatgtatggcaacgagaacgttctttctgaagcggttaccagagtttcagctgatttgattaatggagaaataacaaaatactatgaagtaaagaataaacttgaaaaggtatttgaaaaaggcgaatgctcaaatatagaagagcagaaagaattaagagagttccaaaaccttaagaacagaatagaacttcaagacatatccatatttacggaaataattaatgactatatgtctgaacttgtaaatatggcttatcttcgtgaaagagatcttatgtattatcagcttggctataattatattagattggaatacggtaatgttgaggataagtataaagaacttcaaggcgacaatataaatatcaaatcaggagctcttttgtaccagatagttgcactttacacacatgagttgccgattgtttataaggacaaggatagctataaatatactaataacggtaaaattggcagatttgttaaatcatactgcgaagaagaattcaatgatttagataatacttatttgaagggcttggaattgtttgaagatataaaacttcatgacgacttgcatatgtttagaaatgaaatagatcatcttaagtactttattcgtgcagacaaatctattcttcaaatgtatagtcgtatttacaatggattctttagctacgatttgaagcttaagaagagtgtatcatacatatttgccaatatccttgcgaagtattttattattgccgatacagagatgaagagttcggttgaaaacggaaaaagagtagccatgctttcagtcaagggattggaatcggatgtatttacttataagggcaaaaaacgtgataaagaaggaaaagaaagagacagcaagtatacattgccggttagaagtgatgaattcttaaaagaagtcaagaaacttcttggctataagagtatgtga

SEQ ID NO: 8 - DNA, 3942 bp, synthetic construct
  source 1..3942: mol_type = other DNA
atgccaaacgtaaaattcaccttagtccctgtggactattcaaagccttacgacgaacaacccgattgtaaacgtcacgtcattggagcttatgctaatctggctcgtcacaacatgacactaaccatcaacaccatcatgcaggccatcggcatgcctctttttaatgagaacgaaatagaaaatgctttcaacaagtcccatcgcaaaaaattggaagctcttgacaacatccagaaagtgaaactgcagaaacgcctctatcgtcatttcccgttcttcaagcgaatgaagttggaggatgaagagaaaaagacggttcaactgaaatcgctcatgacagtcatgtcactttttacaagtctgatggctgatatacgcaacaattacacacattaccgaccttataataacaaagaagaacaaaacagacaattagaacttaaaaaagaagtaggcaaaaaactacaatatttgtacgagaatagcagccagacatttaagagcatggaggaacttgatcattccagcaatgaggtgctttcagctcttcgcattcctgaagacgttgtggaacgtttctcgccagacgatcctgattacaagaaactactcaacacgctacatgattccaatataccgaaatggaaaaaatcaggtttgaaactcgacatgaaaacccagataatcaccaagaaatctgtacgctatgtgcgaaaccctaactatcaagcctatatgatggatgaagagaaaggcttgtctgatataggcattatttacttcttgtgcctttttctggacaaacaagtatctttctcactaatggatgaagttggcttcaatcagcagattaagttcacaggtgagcatgcagaacagcagttaatgtatgtcaaagaaatcatgtgcatgaaccgtatccggatggtgaaggccaggatagacagtgagatgtcagacacggcactggcattggatatgttaagtgaactgcgcaaatgtcctcgtccgttgtatgatgtattctgtaaagaggcacgtaacgaattcaaagatgatgccacagtagtttgggaaaacactcacggcgaggaggctgttattactgaagagcaaggtgatataggggaagagacagatgctattgccgcaaatactacgggaaaaaatactcctcgcagtacctttgtacgctgggaagaccgctttccccagttggcactcaagtatatagacttgacaggtatgttcgaccatcttcgttttcaacttaatttaggtaaatatcgctttgccttttaccaacacgacaaagcatacagcgttgataatgctgagcgcctgcgtattcttcaaaaggaattgcatggttttggacgcatccaggaggtgaacgaaatgatgaaggaaaaatggcaggatgtcatggagatcaagaacgttgaggatggacaaatatataaggaacccgatgtagcagggcagaagccttacgtgacccagcagaatgcccaatatgactttgacaccaagagccactccatcggcatccgatgggagggatggcacaacaaccactctgacaatcattatggcgatttggataggagggatatgtttatcccaagattgcccgctaaccctgcatcgcctgagggcgacaagcgtcagactaatcaggcagaagaactgttaccgcctcagtgtatgctgagtctctatgagttgccagctattttgttctatcactatctgcttaaaaaatatcagaaaaatactggattggtggagaagaaaatctccgacttttacaccaacatgaaaaacttcctgacagaagtcagcgagggtaacatattacctgccgatgagaccacgttaatccgtgaattacagacaagagggcttaagttttctgacattcctgtcaaactgaagaaactgctcaagggcgaagtgacagacaatgcaaagcgcatggaggaatcagccctcctgcgtctgcatgagcgtaaagataagaaaagacgggcacttgaaagtttcattgccaaatgcaagatgattggcaccaaagaaaacaaatttaataaaatacgtgccgtcgtcaagactggatccttaggacagctactggctcgcgacatcatggaatggctaacgacagacacaaagaaacgtatgaacctgacaggtcaaaactatgttgccatgcaaacggcactttcgatgatgggacaaagtttcgagttggcacctgaagcaaaggtgacttgtgaaaagatgagaaacatctttgttaaggctaatatcctgcccatgaacgatgacgactttgatgcagattttcatcatccgttcctacttgatgtatttgacgaagagcctgtttctatcgaagacttctacaagatttacttggagaaagaaatcttctacatcgactatctaaccgaacatttcaaaaagtacaaagccaaaggggctgctctgtacataccattcctgcattgcgaaaggcttcgatggaaaaatactgaacagaacggtctgaaggaactggcagctcgctacctgcagcgtcctttgcaattgcctaacggactctttactgatgacattttccatttattggaggatatagccactaagaatgccgattttgcaaaagtgcttgaaaaacagaaaaaagacaaccatcagttgcagcagaatgtagcctacatgattcgaatctacatgaagacagtagagtatgatcagccacaaaacttctataacacgatgcccgtcggagataccaatagtccatatcgtcacatctatcgcatcttcaagaaattcttcggtgaatccatccccaagacaaacaagacgacatcgccagcctacaccatcgaggagattcgtgccattttgaataataagcagctattgaaggataagatagacttctttgtcaaggaagaaaaagagaaactgaaaaaacaacagatacgtgatttcagaaactatgaaaaaaagcagtggaaactgctaaaagcaaaaaacgaagcagcccccaaaggccagcactttaatgtgaaagctgaagttcagaaacgattgaacgagaaacgcgaggagcagcgcaaagcattagatcttttagttatggatgttaaacagaagttggagggtaaactgagaaaagtgaatgataatgagcgagcaatacgccgttataaaacacaggatatcctattgctcttcatggctcgtgaaattctaaaggcaaagagtcaggacgaggactttaccaagggattctgtctgaaatatgttatgagcgactcactgctcgacaaaccaatcgacttccagtggacggtgaatttccagaataaggaaaagaaaactatcgccaagactattgagcaaaaggacatgaagatgaaaaactatggtcagttctacaagtttgccagcgaccaccaacgtctgtcgtcgctactatcacggctacctgccgatatattcgagcgtgcagaaatcgaaaatgagtttgcatactatgacaccaaccgaagtgaggtattccgccaggcatatatcatcgagagcaaagcctaccagttaaagccagagttgactgatgatgcgaatgccaatgaagagtggtttacctatcttgataaaaaaacaaagaagcttcgcgctaagcgcaacaactttggcgagctgctgaagattctggccgctggtggcgatggcgtactggacgatgtcgagaagagtctgctgcaaagcacacgcaatgccttcggacacaacacctacgatgtagatatgcctgttatcttcagcgggaaactggataaaatgaatatccctgaggtggctaacggtattaaggacaaaatcatagaacagactgaacaattgaagaaaaacgtttaa

SEQ ID NO: 9 - DNA, 3378 bp, synthetic construct
  source 1..3378: mol_type = other DNA
atggctatatttgtaattaaggtagaaatccaacgacaaatgacactcgcattcaaaaacgaaaccgaacagaaacccgttctcggagcatacgccgccatggcaagaaacaatgccttccttaccgtgatggatatcatggaccaattgcatatcccccggactgtgctcaccgataattccggaaaagaagtggatccggaatcccatatatggagactcaacttattcccgaagaactataggcttcttcccgaacaggaggccagggcttcccggctccttcgcaatcatttccctttcctcgatctcgcggatgacctcaaggatgttgagaatgaccatggacaggcagcaaggaaaaatgtctcctatgaggacttgtgcaattcctttcttaccatgatggaagtgctgacgcaccttagagatgtcaaccttcactacaagatcaaggacgaaagaatcgcggatttctatttccgccgtgcggagaaagagaccgggcacatcctccgggaagtgctgaaagcagcaccccgaaagatcaaggaccgttacaagggaacagcgctcatggatgaaacctcgttgtccttcttcacggacgggaattacatccaaaagggaaggaaatattctttcaattcccgatgggccttcaatccgcagcggcaaccgcagccgtctgaagtgaaagtcttgaagaacaacgctcccgtcattgacagaaacacgggcataccgcgcatgttcgaaaggctatccactttcggcgagatcctcttcatcgcccttttcacggagaaacggtatatccccgaccttcttagagacagtgggcttgacaacaacttcatggcatcaggcgataacggaaaaatgtcccagcagaggattattcgggagatcatctccgcctacagcatccggctcccggaacgcaagttggatatcgagaccggggcgacacaaatcatgctggacatgctgaacgaactggcgcgctgcccctctgaactgttcgatgttctcccggaaagcgaacgaagatcctttgagataacgggcagtgacggctcccaggtccttatgaagcgttattcggacaggtatgtcccgctggtgttgcgctatttagacgtgactgaagaattcaaacggctcaggttccaggtcaataccggtctgctccggtacgagcaccacggaccgaaggagtatatggacggtgtcgcccggttccgtatcgtccagtcaagcatcaatggattcgggagaatccaggagatggaagcggcacggacagccggcgcgacttacctcggcttccccctcctgaaaactgatgacgatggaaacatgaccgagatgccctacattaccgactcggcggcccggtatgtcctgaacggcgatctgatcggcttatcatttggggacgccgctccgaagattgataccctccctaatggggcgggatttaagtacaaggtgtcctgtcctcaaccggactgctggctaagccggtatgagctcccggcgctggccttctatacgtttctttccagaaaataccatatctcacggagtaccgaggatatcgtcgaggatgctttaaatgaataccgggccttctttgcaggtatcgctgatggaagcatcacatccatggacggggtcggcatccccaggaagaatattccggagaagttgcttgactatctggaggggaggggcaagagaacggatttcaagaaatacaaggagggtctggtcgcaaagatgctcctcaatacggaagcactgctgtcccgcctcaaagaagacttgaaggtaattggaacgaaggataaccgtatcggaaaaaaatcctacgtgcgcatccgccccggtaagcttgccgagttcctcgcagaggatatcgtccggtttcaaggtcatcctgcggggatgcctgaaaagaaactgacaggacagcagtacagcatcctacagggaatgattgccactttccatgaaggactggccgacgcgtgtcggaatgccggcctgctagacggagattccgcccatccattcctttccttggtattcacacgccatgcacagggaatgacatccacagtcgatttctatcgggcataccttgaggagcggcgtacatacctaaagggcgtggttccggacgaggctcccttccttcatcaggaaagaaggaaatggtcggccaacaaggattctgcgtattacaagtccctcgcattgcgatacattaaggatgagaagacgggagacaaggtcggtgtcttcttaccgcggggactgtttgacaatgcggttcacgccatcatcaaagagcattgcccgaacacgtcgaaggtcatcaatgcttcagaacgggcaaatatggccttcatcatacttacctacctggagaaagaactggatgaccaaaaccagggattctatttcaacgaagaaaggctgaaggagtacgggttctcgaaagccatacggaaagagttggaagagagcggcatgaaacgactgtctcatgtcctgcgtcttgagaggaacaccaatccttctggactgtattacgaagctctcagggaagagtccggctggaaagatgaccgtagaaagggtggacaactggataggaagaccgaggaattcgctgaaaaactgcgccatagttataaacgtatgtgcgataatgaaaagactatccgtcgatacatggtgcaggacattacacttttccttctggcaagaagtcttgtccgcatcgcagggaactccgtcaacctatggtcagtagggccagaagggaatggaatcctcgaccagtgggtcgacgtaatgacaccgtataagaaatacataatccgtcagaagggtatcaagatcaaggactacggtgagatctacaagattcttaaggacaggcggattgactccctcctcctgaaccagaagaaacgggtgccggaggctatcgacctggaggaaatcaaggaggaactcgtcacatacaaccgcaaacgcgcctccatggtcagcgctatacaggtgtatgagaaaggtgtcttcgaggagaatcgggagcatttcgacagtatgaccagtcgcttcggcttcaaggaaatccttgaggcagacgtaaggtcaagttccttgacaaaggaggcggttaagaaagtaaggaatgcggtctcccacaatcagtatcccgaccgtatggtgattaaggatggccgtagcatggtactctactcccccgatcttcccgatatggcgaagggtatcgccgaaacgaccgatagactcacgaaatatggaactgatattagtaagcaaactgacgagtga

SEQ ID NO: 10 - DNA, 3648 bp, synthetic construct
  source 1..3648: mol_type = other DNA
atgttacaaacagaaaaaaacgatcgtggagctttttgggctgcttatttcaacacggcaacgaacaacgttcaggcgattcttcaattcgccggaaaaaacgttcagcttgaggaactttcaaatcaggagttcaagctttcagcaaacggaatcggcgatgaagagacaaaagaactcgaatggtcggccgaaagctatccggcaattcaaacgctgaaaatgacaggcgatgaagtcaacattccagaacaaattcgaatcatgaagacgctgtccaagcatcttccttttatgaagagaatctcaactcgcgtgcagaacggcgttcgcaaaaatggcaaaaccgaaaaagcgggagaggaaatgacgccgcagatgtttgctgaaattcttattgggtacgtgaattatctttacgacctgcgtaattatttcacgcactacaaacacgttcccgtctcccggaagaatatgcagtccgagtatttggggattctctttgacgccaatgttggaacggccaaagagcgcttctattcggaagacaagattgccaaagacgacaagcggtttaacaatttccgtatgcacaagggcgcggaatccgtaccggacaaaaaaggcggcatgaaaaaacagccgaaactcaacaaggatttcctgttttatctttgggagacggatccaatcacgcccaaaggcaaagacaatccgtatctcgaactgacagctcaggggctggcgtttttcctgtgtctgtttttggaaaagaagtccgctaacatgcttttggactccgtcggcattgaagaagacgcggaaagtttgattgacggattcggtttcgagaataattccggcgataatcgaacgctgctcaaacggattttcaccattacatgcgcccgacttccgcgcactcgtctggaaagcgagaatctgataagcaatcaggttcttggcctggacattatgaactatcttcataaatgcccgaaagagttttacaatctgctttcacctcaagatcagcataaattccggacgctttcagacgatcacgggacggaaacgctgctgaagcgttttgacgaccggttcccgtatctggctctgaacgttatggacagattggagtgtttcgattctctgcgattctgcattgacatgggaacgttctatttccgttgtcattctcgcgttcagattgacggctcccgtctggaaaaccgccgattaaagaagaagctgacgtgttacacccgccgtcaggacgcgatcgagtattttcaaaccgaacgcgcggcggaaaacacgttctatcaaacggacaatcttgctccggctccgaaggcgtatcgaacggacatgcttccgcagtacgacctcggccgcggtcggaagcaggaaaaccggattggaattgctctgaaatcgttagacgacagccggccgatgttcaatcaaccgacgctggatcccgccgggcaaatcaagccgaaaacgtacaagccggacgcgtggctgtccacgtacgaactggctcccgcgctgtttttgtctttgcatgggaaaggcagcgacgttgaaaagcgaattgaggaattcattcgttcctggaaggggtttgcagactggatgtccaaggcgtcaaaagaacaactcaaagaattgcgttacaattccgccagggaggaattcgaagtttttaagtcccgctttgaaaagcagtttggtttgtcggtaaacgatattccggacgaattccgctattatcttgtcaacggcaagatcaagccgatttatatgcgatttcaaagaggcagcgctgttccaataacgatggacgaagccgctcagatttggctttacaacgaatgtgacaagacccgatcgatcattcggaaatttaacaatgagcaaagctatgattttaagctcggcaaaggcaggcagcgccggtatacatccggaaacatcgccttatggcttgttcgcgattttatgcgcttccagaaagccaacgacaatcctcgagaaggttttggaaaactgaaatcgtctccagacttcaacgtcctccagtcgtctctggcgctgttcaacagccgaaaagacagtctgaaagagattttgaaaaatgctcgactgatttataacccgtccggcaaccacccgtttttggaaaacgtaatcaatcgcagcggggcgttgttgtcaattgaaggattcttcaacgcctatctggacgaacgattggagtggctaagcaatgcctctggacgggaggtttatcagcttcgcaagctgtacgaacgcggagaaaagaagcgcagcgccgccaaaggccttccccctgcaaacgtttatttgggcgaaatggcaaagcagtttctggacgagtccgtttgcttgccgcgaggtctgtttgacgaccttgttcgttcagcgctcaaggaaatctatccggaacagtacgccaaagacgtcccggacggtcagcgcgccaatttcacctggctgatgcaaaagcatctgaaatggtctggagacgatcatcagtggttctataaagagcttaagcagtcgatgacggctgaagagttcaaaaagctgtttgaattgcttggaacaacggacgttcgttacgacaacgcaggcaagaaaacgctcaatgacaaactcgaggagacgtactctcagcgctggtatgatttggaaaaaagcttgaaaaatgataaagaatttaagagcctttcacctaatgataaacagagcgctattgacgaccgcatggggcaggagaatcaaatgtaccgaagcctgctcaatcgtcttcgcgatgtgtggaagcgcattcgaatgtccagcgttcaggacgttgtacttcgcgacgcggtctggcagctgttgggactgccggaaaagtctgtgcgtctgtgcaacgtcaagccggaatacgacctgaaaacgcagcgcatgatacgcgacgacgctggagacggcagcatctccaacgatgcaattcttaatgagacgcattcgttggaaaatatcatcgatcttcccaagggcttgggaacaatcgtgctcaaaggcgaaatgaagctcaagaattttggaaacttctaccgcatgctttccgatcccaaactgccttcgttcctttgcctgtacaaacagtttgggtttaacgccgttgattacagctatctcgaacttcaggagtttgagttctacgatcgcaagtttcgtccggctgttttcaattgggttcatcagcttgaggagacggtgttggaaaagtatccggatttgccgatgaaatatggcaaatttgttgatttttggacaattgccggtaaagcaaacggcgggtatattttcactcaattattgttgacgtcaattcgcaacgccgtttcgcaccagtactatccggaattctgcgttccgtcagattggacaaatcagaaggatatcgacacgtactcgccgcagttcaaagatcagcttttaagcgtcaaggcagaattcctcaaacgacgttctgaagacaaggacgcattgttgtcagaaacgatttacgactacgcgcagtctctgtttgaaaacgcaatcgcctttgttgaacagcaaccgtaa

SEQ ID NO: 11 - DNA, 4143 bp, synthetic construct
  source 1..4143: mol_type = other DNA
atggcaaattttcaaacaccccaaaggcatatttttggtacatatctgaatatagctcgtgccaatttttacaaaacgattcttcatgtcttttctgcatcaggcattgactgttacacaaaaaggggtgacctctttgtaagagaagacaccgtagacaaagtcattagtgcattatacttgattgttaacggagagaatgcaggttatcacgccatcaaagaaatcgttagtaagagctatgacaagcgatggaaagaagacaaagcactacaaggtaatctttctggcagtgaactgaaagctagaaaagaagagtttaaatcgcctctgaaagatgaggggcctgatggcgaagatgcaagaatttgcaaatcattcacattaggaagtgagcaggaagagcgaatgaggaaattgcttttccggcatatacctctattatcacctattatggcagatgttgtagccatgcagttcaaggaaactaccaatgagcatcaagaggcaaataaaacgttacatgatgcaacgcttgccgattgcttcaaagaactgagcaacattgcaaggtgtctcagtgaaagccgcaatttctacacacataaaaatccatacaactcaatagaagcccaaagaactcagctccaattacagaaagtcatcgcaaacaatttggataaagctttcattggcagtcgtagaattgcgaagaaacgtaacagctattctgagaaagacctggcattcttgactggtcatgataatgattgcagaatggaagagatatttgtacttgatgaaaatggtaacaaaatatggaaagtagagaaggataaaaacggaaaagacaaactcgataaagacaagaatcccatttatgtctataagaaggttaaagcaaaagacgggaaaggtagagagaaacttgatgaaaaagggaagccagtttatgagactctacgtgaaaatggggaacctgtacatgaatatgagaaaaaatttgtagaacggaaagatttctatttccgtattaggggtaagcgtgaagtattagctcctgatcttaccccaacaggagaagagtgcgatggcctttctgcatttggaatgctttatttttgttctttattcttgtcaaaagagcaaacggcacaattgtgtacagaatctcgtgtattcgtcacgagcccgtatcagcctgctggtaatttaaagaataatatcattcttaatatgatgttcgtatatgccatacatattccgcgtggcaagcgtcttgacagcgagactgacagccaggcattgggaatggatatgctaaacgaactacgtcgctgccctatagaactttacgatgtactaccatctatcggaaaacgggattttgaagacaatgttaaacatgagaacaatagaacgccggaattgtccaaacgtattcgattaaaagaccgtttcccatatcttgccatgcgctatattgaccaacaacaattatttaaaagaatacgcttccatgtccggctcggtagttttcgtttttgcttctatgataagacttgtattgacggaaagtcacatcctcggcaactccacaaagatattaatggctttggccggttgcaggacatggaaaaagagcgtaaagaacaatatgggcactttttccaacaatcaagagaacagtccatatggcagaaagacgaaaatgcctatgtaaatctaaagcaattggaaccaattaaagctggtgaccagccgcatatcaccgatatgtttgcccagtacaatatccatcaaaaccgaatcgggctcttttggaacaccgatgaagaatgtaaacttgttaataaaacaaatgctcagggagagataatttacaacggatattatcttccaccgcttaactacgtggattctccaacagaaaacaacaaacacaaaagaaaagcacctgttgatatgccggcaccactatgctcattgagcgtttttgagttgcctgctatgttgttctacaattttttaagaaacaccgatagtcttgggggagaggaatttcccgatgttgaagaaattgttattaaacaatacgacaatatccgaaaatttttcttagaggtcaaggatattcagcctactgacaatatagagaatctggctaccatcctaaacgcttatggtttgtctaaacaaagtgttccaaagaagatatatgactatttgtctaataagaacacattaattagtaaagacattcggaaatctacagagaaagaggttaaaaaccgccttagaagagcaatcattagaaagcaacgctttgaacaggatcaggagcacatagaaaacaccaaggataacaagatcggaaaagatagctttgtcagcatccgctatgccaaaattgctgaggaactggcaaagtccatgatggaatggcaaagcggcaatacaaagatgacaggattgaatttcagggttctgactgctgctctggcaaaatttggcgatggtgtaataaagcgagatacgatcatttcaatgcttcaaaaggcggagatcatgggaggagacaacccacattgctttattgaacaagcaatcgagcaggaacaatatgacattgaagaattctatcttgattatattagtgcagagattgaatacctgaagcgttttctgatgattgatggaaaaacgattatattgaaagacgagcaattgcttgatgctcttaggaaagataaagaagtccatgatgatgtcaggattcgtcttaagaacgatgttgatttcggccaactcccttttatccacaagtcacgcttgcgttggcaacaaagcaaaatcgaagaattggcaaacagatatctttatgtaaaagaagaaggtgaagaaacacttggacgtgccactcttttattgcctgatggtatgtttttcccatatataatgaagggatttcagaaatgtcatcaggagctaacaaacagtatagaagccctttctgatgagcagaaaaaaggaattgaaaacaacgtggcttatttgataaatctctattttgagtctaaaggagagaagtcccaatcgttttatgattccacagaaccttcacactataatgacaatataaggcaattggcaccatacaaatatgctagatcttatgaatttttcaagataatcaaaggctggcagattcacctgtcttgcgatgaaatgaaaaagaggttgactggaaaaaagaccatcatcgacaacaaaattaatgctttaaaagagaaaggaaattacatttctttagaggaagccaaaaacgctcttcgaagaaaacttcacaacacgttccgcgacatgcaggacaatgagcgtgtcattcgtcgttataagacacaagatcgcattcttttccttatggcgagagatatgatgggcgaaattgttaacaagaaggcggacttgttcaagttggaaaatgtatgcaaggatgatttccttagtcagaaagttaaggcatccatcgcggtacatttatctatgggagaggtgtttaaaatccagaaagatgagatggcaataaaagattatggtaaattgtatcggctattgagagatgaccgaataaccaaattgttatcttatgccttgtacgaaacaggtgaaacaatcgactatgatgacatcacggatgaactgaaggaatatgaaagttgtcgttctgctgctttcgaggcagttcagatgatagaagatacaagataccaacaagacaaagaggtgctttcaaatcccaatgaaaacaacttctattgtggaaacatacgctataaaaatggaaaagataacggaagagagaatgaggcgaaacgaaacaattttagaacattattagaagatctccaaaaatttacaccagaacaaatggaaatgtttagtcaggaagatagagaattgattatatctatccgtaacgcgtttgggcataatagttacccaaagcaagtcgattttgaaagattaatcaatcaagaaaggaagaataatcccaattttaaaatcgagctaaaacaagttgcatctttcattctggataaattggaagaatatgtaaatcaagtaaatccccaaacatag

SEQ ID NO: 12 - DNA, 3741 bp, synthetic construct
  source 1..3741: mol_type = other DNA
atgagctacaccccctcctcccgccctccgcgccgcccgcagatcgtcgaaggttcgcgcaacaacgcccttcgcatccttaaaatcacgcccgacgaacagaccgcgttcgtgacctacctcaactacgccgtcaacaatctctccgaagtggctggcgtcgcgttttcggacgagaggaaggtccgtgccgacgtcttcaggggagaaccggccgacatccagcgccggatttcccgtctggcggatttcctctggtcgttccgcgagaaggacccgtccgccgacgaatcgggctacaaggccaaactcggcggcggccacgacgacatggcggtttggctaaccgaaaagattgtctccctccgcaacttcttctcccatgtcaatcgacaggactgcacgccgctcgtcatttcgcatgacgaatacgtcttcgtcgagggcatcctcggcggagccgcgcgcgatgcggcgatggggccggggctcaatcccgccaaggcccagaaactgaagctcgcaacgcaccacgtcaaggaggcccacacctacgagttcacacgcaaggggctggtgttcctgacgtgcctcggcctctacaaggacgaagccgaggagttctgccatctcttccacgaaatgaaggtcccggaccgcatcgaggacgcggacctcgatgaagaactgccggacggccgccaccgcgccgtcctcggactggaggacttggacaagttcgccggtctcaagggcaagggccgcgctttgatcgagctgttctcgttctacagctaccgccgcggacgccagtccctggacgcggcgaacctcgacttcatgcagttcgccgacgtcgtcggctgcctcaacaaggttccggcccccgcgttcgaataccttccgctcgaaaaggagcacgcggaactcgaagccctcaaggccgcctcgaccgaatccgaggaaaacaagcgcaccaagtacgtcctgcgccgccgcgaccgcaaccgcttcctttccttcgcggccgcctactgcgaggacttcggcgtcctgccgtccgtccgcttcaagcggctcgacgtacgccggacgaccggccgccaccagtacgtcttcggccccggcgccgacgcggaaacggacgaggaggaaagcaaccgcgtccgcctcaaccgccactacgccatccgccgtgacgcgatcccgttcgagttcgagccggaccgccactacggtccggttcgcatcggcgcgctccgcagcgccgtcagcgccacggagatgcgcagacttctctacctccacgcccagggcgcggacatcgacgccgtgctccgcggctacttcgaggcctaccaccgcgtcctcgaacgcatggtcaacgccggctccctcgccgagatctccctcgacgacgaaaccttcctcgccgacttcgcgacgatctgcggcacgtcgccggaagcgttcaaggcggatccggaggccttccgcaagttcgttccggagagtctccggcgctacttctccaaggacgccggaccccgttcggaacagcgtctccacgcgcttctctgttcgaagctcctggccgcggtcaaccgcacgtgcgacatcctcgtccgtcacgacgccctcgaggcgtggcgcgaggcgtcccgtccctggctcgacgcacgggacgactggcgcggccggatcgacgtctggcgcaaggccaatccgaagaaagatcttcccgacgaactcaagaccttcgaggcctatcttgaaacgctcccggccgacgagcgtcccgcccgtccggaggaaccccgttgccgcgtcggcgaaggcgaaggcgaaatcacgaatccgccgacctggtgccggttctccgacgccgactacgtctcgttcgtcttcgactggttcaacctcttccttccgaacaaccgcaagttccgccagctccccatctccgagcagcacagggaaggcgtcgaagaccatcttttccagatggtccacgccgccatcggcaaattctctctcgaccagaaggggctctggtccctgctcgaaaagaaccgggcggagctcaagccgtatgccgacctcctgcaataccggacggacatacgtgtccttgtggctccgttcgttcgggaactcaatcatcaacttcctgagaatcggcgacttccggaatttgacaatggtgttgacaaagacgaaaaagaaattgcaagcccgattcttaacatcttccgtcatttcaggcagaacgagctctggcaatttttgcagaatttccggccggagttgaagtcttggtgtcggagcatgaagagcaagctccaagacgccatggaaaagaaaccgtctcgaaaagatggcaaaccgcacttcccgaaagttgaagacttgtttgaggtgttgaatcttcgtcgcccctccccgaccctggaagacctcgccgtggaggctgtcaagctctcgaacgacatgttcctcgaagagacgaaccgctggggcgctgccaatccaatgtccgtttcgcgcgacgatttgctcgccgcctgccgccgcttcggcgtccgtcccggaatgcccgtggagtacaagtccctcctcaagaccgtcctcggcatcgactacgatgcctggcgccacgccttcgactacgggaccggccgtccgttcgcggaccgccggctggaggacgaggagcacgtcgccgcccagattccgcttccgaacggcttcgccgaccgtgtcgcggcgtctctcccgcgcaaggtccgcgagcggttccaggctcccggctccgccgcgttcgacttcaacgccgccttccgcgcctggtccccggatccggccgtttcgctccgcgacttctacgacgtcaagccgctcgtcgcgagccagatcgcccgaaagcacccgcccgagggcggggcggcgaccgccgaccccttcgccgccctttccgcaaagacgctcgattccgccgtgcgcgaaatcaaggacgccgaaaaccaggacaaggtcctcctgttcgtggcgatgaaatactgggagcgctttcgcggcggcgacacctactcgaccggcaagctcaagatgccgttctcggagaagaccacgctgcgcgagttcttcgacacgcccgttcagatcgaaaaagatggcctgaccgtttccttccgtcccaacgacgtcaaccgtccggcgttcgcgacgctgttcggcaattcccgcgcggcgaaggactaccgcgccaagatcgcgaagatcctttctccggacgggtcccgcacggccttcgacttctacgagatggtcgtcgccttccgcgagcagaaggctcgcgaccgccacgagcgcctggccttcacgccctacgccgtccatttcgacgccctctgcgaaatccccgcctccgcctacgagaaggcggcggaaggtcgtttcggggaggcgaagaccgaggcgatccgcgccatggagttcgagcgctacaaggcggtcctccccggcttgacccgcgaggactacgatgccgtcgccgacgcccgcaacgccgtttttcacacaggtttcaagttcgactgctccaaggcgatcgccgtctgcaagcgcctcggcgtcctcggaatgatgccgggcgaagccgctccggctggccgcagaacgtcgtcccattctgggccgaaatggtccggcccgcgccggaacggcggttcgaggtggtga

SEQ ID NO: 13 - DNA, 2766 bp, synthetic construct
  source 1..2766: mol_type = other DNA
gtgagcaagaatgataacatcaaatccaaagcaaaagcacttggcttgaagtcaacgtttcaggttggcgacgaggtcgttatgacttctttcggtaaaggtaataaagccatcgttgaaaagattgtacgtggaaccgacgtgcagtctgttccgactgagccgaacttcagcgctgaaatcgaaggtaaaaaatttgaccttgtcggcagagctcatattcagacaaagtcggacaatcctcagtattcaaagaaaagaaccggcgacgatatgataggcgcaaaggcggcacttgaaaagagattcttcggcggtacgtttgacgacaatatacatattcagctcgcttacaatgttcttgatattgaaaagattctttctgtccatatcaacaatattgtttacacgcttaataacatacgaagaaaagacaacgctgaagatgacgattttatcggctatatgagtaccagaaacgactacgacacctttattgaaccgagaaagcacaacattagcgaagacgctgctaaaagcattgacaaatcaagagcctcgtttgaagagtatcttcagccgccggtaaaggattgtctgcattatttcggaaacaccttttttgctccccgtgagattgaaaagacatacacggataatcgcggctttgaaaggaagaaaaaagttaccgtaaactcgcttatagacgagaaggaaatatactatatttttgccctcctcggcggacttcgtcaattctgtacccatgacaacaaaagcggcagaaactggctttattctctcgaggaaaacggaataaactctgatgcaaaggcggtacttgacaagtactataattctgcagttgcaaggattgacgaaagctttgttgacaacgcatctaaaactaatttcaagctgatttttaatgcaatggatgtcagcgacgaagcgcttcaggacaatatagcaaaggcgttttacaagtttacggtttgtaagagctttaaaaacatgggcttttcaattaaaaagctccgtgaacagctgctcgaacttcctgaatatgaagggcttaaagataagcattacgattccgtgcgctcaaagctttatcagattatggacttcgttatttatctgagtttcaaagatgaaaagtttaagaaggataatgaagaaaagattaataatatcgttaacgaactccgtgcgaccttgaacgaagaggataaaggcagagtatatgcctcttatgccgaaaggatgaaatctgagcttaaaccggcaattgcacgccttaagtctgatattgacaaaattaaggacagcagggtgaaagagtttgaacttgatgcgtccgtaaaatatagactctcaaaggtcgtagaaagtgtgcgccttaaggatagggcaacatattttacaaagctcatatatctgactactctgttccttgacggcaaggagattaacgacctcctgactactcttattcatcagtttgagaatatcgcaagttttattgatgttatgaacgacagaggtattgattgccgtttttcggatggatataagctttttgaatcctcaaagcaaattgcgtttgagcttcgcaatgtcaatagctttgctcgtatgacaaggacctcaaaggatgacgaaaacgcaacgcatatgatgtatatagatgctgcagagattctcggcaccgattataccgaagagcagattgaggagcatcttaacctggaaaagaaaagaatgataccaggcacgaaaaaagcggatatgaacttccgtaactttataatcaataacgttataaagtcatcacgcttcaattatcttgtcagatattccaatcctaaaaaaatcagagcgctcgccgataacgagggtgtaatccgttttgtcctgggcgagttgccggatgcgcagattgaccgttatactttactttgcggcttcaatcccgatgccgaccggcaggaaaaaacggacaaacttgcaaaggcaattactggtttgaggtttaacgattttgaaaatgttaaacagggcgcaaatactgagggcgaatctcaggagtcaattgacaaggcacagaagcagggactgatttctttgtacttaaccgttctatatcttttgacaaagaatcttgtttatgtaaactcacgctattttcttgcattccattgtcttgagcgtgatgcgcagctgcttggaagcggtgctgggcatcatgagccttatgttgctcttactcagcgctttattaatgaagataagctcaatgagcatgcctgcgaatatcttaagaccaatattgcaaattcggatgaatatacaattagaatattccgcaataatgctgctcacttgagtgctgttagaaatgcaaatctgtatattgacaagttgaaggaatttaaatcgtactatgagatttatcatttcctttctcaagagaatatttacggtaaatattgcgttgataagaaatatgttacagccgatgaaaatggaagtaaaacaataagtgttaaaattagcaaggattattgtcctcaggtatatatcgacaagtcgcttgaatactttgacaagctcaataaatatggtacgtattgcaaggacttcacgaaggcactcaattcgccattcggctacaacctcgcaagatataagaatctgtctatcgagggcttgtttgaccgaaaccgtcccggagacaaaggtgagaacacgtttgaggactaa

SEQ ID NO: 14 - DNA, 2871 bp, synthetic construct
  source 1..2871: mol_type = other DNA
atggcgaagaaacttagtccgaaagaaataagggaagctgcaaaagcagaaaagatgaaaagcataaaagctgcagaggctgaaagagaaaaagctgctgaagaagcgaaacttaaagctgaagcagaaaagaaagaaaaagcagaaaaaaatgaacgcgaaaaagcactgaagcgcttcagacttgatgaaaaatcacgaatggctcttcctaaaagtgaaagaaagtcacttgccaaagccgctggggtcaaatcagcttttgctgttggaaatgacatttatcttacttcattcgatcgcggtaacgatgcaatcgtagaaaagaaaatcactgatacagtggtaacaaatctgagaagtgatgaatcatttgaggtaaatgaaaatactattacagaaatgtctgttccgataaaaagcaaacgtatatctgatctgtacgctatagctgacaacccgctttacagaaaagattcagcgactaaagttcaaccggacaagcttcttctcaaggatactttagaaaaactatactttggaaaaacttttgatgatacacttcacatccagatcatatacaacattctggacattgaaaaaatacttaccgtttacagcatcaatacgatctactgcttaaacaatctgtttggaaaagaaagcggtgaaaaagaagaccttattagtaaactgacctatcagataacatatgatgaatttaaagaaagtaaagctcataatgaattcatcgatttttataatctgaacaccttagggtattacggcaatatttttttcaaagaaaaaaagaaacgttcacaaaaagaaatctatgatatcatagctcttatagctaccataagacaatggtgtgttcactgtgaagaagacaagcgcacatggctgttcaacacagaaaccgttttaagtaaggaatttcttgatattcttgatgacgtttacgaaagtcttgttgaaaaggtaaacagaaattttctgaaagacaacaaggttaatctgcaaatactcgaagacgtcctggaaataaaagacagtgaatcacgtgaaaaacttatccgacagtactatcgtttcattgttaccaaagaacagaaacttctcgggttctcgattaaaaagcttcgtgaagcaatgcttgaagaaaccgaattcaaaacagacaaaaaatatgatactgttcgttcaaagctttacaagcttatcgattttcttctgtttacaggatacactactgatgaagctgaaaaagaaaaagcactttttcttattaaatctctccgggaatcattgaccgaggaaactaaagacagaatatacaaatcagaagctgctcgtttatggctgaaatacgaaaatactataaccaacaaaatcagagaagctctcaatgaaaaatctataagcgaattaaaaaaagataaaagcttcgatgataaaagtattaccagcatcataacagatgaagtatcagggaaaaaagcaacttatttttcaaaaacaatctatcttcttgctcagttcatcgacggaaaagaagtaaacgatctgaccacttcacttataaacaaatttgataatatacgaagtctcattgatactgccggacagataggacttgactgtaaatttacagaagaatacaaattctttgaaaactccgatcagataagaacagaacttcatgttataaaaaatcttactaacatggagtattacgatactactgtaaaaaaacagatgtacaaagacgccgttcacattctcggaatacaggatgatgtttctgacgcagaacttgaaaagatcatcaattccattcttcttcttaatgaaaacggcaaaccactccctggaaccaaaggtaaaaaaggattccgtaattttatcatttccaatgttttaaaatcacgtagattcatttatctcataaaatactgcaatcctaagaagataagaaaaattgcaggaaacagaaaaatcataaagttcgttttaagccgtataacagactcacaactggaaagatactactattcatgcaatccggaacttaaatccggtatctatccgggacgtgatgatgctgtaaatgatctttctattcttatcgctgacatgaaattcgaagattttaaaaatgttgatcagagcgcaaatgttcatgacaacaacaatgctgccagagaaaaaatgaagtatcagactataatcagtctttatcttacagtttgctatcatctggttaaaaatcttgtaaacataaacgccaggtatgctatggccttccacgcacttgaacgcgacgctcgcctttaccaaattttcagcagtgaagagaattatgtggataatttaaatgctgactatgctatacttactaaaacacttctgaaagacaactatgaaaatgccggaaacctttatctcagaaacaaaaaatggaataaactcacaagagagaatcttgataattacattcctcaggccgctgcaaacttcagaaacgcagtagctcatcttaacccgataagaaatgctgatatgctgcttgaagatattgaagatgtatcatcatattatgccatctatcattacatcatgcagaaaagtgttacaaacagaactataagggtttctaatactactgaagacgaaaagcgaatacttacagattatcagaacaaaatcaaaagacatcatggttataacaaagattttgtaaaagctctctgcgtgccttttgcctataatatagtcaggttcaaatctctttctatatatgaaatgtttgatcgtaattatcatgaaaaaacgtcaccggaaactgatgacagccaaagcccctga

SEQ ID NO: 15 - DNA, 2847 bp, synthetic construct
  source 1..2847: mol_type = other DNA
atggataaggaaaaaacaactgtagaaggaaaaaatactaatcaaaaatctgatgtgttaaaatcgctggcaaaagcaaatggacttaaatcttcttttgttatagggaatgaagttgttatgacttcatttggaagaggaaacagtgcgattcttgagaaaaagataacgggctcgagaatagaaaatcttaatccgaacgttgcgttttatgttaaaaaacatatatcagatagttgtaatcctgatagtggtaaatatgacgtaaagagtaaacgaatgaaggaaaaagctgttgttgatgatccagtatatgtttctccggaaaaagcttcaaatgttcatgctggtcaggatctcattggctgtaagaatgttctggaagaacggtatttcggaaagacttttgatgataatatccacatacagctgatctataatattcttgacatagaaaagatacttgcggttcatgtttcaaatgcaacttttgccattaacaatattttaggaattgaaggcaaggaaaacgaagattttatcggaaatctatcagtgctcaatacttttgatgagttcgagaattatgaaacgcatccaaagtttgcaaacaaaagtgcgataaaagaaaatctcaggaaatccaaagttttttttgataagattaaaaaaggaaataaactcggatatttcggtcaggctttttattatgcaacaggtactggtaaaaatctgatattcaccaagaaatcttcagagacgatatatgaattacttgctcttgtaggaagtctcagacaattttgcgttcatgacgaggtaatggttgataataaagtaaaatcaagaagctggctgtataatgctcaaaaagagttaaagccagattttctaaaagcacttgatgaactgtatagtaaagaagttgaaaaaatagacagtgattttattgtaaataatacagttgatttgcacattattcatgatgcaatagatgttattgacggatcagccgactggcagaaaatcacaaatgaatattatgatttcataataagaaaatgttttaagaatatcggtttttctataaaaagacttcgcgaaacaatgattgaagaacagatgaaagtattatgcggaaaatgcgatagagagaactgtaaaggttatggcaaatgcttcaaaaacaagaagtatgatagcgttcgttccagattaaacagaatcgttgattttataatattcaggcattataatgatgaagcaatcttaaagaatgtaagtcttctcagaacctgtatgagtgaagaagaaaaacagaaacgattttatttgccggaagctaaagctttatggaaaaagtacaattatgtgttcagaaattatgttcttaaaaaacttaatggcaagtcgataagcgggttaaaagagaaagcaatcgaaaattccatagatattaactcagtaaagataagcctcggtgatccggattatttctgtaagtttatttatcttcttacgttcttccttgatggtaaggagataaacgatttacttacaacgcttatcaataagtttgataacattgcaagttttataagtgttatgaaaaatgataagttatctattgactgtgaatttgtaccggaatacagctttttcgcaaacagtgctcaaatcacatctgatctcagagttataaacagttttgcaagaatgcaggcaccggctgagccgtcgaaagatgatatgtaccgtgatgctcttgatatccttggcatggatgacctgtcagaagatggaaagaagcagcttgaagatacagttttatgcagagatgaaaacggtaagtacatgaaaaaagaagataataatcctaaacgtgataccaactttagaaattttcttggaaataacgttcttgcaagtacacgtttcaagtatcttattcgctataataatgccaagaagacacgtgcacttgcaaataataaagcagttatcatctttatgttgaacaagatcaataaacagaatcctgaacagatagttagttattataaggcatgcagagatgacagtgacccagttgcttcggatgcagaagctaaaattgagtttcttgccgaaaagataatgaatgtcagctgtactcagttcagatacgttaaaaacggaactaaagtaagaccggatgaggctaaggaaaaagaaagattcaaagcgataatcggtctttatcttacagtcatgtacctgataaccaagaatatggtttatatcaattcaagatatgtgaccgcatttcattgccttgaaagggacagcgaacttcatggtgttaagtttgatcagaagaaactgcaaccgaatcttacaaaaaagtttattgatccgaaaacttgtggtgactatggcctcagaaataataaacgcgcaagaacttatatcgagcagaatatggataagatgtccaactgtactagttactggaatgaatacagaaatgccgtagcacatctttcggttatcaggaatatgaatcagtatattaaaaatgttaagaatataggcagttgttttgagctgtatcattatattatgcagcgtttcctgttagacaaagagaatattgctgaatcacttagagaatatgatgatttcataaagaagaagggatgctatagaaaagacttcgttaaggctttgaatactccgttcggatacaaccttgcaagatataagaatctttcaattgcagaactgtttgacagaaatgatactgagctggagagaacgaataagcttagaaatgaagcaatcaaagcagatatagaagaaatctga

SEQ ID NO: 16 - protein, 1338 aa, synthetic construct
MKISKVDHTKSAVSVQTAQGQQGILYKDPSTEEMSVEDRVTKRADATKALYAVFNQPKDKRSISGEATTVASSFNYVIKDLKKSKSLNGKLSVESLYEAVGNELKGKHASAEEIDLAITLLLKKSLRRDSFIEALKLVLGKAYKGEKLNEEDKRIIKDDLIVPLIKDYDKSSIREQAVASIKHQNLIAQPDSKSDDAVMVISNIAGASERSTNEKEALRQFISEYAVLDDSVRHDMRVKLRRLVILYFYGMDVVPTGDFDEWEDHVQRGKTADLFIDFAPVGGKTDADRLKDAIRKMNIERYRYSVDAIDQDNTELFFEDMMINKFFIHHIENEVERIYRNTKPGDEFKRSLGYISERVWKGIINYLCIKYIAIGKAVYNCAMAGLGSDQPDIKLGVIDRVYADGISSFDYEIIKAQETLQRETSVYVSFAINHLGAATVNLTEKETDFLTLDNKQIKELAKTGVLRNILQFFGGKSVWKNFEFAPEGGTGNEEIVLLYYLKDILYAMRNENFHFSTASINDGSWDTDLIGRMFAYDCTRAGVGQKNKFYSNNLPMFYKSEDLERALHILYDHYSERASQVPAFNTVFVRKNFSEILKGQNLPMPTSAEESLKYQNAIYYLYKEIYYNVFLSSSESRDYFIKAVKSLRWENSNEENAVKDFQNRINELTGKYSLSQICQLIMTEYNQQNSGSRKKKTAKDEQNKPDIFKHYKMLLYKSIREAMLKYVDDKSEDFGFIKSPVFGKDDNCIALEEFLPDYESTQNAKLIERVKSDFRLQKWYILGRLLNPKQVNQLAGSIRSYIQYSDDVKRRAKENGNKIHVSTESYPYQTVLRVIDLCAKLSGLTTNNIDDYFDGSGDYLSYLARFVEYDPNDIPKIYHDEANPILNRNIIMAKLYGAGDVITNAVEHVNTSMIRDLESYEKKTLGYRSSGVCKDKDEQETLKKYQELKNRVELREIVECSEIINELQGQLINWCYLRERDLMYFQLGFHYTCLKNSSDKPEMYVKAKTVDGTIDGFILHQIAALYTNGLKLYSCGKAVRDDNRKIIHYDLSSGKELKGNDKSAAGKKITDFMGYTSLALNRTENDILPISGDFYYAGLELFENVNEHENIISLRNYIDHFHYYAKHDRSMIDIYSEVFDRFFSYDMKYRKNVPNMMYNILLSHFVKAQFVFGSGMKESGEKTKSQARFDLKDKAGLEPEQLTYKVANSEKPVQLSAKDKQFLKTVALLLYYPEKKTFPEGMYADTRFVEGTSSNKRNNNSSGNRHGNGNHNVGGHNKGYNQGRKNGNWSKDKSGDRNAGKKQTNKNRKDSTSVYKDEGFSNRINIPSEYYSQKPGKK

SEQ ID NO: 17 - protein, 1382 aa, synthetic construct
MKLSKTGKNGWHHRNGVKVNNSKQEGFVYSIPHNDGESTDKFVEDRKKDFKRLYKVFPSVEKARNISEEIAAVIDKTIRNKRTEIWTGKNDYSEMACRFRNLLQRESMFRQPVEVKTAEYMVYGLLRSSLRSEKTEKDLIDFLCHVNDKSSSAGAIFMQELTRDYMGEKINYKSIINQNLVIQPVKTESLKDNIDQEDVLLTVSERKNPEDSAKNYKSIENKALRSFLLEYASLDDNKRKDLRKKLRRIVVLYFYGKSEADGLGENFDEWNDHESRRACEEKFIEFDESTKDNFRSKTSKLIRNANITAYRTSKEIIENNHDGLYFANPDYNFLWLRHLSREVERLTSNINADKTYKLNKGYLSEKSWKGIINYLSIKYIAIGKSVFNFASSGVDSDGSDIQIGEVNREFQNGISSFEYERIKAEETLQRESAVKVAFAARHLASATMNLTPEDSDMLLFDKDKMSQNLKDTGRVLADVLQFFGGQSVWKDYIIKETEKYSSEEEFGTDLLYNLKKCVYALRNDSFHFKTLNNKADWDTDLVAGLFEKDCENMVGLDKDKFYDNNLYKFFKQEDLKKVLDKLYDKIHDRASQIPAFNTVFVRNNFSRFLLSKGITRSFASEEQGRQFASAVYYLFKEIYYNDFIQSGNAKTLFLNYVNSIKIEKAINRYGKEETKRECKPAEDFKTYITLCRNMSFSEICQAVMTEYNQQNNQSRKKKSAFDTAKNKDKFRHYVDILHEGIREAFAAYIGLNDQKNYDGIYGFVKSFNSSDVFTVEKDKFIEGYRSERFQNLISKVRQNPELQKWYIVARLLNPKQVNELSGSIRSYKQYIEDVCRRSIAEKCPVRKNDGKEVSKASFDNDVKELMSIDYMGVVAILEICIRLNGRFSTVCDDYFKDGADGYAEYLEQYLDYQDEKTKDAGVSPSTMLSMFSEEVSADNTDKNQGIIYHDGTNPIMNRNILLSKLYGGANSVIHSVKKVDNRLIADFIKSGKLIQEYNKRGYCINEEEQKNLKRYQALKNRVEFRDIVEYGEILDELQGQLINWSYLRERDLMYFQLGFHYTSLHNSERKFEGYRYITKEDGSVIENAVLHQILSLYINGIPFYYSYADVEGRDRFICCALKKKEPVDGTSNKFEDTGTKMRYVGYYCKEGDNYLGEGIYLAGLELFENIAEHDNIIKLRNYIDHFHYYIEDDRSMLDVYSEVFDRYFSYDIKYQKNVVNMLYNVLLSHFAKAGFEFGEGIKQIGSSKKNEMPLTKKMARILLKSLESDDFTYKIGNTSEAKNQETVVLPARDDLFLDALGKVLNWDGSITDENALQKTEIITGRSGNYLKKSRNSDKKNDGNKRSDIKKSFNKDKNIPEKKEALTSTPFANLFNNLHMDFD

SEQ ID NO: 18 - protein, 1307 aa, synthetic construct
MKISKVNHTKSAVSVSEGSPKGILYEDPTKSGTKDLETRILERNEAAKLLYNPINTSRSRKKTHKIINRSLRAFFNRVKKKTGGSFSWDELKRVSYDSSLDAERDKITDSDIDSVVEACLKKSLSTPECIEAVKQITRVLCGKNTSHDLDDKLIGKLSSKLHDDYSKERLLGNIKKSIENQNMVVQPGQVDGESIFKLTGDDSLKENPEKVSFERFLISYANLDKKFRDCELRKLRRLIVLYFYGETEVDTTDDFDVWADHKKQRNFKWFISDIEFIATYEKYLKELQFEDRRNHHTKISEPEFREKIRQENINRYRNSIAVINKSNEVYFDDPVLNKFWIHHIENSVEKLLKRVNPADSFKLNVAYIGEKVWKEVINYLSIKYIAVGKAVYRFAVDDMTYGVIPDLYKSGISSFDYELIKADESLQRDIAVSVAFAANNMARATVVLDEKSSDFLVESFDLEKSIRTDVALDMAILQFFGGKSSWKQCDALKDCKYIDLLYDMKKMLYSIRNMSFHFISSEEGDNGYKTNGIIPAMFNQEITTYTTILKSKFYSNNLPAFYNDSDLEGEFKLLYKNYVERASQVPSFNSVVVRKSLPDFVKRDLKIKTALSGDDLTKWHSALYYLLKEIYYNLFLASDDAKILFLKAVENNKNSNNSSVSDKNDHRREAGIDFAERIESIKDHSLSEICQIIMTEYNQQNQSRKVKTAQDEKNKKSLFIHYKMLLNLCLRNAFKMFLDRNEFSFLKSIHNREVKSSSDEWTAAFCADWTSNAYSMIQDEINKNPSLQSWYILSRFITTKQLNHLSGDIRHFIQYVEDVKRRAKETGNACKYDLDNKVCIYRKVLQVLDFCNKTSGIVSSEIGDYFKDDDEYAKFVSNYLDFGGTTKLELIAFTNQTVGDDQINIYCNDSKPILNRNIVMAKLFAPTDTISKAIAANGNRVTVDDIEEFYSIKPIAQKFLSDGDSVAKKEKKQLIEELKKTKRYQEIVNRIEFRNIVDYAEMINDLLGQLVSWSYLRERDLLYFQLGFHYLCLINDSYKPDKYRVLRDGERIINNAALYQIVSLYSFNVDTFRDDNDKDKGKKYNNICEYSLNIGLDEEWQFYTAGLELFETITEHDSIKKFRDYIDHFHYYTNQDRSILDMYSEVFDRFFSYDMKFRKNTVVILQNILKSYLVIMPVKFNSKYKSTDNGSSKMRANVDMGEKGLKSEVFTYKYSDSCKVILPARSINYLKDVASILYYPHKTPRDAVDMEDFKKNYEVAQTLNKAKDNHHKSKNDYKNNDRPKDNYSPFKSQFDKLKKKGITFEDN

SEQ ID NO: 19 - protein, 1319 aa, synthetic construct
MKISKVDHTRTAVGVNENGPLGIVYSDPSQNAVQNPEIRVRTRIKKANMLYTVFGPTNDEMDSQRENGIAKEFNKIIKRYNNKIDPKRGEKETDKKIYKMNSDELIKDIKSVFGNYSLNESTRKEIDEALNVLIKRSLRKKETIESLNLLFEKTIKGEEFKAEEKDKIQKYVVDRIVADYSKNTLSKNTIKSIKNQNLVVQPQNKNGEFVFTQAKNRMNGKVNQGSIRISKAQEKDALNDFLDGFAVLDKQMRDKQLMKIRRLVDLYFYGIDEVVKEDFSVWERHEKTKGNDKKIIPFSRTDISTLQIKRGDSEDEKKRKNREKKIIKKSDSAKLDDMIRRWNIDRFRESFSAIDKSDNSLFFDDKNISKFFIHHIENEVERLFNSERLDDYKMHIGYVSEKVWKGIINYLSIKYISIGKSVYNYAMEELNNSSGDVNLGVIDSRYLTGISSFDYEKISAEETLQRETAVYVSFASSNLSRAVFKDGVDCDLMSTKIIDNHDKFDESKVKKRVLQFFGGESSWDGFGKTFLSEEYNEFDFLEDLKTLIYQMRNESFHFNTEKKNVDIKNPKLFSDMFAYECSKACVSEKDKFYSNNLPLFYSEKPLEKVLNKLYTKYNDRKSQVPSFEKVMKRSEFGKYLIKSGVATNFNKEDTDKLESGLYYLYKQIYYNDFLVNDMIAKGIFVDNINNKKLRRNENNKVIKADKGLEDFKKRLNEIKNYSLSEICQIIMTEYNQQNNQKKKSQKNEEIFQHYKLGLYSYLREALIIYINNNSDIYGFIKQPTIKSEGKMPNINEFLPDYSSSQYDDLIAKVSDSFELKKWYVMTRFLNPKQTNHLVGALRNYIQYVESIKRRAEETGNKIYIDCQILESVKDITKVVDMCTRICGNTSNEISDYFDDNDDYAGYLERFLDFEYKESLGSKSSMLGAFCMTKINSEEIKIYHDGTNPILNRNIVLSKLYGANSIISEAVPKVDQNMIKEYYIVADKIKEYRKSGDCKNIDEIKQLKEYQELKNRVEFRDIVEYSEILSELQGQLVNWAYLRERDLMYFQLGFHYVCLKNDSQKPEAYKMIEVPCVDGSSRMINGAILYQIVAMYTYGMNIYYRGHKKDEEYNDSENRWEAFNGSIGERIPRFALYSGYMIKGDNAKYKLSYNIYTSGLELFEVLEEHGNIVDFRNDIDHFNYYQKKDRSMLDYYSEAFDRFFTYDMKYRKNVPNTLSNILASHFLVPSFVFGTSSKKVGNKNYIEKKCAHIRFNTKNPLKPGSFTYVISEDKRVVGPARLKGYVKNVLNILYYPEVPEMELLDSSYIFKEEKKRKLLK

SEQ ID NO: 20 - protein, 1406 aa, synthetic construct
MKISKVRGTQGKGSKLTINAKAAVVINPTGQEGILYDDPSRMGESRKNDKQRESYIKDRIRASQKLYSIFNSNQKIPKNKKTESEKAIDMIIAGFSSEDGASFRLMFKDFAEILDKYAEKSYENRRNHIDESPELSKLGVNISDNQINALSNLLSEESIAIKIKKGTESVKDKVKVSERDIDSAISNCLKKCMCRVKTKKALKALLMKVFDIPYTLDGDVNIRRDFIDYAIEDYCRIRVKNSVSESIKKNNMPVQPTSSEGVTVFQMPSLQETKSTKSKEREAFNHFLSEYADLDENKRKSLRIKLRRLNDLYFYGKDATMALADNEDVDVWEDHAKHGDIKELFIKVQKPQITGDGKADKLAMSQYEDNIRTKYREANITCYRKAVEEIDNDKSLFFEDNMLNMFVLHRIESGVERIYSHIKANEEYKLQTGYVSEKVWKDLINYISIKYIAIGKAVYNYAMDELVSGDKSIEMGKINDNYISGISSFDYELIKAEEMLQRETAVYVAFAARHLAHQTVDLDEKNSDFLLFPDKSRKDKDGKNINDFIKEGINLRSTILQYFGGASSWSDFSFEKYMTDGRDDVDLLTDLQKAIYSMRNDSFHYTSKNHNNDGWNKELIGALFEYEANRLTIIQKDKFYSNNLPMFYDESNLKELLSSLYSKSVERASQVPSFNSVFVRKSFPKVCTQDLSIDVKTMNEEDKLKFYNALYFMFKEIYYNLFLNDSNVLNRFIDISTKTKKNGKGDEGTHYWAEKDFRQRILSIIESRKNYTLSQICQLIMTEYNQQNTGNMRHKSADKNGKNPDSYQHYKMLLLSYLGEAFVEFVKEKYDFVFTPVKRDLMDKEAFLPDFAKTVNPLGDLIERVKESGVLQKWYIVGRFLSPKQANQMLGSLHSYKQYVWDIYRRAEETGTKINKRVSEDTISGVAIRDIDSVLDLCVKMSGTITNNLTDYFKDKEEYAAYINDFLDFEYKTGDYNWALKDFCKEITDEDDKEGIYYDGENPIINRNIVISKLYGEAEFVSKIFKRVNKEDIKVYKDLKKNIEPYQNMGTFETKEQQENVKRFQELKNHIEFRDLVDYSEITNELQGQLVNWIYLRERDLMYFQLGFHYLCLNNNSEKPELYKKIEFKDEKVIDNAVLYQICAMYTNGLPLYYSSTKNANIKEVSAKAGTSTKVDKFYSSGIRANGESYSRDYTTYMAGLELFENTKEHINITMFRNDIEHFRYLVSNTRSMLDVYSEIFDRFFTYDMKYRKNIPNILYNILLAHFVNVQFDFSTGKKNIGTGENIYEKKCAKINIQNNGGIVSEKFTYKLKDEKTIDLPARGRRYMETVARLLYYPETVDEEKMVKDLVIKDNKPFGKKRNNKYSNRKEGASDRKKYEENKARKKDNSFMSGMDGVDWSKLNFK

SEQ ID NO: 21 - protein, 1341 aa, synthetic construct
MKISKVDHTRMAVAKGNELRRDEISGILYKDPTKAGSINFDERFNKLNQSAKILYHVFNGVVTGNKHFINTVKRVNDNLDRVLFTGRNDERKSITDTDVVLRNADRINAFDRISTDERKQIIDELLEIQLRKGLRKGKTGLREILLIGAGVKGRTDRKQDIAKFLEILDEDFNKTKQAKNIKLSIENQGLVVAPVEKGEDRIFDVSGVQKGKSSKKAQEKEALSAFLSDYADLDKSVRTEYLRKIRRLINLYFYVKNDDDLSSAEIPAEVNLEKDFDIWRDHEQKKGEKGDFVDYPDILLADRDEKKRNSKQVKIAEKQLRESIRENNIKRYRFSIKTIEKDDGTYFFADKQISAFWIHHIENAVERILGSINDKKLYRLHLGYLGEKVWKDILNFLSIKYIAVGKAVFNFAMDDLQEKDRDIEPGKISEKALNGLTSFDYEQIKADEMLQREVAVNVAFAANNLARVTVDIPQDENKDKEDILLWNKQDIHKYKKKSQKGILKSTLQFFGGASTWDLKMFEKAYPDQKEDYEEEYLYDIIRIIYALRNKSFHFKTYDQGDRNWNSKLIGMMIEHDAEKVVSVEREKFHSNNLPMFYKDADLEKMLDLLYSDYTGRASQVPAFNTVLVRKNFPEFLRKDMGYKVHFSNPEVENQWHSAVYYLYKEIYYNLFLRDKDVKNLFYTSLKNIGNEVSDKKQKLASDDFASRCKEIKDRDLSEICQMIMTEYNAQNSGNRKVKSQRMIEKNKDIFRHYKMLLIKTLSGAFALYLKQEKFAFIGNAATIPYETTDVKEFLPEWKSGMYASLVDEIKENLDLQEWYITGRFLNGRMLNQLAGSLRSYIQYAEDIERRAAENRNKLFYKSDEKIETCKKAVRVLDLCIKISTRISAEFTDYFDSEDDYADYLENYLSYQDDTIKELSGSSYAALDHFCNKDDLKFDIYVNAGQKPILQRNIVMAKLFGPDSILPEVMEKVTESDIREYYDYLKKVSGYRVKGKCSTVKEQDDLLKFQRLKNAVEFRDVTEYAEVINELLGQLISWSYLRERDLLYFQLGFHYMCLKNKSFKPAEYMDIKRKNGTTIHNAILYQIVSMYINGLDFYSCEKDNDKLEVAAAGKGVGSKISLFIKYSEYLYNDPSYKYEIYNAGLEVFENNDEHDNITDLRKYVDHFKYYASDDSDKKMSLLDLYSEFFDRFFTYDMKYQKNVVNVLENILLRHFVIFYPKFGSGTKEVGVKNCKKEKDRAQIEISEQSLTSEDFMFKLDDKSEGEPKKFPARDERYLQTIAKLLYYPKKDVDLNKFMTKEESMNKKVQFNRKKETNRRQQNNSSSGALSSSMGDLLKNIKL

SEQ ID NO: 22 - protein, 1302 aa, synthetic construct
MKISKVNHVRTGTRIKENNGEGVLYANPSKQTNAVKDLSKHIQDVNQKAQGLYSPLNPVKSLINPKMPKEKKDEINGSYKAFKSVVIGIVKENETGIPDSASVIRTLYEKAKKIDLKVSDASYLSSKLIDKCLRKSLESKSEIAKEILKAIISTDKSAVNSLNAEEVKAFFELVHKDYYKKEQLKAIEKSIENKDVKVQVKTGQNGENHLVLSNADSAKKHYYFDFVKEFATKDKAEREEMIIRFRQLIILFYSGSESYKLSIGSDVGAWTFGSSLPEVTANVDDEIASLIAEYNENIARKNDIQKSIDLKSNQMKNYKFNSPEYKKLDDQVSKLKDEQGDCKHAISDAKRKIKALVENLICTKYRDAVKAEGLTDSDIFWIGYIQQVAQKQFSKKDAYNNYRISTKYLYEVTFNEWISFMASKYIDLGKAVYHFAMPDFSDIKSGKEVHAGKVQPAFEDGITSFDYERIKAKETLARDFSVYATYSSGIFSNAVTDSEYRLKDEKEDALFYKQEDWEQALLPNAKKKLLMYFGGQTKWEDSEIEKLSDLEMTKAFQDMINVIRNSNYHYAGSVLEPGEQSVNIAKMLFEKEFSQLGRIIREKYLSNNVPVYYNVEDINKMMTYLYQGESKREAQIPSFGNVLKKKEMPGFVSKYIPGNLLAKFDSEGMDKFRASLYFVLKEAYYYGFLNETNLKDRFIMAFKNSEKDAKNPEAIENFKARIADMDDSCSFGEICQILMTDYNQQNQGEYKVKSQIKQNQDEKDNKGHKYSHFKMLLYVTLQKAFIDYIFEKQDIYGYIKAPIFKSNFFDGDEPQKFVESWEANLFGDVKKTTETDSYYLAWYVLSHMLPAKQVNQLQGGIKSYIQFVTDINRREKSVLGTEKDNSLVNNIDYYQNILKVLEFVMCFVGKTSNVLTDYFADEDDYALHLYSYVGFAGKKEEKTNSTLSGFCSKSITKAGKVLTDRIGIYHDGTNPIVNNNVVKALMYGNENVLSEAVTRVSADLINGEITKYYEVKNKLEKVFEKGECSNIEEQKELREFQNLKNRIELQDISIFTEIINDYMSELVNMAYLRERDLMYYQLGYNYIRLEYGNVEDKYKELQGDNINIKSGALLYQIVALYTHELPIVYKDKDSYKYTNNGKIGRFVKSYCEEEFNDLDNTYLKGLELFEDIKLHDDLHMFRNEIDHLKYFIRADKSILQMYSRIYNGFFSYDLKLKKSVSYIFANILAKYFIIADTEMKSSVENGKRVAMLSVKGLESDVFTYKGKKRDKEGKERDSKYTLPVRSDEFLKEVKKLLGYKSM

SEQ ID NO: 23 - protein, 1313 aa, synthetic construct
MPNVKFTLVPVDYSKPYDEQPDCKRHVIGAYANLARHNMTLTINTIMQAIGMPLFNENEIENAFNKSHRKKLEALDNIQKVKLQKRLYRHFPFFKRMKLEDEEKKTVQLKSLMTVMSLFTSLMADIRNNYTHYRPYNNKEEQNRQLELKKEVGKKLQYLYENSSQTFKSMEELDHSSNEVLSALRIPEDVVERFSPDDPDYKKLLNTLHDSNIPKWKKSGLKLDMKTQIITKKSVRYVRNPNYQAYMMDEEKGLSDIGIIYFLCLFLDKQVSFSLMDEVGFNQQIKFTGEHAEQQLMYVKEIMCMNRIRMVKARIDSEMSDTALALDMLSELRKCPRPLYDVFCKEARNEFKDDATVVWENTHGEEAVITEEQGDIGEETDAIAANTTGKNTPRSTFVRWEDRFPQLALKYIDLTGMFDHLRFQLNLGKYRFAFYQHDKAYSVDNAERLRILQKELHGFGRIQEVNEMMKEKWQDVMEIKNVEDGQIYKEPDVAGQKPYVTQQNAQYDFDTKSHSIGIRWEGWHNNHSDNHYGDLDRRDMFIPRLPANPASPEGDKRQTNQAEELLPPQCMLSLYELPAILFYHYLLKKYQKNTGLVEKKISDFYTNMKNFLTEVSEGNILPADETTLIRELQTRGLKFSDIPVKLKKLLKGEVTDNAKRMEESALLRLHERKDKKRRALESFIAKCKMIGTKENKFNKIRAVVKTGSLGQLLARDIMEWLTTDTKKRMNLTGQNYVAMQTALSMMGQSFELAPEAKVTCEKMRNIFVKANILPMNDDDFDADFHHPFLLDVFDEEPVSIEDFYKIYLEKEIFYIDYLTEHFKKYKAKGAALYIPFLHCERLRWKNTEQNGLKELAARYLQRPLQLPNGLFTDDIFHLLEDIATKNADFAKVLEKQKKDNHQLQQNVAYMIRIYMKTVEYDQPQNFYNTMPVGDTNSPYRHIYRIFKKFFGESIPKTNKTTSPAYTIEEIRAILNNKQLLKDKIDFFVKEEKEKLKKQQIRDFRNYEKKQWKLLKAKNEAAPKGQHFNVKAEVQKRLNEKREEQRKALDLLVMDVKQKLEGKLRKVNDNERAIRRYKTQDILLLFMAREILKAKSQDEDFTKGFCLKYVMSDSLLDKPIDFQWTVNFQNKEKKTIAKTIEQKDMKMKNYGQFYKFASDHQRLSSLLSRLPADIFERAEIENEFAYYDTNRSEVFRQAYIIESKAYQLKPELTDDANANEEWFTYLDKKTKKLRAKRNNFGELLKILAAGGDGVLDDVEKSLLQSTRNAFGHNTYDVDMPVIFSGKLDKMNIPEVANGIKDKIIEQTEQLKKNV

SEQ ID NO: 24 - protein, 1125 aa, synthetic construct
MAIFVIKVEIQRQMTLAFKNETEQKPVLGAYAAMARNNAFLTVMDIMDQLHIPRTVLTDNSGKEVDPESHIWRLNLFPKNYRLLPEQEARASRLLRNHFPFLDLADDLKDVENDHGQAARKNVSYEDLCNSFLTMMEVLTHLRDVNLHYKIKDERIADFYFRRAEKETGHILREVLKAAPRKIKDRYKGTALMDETSLSFFTDGNYIQKGRKYSFNSRWAFNPQRQPQPSEVKVLKNNAPVIDRNTGIPRMFERLSTFGEILFIALFTEKRYIPDLLRDSGLDNNFMASGDNGKMSQQRIIREIISAYSIRLPERKLDIETGATQIMLDMLNELARCPSELFDVLPESERRSFEITGSDGSQVLMKRYSDRYVPLVLRYLDVTEEFKRLRFQVNTGLLRYEHHGPKEYMDGVARFRIVQSSINGFGRIQEMEAARTAGATYLGFPLLKTDDDGNMTEMPYITDSAARYVLNGDLIGLSFGDAAPKIDTLPNGAGFKYKVSCPQPDCWLSRYELPALAFYTFLSRKYHISRSTEDIVEDALNEYRAFFAGIADGSITSMDGVGIPRKNIPEKLLDYLEGRGKRTDFKKYKEGLVAKMLLNTEALLSRLKEDLKVIGTKDNRIGKKSYVRIRPGKLAEFLAEDIVRFQGHPAGMPEKKLTGQQYSILQGMIATFHEGLADACRNAGLLDGDSAHPFLSLVFTRHAQGMTSTVDFYRAYLEERRTYLKGVVPDEAPFLHQERRKWSANKDSAYYKSLALRYIKDEKTGDKVGVFLPRGLFDNAVHAIIKEHCPNTSKVINASERANMAFIILTYLEKELDDQNQGFYFNEERLKEYGFSKAIRKELEESGMKRLSHVLRLERNTNPSGLYYEALREESGWKDDRRKGGQLDRKTEEFAEKLRHSYKRMCDNEKTIRRYMVQDITLFLLARSLVRIAGNSVNLWSVGPEGNGILDQWVDVMTPYKKYIIRQKGIKIKDYGEIYKILKDRRIDSLLLNQKKRVPEAIDLEEIKEELVTYNRKRASMVSAIQVYEKGVFEENREHFDSMTSRFGFKEILEADVRSSSLTKEAVKKVRNAVSHNQYPDRMVIKDGRSMVLYSPDLPDMAKGIAETTDRLTKYGTDISKQTDE

SEQ ID NO: 25 - protein, 1215 aa, synthetic construct
MLQTEKNDRGAFWAAYFNTATNNVQAILQFAGKNVQLEELSNQEFKLSANGIGDEETKELEWSAESYPAIQTLKMTGDEVNIPEQIRIMKTLSKHLPFMKRISTRVQNGVRKNGKTEKAGEEMTPQMFAEILIGYVNYLYDLRNYFTHYKHVPVSRKNMQSEYLGILFDANVGTAKERFYSEDKIAKDDKRFNNFRMHKGAESVPDKKGGMKKQPKLNKDFLFYLWETDPITPKGKDNPYLELTAQGLAFFLCLFLEKKSANMLLDSVGIEEDAESLIDGFGFENNSGDNRTLLKRIFTITCARLPRTRLESENLISNQVLGLDIMNYLHKCPKEFYNLLSPQDQHKFRTLSDDHGTETLLKRFDDRFPYLALNVMDRLECFDSLRFCIDMGTFYFRCHSRVQIDGSRLENRRLKKKLTCYTRRQDAIEYFQTERAAENTFYQTDNLAPAPKAYRTDMLPQYDLGRGRKQENRIGIALKSLDDSRPMFNQPTLDPAGQIKPKTYKPDAWLSTYELAPALFLSLHGKGSDVEKRIEEFIRSWKGFADWMSKASKEQLKELRYNSAREEFEVFKSRFEKQFGLSVNDIPDEFRYYLVNGKIKPIYMRFQRGSAVPITMDEAAQIWLYNECDKTRSIIRKFNNEQSYDFKLGKGRQRRYTSGNIALWLVRDFMRFQKANDNPREGFGKLKSSPDFNVLQSSLALFNSRKDSLKEILKNARLIYNPSGNHPFLENVINRSGALLSIEGFFNAYLDERLEWLSNASGREVYQLRKLYERGEKKRSAAKGLPPANVYLGEMAKQFLDESVCLPRGLFDDLVRSALKEIYPEQYAKDVPDGQRANFTWLMQKHLKWSGDDHQWFYKELKQSMTAEEFKKLFELLGTTDVRYDNAGKKTLNDKLEETYSQRWYDLEKSLKNDKEFKSLSPNDKQSAIDDRMGQENQMYRSLLNRLRDVWKRIRMSSVQDVVLRDAVWQLLGLPEKSVRLCNVKPEYDLKTQRMIRDDAGDGSISNDAILNETHSLENIIDLPKGLGTIVLKGEMKLKNFGNFYRMLSDPKLPSFLCLYKQFGFNAVDYSYLELQEFEFYDRKFRPAVFNWVHQLEETVLEKYPDLPMKYGKFVDFWTIAGKANGGYIFTQLLLTSIRNAVSHQYYPEFCVPSDWTNQKDIDTYSPQFKDQLLSVKAEFLKRRSEDKDALLSETIYDYAQSLFENAIAFVEQQP

SEQ ID NO: 26 - protein, 1380 aa, synthetic construct
MANFQTPQRHIFGTYLNIARANFYKTILHVFSASGIDCYTKRGDLFVREDTVDKVISALYLIVNGENAGYHAIKEIVSKSYDKRWKEDKALQGNLSGSELKARKEEFKSPLKDEGPDGEDARICKSFTLGSEQEERMRKLLFRHIPLLSPIMADVVAMQFKETTNEHQEANKTLHDATLADCFKELSNIARCLSESRNFYTHKNPYNSIEAQRTQLQLQKVIANNLDKAFIGSRRIAKKRNSYSEKDLAFLTGHDNDCRMEEIFVLDENGNKIWKVEKDKNGKDKLDKDKNPIYVYKKVKAKDGKGREKLDEKGKPVYETLRENGEPVHEYEKKFVERKDFYFRIRGKREVLAPDLTPTGEECDGLSAFGMLYFCSLFLSKEQTAQLCTESRVFVTSPYQPAGNLKNNIILNMMFVYAIHIPRGKRLDSETDSQALGMDMLNELRRCPIELYDVLPSIGKRDFEDNVKHENNRTPELSKRIRLKDRFPYLAMRYIDQQQLFKRIRFHVRLGSFRFCFYDKTCIDGKSHPRQLHKDINGFGRLQDMEKERKEQYGHFFQQSREQSIWQKDENAYVNLKQLEPIKAGDQPHITDMFAQYNIHQNRIGLFWNTDEECKLVNKTNAQGEIIYNGYYLPPLNYVDSPTENNKHKRKAPVDMPAPLCSLSVFELPAMLFYNFLRNTDSLGGEEFPDVEEIVIKQYDNIRKFFLEVKDIQPTDNIENLATILNAYGLSKQSVPKKIYDYLSNKNTLISKDIRKSTEKEVKNRLRRAIIRKQRFEQDQEHIENTKDNKIGKDSFVSIRYAKIAEELAKSMMEWQSGNTKMTGLNFRVLTAALAKFGDGVIKRDTIISMLQKAEIMGGDNPHCFIEQAIEQEQYDIEEFYLDYISAEIEYLKRFLMIDGKTIILKDEQLLDALRKDKEVHDDVRIRLKNDVDFGQLPFIHKSRLRWQQSKIEELANRYLYVKEEGEETLGRATLLLPDGMFFPYIMKGFQKCHQELTNSIEALSDEQKKGIENNVAYLINLYFESKGEKSQSFYDSTEPSHYNDNIRQLAPYKYARSYEFFKIIKGWQIHLSCDEMKKRLTGKKTIIDNKINALKEKGNYISLEEAKNALRRKLHNTFRDMQDNERVIRRYKTQDRILFLMARDMMGEIVNKKADLFKLENVCKDDFLSQKVKASIAVHLSMGEVFKIQKDEMAIKDYGKLYRLLRDDRITKLLSYALYETGETIDYDDITDELKEYESCRSAAFEAVQMIEDTRYQQDKEVLSNPNENNFYCGNIRYKNGKDNGRENEAKRNNFRTLLEDLQKFTPEQMEMFSQEDRELIISIRNAFGHNSYPKQVDFERLINQERKNNPNFKIELKQVASFILDKLEEYVNQVNPQT

SEQ ID NO: 27 - protein, 1246 aa, synthetic construct
MSYTPSSRPPRRPQIVEGSRNNALRILKITPDEQTAFVTYLNYAVNNLSEVAGVAFSDERKVRADVFRGEPADIQRRISRLADFLWSFREKDPSADESGYKAKLGGGHDDMAVWLTEKIVSLRNFFSHVNRQDCTPLVISHDEYVFVEGILGGAARDAAMGPGLNPAKAQKLKLATHHVKEAHTYEFTRKGLVFLTCLGLYKDEAEEFCHLFHEMKVPDRIEDADLDEELPDGRHRAVLGLEDLDKFAGLKGKGRALIELFSFYSYRRGRQSLDAANLDFMQFADVVGCLNKVPAPAFEYLPLEKEHAELEALKAASTESEENKRTKYVLRRRDRNRFLSFAAAYCEDFGVLPSVRFKRLDVRRTTGRHQYVFGPGADAETDEEESNRVRLNRHYAIRRDAIPFEFEPDRHYGPVRIGALRSAVSATEMRRLLYLHAQGADIDAVLRGYFEAYHRVLERMVNAGSLAEISLDDETFLADFATICGTSPEAFKADPEAFRKFVPESLRRYFSKDAGPRSEQRLHALLCSKLLAAVNRTCDILVRHDALEAWREASRPWLDARDDWRGRIDVWRKANPKKDLPDELKTFEAYLETLPADERPARPEEPRCRVGEGEGEITNPPTWCRFSDADYVSFVFDWFNLFLPNNRKFRQLPISEQHREGVEDHLFQMVHAAIGKFSLDQKGLWSLLEKNRAELKPYADLLQYRTDIRVLVAPFVRELNHQLPENRRLPEFDNGVDKDEKEIASPILNIFRHFRQNELWQFLQNFRPELKSWCRSMKSKLQDAMEKKPSRKDGKPHFPKVEDLFEVLNLRRPSPTLEDLAVEAVKLSNDMFLEETNRWGAANPMSVSRDDLLAACRRFGVRPGMPVEYKSLLKTVLGIDYDAWRHAFDYGTGRPFADRRLEDEEHVAAQIPLPNGFADRVAASLPRKVRERFQAPGSAAFDFNAAFRAWSPDPAVSLRDFYDVKPLVASQIARKHPPEGGAATADPFAALSAKTLDSAVREIKDAENQDKVLLFVAMKYWERFRGGDTYSTGKLKMPFSEKTTLREFFDTPVQIEKDGLTVSFRPNDVNRPAFATLFGNSRAAKDYRAKIAKILSPDGSRTAFDFYEMVVAFREQKARDRHERLAFTPYAVHFDALCEIPASAYEKAAEGRFGEAKTEAIRAMEFERYKAVLPGLTREDYDAVADARNAVFHTGFKFDCSKAIAVCKRLGVLGMMPGEAAPAGRRTSSHSGPKWSGPRRNGGSRW

SEQ ID NO: 28 - protein, 921 aa, synthetic construct
VSKNDNIKSKAKALGLKSTFQVGDEVVMTSFGKGNKAIVEKIVRGTDVQSVPTEPNFSAEIEGKKFDLVGRAHIQTKSDNPQYSKKRTGDDMIGAKAALEKRFFGGTFDDNIHIQLAYNVLDIEKILSVHINNIVYTLNNIRRKDNAEDDDFIGYMSTRNDYDTFIEPRKHNISEDAAKSIDKSRASFEEYLQPPVKDCLHYFGNTFFAPREIEKTYTDNRGFERKKKVTVNSLIDEKEIYYIFALLGGLRQFCTHDNKSGRNWLYSLEENGINSDAKAVLDKYYNSAVARIDESFVDNASKTNFKLIFNAMDVSDEALQDNIAKAFYKFTVCKSFKNMGFSIKKLREQLLELPEYEGLKDKHYDSVRSKLYQIMDFVIYLSFKDEKFKKDNEEKINNIVNELRATLNEEDKGRVYASYAERMKSELKPAIARLKSDIDKIKDSRVKEFELDASVKYRLSKVVESVRLKDRATYFTKLIYLTTLFLDGKEINDLLTTLIHQFENIASFIDVMNDRGIDCRFSDGYKLFESSKQIAFELRNVNSFARMTRTSKDDENATHMMYIDAAEILGTDYTEEQIEEHLNLEKKRMIPGTKKADMNFRNFIINNVIKSSRFNYLVRYSNPKKIRALADNEGVIRFVLGELPDAQIDRYTLLCGFNPDADRQEKTDKLAKAITGLRFNDFENVKQGANTEGESQESIDKAQKQGLISLYLTVLYLLTKNLVYVNSRYFLAFHCLERDAQLLGSGAGHHEPYVALTQRFINEDKLNEHACEYLKTNIANSDEYTIRIFRNNAAHLSAVRNANLYIDKLKEFKSYYEIYHFLSQENIYGKYCVDKKYVTADENGSKTISVKISKDYCPQVYIDKSLEYFDKLNKYGTYCKDFTKALNSPFGYNLARYKNLSIEGLFDRNRPGDKGENTFED

SEQ ID NO: 29 - protein, 956 aa, synthetic construct
MAKKLSPKEIREAAKAEKMKSIKAAEAEREKAAEEAKLKAEAEKKEKAEKNEREKALKRFRLDEKSRMALPKSERKSLAKAAGVKSAFAVGNDIYLTSFDRGNDAIVEKKITDTVVTNLRSDESFEVNENTITEMSVPIKSKRISDLYAIADNPLYRKDSATKVQPDKLLLKDTLEKLYFGKTFDDTLHIQIIYNILDIEKILTVYSINTIYCLNNLFGKESGEKEDLISKLTYQITYDEFKESKAHNEFIDFYNLNTLGYYGNIFFKEKKKRSQKEIYDIIALIATIRQWCVHCEEDKRTWLFNTETVLSKEFLDILDDVYESLVEKVNRNFLKDNKVNLQILEDVLEIKDSESREKLIRQYYRFIVTKEQKLLGFSIKKLREAMLEETEFKTDKKYDTVRSKLYKLIDFLLFTGYTTDEAEKEKALFLIKSLRESLTEETKDRIYKSEAARLWLKYENTITNKIREALNEKSISELKKDKSFDDKSITSIITDEVSGKKATYFSKTIYLLAQFIDGKEVNDLTTSLINKFDNIRSLIDTAGQIGLDCKFTEEYKFFENSDQIRTELHVIKNLTNMEYYDTTVKKQMYKDAVHILGIQDDVSDAELEKIINSILLLNENGKPLPGTKGKKGFRNFIISNVLKSRRFIYLIKYCNPKKIRKIAGNRKIIKFVLSRITDSQLERYYYSCNPELKSGIYPGRDDAVNDLSILIADMKFEDFKNVDQSANVHDNNNAAREKMKYQTIISLYLTVCYHLVKNLVNINARYAMAFHALERDARLYQIFSSEENYVDNLNADYAILTKTLLKDNYENAGNLYLRNKKWNKLTRENLDNYIPQAAANFRNAVAHLNPIRNADMLLEDIEDVSSYYAIYHYIMQKSVTNRTIRVSNTTEDEKRILTDYQNKIKRHHGYNKDFVKALCVPFAYNIVRFKSLSIYEMFDRNYHEKTSPETDDSQSP

SEQ ID NO: 30 - protein, 948 aa, synthetic construct
MDKEKTTVEGKNTNQKSDVLKSLAKANGLKSSFVIGNEVVMTSFGRGNSAILEKKITGSRIENLNPNVAFYVKKHISDSCNPDSGKYDVKSKRMKEKAVVDDPVYVSPEKASNVHAGQDLIGCKNVLEERYFGKTFDDNIHIQLIYNILDIEKILAVHVSNATFAINNILGIEGKENEDFIGNLSVLNTFDEFENYETHPKFANKSAIKENLRKSKVFFDKIKKGNKLGYFGQAFYYATGTGKNLIFTKKSSETIYELLALVGSLRQFCVHDEVMVDNKVKSRSWLYNAQKELKPDFLKALDELYSKEVEKIDSDFIVNNTVDLHIIHDAIDVIDGSADWQKITNEYYDFIIRKCFKNIGFSIKRLRETMIEEQMKVLCGKCDRENCKGYGKCFKNKKYDSVRSRLNRIVDFIIFRHYNDEAILKNVSLLRTCMSEEEKQKRFYLPEAKALWKKYNYVFRNYVLKKLNGKSISGLKEKAIENSIDINSVKISLGDPDYFCKFIYLLTFFLDGKEINDLLTTLINKFDNIASFISVMKNDKLSIDCEFVPEYSFFANSAQITSDLRVINSFARMQAPAEPSKDDMYRDALDILGMDDLSEDGKKQLEDTVLCRDENGKYMKKEDNNPKRDTNFRNFLGNNVLASTRFKYLIRYNNAKKTRALANNKAVIIFMLNKINKQNPEQIVSYYKACRDDSDPVASDAEAKIEFLAEKIMNVSCTQFRYVKNGTKVRPDEAKEKERFKAIIGLYLTVMYLITKNMVYINSRYVTAFHCLERDSELHGVKFDQKKLQPNLTKKFIDPKTCGDYGLRNNKRARTYIEQNMDKMSNCTSYWNEYRNAVAHLSVIRNMNQYIKNVKNIGSCFELYHYIMQRFLLDKENIAESLREYDDFIKKKGCYRKDFVKALNTPFGYNLARYKNLSIAELFDRNDTELERTNKLRNEAIKADIEEI

SEQ ID NO: 31 - DNA, 64 bp, synthetic construct
  source 1..64: mol_type = other DNA
gatttagactaccccaaaaacgaaggggactaaaactagattgctgttctaccaagtaatccat

SEQ ID NO: 32 - DNA, 64 bp, synthetic construct
  source 1..64: mol_type = other DNA
gtggcatgaaaaaagcccgacatagcgggcaatcactagattgctgttctaccaagtaatccat

SEQ ID NO: 33 - DNA, 64 bp, synthetic construct
  source 1..64: mol_type = other DNA
gtagaaaagaagatagtccaacatagtggataatcatagattgctgttctaccaagtaatccat

SEQ ID NO: 34 - DNA, 64 bp, synthetic construct
  source 1..64: mol_type = other DNA
ggagatgaaaaaagcccgacatagcgggcaatcgaatagattgctgttctaccaagtaatccat

SEQ ID NO: 35 - DNA, 64 bp, synthetic construct
  source 1..64: mol_type = other DNA
gttaaaagaaaacagcccgacatagcgggcgataactagattgctgttctaccaagtaatccat

SEQ ID NO: 36 - DNA, 63 bp, synthetic construct
  source 1..63: mol_type = other DNA
gaattggagaagatcccgagaaagtgggaaataactagattgctgttctaccaagtaatccat

SEQ ID NO: 37 - DNA, 63 bp, synthetic construct
  source 1..63: mol_type = other DNA
gtttggaaaacagcccgacatagagggcaatagactagattgctgttctaccaagtaatccat

SEQ ID NO: 38 - DNA, 64 bp, synthetic construct
  source 1..64: mol_type = other DNA
gttagatgagaacactccgagataacggagaataactagattgctgttctaccaagtaatccat

SEQ ID NO: 39 - DNA, 66 bp, synthetic construct
  source 1..66: mol_type = other DNA
tagattgctgttctaccaagtaatccatatgttgtcatacccatccaaacgataggcttctacaac

SEQ ID NO: 40 - DNA, 66 bp, synthetic construct
  source 1..66: mol_type = other DNA
tagattgctgttctaccaagtaatccatatgttgtagaagcctatcgtttggatgggtatgacaac

SEQ ID NO: 41 - DNA, 66 bp, synthetic construct
  source 1..66: mol_type = other DNA
tagattgctgttctaccaagtaatccatatgttgtaaagaccttcatttcggaaggaagagacaac

SEQ ID NO: 42 - DNA, 66 bp, synthetic construct
  source 1..66: mol_type = other DNA
tagattgctgttctaccaagtaatccatatgttgtctcttccttccgaaatgaaggtctttacaac

SEQ ID NO: 43 - DNA, 66 bp, synthetic construct
  source 1..66: mol_type = other DNA
tagattgctgttctaccaagtaatccatatgttgtagaagccccttcttcgtggggtaagtgcaac

SEQ ID NO: 44 - DNA, 66 bp, synthetic construct
  source 1..66: mol_type = other DNA
tagattgctgttctaccaagtaatccatatgttgcacttaccccacgaagaaggggcttctacaac

SEQ ID NO: 45 - DNA, 66 bp, synthetic construct
  source 1..66: mol_type = other DNA
tagattgctgttctaccaagtaatccatatgttgtagaagcctatcgtttggataggtatgacaac

SEQ ID NO: 46 - DNA, 66 bp, synthetic construct
  source 1..66: mol_type = other DNA
tagattgctgttctaccaagtaatccatatgttgtcatacctatccaaacgataggcttctacaac

SEQ ID NO: 47 - DNA, 66 bp, synthetic construct
  source 1..66: mol_type = other DNA
tagattgctgttctaccaagtaatccatatgctgtgaactcctgccgaaatggcaggccgaacagc

SEQ ID NO: 48 - DNA, 66 bp, synthetic construct
  source 1..66: mol_type = other DNA
tagattgctgttctaccaagtaatccatatgctgttcggcctgccatttcggcaggagttcacagc

SEQ ID NO: 49 - DNA, 65 bp, synthetic construct
  source 1..65: mol_type = other DNA
ggtttcagacccttacaaaaagggtgtagtacgtttctagattgctgttctaccaagtaatccat

SEQ ID NO: 50 - DNA, 65 bp, synthetic construct
  source 1..65: mol_type = other DNA
gaaacgtactacaccctttttgtaagggtctgaaacctagattgctgttctaccaagtaatccat

SEQ ID NO: 51 - DNA, 64 bp, synthetic construct
  source 1..64: mol_type = other DNA
gtttcagaaccctgtaatttgacagggttgtagttgtagattgctgttctaccaagtaatccat

SEQ ID NO: 52 - DNA, 64 bp, synthetic construct
  source 1..64: mol_type = other DNA
caactacaaccctgtcaaattacagggttctgaaactagattgctgttctaccaagtaatccat

SEQ ID NO: 53 - DNA, 64 bp, synthetic construct
  source 1..64: mol_type = other DNA
gatctataaccctgcatttatgtagggctctaaaactagattgctgttctaccaagtaatccat

SEQ ID NO: 54 - DNA, 64 bp, synthetic construct
  source 1..64: mol_type = other DNA
gttttagagccctacataaatgcagggttatagatctagattgctgttctaccaagtaatccat

SEQ ID NO: 55 - DNA, 36 bp, synthetic construct
  source 1..36: mol_type = other DNA
gtagaaaataagatagtccaacatagtgaataatca

SEQ ID NO: 56 - DNA, 36 bp, synthetic construct
  source 1..36: mol_type = other DNA
gtagaaaagaagatagtccaacatagcggataatca

SEQ ID NO: 57 - DNA, 36 bp, synthetic construct
  source 1..36: mol_type = other DNA
gtagaaaagaagatagtccaacacagtggataatca

SEQ ID NO: 58 - DNA, 36 bp, synthetic construct
  source 1..36: mol_type = other DNA
gtattaacgaagacagtccaacatagtggacactca

SEQ ID NO: 59 - DNA, 36 bp, synthetic construct
  source 1..36: mol_type = other DNA
gaagaaatgaagacagtccaacatggtggataatca

SEQ ID NO: 60 - DNA, 36 bp, synthetic construct
  source 1..36: mol_type = other DNA
gtagaaatgaggatagtccaacatagcaactaatta

SEQ ID NO: 61 - DNA, 38 bp, synthetic construct
  source 1..38: mol_type = other DNA
ggagatgaaaaaagcccgacatagcgggcaatcgaaat

SEQ ID NO: 62 - skipped

SEQ ID NO: 63 - DNA, 38 bp, synthetic construct
  source 1..38: mol_type = other DNA
ggagatgaaaaaagcccgacatagcgggcaatcgaact

SEQ ID NO: 64 - DNA, 38 bp, synthetic construct
  source 1..38: mol_type = other DNA
ggagatgaaaaaagtccaacattatgaagactatagag

SEQ ID NO: 65 - DNA, 36 bp, synthetic construct
  source 1..36: mol_type = other DNA
gttaaaagaaaacagcccgacatagtgggcgataac

SEQ ID NO: 66 - DNA, 36 bp, synthetic construct
  source 1..36: mol_type = other DNA
gttaaaagaaaatagcccgacatagcgggcgataac

SEQ ID NO: 67 - DNA, 36 bp, synthetic construct
  source 1..36: mol_type = other DNA
gttaaaagaaaacagcccgacataacgagcgataac

SEQ ID NO: 68 - DNA, 35 bp, synthetic construct
  source 1..35: mol_type = other DNA
gacttggagaagatcccgagaaagtgggaaataac

SEQ ID NO: 69 - DNA, 35 bp, synthetic construct
  source 1..35: mol_type = other DNA
gaattggagaagatcccgagattaaacacagaaaa

SEQ ID NO: 70 - DNA, 35 bp, synthetic construct
  source 1..35: mol_type = other DNA
gtttggaaaacagcccgacaaagagggcaatagac

SEQ ID NO: 71 - DNA, 36 bp, synthetic construct
  source 1..36: mol_type = other DNA
gttgtaaagaccttcattttggaaggaagagacaac

SEQ ID NO: 72 - DNA, 36 bp, synthetic construct
  source 1..36: mol_type = other DNA
gttgtaaagaccttcattttggaaggaggagacatc

SEQ ID NO: 73 - DNA, 36 bp, synthetic construct
  source 1..36: mol_type = other DNA
gttgtagaagccccttctttgtggggtaggtgcaac

SEQ ID NO: 74 - DNA, 36 bp, synthetic construct
  source 1..36: mol_type = other DNA
gttgtagaagccccttctttgtggggtaagtgcaac

SEQ ID NO: 75 - DNA, 36 bp, synthetic construct
  source 1..36: mol_type = other DNA
gttgtagaagccccttctttgtggggtatgtgcaac

SEQ ID NO: 76 - DNA, 36 bp, synthetic construct
  source 1..36: mol_type = other DNA
gttgtagaagcctatcgtgtggataggtatgacaac

SEQ ID NO: 77 - DNA, 36 bp, synthetic construct
  source 1..36: mol_type = other DNA
gttgtagaagcctatcgtttgggtaggtacgacaaa

SEQ ID NO: 78 - DNA, 37 bp, synthetic construct
  source 1..37: mol_type = other DNA
gtttcagacccttacaaaaagggtgtagtacgtttct

SEQ ID NO: 79 - DNA, 37 bp, synthetic construct
  source 1..37: mol_type = other DNA
gtttcagacccttattaaaagggtgtagtacgtttcg

SEQ ID NO: 80 - DNA, 37 bp, synthetic construct
  source 1..37: mol_type = other DNA
gtttcagacccttactaaaagggtgtagtacaaaaca

SEQ ID NO: 81 - DNA, 37 bp, synthetic construct
  source 1..37: mol_type = other DNA
gttttagacccatgcaaaatgggtgtagtacaaaacc

SEQ ID NO: 82 - DNA, 37 bp, synthetic construct
  source 1..37: mol_type = other DNA
ttgtcttttcccaacaaaaaagggtgtagtacgtttc

SEQ ID NO: 83 - DNA, 36 bp, synthetic construct
  source 1..36: mol_type = other DNA
caactacatctctgtaatctaacagggttgtagttg

SEQ ID NO: 84 - DNA, 36 bp, synthetic construct
  source 1..36: mol_type = other DNA
gtttcagaatcctgtaatttgacagggttgtagttg

SEQ ID NO: 85 - DNA, 36 bp, synthetic construct
  source 1..36: mol_type = other DNA
gatctataaccctgcatttatgcagggctctaaaat

SEQ ID NO: 86 - DNA, 36 bp, synthetic construct
  source 1..36: mol_type = other DNA
gtagaaaagaagatagtccaacatagtggataatca

SEQ ID NO: 87 - DNA, 36 bp, synthetic construct
  source 1..36: mol_type = other DNA
gttaaaagaaaacagcccgacatagcgggcgataac

SEQ ID NO: 88 - DNA, 173 bp, synthetic construct
  source 1..173: mol_type = other DNA
ggccagtgaattcgagctcggtacccggggatcctctagaaatatggattacttggtagaacagcaatctactcgacctgcaggcatgcaagcttggcgtaatcatggtcatagctgtttcctgtgtttatccgctcacaattccacacaacatacgagccggaagcataaag

SEQ ID NO: 89 - DNA, 173 bp, synthetic construct
  source 1..173: mol_type = other DNA
ggccagtgaattcgagctcggtacccggggatcctctagaaatatggattacttggtagaacagcaatctagtcgacctgcaggcatgcaagcttggcgtaatcatggtcatagctgtttcctgtgtttatccgctcacaattccacacaacatacgagccggaagcataaag

SEQ ID NO: 90 - DNA, 173 bp, synthetic construct
  source 1..173: mol_type = other DNA
ggccagtgaattcgagctcggtacccggggatcctctagaaatatggattacttggtagaacagcaatctaatcgacctgcaggcatgcaagcttggcgtaatcatggtcatagctgtttcctgtgtttatccgctcacaattccacacaacatacgagccggaagcataaag

SEQ ID NO: 91 - DNA, 173 bp, synthetic construct
  source 1..173: mol_type = other DNA
  modified_base 71: mod_base = OTHER; note = Modified base is uracil
ggccagtgaattcgagctcggtacccggggatcctctagaaatatggattacttggtagaacagcaatctattcgacctgcaggcatgcaagcttggcgtaatcatggtcatagctgtttcctgtgtttatccgctcacaattccacacaacatacgagccggaagcataaag

SEQ ID NO: 92 - DNA, 18 bp, synthetic construct
  source 1..18: mol_type = other DNA
taatacgactcactatag

SEQ ID NO: 93 - DNA, 18 bp, synthetic construct
  source 1..18: mol_type = other DNA
ctttatgcttccggctcg

SEQ ID NO: 94 - DNA, 36 bp, synthetic construct
  source 1..36: mol_type = other DNA
ggagatgaaaaaagcccgacatagcgggcaatcgaa

SEQ ID NO: 95 - DNA, 35 bp, synthetic construct
  source 1..35: mol_type = other DNA
gaattggagaagatcccgagaaagtgggaaataac

SEQ ID NO: 96 - DNA, 35 bp, synthetic construct
  source 1..35: mol_type = other DNA
gtttggaaaacagcccgacatagagggcaatagac

SEQ ID NO: 97 - DNA, 36 bp, synthetic construct
  source 1..36: mol_type = other DNA
gttgtaaagaccttcatttcggaaggaagagacaac

SEQ ID NO: 98 - DNA, 36 bp, synthetic construct
  source 1..36: mol_type = other DNA
gttgtagaagccccttcttcgtggggtaagtgcaac

SEQ ID NO: 99 - DNA, 36 bp, synthetic construct
  source 1..36: mol_type = other DNA
gttgtagaagcctatcgtttggataggtatgacaac

SEQ ID NO: 100 - DNA, 37 bp, synthetic construct
  source 1..37: mol_type = other DNA
ggtttcagacccttacaaaaagggtgtagtacgtttc

SEQ ID NO: 101 - DNA, 36 bp, synthetic construct
  source 1..36: mol_type = other DNA
gtttcagaaccctgtaatttgacagggttgtagttg

SEQ ID NO: 102 - DNA, 36 bp, synthetic construct
  source 1..36: mol_type = other DNA
gatctataaccctgcatttatgtagggctctaaaac

SEQ ID NO: 103 - DNA, 931 bp, synthetic construct
  source 1..931: mol_type = other DNA
ccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtg

SEQ ID NO: 104 - DNA, 81 bp, synthetic construct
  source 1..81: mol_type = other DNA
taatacgactcactataggagatgaaaaaagcccgacatagcgggcaatcgaattggtgtattcaaggctccctcagttgc

SEQ ID NO: 105 - DNA, 81 bp, synthetic construct
  source 1..81: mol_type = other DNA
gcaactgagggagccttgaatacaccaattcgattgcccgctatgtcgggcttttttcatctcctatagtgagtcgtatta

SEQ ID NO: 106 - DNA, 81 bp, synthetic construct
  source 1..81: mol_type = other DNA
taatacgactcactatagttagatgagaacactccgagataacggagaataacttggtgtattcaaggctccctcagttgc

SEQ ID NO: 107 - DNA, 81 bp, synthetic construct
  source 1..81: mol_type = other DNA
gcaactgagggagccttgaatacaccaagttattctccgttatctcggagtgttctcatctaactatagtgagtcgtatta

SEQ ID NO: 108 - DNA, 82 bp, synthetic construct
  source 1..82: mol_type = other DNA
taatacgactcactatagaaacgtactacaccctttttgtaagggtctgaaaccttgtgcaatttgcggccaatgtttgtaa

SEQ ID NO: 109 - DNA, 82 bp, synthetic construct
  source 1..82: mol_type = other DNA
ttacaaacattggccgcaaattgcacaaggtttcagacccttacaaaaagggtgtagtacgtttctatagtgagtcgtatta

SEQ ID NO: 110 - DNA, 82 bp, synthetic construct
  source 1..82: mol_type = other DNA
taatacgactcactatagcaactacaaccctgtcaaattacagggttctgaaacttgtgcaatttgcggccaatgtttgtaa

SEQ ID NO: 111 - DNA, 82 bp, synthetic construct
  source 1..82: mol_type = other DNA
ttacaaacattggccgcaaattgcacaagtttcagaaccctgtaatttgacagggttgtagttgctatagtgagtcgtatta

SEQ ID NO: 112 - DNA, 82 bp, synthetic construct
  source 1..82: mol_type = other DNA
taatacgactcactatagcaactacaaccctgtcaaattacagggttctgaaacttggtgtattcaaggctccctcagttgc

SEQ ID NO: 113 - DNA, 82 bp, synthetic construct
  source 1..82: mol_type = other DNA
gcaactgagggagccttgaatacaccaagtttcagaaccctgtaatttgacagggttgtagttgctatagtgagtcgtatta

SEQ ID NO: 114 - DNA, 81 bp, synthetic construct
  source 1..81: mol_type = other DNA
taatacgactcactatagatctataaccctgcatttatgtagggctctaaaacttggtgtattcaaggctccctcagttgc

SEQ ID NO: 115 - DNA, 81 bp, synthetic construct
  source 1..81: mol_type = other DNA
gcaactgagggagccttgaatacaccaagttttagagccctacataaatgcagggttatagatctatagtgagtcgtatta

SEQ ID NO: 116 - RNA, 64 nt, synthetic construct
  source 1..64: mol_type = other RNA
ggagatgaaaaaagcccgacatagcgggcaatcgaattggtgtattcaaggctccctcagttgc

SEQ ID NO: 117 - RNA, 64 nt, synthetic construct
  source 1..64: mol_type = other RNA
gttagatgagaacactccgagataacggagaataacttggtgtattcaaggctccctcagttgc

SEQ ID NO: 118 - RNA, 65 nt, synthetic construct
  source 1..65: mol_type = other RNA
gaaacgtactacaccctttttgtaagggtctgaaaccttgtgcaatttgcggccaatgtttgtaa

SEQ ID NO: 119 - RNA, 65 nt, synthetic construct
  source 1..65: mol_type = other RNA
gcaactacaaccctgtcaaattacagggttctgaaacttgtgcaatttgcggccaatgtttgtaa

SEQ ID NO: 120 - RNA, 65 nt, synthetic construct
  source 1..65: mol_type = other RNA
gcaactacaaccctgtcaaattacagggttctgaaacttggtgtattcaaggctccctcagttgc

SEQ ID NO: 121 - RNA, 64 nt, synthetic construct
  source 1..64: mol_type = other RNA
gatctataaccctgcatttatgtagggctctaaaacttggtgtattcaaggctccctcagttgc

(19) United States
(12) Patent Application Publication
(10) Pub. No.: US 2025/0236912 A1
(43) Pub. Date: Jul. 24, 2025

(54) NOVEL CRISPR/CAS13 SYSTEMS AND USES THEREOF

(71) Applicant: CasBio (S) Pte. Ltd., Singapore (SG)

(72) Inventors: Seyed Ghasem HOSSEINI SALKADEH, Killara Sydney (AU); Seyed Hossein MOUSAVI, Karaj (IR)

(73) Assignee: CasBio (S) Pte. Ltd., Singapore (SG)

(21) Appl. No.: 18/703,615

(22) PCT Filed: Nov. 25, 2022
(86) PCT No.: PCT/SG2022/050859

§ 371 Date: Apr. 22, 2024

(30) Foreign Application Priority Data
Nov. 25, 2021 (KR) 10-2021-0164539

(51) Int. Cl. C12Q 1/6876; C12N 9/22; C12N 15/11; C12Q 1/6851; C12Q 1/70

(52) U.S. Cl. CPC C12Q 1/6876; C12N 9/22; C12N 15/11; C12Q 1/6851; C12Q 1/701; C12N 2310/20

(57) ABSTRACT

The present invention relates to the field of RNA editing using novel Cast 3 polypeptides in a CRISPR/Cas13 system. The novel Cast 3 polypeptides have collateral, or ‘trans’ cleavage activity and can be utilised in a nucleic acid detection systems, such as a Cast 3 SARS-CoV-2-based detection assay.

### REFERENCE TO RELATED APPLICATIONS

This application claims the benefit of Korean Patent Application No. 10-2021-164539 filed on 25 Nov. 2021, the entire contents of which, including any sequence listing and drawings, are incorporated in their entirety herein by reference.

### FIELD OF THE INVENTION

The present invention relates to the field of RNA editing, particularly to novel CRISPR effector enzymes and systems and methods for CRISPR based RNA-targeting. In particular the invention relates to a novel CRISPR enzyme (alternatively referred to as a CRISPR protein, a Cas effector protein, a Cas enzyme or a Cas protein) and compositions and systems thereof, and their use in a nucleic acid detection system.

### BACKGROUND OF THE INVENTION

CRISPR-Cas (clustered regularly interspaced short palindromic repeats and CRISPR-associated protein) have revolutionized biological and biomedical sciences in many ways.

CRISPR-Cas-mediated genome editing has emerged as a versatile molecular biological tool with applications expanded to every corner of life science. In addition, CRISPR-Cas enabled assays have already established critical roles in clinical diagnostics and biosensing. The recent discovery of the trans-cleavage activities of Cas12 and Cas13 family proteins, where the target triggered collateral cleavage of non-target ssDNA or ssRNA, made them an ideal toolbox for biosensing. Class 2 CRISPR-Cas systems, including Cas9, Cas12 family, Cas13 family, harness only a single Cas effector to modulate nuclease function and has thus become the most widely studied and utilized CRISPR-Cas systems.

Cas13 (formerly C2c2) is the only family of class 2 Cas enzymes known to exclusively target single-stranded RNA. Cas13, when assembled with a CRISPR RNA (crRNA) forms a crRNA-guided RNA-targeting effector complex. The RNA-guided RNA-targeting CRISPR/Cas13 therefore has a great potential for diagnosis, therapy, and research owing to its biochemical properties including higher RNA digestion efficiency compared to the traditional RNAi, while simultaneously exhibiting much less off-target cleavage compared to RNAi.

Cas13 proteins are present in at least 21 bacterial genomes (Shmakov S et al. (2015) Discovery and functional characterization of diverse class 2 CRISPR-Cas systems. Mol Cell. 60(3):385-97). CRISPR/Cas13 has been used for the highly efficient and specific degradation of RNAs (referred to as cis-cleavage activity) both in vitro and in vivo. This relies on the cleavage of the targeted RNAs by the endogenous RNase activity of the dual higher eukaryotes and prokaryotes nucleotide (HEPN) domains of the protein. They all have two HEPN domains, but the size of the full-length protein can vary depending on which bacteria they come from. For this reason, the CRISPR/Cas13 has different subtypes, including Cas13a, Cas13b, Cas13c, and Cas13d. The protospacer flanking sequence (PFS) for Cas13, which is analogous to the PAM sequence for Cas9, is located at the 3′ end of the spacer sequence and consists of a single A, G, U, or C base pair.

Cas13 protein can also cleave other RNAs non-specifically when activated. When the Cas13 recognizes its target in vitro, it becomes activated and then subsequently promiscuously cleaves RNA species in solution regardless of homology to the crRNA or presence of a PFS. This property, also referred to as collateral cleavage activity, or trans-collateral activity, provides a key characteristic for a nucleic acid detection platform based on the CRISPR/Cas13 system.

As CRISPR-Cas13 systems can be used for a wide range of applications and some CRISPR systems are better suited for certain applications than others, it is critical to pair the appropriate CRISPR/Cas13 systems to the appropriate applications. By finding new CRISPR/Cas13 systems and expanding the CRISPR toolkit, it is possible to enable and improve many of CRISPR/Cas13's applications.

### SUMMARY OF THE INVENTION

Provided herein are new Cas13 polypeptides and compositions thereof, together with CRISPR/Cas13 systems comprising the new Cas13 polypeptides, methods for CRISPR/Cas13 based RNA-targeting, and a nucleic acid detection system using the Cas 13 polypeptides, compositions and systems of the invention. The compositions include either the Cas13 polypeptide itself, or a nucleic acid molecule comprising a sequence encoding a Cas13 polypeptide. There is also provided CRISPR-Cas13 systems comprising the Cas13 polypeptide, or a nucleic acid molecule that encodes a Cas13 polypeptide, together with a sequence encoding a CRISPR RNA (crRNA) comprising one or more spacers and one or more Cas13-specific direct repeats.

One aspect of the invention provides a Cas13 polypeptide, or a nucleotide sequence encoding said Cas13 polypeptide. There is also provided a composition comprising a Cas13 polypeptide, or a nucleotide sequence encoding said Cas13 polypeptide. In preferred embodiments of Cas13 polypeptide compositions, CRISPR/Cas13 systems, methods for CRISPR/Cas13 based RNA-targeting, and nucleic acid detection systems, the Cas13 polypeptide is a Cas13a polypeptide, a Cas13b polypeptide, or a Cas13d polypeptide. Similarly, in the Cas13 polypeptide compositions, CRISPR/Cas13 systems, methods for CRISPR/Cas13 based RNA-targeting, and nucleic acid detection systems that include a nucleic acid molecule comprising a sequence encoding a Cas13 polypeptide, the nucleic acid molecule encodes a Cas13a polypeptide, a Cas13b polypeptide, or a Cas13d polypeptide.

The Cas13 polypeptides of the invention have an amino acid sequence selected from the group consisting of SEQ ID NOS: 16-30, or a sequence being at least 80%, 85%, 90%, 91%, 92%, 93%, 94%, 95%, 96%, 97%, 98%, or 99% identical to SEQ ID NOS: 16-30.

The Cas13 polypeptides of the invention are encoded by a nucleic acid molecule selected from the group consisting of SEQ ID NOS: 1-15, or is selected from a sequence being at least 80%, 85%, 90%, 91%, 92%, 93%, 94%, 95%, 96%, 97%, 98%, or 99% identical to SEQ ID NOS: 1-15.

In embodiments wherein the Cas13 polypeptide is a Cas13a polypeptide, the sequence encoding the Cas13a polypeptide is selected from SEQ ID NO: 1, or SEQ ID NO:2, or SEQ ID NO: 3, or SEQ ID NO:4, or SEQ ID NO: 5, or SEQ ID NO:6, or SEQ ID NO:7, or is selected from a sequence being at least 80%, 85%, 90%, 91%, 92%, 93%, 94%, 95%, 96%, 97%, 98%, or 99% identical to SEQ ID NO: 1, or SEQ ID NO:2, or SEQ ID NO: 3, or SEQ ID NO:4, or SEQ ID NO: 5, or SEQ ID NO:6, or SEQ ID NO:7.

In embodiments wherein the Cas13 polypeptide is a Cas13a polypeptide, the Cas13a polypeptide has an amino acid sequence of SEQ ID NO: 16, or SEQ ID NO:17, or SEQ ID NO: 18, or SEQ ID NO:19, or SEQ ID NO: 20, or SEQ ID NO:21, or SEQ ID NO:22, or is selected from a sequence being at least 80%, 85%, 90%, 91%, 92%, 93%, 94%, 95%, 96%, 97%, 98%, or 99% identical to SEQ ID NO: 16, or SEQ ID NO:17, or SEQ ID NO: 18, or SEQ ID NO:19, or SEQ ID NO: 20, or SEQ ID NO:21, or SEQ ID NO:22.

In embodiments wherein the Cas13 polypeptide is a Cas13b polypeptide, the sequence encoding the Cas13b polypeptide is selected from SEQ ID NO: 8, or SEQ ID NO:9, or SEQ ID NO: 10, or SEQ ID NO:11, or SEQ ID NO: 12, or is selected from a sequence being at least 80%, 85%, 90%, 91%, 92%, 93%, 94%, 95%, 96%, 97%, 98%, or 99% identical to SEQ ID NO: 8, or SEQ ID NO:9, or SEQ ID NO: 10, or SEQ ID NO:11, or SEQ ID NO: 12.

In embodiments wherein the Cas13 polypeptide is a Cas13b polypeptide, the Cas13b polypeptide has an amino acid sequence of SEQ ID NO: 23, or SEQ ID NO:24, or SEQ ID NO: 25, or SEQ ID NO:26, or SEQ ID NO: 27, or is selected from a sequence being at least 80%, 85%, 90%, 91%, 92%, 93%, 94%, 95%, 96%, 97%, 98%, or 99% identical to SEQ ID NO: 23, or SEQ ID NO:24, or SEQ ID NO: 25, or SEQ ID NO:26, or SEQ ID NO: 27.

In embodiments wherein the Cas13 polypeptide is a Cas13d polypeptide, the sequence encoding the Cas13d polypeptide is selected from SEQ ID NO: 13, or SEQ ID NO:14, or SEQ ID NO: 15, or is selected from a sequence being at least 80%, 85%, 90%, 91%, 92%, 93%, 94%, 95%, 96%, 97%, 98%, or 99% identical to SEQ ID NO: 13, or SEQ ID NO:14, or SEQ ID NO: 15.

In embodiments wherein the Cas13 polypeptide is a Cas13d polypeptide, the Cas13d polypeptide has an amino acid sequence of SEQ ID NO: 28, or SEQ ID NO:29, or SEQ ID NO: 30, or is selected from a sequence being at least 80%, 85%, 90%, 91%, 92%, 93%, 94%, 95%, 96%, 97%, 98%, or 99% identical to SEQ ID NO: 28, or SEQ ID NO:29, or SEQ ID NO: 30.

In preferred embodiments of this aspect of the invention, the Cas13 polypeptide is a Cas13a, a Cas13b, or a Cas13d polypeptide, with at least trans cleavage activity and preferably both trans cleavage and cis cleavage activity.

The Cas13 polypeptide of the invention is preferably a Cas13a or Cas13d polypeptide, and more preferably, is Cas13a7, Cas13d13, Cas13d14 and Cas13d15.

In another aspect, there is provided a nucleic acid molecule comprising: (a) a sequence encoding a Cas13 polypeptide wherein the Cas13 polypeptide has an amino acid sequence selected from the group consisting of SEQ ID NOS: 16-30, or a sequence being at least 80%, 85%, 90%, 91%, 92%, 93%, 94%, 95%, 96%, 97%, 98%, or 99% identical to SEQ ID NOS: 16-30; and (b) a sequence encoding a CRISPR RNA (crRNA) comprising one or more spacers and one or more Cas13-specific direct repeats, wherein the crRNA is capable of hybridising with one or more target RNA molecules.

In another aspect, there is provided a CRISPR/Cas13 system for targeting RNA molecules, the system comprising
i) at least one Cas13 polypeptide wherein the Cas13 polypeptide has an amino acid sequence selected from the group consisting of SEQ ID NOS: 16-30, or a sequence being at least 80%, 85%, 90%, 91%, 92%, 93%, 94%, 95%, 96%, 97%, 98%, or 99% identical to SEQ ID NOS: 16-30; or a nucleic acid molecule comprising a sequence encoding the Cas13 polypeptide and
ii) at least one CRISPR RNA (crRNA) or a nucleic acid molecule encoding the crRNA, the crRNA comprising one or more spacers and one or more Cas13-specific direct repeats, wherein the crRNA is capable of hybridising with one or more target RNA molecules.

Preferably the nucleic acid molecule comprising a sequence encoding the Cas13 polypeptide is selected from the group consisting of SEQ ID NOS: 1-15, or is selected from a sequence being at least 80%, 85%, 90%, 91%, 92%, 93%, 94%, 95%, 96%, 97%, 98%, or 99% identical to SEQ ID NOS: 1-15.

In another aspect of the invention there is provided a composition comprising a CRISPR/Cas13 system for targeting RNA molecules, the system comprising i) at least one Cas13 polypeptide wherein the Cas13 polypeptide has an amino acid sequence selected from the group consisting of SEQ ID NOS: 16-30, or a sequence being at least 80%, 85%, 90%, 91%, 92%, 93%, 94%, 95%, 96%, 97%, 98%, or 99% identical to SEQ ID NOS: 16-30; or a nucleic acid molecule comprising a sequence encoding said Cas13 and ii) at least one CRISPR RNA (crRNA) or a nucleic acid molecule encoding said crRNA, the crRNA comprising one or more spacers and one or more Cas13-specific direct repeats, wherein said crRNA is capable of hybridising with one or more target RNA molecules.

Preferably the nucleic acid molecule comprising a sequence encoding the Cas13 polypeptide is selected from the group consisting of SEQ ID NOS: 1-15, or is selected from a sequence being at least 80%, 85%, 90%, 91%, 92%, 93%, 94%, 95%, 96%, 97%, 98%, or 99% identical to SEQ ID NOS: 1-15.

The crRNA in aspects of the invention preferably comprises one or more Cas13 specific direct repeats, and one or more spacers, wherein the spacers are the sequences of the crRNA capable of hybridising with one or more target RNAs. In some embodiments, the crRNA comprises two or more spacers, wherein the two or more spacers are capable of hybridising with different target RNAs.

The nucleic acid molecule components of the compositions and systems described herein may be comprised within one or more vectors. The one or more polynucleotide molecules may further comprise one or more regulatory elements operably configured to express said Cas13 protein, and said guide molecule. Preferably the one or more regulatory elements comprise operably linked promoters.

Accordingly, in another aspect, provided herein are vectors and vector systems comprising any of the nucleic acid molecules of the invention described herein, and in one embodiment of the CRISPR/Cas13 systems of the invention, a system comprising the vector systems.

There is provided a CRISPR/Cas13 system wherein the system comprises a vector system comprising one or more vectors comprising:
i) a first regulatory element operably linked to a nucleic acid molecule comprising a sequence encoding a Cas13 polypeptide wherein the Cas13 polypeptide has an amino acid sequence selected from the group consisting of SEQ ID NOS: 16-30, or a sequence being at least 80%, 85%, 90%, 91%, 92%, 93%, 94%, 95%, 96%, 97%, 98%, or 99% identical to SEQ ID NOS: 16-30; and
ii) a second regulatory element operably linked to a nucleic acid molecule encoding a CRISPR RNA (crRNA), the crRNA comprising one or more spacers and one or more Cas13-specific direct repeats, wherein the crRNA is capable of hybridising with one or more target RNA molecules;
wherein components (i) and (ii) are located on the same or different vectors of the system.

Preferably the nucleic acid molecule comprising a sequence encoding the Cas13 polypeptide is selected from the group consisting of SEQ ID NOS: 1-15, or is selected from a sequence being at least 80%, 85%, 90%, 91%, 92%, 93%, 94%, 95%, 96%, 97%, 98%, or 99% identical to SEQ ID NOS: 1-15.

There is also provided a kit comprising the one or more vectors of the vector system.

Another aspect of the invention provides an in vitro method of modifying a target RNA, the method comprising contacting the target RNA with a ribonucleoprotein (RNP) complex of a CRISPR/Cas13 system, the system comprising:
i) at least one Cas13 polypeptide wherein the Cas13 polypeptide has an amino acid sequence selected from the group consisting of SEQ ID NOS: 16-30, or a sequence being at least 80%, 85%, 90%, 91%, 92%, 93%, 94%, 95%, 96%, 97%, 98%, or 99% identical to SEQ ID NOS: 16-30; and
ii) at least one CRISPR RNA (crRNA), the crRNA comprising one or more spacers and one or more Cas13-specific direct repeats, wherein the crRNA is capable of hybridising with one or more target RNA molecules,
wherein the Cas13 polypeptide and the crRNA form a ribonucleoprotein (RNP) complex, and upon binding of the complex to the target RNA through the one or more spacers, the Cas13 polypeptide modifies the target RNA.

In an alternative embodiment of this aspect of the invention, prior to contacting the target RNA with the RNP complex, the method comprises:
a) expressing from a vector system at least one Cas13 polypeptide and at least one CRISPR RNA (crRNA), the vector system comprising one or more vectors comprising:
i) a first regulatory element operably linked to a nucleic acid molecule comprising a sequence encoding a Cas13 polypeptide wherein the Cas13 polypeptide has an amino acid sequence selected from the group consisting of SEQ ID NOS: 16-30, or a sequence being at least 80%, 85%, 90%, 91%, 92%, 93%, 94%, 95%, 96%, 97%, 98%, or 99% identical to SEQ ID NOS: 16-30; and
ii) a second regulatory element operably linked to a nucleic acid molecule encoding a CRISPR RNA (crRNA), the crRNA comprising one or more spacers and one or more Cas13-specific direct repeats, wherein the crRNA is capable of hybridising with one or more target RNA molecules;
wherein components (i) and (ii) are located on the same or different vectors of the system;
b) isolating the expression products of step (a); and then
c) contacting the target RNA with the isolated expression products of step (b), wherein the Cas13 polypeptide and the crRNA form a complex, and upon binding of the complex to the target RNA through the one or more spacers, the Cas13 polypeptide modifies the target RNA.

Optionally the isolated expression products of step (b) are assembled in to the RNP complex prior to contact with the target RNA in step (c).

Preferably the nucleic acid molecule comprising a sequence encoding the Cas13 polypeptide is selected from the group consisting of SEQ ID NOS: 1-15, or is selected from a sequence being at least 80%, 85%, 90%, 91%, 92%, 93%, 94%, 95%, 96%, 97%, 98%, or 99% identical to SEQ ID NOS: 1-15.

In another aspect of the invention there is provided a nucleic acid detection system for detecting a target RNA in a sample, the system comprising:
i) at least one Cas13 polypeptide wherein the Cas13 polypeptide has an amino acid sequence selected from the group consisting of SEQ ID NOS: 16-30, or a sequence being at least 80%, 85%, 90%, 91%, 92%, 93%, 94%, 95%, 96%, 97%, 98%, or 99% identical to SEQ ID NOS: 16-30, or a nucleic acid molecule comprising a sequence encoding the Cas13 polypeptide and
ii) at least one CRISPR RNA (crRNA) or a nucleic acid molecule encoding the crRNA, the crRNA comprising one or more spacers and one or more Cas13-specific direct repeats, and
iii) a detector RNA
wherein the crRNA is capable of hybridising with one or more target RNA molecules, and the Cas13 polypeptide has at least trans cleavage activity.

The detector RNA can be, for example, a labeled detector RNA such as a fluorescence-emitting dye pair, i.e., a FRET pair and/or a quencher/fluor pair or RNA molecule generating any other detectable signal after collateral cleavage.

Preferably the nucleic acid detection system is in kit form.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF THE DRAWINGS

FIG. 1: Phylogeny analysis of novel CRISPR-Cas13 proteins. Protein sequence alignment of the Cas13s was performed using ClustalW in MEGA11 with default settings. Rumen metagenomics derived Cas13 were compared with previously known (characterised) Cas13a (LbaCas13a and LwaCas13a), Cas13b (PspCas13b), and Cas13d (EsCas13d and RspCas13d) to generate the analysis shown in FIG. 1. The enzymes of the invention are labelled as “C”, followed by a, b or d for the subtype, and a number.

FIG. 2: Fluorescence curves of the Cas13 polyprotein trans-cleavage reactions. The fluorescence was measured in 2 min intervals. Values are shown in the graphs as means±SD (n=3).

FIG. 3: Fluorescence curves of the Cas13 polyprotein trans-cleavage reactions using different ssRNA reporter (ie probe) sequences (Table 6). FIG. 3A is Cas13a3; FIG. 3B is Cas13a7; FIG. 3C is Cas13d13; FIG. 3D is Cas13d14 and FIG. 3E is Cas13d15. The fluorescence was measured in 2 min intervals. Values are shown in the graphs as means±SD (n=3).

FIG. 4: Fluorescence curves of the Cas13 polyprotein trans-cleavage reactions using different activator (PFS) sequences presented in Table 5. FIG. 4A is Cas13a3; FIG. 4B is Cas13a7; FIG. 4C is Cas13d13; FIG. 4D is Cas13d14 and FIG. 4E is Cas13d15. The fluorescence was measured in 2 min intervals. Values are shown in the graphs as means±SD (n=3).

FIG. 5: Screening of the buffers. Detailed compositions of the tested buffers are listed in Table 7. FIG. 5A is Cas13a3; FIG. 5B is Cas13a7; FIG. 5C is Cas13d13; FIG. 5D is Cas13d14 and FIG. 5E is Cas13d15. End-point fluorescence signal measured after 60 min. Values are shown in the graphs as means±SD (n=3).

FIG. 6: Screening of the buffer pH. FIG. 6A is Cas13a3; FIG. 6B is Cas13a7; FIG. 6C is Cas13d13; FIG. 6D is Cas13d14 and FIG. 6E is Cas13d15. End-point fluorescence signal measured after 60 min. Values are shown in the graphs as means±SD (n=3).

FIG. 7: Probe concentration assay. FIG. 7A is Cas13a3; FIG. 7B is Cas13a7; FIG. 7C is Cas13d13; FIG. 7D is Cas13d14 and FIG. 7E is Cas13d15. The fluorescence was measured in 1 min intervals. Values are shown in the graphs as means±SD (n=3).

FIG. 8: The limit of detection (LoD) of Cas13-based nucleic acid detection. The reaction systems were comprised of 50 nM crRNA, 100 nM Cas13, 1000 nM reporter, and different concentrations of target RNA (between 1 nM and 100 fM in 10-fold dilutions), for each enzyme in the corresponding reaction buffer. The fluorescence was measured in 2 min intervals. Values are shown in the graphs as means±SD (n=3).

FIG. 9: Table summarising the optimisation of cleavage conditions. Reactions were incubated for 1 h at 37° C. fluorescence was measured in 1 or 2 min intervals. Real-time or end-point fluorescence measurements were collected on a microplate reader. All experiments were performed using three independent replicates and results were shown in the graphs as means±SD (n=3); Conc.: concentration; NA: not applicable.

FIG. 10; Cas13-based detection of SARS-CoV2 N gene using crRNAs presented in Table 11. Cd14-CoV crRNA 1 was used for Cas13d14. End-point fluorescence signal measured after 60 min. Values are shown in the graphs as means±SD (n=3).

FIG. 11: Cas13d14 based detection of SARS-CoV2 N gene using two different crRNAs (Table 11). Fluorescence curves of the Cas13d14 trans-cleavage reactions. The fluorescence was measured in 2 min intervals. Values are shown in the graphs as means SD (n=3).

FIG. 12: Cas13d14 visual-based detection of SARS-CoV2 N gene using Cd14-CoV crRNA 1. The image of strip of reactions were captured using SYNGENE transilluminator (420 nm wavelength), 15 minutes after beginning of collateral activity.

FIG. 13: Representative denaturing gel showing the targeted in vitro RNase cleavage activity of the Cas13 polypeptides when incubated with the ssRNA target of SARS-CoV2 N gene and different crRNAs (labelled as crRNAs in the figure). RNA cleavage activity is most evident with Cd13-crRNA 1 relative to other enzymes and crRNAs and no crRNA control. The presence and absence of crRNAs has been designated as ‘+’ and ‘−’, respectively. Cd14-CoV crRNA 1 and Cd14-CoV crRNA 2 were shown by ‘+1’ and ‘+2’, respectively. The reaction containing Cas13d14 without crRNA was used as control.

### DESCRIPTION OF SEQUENCES

TABLE 1
Sequences of the invention

| SEQ ID | | |
|---|---|---|
| NO | Description | Sequence |
| 1 | Ca1 | ATGAAGATTTCGAAGGTAGATCATACTAAGAGTGCGGTGAGTGTA |
| | ContigID: | CAAACTGCACAGGGACAGCAAGGTATTCTTTATAAGGATCCGTCC |
| | k127_1867445 | ACAGAAGAGATGAGCGTGGAAGATCGCGTCACCAAAAGAGCAGAT |
| | | GCTACGAAAGCATTATACGCTGTGTTCAATCAACCTAAGGATAAA |
| | | CGAAGCATTTCAGGGGAAGCAACAACGGTTGCATCTTCTTTTAAC |
| | | TATGTGATCAAAGACCTGAAAAAAAGCAAATCACTTAATGGGAAG |
| | | TTGAGTGTGGAGAGCCTCTATGAAGCTGTGGGAAATGAACTTAAA |
| | | GGAAAACATGCGAGTGCTGAAGAGATAGATTTGGCAATTACATTA |
| | | TTGCTGAAGAAAAGTCTGCGTAGAGATTCCTTTATAGAGGCGTTA |
| | | AAGCTGGTGCTGGGTAAAGCGTATAAAGGAGAAAAGCTAAATGAG |
| | | GAAGATAAGAGAATAATCAAGGACGATCTGATAGTTCCTTTGATA |
| | | AAGGATTATGATAAATCATCTATCAGAGAGCAGGCAGTGGCCTCG |
| | | ATCAAACATCAGAACCTTATCGCACAGCCGGATTCCAAGTCTGAT |
| | | GACGCAGTAATGGTTATATCCAATATAGCCGGGGCTTCTGAGAGA |
| | | AGTACGAATGAGAAAGAAGCTTTAAGGCAGTTTATATCTGAATAT |
| | | GCAGTGCTAGATGACAGTGTTCGTCATGATATGCGTGTTAAACTC |
| | | CGTCGTCTGGTGATCCTGTATTTTTATGGCATGGATGTAGTTCCG |
| | | ACGGGTGATTTTGACGAATGGGAAGATCATGTCCAGAGGGGAAAG |
| | | ACCGCTGATCTGTTCATAGACTTTGCACCTGTCGGAGGTAAGACA |
| | | GATGCTGATAGATTAAAGGATGCTATTCGGAAAATGAACATTGAA |
| | | AGATACCGATATAGCGTAGATGCGATAGATCAGGATAATACAGAA |
| | | CTCTTCTTCGAAGATATGATGATCAACAAATTCTTTATTCATCAT |
| | | ATTGAAAACGAAGTAGAGCGGATATATAGGAATACTAAGCCTGGT |
| | | GATGAGTTCAAGAGAAGCTTAGGCTATATCAGCGAGCGTGTGTGG |
| | | AAGGGAATTATTAACTATCTGTGTATTAAGTATATAGCAATAGGA |
| | | AAGGCTGTATATAATTGCGCTATGGCTGGGTTGGGCTCTGATCAG |
| | | CCCGATATTAAATTGGGAGTCATCGACAGAGTGTATGCTGATGGC |
| | | ATCAGTTCCTTTGATTATGAGATTATCAAGGCACAGGAAACACTC |
| | | CAGAGAGAAACCTCTGTATACGTTTCATTTGCGATTAATCATCTT |
| | | GGGGCTGCTACTGTTAATCTGACTGAAAAAGAGACCGATTTTCTT |
| | | ACACTTGATAATAAACAGATTAAGGAACTGGCAAAGACAGGTGTT |
| | | CTCAGGAATATTCTTCAGTTTTTTGGTGGTAAATCCGTATGGAAA |
| | | AATTTCGAGTTTGCTCCTGAAGGGGGTACCGGCAATGAAGAAATT |
| | | GTATTGTTATATTACCTTAAAGACATCCTGTATGCGATGAGAAAT |
| | | GAGAACTTTCATTTTTCAACAGCCAGCATTAATGATGGCTCATGG |
| | | GACACTGATCTGATCGGCAGGATGTTTGCATACGACTGTACCAGA |
| | | GCAGGGGTGGGACAAAAGAACAAATTCTATTCCAACAACCTTCCA |
| | | ATGTTCTATAAATCAGAGGACCTGGAACGAGCATTACATATTTTG |
| | | TATGATCACTATAGCGAGCGAGCTTCGCAGGTTCCGGCGTTTAAT |
| | | ACGGTATTTGTGAGAAAGAACTTTTCTGAAATACTTAAGGGGCAG |
| | | AATCTGCCTATGCCAACTTCAGCTGAAGAATCTCTTAAATATCAG |
| | | AATGCGATCTATTATCTCTATAAAGAGATTTATTATAACGTATTT |
| | | CTGAGTTCATCAGAAAGCCGAGATTATTTCATTAAAGCAGTCAAG |
| | | TCACTTAGGTGGGAAAACTCGAATGAAGAGAATGCCGTAAAAGAC |
| | | TTTCAAAATCGAATTAATGAACTGACCGGAAAATACAGTTTATCC |
| | | CAGATTTGTCAGTTGATCATGACAGAATACAATCAGCAAAACAGC |
| | | GGAAGCAGAAAAAAGAAGACTGCTAAGGATGAACAGAATAAACCG |
| | | GATATTTTCAAGCACTATAAGATGCTTTTGTATAAGAGCATTCGG |
| | | GAAGCAATGCTTAAGTATGTGGACGATAAATCAGAGGACTTTGGT |
| | | TTTATAAAAAGTCCGGTATTCGGAAAGGATGACAACTGCATTGCG |
| | | TTAGAAGAATTCCTCCCTGATTATGAATCGACGCAAAATGCAAAA |
| | | CTGATAGAACGCGTGAAGTCAGATTTCAGACTTCAGAAATGGTAC |
| | | ATTTTGGGAAGACTGCTCAATCCTAAACAGGTAAATCAGCTTGCC |
| | | GGATCTATCAGGAGTTATATTCAGTATTCAGATGATGTAAAGAGA |
| | | CGTGCAAAAGAAAATGGTAATAAGATTCATGTATCCACGGAATCG |
| | | TATCCTTATCAGACTGTTTTGAGAGTCATTGATCTGTGTGCGAAA |
| | | TTGAGTGGACTTACCACCAATAACATAGATGACTATTTTGATGGT |
| | | AGTGGGGATTATCTGTCATACCTTGCTCGCTTTGTGGAGTATGAT |
| | | CCGAATGATATACCGAAGATATATCATGATGAAGCTAATCCCATT |
| | | CTTAATCGCAACATCATTATGGCTAAATTATATGGCGCAGGAGAT |
| | | GTTATTACCAATGCAGTAGAACATGTCAATACAAGTATGATCAGA |
| | | GATCTTGAATCATATGAGAAGAAAACGTTGGGGTATCGCTCTTCT |
| | | GGAGTATGTAAGGACAAGGATGAACAGGAAACGCTAAAAAAATAT |
| | | CAGGAATTAAAGAACCGGGTAGAGCTTCGAGAGATAGTGGAGTGT |
| | | TCTGAAATTATAAATGAACTTCAGGGACAGCTGATCAACTGGTGT |
| | | TATCTGAGAGAGAGGGATCTGATGTATTTCCAGCTGGGGTTCCAT |
| | | TATACCTGTTTGAAGAATTCATCCGATAAACCGGAGATGTATGTA |
| | | AAAGCGAAAACCGTAGATGGAACTATTGATGGATTTATCCTGCAC |
| | | CAGATCGCCGCATTATATACGAATGGACTAAAGCTGTATTCTTGC |
| | | GGTAAAGCTGTAAGAGATGATAATAGAAAGATAATTCATTATGAC |
| | | CTGAGCAGTGGAAAAGAATTAAAGGGCAATGATAAGAGTGCAGCC |
| | | GGGAAAAAGATTACTGACTTTATGGGGTATACGTCATTAGCGTTA |
| | | AACAGGACAGAGAATGACATACTCCCTATATCCGGTGATTTTTAT |
| | | TATGCCGGGCTGGAACTGTTTGAAAACGTAAATGAACACGAGAAT |
| | | ATCATAAGCCTCAGGAATTATATCGATCACTTCCACTATTATGCA |
| | | AAACATGACAGAAGCATGATCGATATCTACAGCGAGGTATTTGAC |
| | | AGATTCTTCTCCTATGACATGAAATATCGTAAGAATGTGCCTAAT |
| | | ATGATGTACAACATTCTGCTGTCGCATTTTGTGAAAGCACAATTC |
| | | GTATTCGGATCAGGAATGAAGGAGTCCGGAGAAAAGACCAAGTCC |
| | | CAGGCCAGATTTGACTTGAAGGATAAAGCCGGGCTTGAGCCCGAA |
| | | CAATTGACATATAAGGTTGCGAATTCTGAGAAACCGGTGCAACTT |
| | | TCTGCTAAAGACAAACAATTCCTGAAGACGGTTGCATTATTGTTG |
| | | TATTATCCGGAAAAAAAGACGTTTCCGGAGGGGATGTATGCCGAT |
| | | ACCAGGTTTGTAGAAGGGACATCATCAAATAAAAGGAATAACAAT |
| | | TCTTCAGGCAACCGACATGGCAATGGTAATCACAATGTAGGAGGA |
| | | CATAACAAAGGGTACAATCAGGGCAGGAAAAACGGCAATTGGTCT |
| | | AAGGACAAATCCGGAGACAGAAATGCCGGAAAGAAACAAACAAAT |
| | | AAAAACCGGAAAGATAGTACCAGCGTCTATAAAGATGAAGGATTC |
| | | AGTAACAGAATAAATATTCCTTCTGAGTATTATTCTCAGAAACCG |
| | | GGGAAAAAGTAG |
| 2 | Ca2 | ATGAAATTATCAAAAACTGGAAAAAACGGCTGGCATCACAGAAAC |
| | ContigID: | GGAGTAAAGGTTAACAACAGTAAACAGGAGGGATTTGTTTACAGT |
| | k127_4200118 | ATTCCTCATAATGATGGTGAGAGTACAGATAAGTTTGTTGAAGAC |
| | | AGAAAGAAAGATTTTAAGAGACTTTATAAGGTATTTCCTTCTGTT |
| | | GAAAAAGCCAGAAACATAAGTGAAGAGATTGCTGCAGTCATAGAT |
| | | AAAACGATCAGAAATAAAAGAACAGAAATATGGACCGGAAAAAAT |
| | | GATTATTCTGAAATGGCCTGCAGATTCAGAAATCTGCTTCAGCGC |
| | | GAATCTATGTTCAGACAGCCTGTAGAAGTAAAAACCGCTGAATAT |
| | | ATGGTTTATGGTCTTTTGCGATCAAGCCTGCGTTCTGAAAAAACG |
| | | GAGAAGGATCTTATAGATTTTTTATGCCATGTGAATGACAAGTCC |
| | | TCATCAGCTGGTGCAATATTTATGCAGGAGCTGACCAGGGATTAT |
| | | ATGGGTGAAAAGATCAATTATAAGTCGATAATAAACCAGAATCTC |
| | | GTAATCCAGCCTGTTAAGACAGAATCCTTAAAAGATAACATTGAT |
| | | CAGGAGGATGTTTTACTAACTGTTTCCGAAAGGAAAAATCCTGAA |
| | | GACTCGGCAAAGAATTATAAAAGTATTGAAAATAAAGCATTACGG |
| | | AGCTTTTTATTGGAATATGCCAGCCTTGATGATAATAAGCGTAAG |
| | | GATCTGAGAAAAAAGCTCCGCAGGATAGTTGTACTCTATTTTTAT |
| | | GGAAAGTCCGAAGCAGACGGCTTAGGAGAAAACTTTGATGAGTGG |
| | | AATGACCATGAATCCAGGCGTGCATGTGAGGAGAAGTTCATTGAA |
| | | TTTGATGAGAGTACCAAGGATAATTTCAGATCCAAAACATCTAAG |
| | | CTGATAAGAAATGCCAATATCACAGCATACAGGACTTCAAAAGAA |
| | | ATCATTGAAAACAATCATGACGGGCTGTATTTTGCCAATCCTGAT |
| | | TATAATTTCCTTTGGTTAAGGCATTTATCCAGGGAGGTTGAGCGT |
| | | CTGACATCAAATATCAATGCTGATAAGACGTATAAGCTGAATAAG |
| | | GGCTATTTAAGTGAAAAGTCCTGGAAGGGAATAATCAATTATCTT |
| | | AGCATAAAGTATATAGCTATAGGCAAATCTGTATTCAATTTCGCA |
| | | TCTTCCGGAGTAGATTCTGATGGCAGTGATATACAGATTGGCGAA |
| | | GTAAACAGGGAGTTCCAAAACGGAATAAGTTCATTTGAATATGAG |
| | | AGGATAAAAGCCGAAGAAACACTTCAGAGAGAAAGTGCGGTAAAA |
| | | GTTGCCTTTGCTGCAAGACATCTTGCATCGGCTACGATGAATCTT |
| | | ACACCGGAAGATTCAGATATGCTCCTGTTTGATAAGGACAAAATG |
| | | TCTCAGAATCTAAAAGATACAGGCAGGGTACTGGCTGATGTATTG |
| | | CAGTTTTTTGGAGGACAGTCTGTCTGGAAGGATTATATTATAAAA |
| | | GAAACTGAAAAGTATTCAAGTGAAGAAGAATTCGGAACTGATTTA |
| | | TTATATAATCTGAAAAAATGCGTCTATGCCTTAAGAAATGACAGT |
| | | TTCCATTTCAAAACTTTGAATAATAAGGCTGATTGGGATACTGAC |
| | | CTTGTAGCAGGATTGTTTGAAAAAGACTGTGAAAACATGGTAGGC |
| | | CTTGATAAAGATAAGTTTTATGATAATAACCTTTACAAGTTCTTT |
| | | AAACAGGAAGACCTGAAAAAAGTATTAGACAAACTGTATGATAAA |
| | | ATCCATGACAGGGCATCTCAGATACCTGCTTTCAATACAGTGTTT |
| | | GTAAGAAATAATTTCAGCAGGTTCCTGTTGTCTAAAGGTATCACA |
| | | CGCTCATTTGCGTCAGAGGAGCAGGGAAGACAGTTTGCAAGTGCA |
| | | GTATATTATCTTTTCAAAGAAATCTATTACAATGATTTTATCCAG |
| | | TCAGGAAATGCTAAAACGCTGTTCCTGAATTATGTTAATTCAATC |
| | | AAAATCGAGAAAGCAATTAACAGATACGGGAAAGAAGAAACTAAA |
| | | AGAGAATGCAAACCGGCAGAAGATTTCAAGACATATATAACGTTG |
| | | TGCCGGAATATGAGTTTCTCTGAGATATGTCAGGCAGTAATGACT |
| | | GAGTATAACCAGCAGAACAACCAGTCCAGAAAGAAAAAATCAGCT |
| | | TTTGATACAGCAAAAAACAAAGATAAATTCAGACATTATGTAGAT |
| | | ATTTTACATGAAGGGATCAGGGAAGCATTTGCAGCATATATTGGT |
| | | CTCAATGATCAGAAAAATTATGATGGTATATATGGCTTTGTAAAG |
| | | TCATTTAACAGCAGCGATGTGTTCACAGTCGAAAAGGACAAGTTT |
| | | ATTGAAGGGTACAGATCTGAACGTTTCCAAAATCTGATCAGTAAA |
| | | GTGAGACAGAATCCTGAACTTCAAAAGTGGTATATTGTGGCTAGA |
| | | CTTCTGAATCCCAAACAGGTAAATGAACTTTCCGGATCGATAAGA |
| | | AGCTATAAGCAGTATATTGAAGATGTATGCCGCAGGTCGATCGCT |
| | | GAAAAATGTCCTGTAAGGAAAAATGATGGAAAAGAGGTAAGCAAA |
| | | GCTTCTTTTGATAATGATGTAAAAGAACTGATGTCCATCGATTAT |
| | | ATGGGTGTTGTAGCGATATTGGAGATATGTATACGTCTTAACGGA |
| | | AGATTTTCAACAGTATGCGATGATTATTTTAAGGATGGTGCTGAC |
| | | GGCTATGCTGAATATCTGGAGCAGTATCTTGATTATCAAGATGAA |
| | | AAAACTAAGGATGCCGGTGTAAGTCCTTCAACCATGCTGTCGATG |
| | | TTTTCAGAAGAAGTCTCAGCGGATAATACTGATAAGAATCAGGGA |
| | | ATCATATATCATGACGGGACTAATCCCATTATGAACAGAAATATT |
| | | CTCTTATCGAAACTCTATGGTGGAGCAAATTCGGTAATACATTCT |
| | | GTAAAGAAGGTGGATAACCGCCTGATAGCAGATTTTATTAAAAGC |
| | | GGTAAGCTTATACAGGAATACAATAAGAGAGGATACTGTATTAAT |
| | | GAGGAGGAACAGAAAAATCTGAAGAGATATCAGGCTCTTAAGAAC |
| | | AGGGTTGAGTTCCGAGATATAGTAGAATATGGCGAAATCCTGGAT |
| | | GAACTGCAAGGGCAACTGATTAACTGGAGTTATTTAAGGGAACGT |
| | | GATCTGATGTATTTCCAGCTTGGTTTCCATTATACGAGCCTTCAT |
| | | AATTCTGAAAGAAAGTTTGAAGGGTACAGATATATAACTAAAGAA |
| | | GACGGAAGTGTTATAGAGAATGCAGTACTGCACCAGATTCTATCA |
| | | TTATATATTAACGGAATACCGTTCTATTACAGCTACGCCGATGTG |
| | | GAAGGCAGAGACCGGTTTATTTGCTGTGCACTAAAGAAGAAAGAA |
| | | CCTGTTGATGGTACCAGCAACAAGTTTGAAGATACCGGAACAAAG |
| | | ATGAGATATGTAGGATATTACTGCAAAGAAGGTGATAATTATCTG |
| | | GGTGAAGGGATATATCTTGCAGGTCTGGAACTGTTTGAGAATATT |
| | | GCTGAACATGATAATATCATCAAGCTTAGAAACTACATAGATCAT |
| | | TTCCATTATTATATTGAAGACGACAGGTCGATGCTGGATGTTTAC |
| | | AGTGAAGTTTTTGACAGATATTTTTCATATGATATTAAATATCAG |
| | | AAGAATGTTGTTAATATGCTGTATAATGTCCTTTTGAGTCATTTC |
| | | GCTAAAGCCGGATTTGAATTTGGCGAAGGAATAAAACAGATAGGA |
| | | AGCAGCAAAAAGAACGAAATGCCGTTAACCAAAAAGATGGCGCGC |
| | | ATTCTGCTGAAATCACTTGAATCGGATGATTTTACTTATAAGATT |
| | | GGTAATACATCTGAAGCAAAGAATCAGGAAACGGTCGTTCTTCCT |
| | | GCCAGAGATGATCTTTTCCTTGATGCTCTTGGAAAAGTACTTAAT |
| | | TGGGATGGCAGTATTACTGATGAAAACGCTTTACAGAAAACAGAG |
| | | ATTATTACAGGAAGAAGTGGCAATTATCTGAAGAAATCAAGGAAC |
| | | AGTGATAAGAAGAATGATGGTAATAAAAGAAGTGATATAAAAAAA |
| | | TCATTCAATAAAGATAAAAACATACCCGAAAAGAAAGAAGCACTG |
| | | ACCAGTACTCCGTTTGCAAACCTGTTTAATAATTTACATATGGAT |
| | | TTTGATTAA |
| 3 | Ca3 | ATGAAGATATCCAAGGTTAATCATACTAAGTCAGCCGTTAGTGTT |
| | ContigID: | TCTGAAGGTTCTCCCAAGGGAATACTGTATGAGGATCCGACCAAA |
| | k127_751200 | AGCGGAACGAAGGATCTTGAAACTCGAATACTAGAGCGTAACGAA |
| | | GCTGCGAAATTACTGTATAACCCTATAAATACATCGAGAAGCAGA |
| | | AAGAAAACACATAAAATAATCAATAGAAGTCTTAGGGCTTTCTTC |
| | | AATCGAGTAAAGAAGAAAACCGGTGGGAGTTTCTCTTGGGATGAA |
| | | TTGAAAAGAGTATCGTATGATTCTTCTTTGGATGCAGAGCGTGAT |
| | | AAGATTACTGATTCTGATATAGATTCTGTTGTTGAAGCCTGTTTG |
| | | AAGAAGAGTCTTTCAACCCCGGAATGTATAGAGGCGGTAAAGCAG |
| | | ATTACAAGAGTTTTGTGTGGCAAGAATACCTCCCATGATCTGGAT |
| | | GACAAACTTATAGGGAAATTGTCTTCCAAATTACATGATGACTAC |
| | | TCGAAGGAACGATTGCTGGGTAACATCAAGAAGTCGATTGAAAAC |
| | | CAGAATATGGTAGTTCAACCCGGACAGGTTGACGGAGAAAGCATT |
| | | TTTAAATTAACTGGTGATGATTCTTTGAAAGAGAATCCGGAGAAG |
| | | GTTTCTTTCGAAAGATTCCTTATTAGCTATGCCAATCTCGACAAG |
| | | AAATTCCGTGATTGTGAATTGCGCAAGTTAAGAAGGCTGATAGTT |
| | | CTATATTTTTATGGTGAGACAGAAGTAGATACGACAGATGATTTT |
| | | GACGTTTGGGCAGATCACAAAAAGCAGCGTAATTTTAAATGGTTT |
| | | ATTTCAGATATCGAGTTTATTGCTACATATGAAAAATACCTTAAA |
| | | GAATTACAGTTTGAGGATAGAAGGAATCATCATACAAAGATATCG |
| | | GAACCTGAGTTCAGGGAGAAGATAAGACAAGAAAACATAAACAGA |
| | | TATAGGAATTCAATTGCAGTAATTAACAAATCAAATGAAGTGTAC |
| | | TTTGATGATCCTGTCCTTAACAAATTCTGGATCCATCATATAGAA |
| | | AATTCTGTTGAAAAACTGCTGAAAAGGGTAAATCCCGCCGATTCA |
| | | TTTAAGCTAAATGTTGCATATATAGGCGAGAAAGTCTGGAAAGAG |
| | | GTTATTAACTATCTGAGTATTAAATATATTGCTGTTGGTAAGGCT |
| | | GTCTATCGTTTTGCAGTTGATGATATGACCTATGGCGTCATACCG |
| | | GATCTGTATAAGTCAGGAATAAGCTCATTTGATTATGAACTCATA |
| | | AAAGCCGACGAATCGCTTCAAAGGGATATTGCCGTTTCGGTAGCA |
| | | TTTGCTGCCAATAATATGGCACGAGCTACTGTTGTGCTCGATGAG |
| | | AAGAGCAGCGATTTTCTTGTTGAGAGTTTTGATCTGGAAAAATCC |
| | | ATTAGAACAGATGTTGCGCTCGATATGGCGATACTGCAATTTTTC |
| | | GGGGGCAAATCGTCTTGGAAACAGTGCGACGCATTGAAAGATTGT |
| | | AAATATATCGATCTTCTTTATGACATGAAGAAAATGCTCTATTCC |
| | | ATTCGTAATATGAGTTTTCATTTTATTTCATCTGAAGAAGGAGAT |
| | | AACGGTTATAAGACTAACGGGATAATCCCGGCGATGTTCAATCAA |
| | | GAAATAACAACCTATACGACGATTCTTAAGAGCAAGTTCTATTCT |
| | | AACAATCTGCCTGCTTTTTATAATGATTCAGATTTGGAAGGAGAA |
| | | TTTAAACTTCTATATAAAAATTACGTAGAAAGAGCATCTCAAGTT |
| | | CCTTCTTTTAATAGTGTTGTTGTCAGAAAGAGTCTTCCGGATTTT |
| | | GTAAAAAGAGATCTGAAGATTAAAACAGCTCTCTCGGGAGATGAT |
| | | CTCACAAAGTGGCATAGCGCTCTATACTATCTTCTCAAGGAAATC |
| | | TATTACAATCTCTTCCTTGCAAGTGATGATGCAAAGATTTTGTTT |
| | | TTGAAAGCAGTTGAAAATAATAAGAATTCTAATAATAGCAGTGTT |
| | | TCTGATAAGAATGACCATCGAAGAGAAGCTGGAATTGATTTTGCC |
| | | GAGCGAATAGAGAGTATAAAAGATCATAGTCTTTCAGAGATATGT |
| | | CAGATCATTATGACCGAGTATAACCAGCAGAATCAAAGCAGAAAG |
| | | GTTAAGACGGCTCAAGATGAGAAGAATAAGAAATCTTTATTCATT |
| | | CACTACAAGATGTTGCTTAATCTTTGCCTTCGGAATGCATTTAAG |
| | | ATGTTCCTTGATCGCAACGAGTTCTCATTCTTAAAGAGTATTCAT |
| | | AATCGCGAAGTTAAGAGTTCCTCTGATGAATGGACCGCTGCTTTT |
| | | TGCGCGGATTGGACATCAAATGCATATAGCATGATTCAAGATGAG |
| | | ATTAATAAAAATCCTTCGTTGCAAAGCTGGTATATACTATCAAGA |
| | | TTCATTACAACAAAACAGCTTAACCATCTTAGTGGAGATATCAGA |
| | | CATTTTATTCAGTATGTAGAGGACGTTAAGAGGCGAGCCAAAGAG |
| | | ACAGGAAATGCATGTAAATACGATCTTGATAATAAGGTTTGTATA |
| | | TACCGTAAAGTACTTCAGGTTCTGGATTTTTGTAACAAAACAAGC |
| | | GGAATAGTATCTTCAGAGATTGGTGACTATTTTAAAGATGATGAT |
| | | GAATATGCAAAGTTTGTTAGTAACTATCTTGATTTTGGTGGAACG |
| | | ACGAAGCTGGAATTGATTGCGTTTACAAACCAGACTGTTGGAGAT |
| | | GATCAGATCAATATATATTGCAACGATTCAAAGCCAATCCTCAAC |
| | | AGGAATATTGTAATGGCAAAACTGTTTGCCCCTACAGATACGATA |
| | | AGTAAGGCGATAGCTGCAAATGGAAACCGAGTAACTGTCGATGAT |
| | | ATTGAGGAGTTCTATTCTATTAAACCGATTGCTCAGAAATTTCTT |
| | | TCTGATGGTGATAGTGTTGCCAAGAAAGAAAAGAAGCAACTTATA |
| | | GAGGAACTTAAGAAAACAAAGCGATATCAAGAGATAGTTAATCGC |
| | | ATTGAGTTCCGCAATATTGTAGATTATGCAGAGATGATAAATGAC |
| | | TTGCTTGGTCAGTTGGTCAGTTGGTCATATCTGAGGGAAAGAGAT |
| | | CTGTTGTATTTCCAGTTGGGTTTCCATTATCTTTGTCTTATCAAT |
| | | GATTCTTACAAGCCGGATAAGTATAGAGTCCTTCGTGATGGAGAG |
| | | CGGATAATAAACAATGCCGCACTATATCAGATCGTTTCGTTGTAT |
| | | TCCTTTAATGTAGATACTTTTAGAGATGATAATGATAAAGATAAA |
| | | GGTAAAAAATATAATAATATATGTGAGTACAGCCTGAATATTGGT |
| | | TTAGATGAAGAATGGCAATTCTATACTGCCGGGCTTGAACTATTC |
| | | GAGACTATCACTGAACATGATTCTATTAAGAAGTTTAGGGATTAT |
| | | ATTGATCACTTCCATTACTACACAAATCAGGATAGAAGCATTCTG |
| | | GATATGTATAGCGAGGTCTTTGACCGATTCTTCTCTTATGATATG |
| | | AAATTCCGTAAAAACACTGTCGTTATCTTGCAGAATATACTGAAA |
| | | TCTTATTTAGTAATAATGCCGGTTAAATTTAATTCGAAGTATAAA |
| | | TCAACAGATAACGGTAGTAGTAAAATGCGTGCCAATGTTGACATG |
| | | GGTGAAAAAGGCTTAAAGTCAGAGGTGTTTACTTATAAGTATTCT |
| | | GACAGCTGTAAGGTCATCTTGCCTGCAAGGTCGATTAATTATCTT |
| | | AAGGATGTTGCATCTATCCTGTATTATCCTCATAAAACGCCTCGT |
| | | GATGCTGTTGATATGGAAGATTTTAAAAAGAACTATGAGGTTGCA |
| | | CAAACTTTAAATAAGGCAAAAGACAATCATCATAAGTCTAAAAAT |
| | | GATTATAAGAATAATGATCGTCCTAAAGATAATTATTCTCCGTTT |
| | | AAGAGCCAGTTTGATAAATTGAAAAAGAAGGGTATTACATTTGAA |
| | | GATAACTGA |
| 4 | Ca4 | ATGAAAATATCAAAAGTAGATCATACAAGAACGGCAGTTGGGGTG |
| | ContigID: | AATGAGAATGGACCATTGGGAATTGTTTATTCAGATCCATCTCAG |
| | k127_5935133 | AATGCTGTTCAAAATCCAGAGATTAGAGTTAGGACTAGAATTAAA |
| | | AAAGCAAATATGCTTTATACGGTTTTTGGACCTACGAATGATGAG |
| | | ATGGATTCTCAACGAGAGAATGGAATAGCAAAAGAATTCAATAAA |
| | | ATAATCAAAAGATATAATAATAAAATAGATCCCAAGAGGGGAGAA |
| | | AAGGAAACTGATAAAAAGATATATAAAATGAATTCTGATGAACTG |
| | | ATTAAAGATATTAAATCGGTTTTTGGCAATTATTCTTTAAATGAA |
| | | TCAACGAGAAAAGAAATTGATGAAGCGTTAAATGTATTAATTAAA |
| | | CGTTCTCTTAGAAAAAAGGAAACTATTGAATCATTAAATCTCTTA |
| | | TTTGAAAAAACAATAAAGGGAGAGGAGTTTAAGGCGGAAGAGAAG |
| | | GATAAGATTCAAAAGTATGTGGTTGATCGTATAGTTGCTGACTAT |
| | | TCAAAGAATACGCTATCAAAAAATACTATAAAATCAATAAAAAAT |
| | | CAAAATCTTGTGGTCCAACCTCAAAATAAAAATGGCGAATTTGTT |
| | | TTTACACAGGCAAAAAATAGGATGAATGGGAAAGTAAATCAAGGA |
| | | AGTATAAGGATTTCAAAAGCTCAGGAAAAAGATGCTTTAAATGAT |
| | | TTTCTTGATGGTTTTGCAGTGCTTGATAAACAAATGAGAGATAAG |
| | | CAACTCATGAAAATAAGACGGTTAGTTGATTTATACTTTTATGGG |
| | | ATTGATGAGGTTGTAAAGGAAGACTTTTCAGTTTGGGAAAGACAT |
| | | GAAAAAACAAAAGGTAATGATAAAAAAATCATTCCTTTTTCTCGT |
| | | ACTGATATATCGACTTTACAAATAAAACGAGGAGATTCTGAGGAC |
| | | GAGAAAAAAAGGAAGAATAGAGAAAAGAAAATAATTAAAAAATCT |
| | | GATAGCGCTAAGTTGGACGATATGATAAGAAGGTGGAACATAGAT |
| | | AGATTCCGTGAATCGTTTAGTGCTATTGATAAAAGTGATAACAGT |
| | | TTGTTTTTTGATGATAAAAACATTTCTAAATTCTTTATTCATCAT |
| | | ATTGAAAATGAAGTAGAAAGGTTATTTAATTCAGAGAGATTGGAT |
| | | GATTATAAAATGCATATTGGTTATGTTAGCGAGAAGGTATGGAAG |
| | | GGAATTATTAATTATCTAAGTATAAAGTATATATCAATAGGCAAA |
| | | TCTGTTTATAATTATGCAATGGAGGAGTTAAATAATTCATCAGGA |
| | | GATGTAAATCTTGGTGTGATAGATAGTAGATATTTGACAGGTATT |
| | | AGTTCCTTCGATTATGAAAAGATAAGTGCTGAAGAAACTCTTCAA |
| | | CGAGAGACAGCAGTATATGTTTCATTTGCTTCTAGTAATTTGTCG |
| | | AGAGCTGTTTTTAAAGATGGAGTAGATTGTGACTTAATGTCGACT |
| | | AAGATTATAGATAATCACGATAAGTTTGATGAAAGCAAAGTAAAA |
| | | AAAAGAGTTCTACAGTTTTTTGGAGGAGAAAGCTCTTGGGATGGT |
| | | TTTGGAAAGACGTTTTTATCTGAAGAATATAATGAATTCGATTTT |
| | | TTGGAAGATCTAAAGACACTTATTTACCAGATGAGAAATGAGAGT |
| | | TTTCATTTTAATACTGAAAAGAAAAATGTAGATATAAAAAATCCT |
| | | AAACTTTTTTCAGATATGTTTGCTTATGAATGCAGTAAAGCGTGT |
| | | GTGTCTGAAAAAGATAAATTTTATTCGAATAATTTACCTCTCTTC |
| | | TATTCAGAAAAACCTCTTGAGAAAGTTCTGAATAAACTATACACC |
| | | AAGTATAATGATCGTAAGTCGCAAGTTCCGTCTTTTGAGAAGGTA |
| | | ATGAAAAGAAGTGAATTTGGAAAATATCTCATAAAATCTGGAGTT |
| | | GCCACCAACTTTAATAAAGAGGATACAGATAAACTTGAGTCGGGA |
| | | CTATATTACCTGTATAAGCAGATATATTATAATGATTTCCTAGTT |
| | | AACGATATGATTGCAAAGGGGATATTTGTAGATAATATAAACAAT |
| | | AAGAAACTAAGAAGAAATGAGAATAATAAAGTAATCAAAGCTGAT |
| | | AAAGGGCTTGAAGATTTTAAGAAACGCTTGAATGAAATAAAAAAT |
| | | TATTCTCTTTCTGAAATATGCCAAATTATAATGACAGAATATAAT |
| | | CAGCAAAATAATCAAAAGAAGAAATCTCAGAAAAATGAGGAGATA |
| | | TTCCAGCATTATAAATTGGGACTTTATTCATACCTTCGCGAGGCG |
| | | TTAATTATATATATTAATAATAATAGTGATATTTATGGTTTTATA |
| | | AAACAACCTACTATTAAATCTGAAGGTAAAATGCCGAATATTAAT |
| | | GAGTTTCTACCAGATTATTCTTCAAGTCAGTATGATGATTTGATA |
| | | GCAAAGGTCTCCGATTCTTTTGAACTGAAAAAGTGGTATGTAATG |
| | | ACTAGATTCTTAAATCCTAAACAGACTAATCATTTGGTTGGAGCG |
| | | TTAAGGAATTATATACAGTATGTGGAAAGCATAAAAAGAAGGGCG |
| | | GAAGAAACAGGAAATAAAATATATATAGATTGTCAGATTTTAGAA |
| | | TCTGTAAAAGATATCACAAAAGTAGTAGATATGTGTACCAGAATA |
| | | TGTGGTAATACTTCTAATGAAATCTCTGATTACTTTGATGATAAC |
| | | GATGACTATGCAGGTTATCTAGAACGTTTTTTAGACTTTGAATAT |
| | | AAAGAATCTTTGGGCTCGAAATCATCAATGCTTGGAGCATTCTGT |
| | | ATGACCAAAATTAATAGTGAAGAGATAAAGATTTACCATGATGGA |
| | | ACAAATCCTATACTTAATCGAAATATCGTATTATCTAAACTATAT |
| | | GGAGCAAATAGTATAATTTCAGAGGCTGTTCCAAAAGTTGATCAA |
| | | AATATGATAAAAGAATACTATATTGTGGCTGATAAAATTAAAGAA |
| | | TATCGAAAGAGTGGCGATTGTAAAAACATTGATGAGATTAAACAG |
| | | CTTAAAGAATACCAAGAATTAAAGAATAGGGTTGAATTTAGAGAT |
| | | ATTGTTGAATATTCAGAAATACTTAGTGAGCTGCAAGGACAGCTC |
| | | GTTAATTGGGCATATCTTAGGGAACGAGATTTGATGTATTTCCAA |
| | | TTAGGGTTTCATTACGTATGTCTCAAGAATGATAGTCAAAAGCCA |
| | | GAAGCATATAAAATGATTGAGGTGCCTTGCGTTGATGGGTCTTCT |
| | | CGAATGATAAATGGTGCTATTCTTTATCAGATAGTCGCAATGTAT |
| | | ACTTATGGAATGAATATATACTATAGGGGGCATAAAAAAGATGAG |
| | | GAGTACAATGATAGTGAAAATCGATGGGAAGCCTTCAATGGTTCA |
| | | ATAGGTGAGAGAATTCCAAGATTTGCCTTGTATTCTGGATATATG |
| | | ATTAAGGGAGACAATGCTAAGTACAAACTATCATATAATATTTAT |
| | | ACATCAGGGTTGGAACTATTTGAAGTTCTTGAAGAACATGGTAAC |
| | | ATAGTTGATTTTAGGAACGATATAGACCATTTTAACTACTATCAA |
| | | AAGAAGGATAGAAGTATGCTTGATTATTATAGTGAGGCTTTTGAT |
| | | AGATTTTTTACATATGATATGAAGTATCGGAAAAATGTTCCTAAT |
| | | ACATTATCTAATATTTTAGCGTCTCATTTTCTTGTTCCTTCTTTT |
| | | GTGTTTGGGACTTCTTCAAAAAAAGTGGGGAACAAAAACTATATT |
| | | GAAAAGAAGTGTGCTCACATTAGATTTAATACTAAGAATCCGTTA |
| | | AAACCAGGCAGTTTTACATATGTAATTTCGGAAGATAAACGTGTA |
| | | GTAGGACCAGCGAGACTGAAGGGGTATGTAAAAAATGTATTGAAT |
| | | ATTTTATATTATCCAGAAGTGCCTGAAATGGAGCTTTTAGATTCC |
| | | TCTTATATATTTAAAGAAGAAAAAAAGAGAAAACTTCTCAAATAA |
| 5 | Ca5 | ATGAAGATTTCTAAAGTAAGAGGAACTCAGGGCAAAGGAAGTAAA |
| | ContigID: | CTTACAATTAATGCAAAGGCAGCAGTTGTAATTAACCCAACCGGT |
| | k141_14579520 | CAGGAGGGTATTCTCTATGATGATCCGTCAAGAATGGGCGAATCA |
| | | AGAAAAAATGACAAGCAGAGAGAATCATACATCAAGGATCGTATT |
| | | CGTGCCTCTCAAAAATTGTATTCAATTTTCAATAGTAATCAAAAA |
| | | ATCCCCAAAAACAAAAAAACTGAATCTGAAAAAGCAATTGATATG |
| | | ATTATTGCTGGTTTTTCATCAGAAGATGGCGCCAGTTTTCGTTTA |
| | | ATGTTTAAAGATTTCGCTGAAATTCTGGATAAATATGCAGAAAAA |
| | | AGTTATGAAAACAGAAGAAATCATATAGACGAATCTCCCGAATTA |
| | | TCAAAGCTTGGGGTAAATATAAGTGACAATCAGATAAACGCCCTT |
| | | TCTAATCTATTAAGTGAAGAAAGTATAGCTATAAAAATAAAAAAA |
| | | GGAACTGAATCAGTAAAAGATAAGGTAAAGGTTAGCGAGAGGGAT |
| | | ATTGATTCGGCAATATCAAATTGCCTAAAGAAATGTATGTGCAGG |
| | | GTAAAAACAAAGAAAGCGTTAAAGGCTCTTCTTATGAAAGTTTTT |
| | | GATATCCCATATACTTTAGATGGAGACGTCAATATTAGAAGAGAT |
| | | TTTATTGATTATGCGATAGAAGATTATTGCCGTATTCGTGTAAAA |
| | | AACAGTGTCTCTGAATCAATTAAAAAGAATAATATGCCGGTTCAG |
| | | CCGACGAGTTCGGAAGGTGTTACTGTTTTTCAGATGCCTTCTTTG |
| | | CAGGAAACAAAAAGTACCAAGAGTAAAGAAAGGGAAGCATTTAAT |
| | | CATTTCCTGTCAGAATATGCAGATTTAGATGAGAATAAGAGAAAG |
| | | TCGTTACGAATAAAACTCCGAAGACTTAATGATTTATATTTTTAT |
| | | GGAAAAGACGCAACTATGGCATTGGCTGATAACGAGGATGTGGAC |
| | | GTTTGGGAAGACCATGCAAAACATGGCGATATTAAAGAGTTATTT |
| | | ATAAAAGTTCAAAAACCACAAATCACAGGTGACGGAAAAGCGGAT |
| | | AAGCTGGCTATGAGTCAGTATGAAGATAATATTCGAACTAAATAT |
| | | AGAGAAGCAAATATTACTTGTTACAGAAAAGCAGTAGAAGAAATA |
| | | GATAATGACAAGAGTCTATTCTTTGAAGATAATATGCTCAATATG |
| | | TTTGTGCTTCACAGAATTGAGAGTGGTGTAGAACGAATATATTCG |
| | | CATATTAAAGCCAATGAGGAGTACAAGCTTCAAACGGGATATGTA |
| | | AGTGAAAAAGTATGGAAAGATCTGATTAACTATATTTCCATAAAA |
| | | TATATTGCTATTGGAAAAGCGGTGTATAACTATGCCATGGATGAA |
| | | CTTGTCAGTGGAGATAAGAGTATTGAAATGGGCAAGATTAATGAC |
| | | AATTATATTTCCGGTATAAGTTCCTTTGACTATGAGCTTATTAAG |
| | | GCTGAGGAGATGCTTCAGAGAGAAACTGCTGTTTATGTTGCATTT |
| | | GCAGCAAGACATCTTGCTCATCAGACAGTCGACTTAGACGAGAAA |
| | | AATTCAGATTTTTTACTATTTCCGGATAAGAGTAGAAAAGATAAA |
| | | GATGGTAAAAATATAAATGATTTTATAAAAGAAGGTATTAATCTT |
| | | CGTTCTACTATATTGCAGTATTTTGGTGGGGCATCTTCATGGAGT |
| | | GATTTTTCATTTGAAAAGTATATGACAGATGGTCGCGATGATGTA |
| | | GATCTGCTTACCGATTTACAGAAAGCAATTTATTCTATGCGAAAT |
| | | GACAGTTTTCATTATACATCTAAAAACCATAATAATGATGGTTGG |
| | | AATAAAGAATTAATTGGAGCATTGTTTGAATATGAGGCTAATCGG |
| | | CTGACTATAATACAGAAAGATAAATTTTATTCAAACAATTTACCA |
| | | ATGTTTTATGATGAAAGTAATTTGAAGGAATTATTATCATCTCTA |
| | | TACAGTAAATCTGTAGAAAGGGCTTCTCAGGTTCCTTCATTTAAC |
| | | AGTGTATTTGTCAGAAAATCATTCCCAAAAGTCTGTACACAAGAC |
| | | TTATCTATTGATGTAAAGACAATGAATGAAGAGGATAAACTTAAG |
| | | TTTTATAATGCTCTTTATTTTATGTTTAAAGAGATTTACTACAAT |
| | | CTTTTTCTTAATGATTCAAACGTTTTAAATCGTTTTATTGATATT |
| | | TCGACAAAAACAAAAAAGAACGGAAAGGGAGATGAAGGTACTCAT |
| | | TATTGGGCAGAAAAAGATTTCAGACAGAGGATTCTCTCTATAATA |
| | | GAGAGCAGAAAAAATTATACTCTTTCACAGATATGTCAGTTAATT |
| | | ATGACTGAATATAATCAGCAGAATACAGGCAATATGAGACATAAG |
| | | TCTGCTGATAAAAACGGTAAAAATCCTGACAGTTATCAGCATTAC |
| | | AAAATGCTTCTATTGTCATATCTTGGAGAAGCGTTTGTTGAATTT |
| | | GTAAAAGAAAAATATGATTTTGTATTCACACCGGTTAAAAGGGAT |
| | | TTGATGGATAAAGAGGCATTTTTGCCTGATTTCGCAAAGACAGTC |
| | | AATCCTCTTGGCGATTTAATTGAAAGAGTGAAAGAATCCGGTGTA |
| | | TTGCAAAAATGGTATATAGTAGGCAGATTCCTCAGTCCCAAACAG |
| | | GCTAATCAGATGCTTGGTTCTTTGCACAGTTATAAGCAGTATGTG |
| | | TGGGATATATATCGAAGAGCAGAAGAGACTGGTACGAAAATTAAT |
| | | AAACGTGTTTCAGAAGATACCATATCAGGAGTTGCCATTAGAGAT |
| | | ATAGACAGCGTGCTTGATCTTTGTGTAAAGATGTCCGGAACTATT |
| | | ACGAATAATCTGACAGATTATTTCAAAGACAAGGAAGAATATGCA |
| | | GCTTATATTAACGATTTTCTTGATTTTGAGTATAAAACCGGAGAT |
| | | TACAATTGGGCTCTTAAAGACTTTTGTAAAGAAATAACGGATGAA |
| | | GATGACAAAGAAGGTATTTATTATGACGGGGAAAATCCGATAATA |
| | | AATCGTAATATTGTTATATCAAAACTGTATGGCGAAGCGGAATTT |
| | | GTTTCAAAAATCTTCAAAAGAGTAAATAAAGAAGATATAAAAGTA |
| | | TATAAAGACCTTAAGAAAAATATCGAACCATATCAGAATATGGGA |
| | | ACATTTGAAACAAAAGAGCAGCAGGAAAATGTTAAACGTTTCCAG |
| | | GAATTAAAGAATCATATAGAATTCAGAGATCTTGTTGATTACAGC |
| | | GAAATAACAAATGAACTTCAGGGGCAGCTTGTAAACTGGATTTAT |
| | | TTGCGTGAACGAGATTTAATGTATTTCCAGCTTGGATTCCATTAT |
| | | TTATGTTTGAATAATAACAGTGAGAAACCGGAGCTATATAAGAAA |
| | | ATAGAATTCAAGGACGAAAAAGTCATTGATAATGCCGTACTTTAT |
| | | CAAATTTGTGCTATGTATACCAACGGTTTGCCATTATACTATAGC |
| | | AGTACCAAAAATGCAAACATAAAGGAAGTGAGTGCAAAAGCGGGA |
| | | ACGAGCACAAAAGTAGATAAATTCTATTCATCTGGAATCAGAGCT |
| | | AATGGTGAAAGTTATTCGAGAGACTATACAACTTATATGGCTGGT |
| | | TTGGAGCTATTCGAAAATACAAAGGAACATATAAATATTACTATG |
| | | TTTAGAAATGATATTGAGCATTTCAGATATTTAGTTTCAAACACA |
| | | AGAAGTATGTTGGATGTATACAGTGAAATATTTGATCGGTTCTTT |
| | | ACATATGATATGAAATACAGGAAAAATATACCAAACATTTTATAT |
| | | AATATTTTATTGGCCCATTTTGTGAATGTTCAATTCGATTTTTCT |
| | | ACAGGGAAAAAGAATATTGGAACAGGGGAGAATATATATGAAAAG |
| | | AAATGCGCGAAAATAAATATCCAAAATAATGGTGGAATTGTATCT |
| | | GAGAAGTTTACTTATAAATTAAAGGACGAGAAAACGATTGATTTG |
| | | CCTGCAAGGGGACGAAGGTACATGGAAACCGTAGCAAGGTTATTA |
| | | TACTATCCTGAAACAGTTGATGAGGAGAAAATGGTAAAAGATTTG |
| | | GTCATTAAAGATAATAAGCCATTTGGAAAGAAACGAAATAATAAG |
| | | TATAGTAACAGAAAAGAAGGTGCTTCGGATAGAAAAAAATATGAA |
| | | GAGAATAAAGCCAGGAAAAAAGATAATAGTTTTATGTCCGGAATG |
| | | GACGGTGTAGATTGGTCTAAATTAAATTTTAAATAA |
| 6 | Ca6 | ATGAAAATATCAAAAGTTGATCACACCAGGATGGCGGTTGCTAAA |
| | ContigID: | GGTAATGAACTTAGGAGAGATGAGATCAGTGGAATCCTCTATAAG |
| | k141_10995992 | GATCCGACAAAGGCAGGGAGTATAAACTTTGATGAACGGTTCAAT |
| | | AAATTGAATCAATCGGCAAAAATCCTGTATCACGTGTTCAATGGA |
| | | GTTGTTACAGGAAACAAACATTTTATTAATACTGTTAAAAGGGTT |
| | | AATGACAATTTAGACAGGGTATTATTCACAGGTAGGAACGATGAA |
| | | AGAAAATCTATCACAGATACAGATGTTGTTCTGAGAAATGCGGAT |
| | | AGGATCAATGCATTCGATAGGATTTCAACAGACGAGAGAAAACAG |
| | | ATAATTGATGAGTTATTGGAGATCCAACTGAGAAAGGGCTTGAGA |
| | | AAGGGAAAGACCGGACTTAGAGAGATATTGCTGATAGGTGCCGGA |
| | | GTAAAAGGCAGAACTGACAGGAAACAGGATATAGCTAAGTTCCTT |
| | | GAGATCTTGGATGAAGATTTCAATAAGACAAAGCAGGCTAAGAAT |
| | | ATAAAGTTGTCCATAGAGAATCAGGGATTGGTAGTAGCGCCTGTA |
| | | GAAAAAGGAGAGGACAGGATCTTTGATGTCAGCGGGGTTCAGAAA |
| | | GGAAAAAGCAGCAAAAAAGCTCAGGAGAAAGAAGCTCTGTCTGCA |
| | | TTTCTGTCAGATTATGCTGATCTGGACAAGAGCGTCAGGACTGAG |
| | | TATCTTCGTAAGATCAGAAGACTGATAAATCTATATTTCTACGTC |
| | | AAAAACGATGACGATCTGTCTTCAGCAGAAATTCCGGCAGAAGTG |
| | | AATCTGGAAAAGGACTTTGATATCTGGAGAGATCACGAACAAAAA |
| | | AAGGGAGAAAAAGGAGACTTTGTTGACTACCCGGACATACTTTTG |
| | | GCAGATCGTGATGAGAAGAAAAGAAACAGTAAACAGGTAAAAATT |
| | | GCAGAGAAGCAATTAAGGGAGTCAATACGCGAAAATAATATAAAA |
| | | CGGTATAGATTTAGCATAAAGACAATCGAAAAAGATGATGGAACA |
| | | TACTTCTTTGCAGATAAGCAGATAAGTGCATTCTGGATTCACCAT |
| | | ATCGAAAATGCGGTTGAACGAATATTAGGATCGATTAATGACAAA |
| | | AAACTGTACAGATTGCATTTAGGATATCTTGGAGAAAAAGTCTGG |
| | | AAGGATATACTTAATTTCCTTAGCATAAAATACATCGCTGTGGGT |
| | | AAGGCGGTATTTAATTTTGCAATGGATGATCTGCAGGAGAAGGAT |
| | | AGAGATATCGAACCCGGCAAGATATCAGAAAAAGCATTAAATGGA |
| | | TTGACATCATTTGATTATGAACAGATAAAGGCTGATGAAATGCTG |
| | | CAGAGGGAAGTAGCTGTCAATGTGGCATTCGCAGCAAATAATCTG |
| | | GCCAGGGTAACTGTAGATATTCCTCAAGATGAAAACAAGGACAAA |
| | | GAGGATATCCTTCTTTGGAATAAGCAGGACATACACAAATACAAA |
| | | AAGAAGTCTCAGAAAGGTATTCTGAAGTCTACTCTGCAGTTTTTT |
| | | GGAGGCGCTTCAACCTGGGATCTTAAAATGTTTGAGAAGGCATAT |
| | | CCGGACCAGAAAGAGGATTACGAAGAAGAATATCTATATGACATT |
| | | ATCCGGATCATTTATGCACTCAGGAATAAGAGCTTTCATTTCAAG |
| | | ACATATGATCAGGGTGACAGGAATTGGAACAGCAAACTGATCGGA |
| | | ATGATGATCGAGCATGATGCTGAGAAAGTTGTTTCTGTTGAGAGA |
| | | GAAAAGTTCCATTCCAATAATCTGCCGATGTTTTATAAAGACGCT |
| | | GATCTAGAGAAGATGTTGGATCTCTTATACAGCGACTATACAGGA |
| | | CGAGCATCGCAGGTTCCGGCATTTAACACTGTTTTGGTTCGAAAG |
| | | AATTTCCCGGAATTTCTTAGGAAAGACATGGGCTATAAGGTTCAT |
| | | TTCAGCAATCCTGAGGTAGAGAATCAGTGGCACAGTGCGGTGTAT |
| | | TACCTATATAAAGAGATTTATTACAATCTGTTTTTGAGGGATAAA |
| | | GATGTAAAGAATCTTTTTTATACTTCGTTAAAGAATATAGGCAAT |
| | | GAAGTTTCGGACAAAAAACAAAAGCTGGCTTCGGATGATTTTGCG |
| | | TCCAGATGTAAAGAAATAAAGGATAGAGACCTTTCGGAAATCTGT |
| | | CAGATGATAATGACAGAATATAACGCTCAGAACTCCGGCAATAGA |
| | | AAAGTTAAATCTCAGCGTATGATCGAGAAAAATAAGGATATTTTC |
| | | AGACATTATAAAATGCTGTTGATAAAGACTCTATCCGGTGCTTTT |
| | | GCACTTTACTTAAAGCAGGAAAAATTCGCATTTATCGGAAATGCG |
| | | GCAACGATACCGTATGAAACAACTGATGTGAAGGAATTTTTGCCT |
| | | GAATGGAAATCCGGAATGTATGCATCGCTTGTAGATGAGATAAAG |
| | | GAGAATCTTGATCTTCAAGAATGGTATATCACTGGGCGATTCCTC |
| | | AATGGAAGGATGCTTAATCAGTTGGCAGGAAGCCTGCGTTCATAC |
| | | ATACAGTATGCAGAAGACATAGAACGTCGTGCAGCAGAAAATAGG |
| | | AATAAGCTTTTCTATAAGTCTGACGAAAAGATTGAGACATGTAAA |
| | | AAGGCAGTTAGAGTACTTGATCTCTGCATAAAAATTTCCACAAGA |
| | | ATATCTGCAGAGTTTACAGACTATTTTGATAGCGAAGATGATTAT |
| | | GCAGATTATCTTGAAAATTATCTTAGCTATCAGGATGATACGATT |
| | | AAGGAATTATCCGGATCTTCGTATGCTGCGCTTGATCATTTTTGC |
| | | AACAAAGATGATCTGAAATTTGATATCTATGTAAATGCTGGACAG |
| | | AAGCCGATCCTGCAGAGAAATATCGTGATGGCAAAGCTTTTCGGA |
| | | CCTGATAGTATTTTACCGGAGGTTATGGAAAAGGTCACAGAAAGT |
| | | GACATACGGGAATACTATGACTATCTGAAAAAAGTATCAGGTTAT |
| | | CGCGTAAAGGGAAAATGCAGTACCGTGAAAGAACAGGATGATCTG |
| | | CTGAAGTTTCAGAGATTGAAAAATGCAGTAGAATTCCGGGATGTT |
| | | ACTGAGTATGCAGAGGTTATCAATGAGCTTTTAGGACAGCTGATC |
| | | AGCTGGTCATATCTTAGAGAGAGGGATCTGCTGTATTTCCAGTTG |
| | | GGATTCCATTATATGTGTCTGAAAAACAAATCTTTCAAGCCGGCA |
| | | GAATATATGGATATTAAGAGAAAGAATGGTACAACTATACATAAT |
| | | GCGATTTTGTACCAGATCGTATCTATGTATATTAATGGATTAGAT |
| | | TTCTACAGCTGTGAAAAAGATAATGACAAGCTAGAAGTGGCGGCA |
| | | GCAGGAAAGGGAGTAGGAAGTAAGATATCGCTTTTTATAAAGTAC |
| | | TCAGAGTATTTGTATAATGATCCGTCATATAAGTATGAGATCTAT |
| | | AATGCAGGATTAGAAGTTTTTGAAAACAATGATGAGCATGATAAT |
| | | ATTACGGATCTTAGAAAGTATGTAGATCATTTTAAGTATTATGCG |
| | | TCGGATGATTCTGATAAAAAAATGAGCCTGCTTGATCTTTATAGT |
| | | GAATTCTTTGATCGATTCTTTACATATGATATGAAGTATCAGAAG |
| | | AATGTGGTGAATGTGTTAGAAAACATCCTTTTGAGGCATTTTGTC |
| | | ATTTTCTATCCAAAGTTTGGATCAGGAACAAAAGAGGTTGGAGTC |
| | | AAGAACTGTAAAAAAGAAAAAGATAGGGCTCAGATTGAAATAAGT |
| | | GAACAGAGCCTTACTTCGGAAGACTTTATGTTTAAGCTTGATGAC |
| | | AAATCGGAAGGAGAACCAAAGAAGTTTCCGGCAAGGGATGAACGT |
| | | TATCTCCAGACGATCGCCAAGTTGCTTTATTATCCTAAAAAAGAT |
| | | GTTGATTTGAACAAATTCATGACAAAAGAAGAATCAATGAATAAA |
| | | AAAGTTCAGTTCAATAGAAAAAAGGAAACAAACAGGAGACAACAG |
| | | AATAATTCATCAAGCGGAGCATTATCTTCATCTATGGGTGATTTA |
| | | TTAAAGAACATCAAATTGTAA |
| 7 | Ca7 | ATGAAAATATCTAAAGTTAATCATGTGAGAACAGGAACGAGAATT |
| | ContigID: | AAAGAAAACAATGGTGAAGGAGTATTATATGCTAATCCTTCAAAA |
| | k141_12677984 | CAGACAAATGCCGTAAAAGATTTATCTAAGCATATCCAGGATGTA |
| | | AATCAGAAAGCTCAAGGATTGTATTCTCCATTAAACCCGGTAAAA |
| | | TCTCTTATTAATCCTAAAATGCCAAAAGAAAAGAAGGATGAGATT |
| | | AATGGTTCATACAAAGCTTTTAAGAGCGTTGTTATCGGTATTGTA |
| | | AAAGAAAATGAAACAGGAATTCCGGATTCTGCTTCTGTAATTAGG |
| | | ACTTTATATGAAAAAGCTAAAAAAATAGATCTAAAAGTTTCGGAT |
| | | GCATCTTATTTGTCTTCGAAGCTGATAGACAAGTGCTTAAGAAAA |
| | | AGTCTCGAGTCAAAATCTGAGATTGCAAAAGAAATATTAAAGGCA |
| | | ATTATTTCTACGGATAAAAGTGCGGTTAATTCGCTTAATGCCGAA |
| | | GAAGTAAAGGCTTTCTTTGAACTGGTTCATAAGGATTATTATAAG |
| | | AAAGAACAACTCAAAGCAATTGAAAAGTCTATAGAAAATAAAGAT |
| | | GTCAAAGTTCAGGTAAAAACAGGACAAAATGGCGAAAATCATCTT |
| | | GTTCTTTCAAATGCTGATAGTGCGAAAAAGCATTATTATTTTGAT |
| | | TTTGTAAAAGAATTTGCCACAAAAGACAAAGCTGAAAGAGAAGAA |
| | | ATGATTATCAGATTTCGTCAGTTGATTATTCTTTTTTATTCGGGT |
| | | TCAGAGTCTTATAAACTTTCGATTGGCTCTGATGTTGGGGCTTGG |
| | | ACTTTTGGTTCGTCTCTTCCTGAAGTTACAGCCAATGTCGACGAT |
| | | GAAATTGCTTCTTTGATTGCAGAATATAATGAAAATATTGCGCGC |
| | | AAAAACGATATTCAAAAATCGATTGATTTGAAATCCAATCAAATG |
| | | AAGAATTATAAGTTTAATTCTCCTGAATATAAAAAATTAGATGAT |
| | | CAGGTTTCAAAACTAAAGGATGAACAGGGAGATTGTAAGCATGCA |
| | | ATATCCGACGCAAAAAGAAAGATTAAAGCTCTTGTTGAGAATCTG |
| | | ATATGCACAAAATATCGTGATGCTGTTAAGGCAGAGGGCTTAACT |
| | | GATTCTGATATATTCTGGATAGGATATATTCAGCAGGTTGCTCAA |
| | | AAACAGTTTAGCAAGAAGGACGCATATAACAATTACAGAATATCA |
| | | ACTAAATACCTGTATGAAGTTACATTTAATGAGTGGATTTCGTTC |
| | | ATGGCATCAAAATACATTGATCTTGGAAAGGCAGTATATCATTTT |
| | | GCGATGCCTGACTTTAGCGATATTAAATCAGGTAAGGAAGTCCAT |
| | | GCGGGAAAAGTACAACCCGCTTTTGAAGATGGAATTACAAGCTTC |
| | | GATTATGAAAGAATTAAAGCCAAGGAAACATTGGCAAGAGATTTT |
| | | TCAGTGTATGCCACTTATTCATCAGGCATTTTCTCCAATGCTGTT |
| | | ACAGATAGCGAATATAGGCTAAAAGATGAAAAAGAAGATGCTTTG |
| | | TTTTATAAGCAGGAAGATTGGGAGCAAGCGCTTTTGCCCAATGCT |
| | | AAGAAGAAGCTTCTTATGTATTTTGGCGGTCAAACAAAATGGGAG |
| | | GACTCTGAAATTGAAAAGTTGTCGGATCTAGAGATGACTAAAGCA |
| | | TTTCAGGATATGATAAACGTCATCCGTAACTCGAACTATCATTAT |
| | | GCAGGAAGTGTGTTGGAACCCGGTGAGCAAAGCGTTAACATTGCA |
| | | AAAATGCTTTTTGAAAAAGAGTTTTCCCAGCTTGGAAGAATAATA |
| | | AGGGAAAAGTATTTATCCAATAATGTTCCCGTATATTACAACGTT |
| | | GAAGATATTAATAAGATGATGACTTATCTATATCAGGGTGAATCC |
| | | AAGAGAGAAGCACAGATTCCATCATTTGGCAATGTTCTTAAAAAG |
| | | AAAGAAATGCCCGGATTTGTATCTAAGTATATTCCCGGAAACTTA |
| | | CTTGCTAAGTTTGATTCCGAAGGTATGGACAAGTTCAGAGCATCT |
| | | CTTTACTTTGTATTGAAGGAAGCGTATTATTATGGATTTTTGAAT |
| | | GAGACTAATCTTAAAGACAGATTTATTATGGCATTTAAAAATTCC |
| | | GAAAAAGATGCCAAGAATCCTGAAGCTATTGAAAACTTCAAAGCA |
| | | CGAATCGCTGATATGGATGATTCATGTTCGTTTGGTGAGATATGT |
| | | CAGATTCTTATGACAGATTACAATCAGCAAAATCAGGGTGAATAT |
| | | AAGGTAAAGTCTCAGATAAAACAAAATCAAGATGAGAAAGACAAC |
| | | AAAGGTCATAAGTATTCTCATTTCAAAATGCTTTTGTATGTAACT |
| | | CTGCAAAAAGCGTTTATTGATTATATTTTTGAAAAACAGGATATA |
| | | TATGGCTATATTAAAGCTCCGATTTTCAAAAGTAATTTCTTTGAC |
| | | GGAGATGAACCTCAAAAGTTTGTAGAATCATGGGAAGCCAATCTG |
| | | TTTGGCGATGTAAAGAAAACAACAGAAACAGACTCGTACTATTTG |
| | | GCATGGTATGTGCTTTCTCATATGCTTCCGGCAAAGCAGGTTAAT |
| | | CAACTTCAAGGCGGAATAAAGAGTTATATTCAATTTGTCACAGAT |
| | | ATAAACAGACGTGAAAAGAGTGTACTCGGAACAGAAAAAGATAAT |
| | | AGCTTGGTCAATAATATAGATTATTATCAAAATATACTTAAGGTC |
| | | CTTGAGTTTGTAATGTGTTTTGTTGGAAAAACATCCAATGTTTTG |
| | | ACAGATTACTTTGCTGATGAAGATGACTATGCATTGCATTTGTAT |
| | | TCCTATGTCGGATTCGCTGGTAAGAAAGAGGAGAAAACCAATTCT |
| | | ACTCTTTCCGGTTTTTGCAGTAAATCTATAACAAAAGCAGGAAAA |
| | | GTATTAACGGACAGAATAGGAATATATCATGATGGAACTAACCCT |
| | | ATTGTAAATAATAATGTCGTTAAAGCGCTCATGTATGGCAACGAG |
| | | AACGTTCTTTCTGAAGCGGTTACCAGAGTTTCAGCTGATTTGATT |
| | | AATGGAGAAATAACAAAATACTATGAAGTAAAGAATAAACTTGAA |
| | | AAGGTATTTGAAAAAGGCGAATGCTCAAATATAGAAGAGCAGAAA |
| | | GAATTAAGAGAGTTCCAAAACCTTAAGAACAGAATAGAACTTCAA |
| | | GACATATCCATATTTACGGAAATAATTAATGACTATATGTCTGAA |
| | | CTTGTAAATATGGCTTATCTTCGTGAAAGAGATCTTATGTATTAT |
| | | CAGCTTGGCTATAATTATATTAGATTGGAATACGGTAATGTTGAG |
| | | GATAAGTATAAAGAACTTCAAGGCGACAATATAAATATCAAATCA |
| | | GGAGCTCTTTTGTACCAGATAGTTGCACTTTACACACATGAGTTG |
| | | CCGATTGTTTATAAGGACAAGGATAGCTATAAATATACTAATAAC |
| | | GGTAAAATTGGCAGATTTGTTAAATCATACTGCGAAGAAGAATTC |
| | | AATGATTTAGATAATACTTATTTGAAGGGCTTGGAATTGTTTGAA |
| | | GATATAAAACTTCATGACGACTTGCATATGTTTAGAAATGAAATA |
| | | GATCATCTTAAGTACTTTATTCGTGCAGACAAATCTATTCTTCAA |
| | | ATGTATAGTCGTATTTACAATGGATTCTTTAGCTACGATTTGAAG |
| | | CTTAAGAAGAGTGTATCATACATATTTGCCAATATCCTTGCGAAG |
| | | TATTTTATTATTGCCGATACAGAGATGAAGAGTTCGGTTGAAAAC |
| | | GGAAAAAGAGTAGCCATGCTTTCAGTCAAGGGATTGGAATCGGAT |
| | | GTATTTACTTATAAGGGCAAAAAACGTGATAAAGAAGGAAAAGAA |
| | | AGAGACAGCAAGTATACATTGCCGGTTAGAAGTGATGAATTCTTA |
| | | AAAGAAGTCAAGAAACTTCTTGGCTATAAGAGTATGTGA |
| 8 | Cb8 | ATGCCAAACGTAAAATTCACCTTAGTCCCTGTGGACTATTCAAAG |
| | ContigID: | CCTTACGACGAACAACCCGATTGTAAACGTCACGTCATTGGAGCT |
| | k127_4804511 | TATGCTAATCGGCATGCCTCTTTTTAATGAGAACGAAATAGAAAA |
| | | TGCTTTCAACAAGTCTGGCTCGTCACAACATGACACTAACCATCA |
| | | ACACCATCATGCAGGCCATCCCATCGCAAAAAATTGGAAGCTCTT |
| | | GACAACATCCAGAAAGTGAAACTGCAGAAACGCCTCTATCGTCAT |
| | | TTCCCGTTCTTCAAGCGAATGAAGTTGGAGGATGAAGAGAAAAAG |
| | | ACGGTTCAACTGAAATCGCTCATGACAGTCATGTCACTTTTTACA |
| | | AGTCTGATGGCTGATATACGCAACAATTACACACATTACCGACCT |
| | | TATAATAACAAAGAAGAACAAAACAGACAATTAGAACTTAAAAAA |
| | | GAAGTAGGCAAAAAACTACAATATTTGTACGAGAATAGCAGCCAG |
| | | ACATTTAAGAGCATGGAGGAACTTGATCATTCCAGCAATGAGGTG |
| | | CTTTCAGCTCTTCGCATTCCTGAAGACGTTGTGGAACGTTTCTCG |
| | | CCAGACGATCCTGATTACAAGAAACTACTCAACACGCTACATGAT |
| | | TCCAATATACCGAAATGGAAAAAATCAGGTTTGAAACTCGACATG |
| | | AAAACCCAGATAATCACCAAGAAATCTGTACGCTATGTGCGAAAC |
| | | CCTAACTATCAAGCCTATATGATGGATGAAGAGAAAGGCTTGTCT |
| | | GATATAGGCATTATTTACTTCTTGTGCCTTTTTCTGGACAAACAA |
| | | GTATCTTTCTCACTAATGGATGAAGTTGGCTTCAATCAGCAGATT |
| | | AAGTTCACAGGTGAGCATGCAGAACAGCAGTTAATGTATGTCAAA |
| | | GAAATCATGTGCATGAACCGTATCCGGATGGTGAAGGCCAGGATA |
| | | GACAGTGAGATGTCAGACACGGCACTGGCATTGGATATGTTAAGT |
| | | GAACTGCGCAAATGTCCTCGTCCGTTGTATGATGTATTCTGTAAA |
| | | GAGGCACGTAACGAATTCAAAGATGATGCCACAGTAGTTTGGGAA |
| | | AACACTCACGGCGAGGAGGCTGTTATTACTGAAGAGCAAGGTGAT |
| | | ATAGGGGAAGAGACAGATGCTATTGCCGCAAATACTACGGGAAAA |
| | | AATACTCCTCGCAGTACCTTTGTACGCTGGGAAGACCGCTTTCCC |
| | | CAGTTGGCACTCAAGTATATAGACTTGACAGGTATGTTCGACCAT |
| | | CTTCGTTTTCAACTTAATTTAGGTAAATATCGCTTTGCCTTTTAC |
| | | CAACACGACAAAGCATACAGCGTTGATAATGCTGAGCGCCTGCGT |
| | | ATTCTTCAAAAGGAATTGCATGGTTTTGGACGCATCCAGGAGGTG |
| | | AACGAAATGATGAAGGAAAAATGGCAGGATGTCATGGAGATCAAG |
| | | AACGTTGAGGATGGACAAATATATAAGGAACCCGATGTAGCAGGG |
| | | CAGAAGCCTTACGTGACCCAGCAGAATGCCCAATATGACTTTGAC |
| | | ACCAAGAGCCACTCCATCGGCATCCGATGGGAGGGATGGCACAAC |
| | | AACCACTCTGACAATCATTATGGCGATTTGGATAGGAGGGATATG |
| | | TTTATCCCAAGATTGCCCGCTAACCCTGCATCGCCTGAGGGCGAC |
| | | AAGCGTCAGACTAATCAGGCAGAAGAACTGTTACCGCCTCAGTGT |
| | | ATGCTGAGTCTCTATGAGTTGCCAGCTATTTTGTTCTATCACTAT |
| | | CTGCTTAAAAAATATCAGAAAAATACTGGATTGGTGGAGAAGAAA |
| | | ATCTCCGACTTTTACACCAACATGAAAAACTTCCTGACAGAAGTC |
| | | AGCGAGGGTAACATATTACCTGCCGATGAGACCACGTTAATCCGT |
| | | GAATTACAGACAAGAGGGCTTAAGTTTTCTGACATTCCTGTCAAA |
| | | CTGAAGAAACTGCTCAAGGGCGAAGTGACAGACAATGCAAAGCGC |
| | | ATGGAGGAATCAGCCCTCCTGCGTCTGCATGAGCGTAAAGATAAG |
| | | AAAAGACGGGCACTTGAAAGTTTCATTGCCAAATGCAAGATGATT |
| | | GGCACCAAAGAAAACAAATTTAATAAAATACGTGCCGTCGTCAAG |
| | | ACTGGATCCTTAGGACAGCTACTGGCTCGCGACATCATGGAATGG |
| | | CTAACGACAGACACAAAGAAACGTATGAACCTGACAGGTCAAAAC |
| | | TATGTTGCCATGCAAACGGCACTTTCGATGATGGGACAAAGTTTC |
| | | GAGTTGGCACCTGAAGCAAAGGTGACTTGTGAAAAGATGAGAAAC |
| | | ATCTTTGTTAAGGCTAATATCCTGCCCATGAACGATGACGACTTT |
| | | GATGCAGATTTTCATCATCCGTTCCTACTTGATGTATTTGACGAA |
| | | GAGCCTGTTTCTATCGAAGACTTCTACAAGATTTACTTGGAGAAA |
| | | GAAATCTTCTACATCGACTATCTAACCGAACATTTCAAAAAGTAC |
| | | AAAGCCAAAGGGGCTGCTCTGTACATACCATTCCTGCATTGCGAA |
| | | AGGCTTCGATGGAAAAATACTGAACAGAACGGTCTGAAGGAACTG |
| | | GCAGCTCGCTACCTGCAGCGTCCTTTGCAATTGCCTAACGGACTC |
| | | TTTACTGATGACATTTTCCATTTATTGGAGGATATAGCCACTAAG |
| | | AATGCCGATTTTGCAAAAGTGCTTGAAAAACAGAAAAAAGACAAC |
| | | CATCAGTTGCAGCAGAATGTAGCCTACATGATTCGAATCTACATG |
| | | AAGACAGTAGAGTATGATCAGCCACAAAACTTCTATAACACGATG |
| | | CCCGTCGGAGATACCAATAGTCCATATCGTCACATCTATCGCATC |
| | | TTCAAGAAATTCTTCGGTGAATCCATCCCCAAGACAAACAAGACG |
| | | ACATCGCCAGCCTACACCATCGAGGAGATTCGTGCCATTTTGAAT |
| | | AATAAGCAGCTATTGAAGGATAAGATAGACTTCTTTGTCAAGGAA |
| | | GAAAAAGAGAAACTGAAAAAACAACAGATACGTGATTTCAGAAAC |
| | | TATGAAAAAAAGCAGTGGAAACTGCTAAAAGCAAAAAACGAAGCA |
| | | GCCCCCAAAGGCCAGCACTTTAATGTGAAAGCTGAAGTTCAGAAA |
| | | CGATTGAACGAGAAACGCGAGGAGCAGCGCAAAGCATTAGATCTT |
| | | TTAGTTATGGATGTTAAACAGAAGTTGGAGGGTAAACTGAGAAAA |
| | | GTGAATGATAATGAGCGAGCAATACGCCGTTATAAAACACAGGAT |
| | | ATCCTATTGCTCTTCATGGCTCGTGAAATTCTAAAGGCAAAGAGT |
| | | CAGGACGAGGACTTTACCAAGGGATTCTGTCTGAAATATGTTATG |
| | | AGCGACTCACTGCTCGACAAACCAATCGACTTCCAGTGGACGGTG |
| | | AATTTCCAGAATAAGGAAAAGAAAACTATCGCCAAGACTATTGAG |
| | | CAAAAGGACATGAAGATGAAAAACTATGGTCAGTTCTACAAGTTT |
| | | GCCAGCGACCACCAACGTCTGTCGTCGCTACTATCACGGCTACCT |
| | | GCCGATATATTCGAGCGTGCAGAAATCGAAAATGAGTTTGCATAC |
| | | TATGACACCAACCGAAGTGAGGTATTCCGCCAGGCATATATCATC |
| | | GAGAGCAAAGCCTACCAGTTAAAGCCAGAGTTGACTGATGATGCG |
| | | AATGCCAATGAAGAGTGGTTTACCTATCTTGATAAAAAAACAAAG |
| | | AAGCTTCGCGCTAAGCGCAACAACTTTGGCGAGCTGCTGAAGATT |
| | | CTGGCCGCTGGTGGCGATGGCGTACTGGACGATGTCGAGAAGAGT |
| | | CTGCTGCAAAGCACACGCAATGCCTTCGGACACAACACCTACGAT |
| | | GTAGATATGCCTGTTATCTTCAGCGGGAAACTGGATAAAATGAAT |
| | | ATCCCTGAGGTGGCTAACGGTATTAAGGACAAAATCATAGAACAG |
| | | ACTGAACAATTGAAGAAAAACGTTTAA |
| 9 | Cb9 | ATGGCTATATTTGTAATTAAGGTAGAAATCCAACGACAAATGACA |
| | ContigID: | CTCGCATTCAAAAACGAAACCGAACAGAAACCCGTTCTCGGAGCA |
| | k127_1483864 | TACGCCGCCATGGCAAGAAACAATGCCTTCCTTACCGTGATGGAT |
| | | ATCATGGACCAATTGCATATCCCCCGGACTGTGCTCACCGATAAT |
| | | TCCGGAAAAGAAGTGGATCCGGAATCCCATATATGGAGACTCAAC |
| | | TTATTCCCGAAGAACTATAGGCTTCTTCCCGAACAGGAGGCCAGG |
| | | GCTTCCCGGCTCCTTCGCAATCATTTCCCTTTCCTCGATCTCGCG |
| | | GATGACCTCAAGGATGTTGAGAATGACCATGGACAGGCAGCAAGG |
| | | AAAAATGTCTCCTATGAGGACTTGTGCAATTCCTTTCTTACCATG |
| | | ATGGAAGTGCTGACGCACCTTAGAGATGTCAACCTTCACTACAAG |
| | | ATCAAGGACGAAAGAATCGCGGATTTCTATTTCCGCCGTGCGGAG |
| | | AAAGAGACCGGGCACATCCTCCGGGAAGTGCTGAAAGCAGCACCC |
| | | CGAAAGATCAAGGACCGTTACAAGGGAACAGCGCTCATGGATGAA |
| | | ACCTCGTTGTCCTTCTTCACGGACGGGAATTACATCCAAAAGGGA |
| | | AGGAAATATTCTTTCAATTCCCGATGGGCCTTCAATCCGCAGCGG |
| | | CAACCGCAGCCGTCTGAAGTGAAAGTCTTGAAGAACAACGCTCCC |
| | | GTCATTGACAGAAACACGGGCATACCGCGCATGTTCGAAAGGCTA |
| | | TCCACTTTCGGCGAGATCCTCTTCATCGCCCTTTTCACGGAGAAA |
| | | CGGTATATCCCCGACCTTCTTAGAGACAGTGGGCTTGACAACAAC |
| | | TTCATGGCATCAGGCGATAACGGAAAAATGTCCCAGCAGAGGATT |
| | | ATTCGGGAGATCATCTCCGCCTACAGCATCCGGCTCCCGGAACGC |
| | | AAGTTGGATATCGAGACCGGGGCGACACAAATCATGCTGGACATG |
| | | CTGAACGAACTGGCGCGCTGCCCCTCTGAACTGTTCGATGTTCTC |
| | | CCGGAAAGCGAACGAAGATCCTTTGAGATAACGGGCAGTGACGGC |
| | | TCCCAGGTCCTTATGAAGCGTTATTCGGACAGGTATGTCCCGCTG |
| | | GTGTTGCGCTATTTAGACGTGACTGAAGAATTCAAACGGCTCAGG |
| | | TTCCAGGTCAATACCGGTCTGCTCCGGTACGAGCACCACGGACCG |
| | | AAGGAGTATATGGACGGTGTCGCCCGGTTCCGTATCGTCCAGTCA |
| | | AGCATCAATGGATTCGGGAGAATCCAGGAGATGGAAGCGGCACGG |
| | | ACAGCCGGCGCGACTTACCTCGGCTTCCCCCTCCTGAAAACTGAT |
| | | GACGATGGAAACATGACCGAGATGCCCTACATTACCGACTCGGCG |
| | | GCCCGGTATGTCCTGAACGGCGATCTGATCGGCTTATCATTTGGG |
| | | GACGCCGCTCCGAAGATTGATACCCTCCCTAATGGGGCGGGATTT |
| | | AAGTACAAGGTGTCCTGTCCTCAACCGGACTGCTGGCTAAGCCGG |
| | | TATGAGCTCCCGGCGCTGGCCTTCTATACGTTTCTTTCCAGAAAA |
| | | TACCATATCTCACGGAGTACCGAGGATATCGTCGAGGATGCTTTA |
| | | AATGAATACCGGGCCTTCTTTGCAGGTATCGCTGATGGAAGCATC |
| | | ACATCCATGGACGGGGTCGGCATCCCCAGGAAGAATATTCCGGAG |
| | | AAGTTGCTTGACTATCTGGAGGGGAGGGGCAAGAGAACGGATTTC |
| | | AAGAAATACAAGGAGGGTCTGGTCGCAAAGATGCTCCTCAATACG |
| | | GAAGCACTGCTGTCCCGCCTCAAAGAAGACTTGAAGGTAATTGGA |
| | | ACGAAGGATAACCGTATCGGAAAAAAATCCTACGTGCGCATCCGC |
| | | CCCGGTAAGCTTGCCGAGTTCCTCGCAGAGGATATCGTCCGGTTT |
| | | CAAGGTCATCCTGCGGGGATGCCTGAAAAGAAACTGACAGGACAG |
| | | CAGTACAGCATCCTACAGGGAATGATTGCCACTTTCCATGAAGGA |
| | | CTGGCCGACGCGTGTCGGAATGCCGGCCTGCTAGACGGAGATTCC |
| | | GCCCATCCATTCCTTTCCTTGGTATTCACACGCCATGCACAGGGA |
| | | ATGACATCCACAGTCGATTTCTATCGGGCATACCTTGAGGAGCGG |
| | | CGTACATACCTAAAGGGCGTGGTTCCGGACGAGGCTCCCTTCCTT |
| | | CATCAGGAAAGAAGGAAATGGTCGGCCAACAAGGATTCTGCGTAT |
| | | TACAAGTCCCTCGCATTGCGATACATTAAGGATGAGAAGACGGGA |
| | | GACAAGGTCGGTGTCTTCTTACCGCGGGGACTGTTTGACAATGCG |
| | | GTTCACGCCATCATCAAAGAGCATTGCCCGAACACGTCGAAGGTC |
| | | ATCAATGCTTCAGAACGGGCAAATATGGCCTTCATCATACTTACC |
| | | TACCTGGAGAAAGAACTGGATGACCAAAACCAGGGATTCTATTTC |
| | | AACGAAGAAAGGCTGAAGGAGTACGGGTTCTCGAAAGCCATACGG |
| | | AAAGAGTTGGAAGAGAGCGGCATGAAACGACTGTCTCATGTCCTG |
| | | CGTCTTGAGAGGAACACCAATCCTTCTGGACTGTATTACGAAGCT |
| | | CTCAGGGAAGAGTCCGGCTGGAAAGATGACCGTAGAAAGGGTGGA |
| | | CAACTGGATAGGAAGACCGAGGAATTCGCTGAAAAACTGCGCCAT |
| | | AGTTATAAACGTATGTGCGATAATGAAAAGACTATCCGTCGATAC |
| | | ATGGTGCAGGACATTACACTTTTCCTTCTGGCAAGAAGTCTTGTC |
| | | CGCATCGCAGGGAACTCCGTCAACCTATGGTCAGTAGGGCCAGAA |
| | | GGGAATGGAATCCTCGACCAGTGGGTCGACGTAATGACACCGTAT |
| | | AAGAAATACATAATCCGTCAGAAGGGTATCAAGATCAAGGACTAC |
| | | GGTGAGATCTACAAGATTCTTAAGGACAGGCGGATTGACTCCCTC |
| | | CTCCTGAACCAGAAGAAACGGGTGCCGGAGGCTATCGACCTGGAG |
| | | GAAATCAAGGAGGAACTCGTCACATACAACCGCAAACGCGCCTCC |
| | | ATGGTCAGCGCTATACAGGTGTATGAGAAAGGTGTCTTCGAGGAG |
| | | AATCGGGAGCATTTCGACAGTATGACCAGTCGCTTCGGCTTCAAG |
| | | GAAATCCTTGAGGCAGACGTAAGGTCAAGTTCCTTGACAAAGGAG |
| | | GCGGTTAAGAAAGTAAGGAATGCGGTCTCCCACAATCAGTATCCC |
| | | GACCGTATGGTGATTAAGGATGGCCGTAGCATGGTACTCTACTCC |
| | | CCCGATCTTCCCGATATGGCGAAGGGTATCGCCGAAACGACCGAT |
| | | AGACTCACGAAATATGGAACTGATATTAGTAAGCAAACTGACGAG |
| | | TGA |
| 10 | Cb10 | ATGTTACAAACAGAAAAAAACGATCGTGGAGCTTTTTGGGCTGCT |
| | Contig ID: | TATTTCAACACGGCAACGAACAACGTTCAGGCGATTCTTCAATTC |
| | Cas13/21_ | GCCGGAAAAAACGTTCAGCTTGAGGAACTTTCAAATCAGGAGTTC |
| | contig | AAGCTTTCAGCAAACGGAATCGGCGATGAAGAGACAAAAGAACTC |
| | -81_616 | GAATGGTCGGCCGAAAGCTATCCGGCAATTCAAACGCTGAAAATG |
| | | ACAGGCGATGAAGTCAACATTCCAGAACAAATTCGAATCATGAAG |
| | | ACGCTGTCCAAGCATCTTCCTTTTATGAAGAGAATCTCAACTCGC |
| | | GTGCAGAACGGCGTTCGCAAAAATGGCAAAACCGAAAAAGCGGGA |
| | | GAGGAAATGACGCCGCAGATGTTTGCTGAAATTCTTATTGGGTAC |
| | | GTGAATTATCTTTACGACCTGCGTAATTATTTCACGCACTACAAA |
| | | CACGTTCCCGTCTCCCGGAAGAATATGCAGTCCGAGTATTTGGGG |
| | | ATTCTCTTTGACGCCAATGTTGGAACGGCCAAAGAGCGCTTCTAT |
| | | TCGGAAGACAAGATTGCCAAAGACGACAAGCGGTTTAACAATTTC |
| | | CGTATGCACAAGGGCGCGGAATCCGTACCGGACAAAAAAGGCGGC |
| | | ATGAAAAAACAGCCGAAACTCAACAAGGATTTCCTGTTTTATCTT |
| | | TGGGAGACGGATCCAATCACGCCCAAAGGCAAAGACAATCCGTAT |
| | | CTCGAACTGACAGCTCAGGGGCTGGCGTTTTTCCTGTGTCTGTTT |
| | | TTGGAAAAGAAGTCCGCTAACATGCTTTTGGACTCCGTCGGCATT |
| | | GAAGAAGACGCGGAAAGTTTGATTGACGGATTCGGTTTCGAGAAT |
| | | AATTCCGGCGATAATCGAACGCTGCTCAAACGGATTTTCACCATT |
| | | ACATGCGCCCGACTTCCGCGCACTCGTCTGGAAAGCGAGAATCTG |
| | | ATAAGCAATCAGGTTCTTGGCCTGGACATTATGAACTATCTTCAT |
| | | AAATGCCCGAAAGAGTTTTACAATCTGCTTTCACCTCAAGATCAG |
| | | CATAAATTCCGGACGCTTTCAGACGATCACGGGACGGAAACGCTG |
| | | CTGAAGCGTTTTGACGACCGGTTCCCGTATCTGGCTCTGAACGTT |
| | | ATGGACAGATTGGAGTGTTTCGATTCTCTGCGATTCTGCATTGAC |
| | | ATGGGAACGTTCTATTTCCGTTGTCATTCTCGCGTTCAGATTGAC |
| | | GGCTCCCGTCTGGAAAACCGCCGATTAAAGAAGAAGCTGACGTGT |
| | | TACACCCGCCGTCAGGACGCGATCGAGTATTTTCAAACCGAACGC |
| | | GCGGCGGAAAACACGTTCTATCAAACGGACAATCTTGCTCCGGCT |
| | | CCGAAGGCGTATCGAACGGACATGCTTCCGCAGTACGACCTCGGC |
| | | CGCGGTCGGAAGCAGGAAAACCGGATTGGAATTGCTCTGAAATCG |
| | | TTAGACGACAGCCGGCCGATGTTCAATCAACCGACGCTGGATCCC |
| | | GCCGGGCAAATCAAGCCGAAAACGTACAAGCCGGACGCGTGGCTG |
| | | TCCACGTACGAACTGGCTCCCGCGCTGTTTTTGTCTTTGCATGGG |
| | | AAAGGCAGCGACGTTGAAAAGCGAATTGAGGAATTCATTCGTTCC |
| | | TGGAAGGGGTTTGCAGACTGGATGTCCAAGGCGTCAAAAGAACAA |
| | | CTCAAAGAATTGCGTTACAATTCCGCCAGGGAGGAATTCGAAGTT |
| | | TTTAAGTCCCGCTTTGAAAAGCAGTTTGGTTTGTCGGTAAACGAT |
| | | ATTCCGGACGAATTCCGCTATTATCTTGTCAACGGCAAGATCAAG |
| | | CCGATTTATATGCGATTTCAAAGAGGCAGCGCTGTTCCAATAACG |
| | | ATGGACGAAGCCGCTCAGATTTGGCTTTACAACGAATGTGACAAG |
| | | ACCCGATCGATCATTCGGAAATTTAACAATGAGCAAAGCTATGAT |
| | | TTTAAGCTCGGCAAAGGCAGGCAGCGCCGGTATACATCCGGAAAC |
| | | ATCGCCTTATGGCTTGTTCGCGATTTTATGCGCTTCCAGAAAGCC |
| | | AACGACAATCCTCGAGAAGGTTTTGGAAAACTGAAATCGTCTCCA |
| | | GACTTCAACGTCCTCCAGTCGTCTCTGGCGCTGTTCAACAGCCGA |
| | | AAAGACAGTCTGAAAGAGATTTTGAAAAATGCTCGACTGATTTAT |
| | | AACCCGTCCGGCAACCACCCGTTTTTGGAAAACGTAATCAATCGC |
| | | AGCGGGGCGTTGTTGTCAATTGAAGGATTCTTCAACGCCTATCTG |
| | | GACGAACGATTGGAGTGGCTAAGCAATGCCTCTGGACGGGAGGTT |
| | | TATCAGCTTCGCAAGCTGTACGAACGCGGAGAAAAGAAGCGCAGC |
| | | GCCGCCAAAGGCCTTCCCCCTGCAAACGTTTATTTGGGCGAAATG |
| | | GCAAAGCAGTTTCTGGACGAGTCCGTTTGCTTGCCGCGAGGTCTG |
| | | TTTGACGACCTTGTTCGTTCAGCGCTCAAGGAAATCTATCCGGAA |
| | | CAGTACGCCAAAGACGTCCCGGACGGTCAGCGCGCCAATTTCACC |
| | | TGGCTGATGCAAAAGCATCTGAAATGGTCTGGAGACGATCATCAG |
| | | TGGTTCTATAAAGAGCTTAAGCAGTCGATGACGGCTGAAGAGTTC |
| | | AAAAAGCTGTTTGAATTGCTTGGAACAACGGACGTTCGTTACGAC |
| | | AACGCAGGCAAGAAAACGCTCAATGACAAACTCGAGGAGACGTAC |
| | | TCTCAGCGCTGGTATGATTTGGAAAAAAGCTTGAAAAATGATAAA |
| | | GAATTTAAGAGCCTTTCACCTAATGATAAACAGAGCGCTATTGAC |
| | | GACCGCATGGGGCAGGAGAATCAAATGTACCGAAGCCTGCTCAAT |
| | | CGTCTTCGCGATGTGTGGAAGCGCATTCGAATGTCCAGCGTTCAG |
| | | GACGTTGTACTTCGCGACGCGGTCTGGCAGCTGTTGGGACTGCCG |
| | | GAAAAGTCTGTGCGTCTGTGCAACGTCAAGCCGGAATACGACCTG |
| | | AAAACGCAGCGCATGATACGCGACGACGCTGGAGACGGCAGCATC |
| | | TCCAACGATGCAATTCTTAATGAGACGCATTCGTTGGAAAATATC |
| | | ATCGATCTTCCCAAGGGCTTGGGAACAATCGTGCTCAAAGGCGAA |
| | | ATGAAGCTCAAGAATTTTGGAAACTTCTACCGCATGCTTTCCGAT |
| | | CCCAAACTGCCTTCGTTCCTTTGCCTGTACAAACAGTTTGGGTTT |
| | | AACGCCGTTGATTACAGCTATCTCGAACTTCAGGAGTTTGAGTTC |
| | | TACGATCGCAAGTTTCGTCCGGCTGTTTTCAATTGGGTTCATCAG |
| | | CTTGAGGAGACGGTGTTGGAAAAGTATCCGGATTTGCCGATGAAA |
| | | TATGGCAAATTTGTTGATTTTTGGACAATTGCCGGTAAAGCAAAC |
| | | GGCGGGTATATTTTCACTCAATTATTGTTGACGTCAATTCGCAAC |
| | | GCCGTTTCGCACCAGTACTATCCGGAATTCTGCGTTCCGTCAGAT |
| | | TGGACAAATCAGAAGGATATCGACACGTACTCGCCGCAGTTCAAA |
| | | GATCAGCTTTTAAGCGTCAAGGCAGAATTCCTCAAACGACGTTCT |
| | | GAAGACAAGGACGCATTGTTGTCAGAAACGATTTACGACTACGCG |
| | | CAGTCTCTGTTTGAAAACGCAATCGCCTTTGTTGAACAGCAACCG |
| | | TAA |
| 11 | Cb11 | ATGGCAAATTTTCAAACACCCCAAAGGCATATTTTTGGTACATAT |
| | ContigID: | CTGAATATAGCTCGTGCCAATTTTTACAAAACGATTCTTCATGTC |
| | k141_16137484 | TTTTCTGCATCAGGCATTGACTGTTACACAAAAAGGGGTGACCTC |
| | | TTTGTAAGAGAAGACACCGTAGACAAAGTCATTAGTGCATTATAC |
| | | TTGATTGTTAACGGAGAGAATGCAGGTTATCACGCCATCAAAGAA |
| | | ATCGTTAGTAAGAGCTATGACAAGCGATGGAAAGAAGACAAAGCA |
| | | CTACAAGGTAATCTTTCTGGCAGTGAACTGAAAGCTAGAAAAGAA |
| | | GAGTTTAAATCGCCTCTGAAAGATGAGGGGCCTGATGGCGAAGAT |
| | | GCAAGAATTTGCAAATCATTCACATTAGGAAGTGAGCAGGAAGAG |
| | | CGAATGAGGAAATTGCTTTTCCGGCATATACCTCTATTATCACCT |
| | | ATTATGGCAGATGTTGTAGCCATGCAGTTCAAGGAAACTACCAAT |
| | | GAGCATCAAGAGGCAAATAAAACGTTACATGATGCAACGCTTGCC |
| | | GATTGCTTCAAAGAACTGAGCAACATTGCAAGGTGTCTCAGTGAA |
| | | AGCCGCAATTTCTACACACATAAAAATCCATACAACTCAATAGAA |
| | | GCCCAAAGAACTCAGCTCCAATTACAGAAAGTCATCGCAAACAAT |
| | | TTGGATAAAGCTTTCATTGGCAGTCGTAGAATTGCGAAGAAACGT |
| | | AACAGCTATTCTGAGAAAGACCTGGCATTCTTGACTGGTCATGAT |
| | | AATGATTGCAGAATGGAAGAGATATTTGTACTTGATGAAAATGGT |
| | | AACAAAATATGGAAAGTAGAGAAGGATAAAAACGGAAAAGACAAA |
| | | CTCGATAAAGACAAGAATCCCATTTATGTCTATAAGAAGGTTAAA |
| | | GCAAAAGACGGGAAAGGTAGAGAGAAACTTGATGAAAAAGGGAAG |
| | | CCAGTTTATGAGACTCTACGTGAAAATGGGGAACCTGTACATGAA |
| | | TATGAGAAAAAATTTGTAGAACGGAAAGATTTCTATTTCCGTATT |
| | | AGGGGTAAGCGTGAAGTATTAGCTCCTGATCTTACCCCAACAGGA |
| | | GAAGAGTGCGATGGCCTTTCTGCATTTGGAATGCTTTATTTTTGT |
| | | TCTTTATTCTTGTCAAAAGAGCAAACGGCACAATTGTGTACAGAA |
| | | TCTCGTGTATTCGTCACGAGCCCGTATCAGCCTGCTGGTAATTTA |
| | | AAGAATAATATCATTCTTAATATGATGTTCGTATATGCCATACAT |
| | | ATTCCGCGTGGCAAGCGTCTTGACAGCGAGACTGACAGCCAGGCA |
| | | TTGGGAATGGATATGCTAAACGAACTACGTCGCTGCCCTATAGAA |
| | | CTTTACGATGTACTACCATCTATCGGAAAACGGGATTTTGAAGAC |
| | | AATGTTAAACATGAGAACAATAGAACGCCGGAATTGTCCAAACGT |
| | | ATTCGATTAAAAGACCGTTTCCCATATCTTGCCATGCGCTATATT |
| | | GACCAACAACAATTATTTAAAAGAATACGCTTCCATGTCCGGCTC |
| | | GGTAGTTTTCGTTTTTGCTTCTATGATAAGACTTGTATTGACGGA |
| | | AAGTCACATCCTCGGCAACTCCACAAAGATATTAATGGCTTTGGC |
| | | CGGTTGCAGGACATGGAAAAAGAGCGTAAAGAACAATATGGGCAC |
| | | TTTTTCCAACAATCAAGAGAACAGTCCATATGGCAGAAAGACGAA |
| | | AATGCCTATGTAAATCTAAAGCAATTGGAACCAATTAAAGCTGGT |
| | | GACCAGCCGCATATCACCGATATGTTTGCCCAGTACAATATCCAT |
| | | CAAAACCGAATCGGGCTCTTTTGGAACACCGATGAAGAATGTAAA |
| | | CTTGTTAATAAAACAAATGCTCAGGGAGAGATAATTTACAACGGA |
| | | TATTATCTTCCACCGCTTAACTACGTGGATTCTCCAACAGAAAAC |
| | | AACAAACACAAAAGAAAAGCACCTGTTGATATGCCGGCACCACTA |
| | | TGCTCATTGAGCGTTTTTGAGTTGCCTGCTATGTTGTTCTACAAT |
| | | TTTTTAAGAAACACCGATAGTCTTGGGGGAGAGGAATTTCCCGAT |
| | | GTTGAAGAAATTGTTATTAAACAATACGACAATATCCGAAAATTT |
| | | TTCTTAGAGGTCAAGGATATTCAGCCTACTGACAATATAGAGAAT |
| | | CTGGCTACCATCCTAAACGCTTATGGTTTGTCTAAACAAAGTGTT |
| | | CCAAAGAAGATATATGACTATTTGTCTAATAAGAACACATTAATT |
| | | AGTAAAGACATTCGGAAATCTACAGAGAAAGAGGTTAAAAACCGC |
| | | CTTAGAAGAGCAATCATTAGAAAGCAACGCTTTGAACAGGATCAG |
| | | GAGCACATAGAAAACACCAAGGATAACAAGATCGGAAAAGATAGC |
| | | TTTGTCAGCATCCGCTATGCCAAAATTGCTGAGGAACTGGCAAAG |
| | | TCCATGATGGAATGGCAAAGCGGCAATACAAAGATGACAGGATTG |
| | | AATTTCAGGGTTCTGACTGCTGCTCTGGCAAAATTTGGCGATGGT |
| | | GTAATAAAGCGAGATACGATCATTTCAATGCTTCAAAAGGCGGAG |
| | | ATCATGGGAGGAGACAACCCACATTGCTTTATTGAACAAGCAATC |
| | | GAGCAGGAACAATATGACATTGAAGAATTCTATCTTGATTATATT |
| | | AGTGCAGAGATTGAATACCTGAAGCGTTTTCTGATGATTGATGGA |
| | | AAAACGATTATATTGAAAGACGAGCAATTGCTTGATGCTCTTAGG |
| | | AAAGATAAAGAAGTCCATGATGATGTCAGGATTCGTCTTAAGAAC |
| | | GATGTTGATTTCGGCCAACTCCCTTTTATCCACAAGTCACGCTTG |
| | | CGTTGGCAACAAAGCAAAATCGAAGAATTGGCAAACAGATATCTT |
| | | TATGTAAAAGAAGAAGGTGAAGAAACACTTGGACGTGCCACTCTT |
| | | TTATTGCCTGATGGTATGTTTTTCCCATATATAATGAAGGGATTT |
| | | CAGAAATGTCATCAGGAGCTAACAAACAGTATAGAAGCCCTTTCT |
| | | GATGAGCAGAAAAAAGGAATTGAAAACAACGTGGCTTATTTGATA |
| | | AATCTCTATTTTGAGTCTAAAGGAGAGAAGTCCCAATCGTTTTAT |
| | | GATTCCACAGAACCTTCACACTATAATGACAATATAAGGCAATTG |
| | | GCACCATACAAATATGCTAGATCTTATGAATTTTTCAAGATAATC |
| | | AAAGGCTGGCAGATTCACCTGTCTTGCGATGAAATGAAAAAGAGG |
| | | TTGACTGGAAAAAAGACCATCATCGACAACAAAATTAATGCTTTA |
| | | AAAGAGAAAGGAAATTACATTTCTTTAGAGGAAGCCAAAAACGCT |
| | | CTTCGAAGAAAACTTCACAACACGTTCCGCGACATGCAGGACAAT |
| | | GAGCGTGTCATTCGTCGTTATAAGACACAAGATCGCATTCTTTTC |
| | | CTTATGGCGAGAGATATGATGGGCGAAATTGTTAACAAGAAGGCG |
| | | GACTTGTTCAAGTTGGAAAATGTATGCAAGGATGATTTCCTTAGT |
| | | CAGAAAGTTAAGGCATCCATCGCGGTACATTTATCTATGGGAGAG |
| | | GTGTTTAAAATCCAGAAAGATGAGATGGCAATAAAAGATTATGGT |
| | | AAATTGTATCGGCTATTGAGAGATGACCGAATAACCAAATTGTTA |
| | | TCTTATGCCTTGTACGAAACAGGTGAAACAATCGACTATGATGAC |
| | | ATCACGGATGAACTGAAGGAATATGAAAGTTGTCGTTCTGCTGCT |
| | | TTCGAGGCAGTTCAGATGATAGAAGATACAAGATACCAACAAGAC |
| | | AAAGAGGTGCTTTCAAATCCCAATGAAAACAACTTCTATTGTGGA |
| | | AACATACGCTATAAAAATGGAAAAGATAACGGAAGAGAGAATGAG |
| | | GCGAAACGAAACAATTTTAGAACATTATTAGAAGATCTCCAAAAA |
| | | TTTACACCAGAACAAATGGAAATGTTTAGTCAGGAAGATAGAGAA |
| | | TTGATTATATCTATCCGTAACGCGTTTGGGCATAATAGTTACCCA |
| | | AAGCAAGTCGATTTTGAAAGATTAATCAATCAAGAAAGGAAGAAT |
| | | AATCCCAATTTTAAAATCGAGCTAAAACAAGTTGCATCTTTCATT |
| | | CTGGATAAATTGGAAGAATATGTAAATCAAGTAAATCCCCAAACA |
| | | TAG |
| 12 | Cb12 | ATGAGCTACACCCCCTCCTCCCGCCCTCCGCGCCGCCCGCAGATC |
| | ContigID: | GTCGAAGGTTCGCGCAACAACGCCCTTCGCATCCTTAAAATCACG |
| | k127_333529 | CCCGACGAACAGACCGCGTTCGTGACCTACCTCAACTACGCCGTC |
| | | AACAATCTCTCCGAAGTGGCTGGCGTCGCGTTTTCGGACGAGAGG |
| | | AAGGTCCGTGCCGACGTCTTCAGGGGAGAACCGGCCGACATCCAG |
| | | CGCCGGATTTCCCGTCTGGCGGATTTCCTCTGGTCGTTCCGCGAG |
| | | AAGGACCCGTCCGCCGACGAATCGGGCTACAAGGCCAAACTCGGC |
| | | GGCGGCCACGACGACATGGCGGTTTGGCTAACCGAAAAGATTGTC |
| | | TCCCTCCGCAACTTCTTCTCCCATGTCAATCGACAGGACTGCACG |
| | | CCGCTCGTCATTTCGCATGACGAATACGTCTTCGTCGAGGGCATC |
| | | CTCGGCGGAGCCGCGCGCGATGCGGCGATGGGGCCGGGGCTCAAT |
| | | CCCGCCAAGGCCCAGAAACTGAAGCTCGCAACGCACCACGTCAAG |
| | | GAGGCCCACACCTACGAGTTCACACGCAAGGGGCTGGTGTTCCTG |
| | | ACGTGCCTCGGCCTCTACAAGGACGAAGCCGAGGAGTTCTGCCAT |
| | | CTCTTCCACGAAATGAAGGTCCCGGACCGCATCGAGGACGCGGAC |
| | | CTCGATGAAGAACTGCCGGACGGCCGCCACCGCGCCGTCCTCGGA |
| | | CTGGAGGACTTGGACAAGTTCGCCGGTCTCAAGGGCAAGGGCCGC |
| | | GCTTTGATCGAGCTGTTCTCGTTCTACAGCTACCGCCGCGGACGC |
| | | CAGTCCCTGGACGCGGCGAACCTCGACTTCATGCAGTTCGCCGAC |
| | | GTCGTCGGCTGCCTCAACAAGGTTCCGGCCCCCGCGTTCGAATAC |
| | | CTTCCGCTCGAAAAGGAGCACGCGGAACTCGAAGCCCTCAAGGCC |
| | | GCCTCGACCGAATCCGAGGAAAACAAGCGCACCAAGTACGTCCTG |
| | | CGCCGCCGCGACCGCAACCGCTTCCTTTCCTTCGCGGCCGCCTAC |
| | | TGCGAGGACTTCGGCGTCCTGCCGTCCGTCCGCTTCAAGCGGCTC |
| | | GACGTACGCCGGACGACCGGCCGCCACCAGTACGTCTTCGGCCCC |
| | | GGCGCCGACGCGGAAACGGACGAGGAGGAAAGCAACCGCGTCCGC |
| | | CTCAACCGCCACTACGCCATCCGCCGTGACGCGATCCCGTTCGAG |
| | | TTCGAGCCGGACCGCCACTACGGTCCGGTTCGCATCGGCGCGCTC |
| | | CGCAGCGCCGTCAGCGCCACGGAGATGCGCAGACTTCTCTACCTC |
| | | CACGCCCAGGGCGCGGACATCGACGCCGTGCTCCGCGGCTACTTC |
| | | GAGGCCTACCACCGCGTCCTCGAACGCATGGTCAACGCCGGCTCC |
| | | CTCGCCGAGATCTCCCTCGACGACGAAACCTTCCTCGCCGACTTC |
| | | GCGACGATCTGCGGCACGTCGCCGGAAGCGTTCAAGGCGGATCCG |
| | | GAGGCCTTCCGCAAGTTCGTTCCGGAGAGTCTCCGGCGCTACTTC |
| | | TCCAAGGACGCCGGACCCCGTTCGGAACAGCGTCTCCACGCGCTT |
| | | CTCTGTTCGAAGCTCCTGGCCGCGGTCAACCGCACGTGCGACATC |
| | | CTCGTCCGTCACGACGCCCTCGAGGCGTGGCGCGAGGCGTCCCGT |
| | | CCCTGGCTCGACGCACGGGACGACTGGCGCGGCCGGATCGACGTC |
| | | TGGCGCAAGGCCAATCCGAAGAAAGATCTTCCCGACGAACTCAAG |
| | | ACCTTCGAGGCCTATCTTGAAACGCTCCCGGCCGACGAGCGTCCC |
| | | GCCCGTCCGGAGGAACCCCGTTGCCGCGTCGGCGAAGGCGAAGGC |
| | | GAAATCACGAATCCGCCGACCTGGTGCCGGTTCTCCGACGCCGAC |
| | | TACGTCTCGTTCGTCTTCGACTGGTTCAACCTCTTCCTTCCGAAC |
| | | AACCGCAAGTTCCGCCAGCTCCCCATCTCCGAGCAGCACAGGGAA |
| | | GGCGTCGAAGACCATCTTTTCCAGATGGTCCACGCCGCCATCGGC |
| | | AAATTCTCTCTCGACCAGAAGGGGCTCTGGTCCCTGCTCGAAAAG |
| | | AACCGGGCGGAGCTCAAGCCGTATGCCGACCTCCTGCAATACCGG |
| | | ACGGACATACGTGTCCTTGTGGCTCCGTTCGTTCGGGAACTCAAT |
| | | CATCAACTTCCTGAGAATCGGCGACTTCCGGAATTTGACAATGGT |
| | | GTTGACAAAGACGAAAAAGAAATTGCAAGCCCGATTCTTAACATC |
| | | TTCCGTCATTTCAGGCAGAACGAGCTCTGGCAATTTTTGCAGAAT |
| | | TTCCGGCCGGAGTTGAAGTCTTGGTGTCGGAGCATGAAGAGCAAG |
| | | CTCCAAGACGCCATGGAAAAGAAACCGTCTCGAAAAGATGGCAAA |
| | | CCGCACTTCCCGAAAGTTGAAGACTTGTTTGAGGTGTTGAATCTT |
| | | CGTCGCCCCTCCCCGACCCTGGAAGACCTCGCCGTGGAGGCTGTC |
| | | AAGCTCTCGAACGACATGTTCCTCGAAGAGACGAACCGCTGGGGC |
| | | GCTGCCAATCCAATGTCCGTTTCGCGCGACGATTTGCTCGCCGCC |
| | | TGCCGCCGCTTCGGCGTCCGTCCCGGAATGCCCGTGGAGTACAAG |
| | | TCCCTCCTCAAGACCGTCCTCGGCATCGACTACGATGCCTGGCGC |
| | | CACGCCTTCGACTACGGGACCGGCCGTCCGTTCGCGGACCGCCGG |
| | | CTGGAGGACGAGGAGCACGTCGCCGCCCAGATTCCGCTTCCGAAC |
| | | GGCTTCGCCGACCGTGTCGCGGCGTCTCTCCCGCGCAAGGTCCGC |
| | | GAGCGGTTCCAGGCTCCCGGCTCCGCCGCGTTCGACTTCAACGCC |
| | | GCCTTCCGCGCCTGGTCCCCGGATCCGGCCGTTTCGCTCCGCGAC |
| | | TTCTACGACGTCAAGCCGCTCGTCGCGAGCCAGATCGCCCGAAAG |
| | | CACCCGCCCGAGGGGGGGGCGGCGACCGCCGACCCCTTCGCCGCC |
| | | CTTTCCGCAAAGACGCTCGATTCCGCCGTGCGCGAAATCAAGGAC |
| | | GCCGAAAACCAGGACAAGGTCCTCCTGTTCGTGGCGATGAAATAC |
| | | TGGGAGCGCTTTCGCGGCGGCGACACCTACTCGACCGGCAAGCTC |
| | | AAGATGCCGTTCTCGGAGAAGACCACGCTGCGCGAGTTCTTCGAC |
| | | ACGCCCGTTCAGATCGAAAAAGATGGCCTGACCGTTTCCTTCCGT |
| | | CCCAACGACGTCAACCGTCCGGCGTTCGCGACGCTGTTCGGCAAT |
| | | TCCCGCGCGGCGAAGGACTACCGCGCCAAGATCGCGAAGATCCTT |
| | | TCTCCGGACGGGTCCCGCACGGCCTTCGACTTCTACGAGATGGTC |
| | | GTCGCCTTCCGCGAGCAGAAGGCTCGCGACCGCCACGAGCGCCTG |
| | | GCCTTCACGCCCTACGCCGTCCATTTCGACGCCCTCTGCGAAATC |
| | | CCCGCCTCCGCCTACGAGAAGGCGGCGGAAGGTCGTTTCGGGGAG |
| | | GCGAAGACCGAGGCGATCCGCGCCATGGAGTTCGAGCGCTACAAG |
| | | GCGGTCCTCCCCGGCTTGACCCGCGAGGACTACGATGCCGTCGCC |
| | | GACGCCCGCAACGCCGTTTTTCACACAGGTTTCAAGTTCGACTGC |
| | | TCCAAGGCGATCGCCGTCTGCAAGCGCCTCGGCGTCCTCGGAATG |
| | | ATGCCGGGCGAAGCCGCTCCGGCTGGCCGCAGAACGTCGTCCCAT |
| | | TCTGGGCCGAAATGGTCCGGCCCGCGCCGGAACGGCGGTTCGAGG |
| | | TGGTGA |
| 13 | Cd13 | GTGAGCAAGAATGATAACATCAAATCCAAAGCAAAAGCACTTGGC |
| | ContigID: | TTGAAGTCAACGTTTCAGGTTGGCGACGAGGTCGTTATGACTTCT |
| | k127_2411982 | TTCGGTAAAGGTAATAAAGCCATCGTTGAAAAGATTGTACGTGGA |
| | | ACCGACGTGCAGTCTGTTCCGACTGAGCCGAACTTCAGCGCTGAA |
| | | ATCGAAGGTAAAAAATTTGACCTTGTCGGCAGAGCTCATATTCAG |
| | | ACAAAGTCGGACAATCCTCAGTATTCAAAGAAAAGAACCGGCGAC |
| | | GATATGATAGGCGCAAAGGCGGCACTTGAAAAGAGATTCTTCGGC |
| | | GGTACGTTTGACGACAATATACATATTCAGCTCGCTTACAATGTT |
| | | CTTGATATTGAAAAGATTCTTTCTGTCCATATCAACAATATTGTT |
| | | TACACGCTTAATAACATACGAAGAAAAGACAACGCTGAAGATGAC |
| | | GATTTTATCGGCTATATGAGTACCAGAAACGACTACGACACCTTT |
| | | ATTGAACCGAGAAAGCACAACATTAGCGAAGACGCTGCTAAAAGC |
| | | ATTGACAAATCAAGAGCCTCGTTTGAAGAGTATCTTCAGCCGCCG |
| | | GTAAAGGATTGTCTGCATTATTTCGGAAACACCTTTTTTGCTCCC |
| | | CGTGAGATTGAAAAGACATACACGGATAATCGCGGCTTTGAAAGG |
| | | AAGAAAAAAGTTACCGTAAACTCGCTTATAGACGAGAAGGAAATA |
| | | TACTATATTTTTGCCCTCCTCGGCGGACTTCGTCAATTCTGTACC |
| | | CATGACAACAAAAGCGGCAGAAACTGGCTTTATTCTCTCGAGGAA |
| | | AACGGAATAAACTCTGATGCAAAGGCGGTACTTGACAAGTACTAT |
| | | AATTCTGCAGTTGCAAGGATTGACGAAAGCTTTGTTGACAACGCA |
| | | TCTAAAACTAATTTCAAGCTGATTTTTAATGCAATGGATGTCAGC |
| | | GACGAAGCGCTTCAGGACAATATAGCAAAGGCGTTTTACAAGTTT |
| | | ACGGTTTGTAAGAGCTTTAAAAACATGGGCTTTTCAATTAAAAAG |
| | | CTCCGTGAACAGCTGCTCGAACTTCCTGAATATGAAGGGCTTAAA |
| | | GATAAGCATTACGATTCCGTGCGCTCAAAGCTTTATCAGATTATG |
| | | GACTTCGTTATTTATCTGAGTTTCAAAGATGAAAAGTTTAAGAAG |
| | | GATAATGAAGAAAAGATTAATAATATCGTTAACGAACTCCGTGCG |
| | | ACCTTGAACGAAGAGGATAAAGGCAGAGTATATGCCTCTTATGCC |
| | | GAAAGGATGAAATCTGAGCTTAAACCGGCAATTGCACGCCTTAAG |
| | | TCTGATATTGACAAAATTAAGGACAGCAGGGTGAAAGAGTTTGAA |
| | | CTTGATGCGTCCGTAAAATATAGACTCTCAAAGGTCGTAGAAAGT |
| | | GTGCGCCTTAAGGATAGGGCAACATATTTTACAAAGCTCATATAT |
| | | CTGACTACTCTGTTCCTTGACGGCAAGGAGATTAACGACCTCCTG |
| | | ACTACTCTTATTCATCAGTTTGAGAATATCGCAAGTTTTATTGAT |
| | | GTTATGAACGACAGAGGTATTGATTGCCGTTTTTCGGATGGATAT |
| | | AAGCTTTTTGAATCCTCAAAGCAAATTGCGTTTGAGCTTCGCAAT |
| | | GTCAATAGCTTTGCTCGTATGACAAGGACCTCAAAGGATGACGAA |
| | | AACGCAACGCATATGATGTATATAGATGCTGCAGAGATTCTCGGC |
| | | ACCGATTATACCGAAGAGCAGATTGAGGAGCATCTTAACCTGGAA |
| | | AAGAAAAGAATGATACCAGGCACGAAAAAAGCGGATATGAACTTC |
| | | CGTAACTTTATAATCAATAACGTTATAAAGTCATCACGCTTCAAT |
| | | TATCTTGTCAGATATTCCAATCCTAAAAAAATCAGAGCGCTCGCC |
| | | GATAACGAGGGTGTAATCCGTTTTGTCCTGGGCGAGTTGCCGGAT |
| | | GCGCAGATTGACCGTTATACTTTACTTTGCGGCTTCAATCCCGAT |
| | | GCCGACCGGCAGGAAAAAACGGACAAACTTGCAAAGGCAATTACT |
| | | GGTTTGAGGTTTAACGATTTTGAAAATGTTAAACAGGGCGCAAAT |
| | | ACTGAGGGCGAATCTCAGGAGTCAATTGACAAGGCACAGAAGCAG |
| | | GGACTGATTTCTTTGTACTTAACCGTTCTATATCTTTTGACAAAG |
| | | AATCTTGTTTATGTAAACTCACGCTATTTTCTTGCATTCCATTGT |
| | | CTTGAGCGTGATGCGCAGCTGCTTGGAAGCGGTGCTGGGCATCAT |
| | | GAGCCTTATGTTGCTCTTACTCAGCGCTTTATTAATGAAGATAAG |
| | | CTCAATGAGCATGCCTGCGAATATCTTAAGACCAATATTGCAAAT |
| | | TCGGATGAATATACAATTAGAATATTCCGCAATAATGCTGCTCAC |
| | | TTGAGTGCTGTTAGAAATGCAAATCTGTATATTGACAAGTTGAAG |
| | | GAATTTAAATCGTACTATGAGATTTATCATTTCCTTTCTCAAGAG |
| | | AATATTTACGGTAAATATTGCGTTGATAAGAAATATGTTACAGCC |
| | | GATGAAAATGGAAGTAAAACAATAAGTGTTAAAATTAGCAAGGAT |
| | | TATTGTCCTCAGGTATATATCGACAAGTCGCTTGAATACTTTGAC |
| | | AAGCTCAATAAATATGGTACGTATTGCAAGGACTTCACGAAGGCA |
| | | CTCAATTCGCCATTCGGCTACAACCTCGCAAGATATAAGAATCTG |
| | | TCTATCGAGGGCTTGTTTGACCGAAACCGTCCCGGAGACAAAGGT |
| | | GAGAACACGTTTGAGGACTAA |
| 14 | Cd14 | ATGGCGAAGAAACTTAGTCCGAAAGAAATAAGGGAAGCTGCAAAA |
| | ContigID: | GCAGAAAAGATGAAAAGCATAAAAGCTGCAGAGGCTGAAAGAGAA |
| | k141_15335538 | AAAGCTGCTGAAGAAGCGAAACTTAAAGCTGAAGCAGAAAAGAAA |
| | | GAAAAAGCAGAAAAAAATGAACGCGAAAAAGCACTGAAGCGCTTC |
| | | AGACTTGATGAAAAATCACGAATGGCTCTTCCTAAAAGTGAAAGA |
| | | AAGTCACTTGCCAAAGCCGCTGGGGTCAAATCAGCTTTTGCTGTT |
| | | GGAAATGACATTTATCTTACTTCATTCGATCGCGGTAACGATGCA |
| | | ATCGTAGAAAAGAAAATCACTGATACAGTGGTAACAAATCTGAGA |
| | | AGTGATGAATCATTTGAGGTAAATGAAAATACTATTACAGAAATG |
| | | TCTGTTCCGATAAAAAGCAAACGTATATCTGATCTGTACGCTATA |
| | | GCTGACAACCCGCTTTACAGAAAAGATTCAGCGACTAAAGTTCAA |
| | | CCGGACAAGCTTCTTCTCAAGGATACTTTAGAAAAACTATACTTT |
| | | GGAAAAACTTTTGATGATACACTTCACATCCAGATCATATACAAC |
| | | ATTCTGGACATTGAAAAAATACTTACCGTTTACAGCATCAATACG |
| | | ATCTACTGCTTAAACAATCTGTTTGGAAAAGAAAGCGGTGAAAAA |
| | | GAAGACCTTATTAGTAAACTGACCTATCAGATAACATATGATGAA |
| | | TTTAAAGAAAGTAAAGCTCATAATGAATTCATCGATTTTTATAAT |
| | | CTGAACACCTTAGGGTATTACGGCAATATTTTTTTCAAAGAAAAA |
| | | AAGAAACGTTCACAAAAAGAAATCTATGATATCATAGCTCTTATA |
| | | GCTACCATAAGACAATGGTGTGTTCACTGTGAAGAAGACAAGCGC |
| | | ACATGGCTGTTCAACACAGAAACCGTTTTAAGTAAGGAATTTCTT |
| | | GATATTCTTGATGACGTTTACGAAAGTCTTGTTGAAAAGGTAAAC |
| | | AGAAATTTTCTGAAAGACAACAAGGTTAATCTGCAAATACTCGAA |
| | | GACGTCCTGGAAATAAAAGACAGTGAATCACGTGAAAAACTTATC |
| | | CGACAGTACTATCGTTTCATTGTTACCAAAGAACAGAAACTTCTC |
| | | GGGTTCTCGATTAAAAAGCTTCGTGAAGCAATGCTTGAAGAAACC |
| | | GAATTCAAAACAGACAAAAAATATGATACTGTTCGTTCAAAGCTT |
| | | TACAAGCTTATCGATTTTCTTCTGTTTACAGGATACACTACTGAT |
| | | GAAGCTGAAAAAGAAAAAGCACTTTTTCTTATTAAATCTCTCCGG |
| | | GAATCATTGACCGAGGAAACTAAAGACAGAATATACAAATCAGAA |
| | | GCTGCTCGTTTATGGCTGAAATACGAAAATACTATAACCAACAAA |
| | | ATCAGAGAAGCTCTCAATGAAAAATCTATAAGCGAATTAAAAAAA |
| | | GATAAAAGCTTCGATGATAAAAGTATTACCAGCATCATAACAGAT |
| | | GAAGTATCAGGGAAAAAAGCAACTTATTTTTCAAAAACAATCTAT |
| | | CTTCTTGCTCAGTTCATCGACGGAAAAGAAGTAAACGATCTGACC |
| | | ACTTCACTTATAAACAAATTTGATAATATACGAAGTCTCATTGAT |
| | | ACTGCCGGACAGATAGGACTTGACTGTAAATTTACAGAAGAATAC |
| | | AAATTCTTTGAAAACTCCGATCAGATAAGAACAGAACTTCATGTT |
| | | ATAAAAAATCTTACTAACATGGAGTATTACGATACTACTGTAAAA |
| | | AAACAGATGTACAAAGACGCCGTTCACATTCTCGGAATACAGGAT |
| | | GATGTTTCTGACGCAGAACTTGAAAAGATCATCAATTCCATTCTT |
| | | CTTCTTAATGAAAACGGCAAACCACTCCCTGGAACCAAAGGTAAA |
| | | AAAGGATTCCGTAATTTTATCATTTCCAATGTTTTAAAATCACGT |
| | | AGATTCATTTATCTCATAAAATACTGCAATCCTAAGAAGATAAGA |
| | | AAAATTGCAGGAAACAGAAAAATCATAAAGTTCGTTTTAAGCCGT |
| | | ATAACAGACTCACAACTGGAAAGATACTACTATTCATGCAATCCG |
| | | GAACTTAAATCCGGTATCTATCCGGGACGTGATGATGCTGTAAAT |
| | | GATCTTTCTATTCTTATCGCTGACATGAAATTCGAAGATTTTAAA |
| | | AATGTTGATCAGAGCGCAAATGTTCATGACAACAACAATGCTGCC |
| | | AGAGAAAAAATGAAGTATCAGACTATAATCAGTCTTTATCTTACA |
| | | GTTTGCTATCATCTGGTTAAAAATCTTGTAAACATAAACGCCAGG |
| | | TATGCTATGGCCTTCCACGCACTTGAACGCGACGCTCGCCTTTAC |
| | | CAAATTTTCAGCAGTGAAGAGAATTATGTGGATAATTTAAATGCT |
| | | GACTATGCTATACTTACTAAAACACTTCTGAAAGACAACTATGAA |
| | | AATGCCGGAAACCTTTATCTCAGAAACAAAAAATGGAATAAACTC |
| | | ACAAGAGAGAATCTTGATAATTACATTCCTCAGGCCGCTGCAAAC |
| | | TTCAGAAACGCAGTAGCTCATCTTAACCCGATAAGAAATGCTGAT |
| | | ATGCTGCTTGAAGATATTGAAGATGTATCATCATATTATGCCATC |
| | | TATCATTACATCATGCAGAAAAGTGTTACAAACAGAACTATAAGG |
| | | GTTTCTAATACTACTGAAGACGAAAAGCGAATACTTACAGATTAT |
| | | CAGAACAAAATCAAAAGACATCATGGTTATAACAAAGATTTTGTA |
| | | AAAGCTCTCTGCGTGCCTTTTGCCTATAATATAGTCAGGTTCAAA |
| | | TCTCTTTCTATATATGAAATGTTTGATCGTAATTATCATGAAAAA |
| | | ACGTCACCGGAAACTGATGACAGCCAAAGCCCCTGA |
| 15 | Cd15 | ATGGATAAGGAAAAAACAACTGTAGAAGGAAAAAATACTAATCAA |
| | ContigID: | AAATCTGATGTGTTAAAATCGCTGGCAAAAGCAAATGGACTTAAA |
| | Cas13/23_ | TCTTCTTTTGTTATAGGGAATGAAGTTGTTATGACTTCATTTGGA |
| | contig | AGAGGAAACAGTGCGATTCTTGAGAAAAAGATAACGGGCTCGAGA |
| | -81_4932 | ATAGAAAATCTTAATCCGAACGTTGCGTTTTATGTTAAAAAACAT |
| | | ATATCAGATAGTTGTAATCCTGATAGTGGTAAATATGACGTAAAG |
| | | AGTAAACGAATGAAGGAAAAAGCTGTTGTTGATGATCCAGTATAT |
| | | GTTTCTCCGGAAAAAGCTTCAAATGTTCATGCTGGTCAGGATCTC |
| | | ATTGGCTGTAAGAATGTTCTGGAAGAACGGTATTTCGGAAAGACT |
| | | TTTGATGATAATATCCACATACAGCTGATCTATAATATTCTTGAC |
| | | ATAGAAAAGATACTTGCGGTTCATGTTTCAAATGCAACTTTTGCC |
| | | ATTAACAATATTTTAGGAATTGAAGGCAAGGAAAACGAAGATTTT |
| | | ATCGGAAATCTATCAGTGCTCAATACTTTTGATGAGTTCGAGAAT |
| | | TATGAAACGCATCCAAAGTTTGCAAACAAAAGTGCGATAAAAGAA |
| | | AATCTCAGGAAATCCAAAGTTTTTTTTGATAAGATTAAAAAAGGA |
| | | AATAAACTCGGATATTTCGGTCAGGCTTTTTATTATGCAACAGGT |
| | | ACTGGTAAAAATCTGATATTCACCAAGAAATCTTCAGAGACGATA |
| | | TATGAATTACTTGCTCTTGTAGGAAGTCTCAGACAATTTTGCGTT |
| | | CATGACGAGGTAATGGTTGATAATAAAGTAAAATCAAGAAGCTGG |
| | | CTGTATAATGCTCAAAAAGAGTTAAAGCCAGATTTTCTAAAAGCA |
| | | CTTGATGAACTGTATAGTAAAGAAGTTGAAAAAATAGACAGTGAT |
| | | TTTATTGTAAATAATACAGTTGATTTGCACATTATTCATGATGCA |
| | | ATAGATGTTATTGACGGATCAGCCGACTGGCAGAAAATCACAAAT |
| | | GAATATTATGATTTCATAATAAGAAAATGTTTTAAGAATATCGGT |
| | | TTTTCTATAAAAAGACTTCGCGAAACAATGATTGAAGAACAGATG |
| | | AAAGTATTATGCGGAAAATGCGATAGAGAGAACTGTAAAGGTTAT |
| | | GGCAAATGCTTCAAAAACAAGAAGTATGATAGCGTTCGTTCCAGA |
| | | TTAAACAGAATCGTTGATTTTATAATATTCAGGCATTATAATGAT |
| | | GAAGCAATCTTAAAGAATGTAAGTCTTCTCAGAACCTGTATGAGT |
| | | GAAGAAGAAAAACAGAAACGATTTTATTTGCCGGAAGCTAAAGCT |
| | | TTATGGAAAAAGTACAATTATGTGTTCAGAAATTATGTTCTTAAA |
| | | AAACTTAATGGCAAGTCGATAAGCGGGTTAAAAGAGAAAGCAATC |
| | | GAAAATTCCATAGATATTAACTCAGTAAAGATAAGCCTCGGTGAT |
| | | CCGGATTATTTCTGTAAGTTTATTTATCTTCTTACGTTCTTCCTT |
| | | GATGGTAAGGAGATAAACGATTTACTTACAACGCTTATCAATAAG |
| | | TTTGATAACATTGCAAGTTTTATAAGTGTTATGAAAAATGATAAG |
| | | TTATCTATTGACTGTGAATTTGTACCGGAATACAGCTTTTTCGCA |
| | | AACAGTGCTCAAATCACATCTGATCTCAGAGTTATAAACAGTTTT |
| | | GCAAGAATGCAGGCACCGGCTGAGCCGTCGAAAGATGATATGTAC |
| | | CGTGATGCTCTTGATATCCTTGGCATGGATGACCTGTCAGAAGAT |
| | | GGAAAGAAGCAGCTTGAAGATACAGTTTTATGCAGAGATGAAAAC |
| | | GGTAAGTACATGAAAAAAGAAGATAATAATCCTAAACGTGATACC |
| | | AACTTTAGAAATTTTCTTGGAAATAACGTTCTTGCAAGTACACGT |
| | | TTCAAGTATCTTATTCGCTATAATAATGCCAAGAAGACACGTGCA |
| | | CTTGCAAATAATAAAGCAGTTATCATCTTTATGTTGAACAAGATC |
| | | AATAAACAGAATCCTGAACAGATAGTTAGTTATTATAAGGCATGC |
| | | AGAGATGACAGTGACCCAGTTGCTTCGGATGCAGAAGCTAAAATT |
| | | GAGTTTCTTGCCGAAAAGATAATGAATGTCAGCTGTACTCAGTTC |
| | | AGATACGTTAAAAACGGAACTAAAGTAAGACCGGATGAGGCTAAG |
| | | GAAAAAGAAAGATTCAAAGCGATAATCGGTCTTTATCTTACAGTC |
| | | ATGTACCTGATAACCAAGAATATGGTTTATATCAATTCAAGATAT |
| | | GTGACCGCATTTCATTGCCTTGAAAGGGACAGCGAACTTCATGGT |
| | | GTTAAGTTTGATCAGAAGAAACTGCAACCGAATCTTACAAAAAAG |
| | | TTTATTGATCCGAAAACTTGTGGTGACTATGGCCTCAGAAATAAT |
| | | AAACGCGCAAGAACTTATATCGAGCAGAATATGGATAAGATGTCC |
| | | AACTGTACTAGTTACTGGAATGAATACAGAAATGCCGTAGCACAT |
| | | CTTTCGGTTATCAGGAATATGAATCAGTATATTAAAAATGTTAAG |
| | | AATATAGGCAGTTGTTTTGAGCTGTATCATTATATTATGCAGCGT |
| | | TTCCTGTTAGACAAAGAGAATATTGCTGAATCACTTAGAGAATAT |
| | | GATGATTTCATAAAGAAGAAGGGATGCTATAGAAAAGACTTCGTT |
| | | AAGGCTTTGAATACTCCGTTCGGATACAACCTTGCAAGATATAAG |
| | | AATCTTTCAATTGCAGAACTGTTTGACAGAAATGATACTGAGCTG |
| | | GAGAGAACGAATAAGCTTAGAAATGAAGCAATCAAAGCAGATATA |
| | | GAAGAAATCTGA |
| 16 | Ca1 | MKISKVDHTKSAVSVQTAQGQQGILYKDPSTEEMSVEDRVTKRAD |
| | ContigID: | ATKALYAVFNQPKDKRSISGEATTVASSFNYVIKDLKKSKSLNGK |
| | k127_1867445 | LSVESLYEAVGNELKGKHASAEEIDLAITLLLKKSLRRDSFIEAL |
| | | KLVLGKAYKGEKLNEEDKRIIKDDLIVPLIKDYDKSSIREQAVAS |
| | | IKHQNLIAQPDSKSDDAVMVISNIAGASERSTNEKEALRQFISEY |
| | | AVLDDSVRHDMRVKLRRLVILYFYGMDVVPTGDFDEWEDHVQRGK |
| | | TADLFIDFAPVGGKTDADRLKDAIRKMNIERYRYSVDAIDQDNTE |
| | | LFFEDMMINKFFIHHIENEVERIYRNTKPGDEFKRSLGYISERVW |
| | | KGIINYLCIKYIAIGKAVYNCAMAGLGSDQPDIKLGVIDRVYADG |
| | | ISSFDYEIIKAQETLQRETSVYVSFAINHLGAATVNLTEKETDFL |
| | | TLDNKQIKELAKTGVLRNILQFFGGKSVWKNFEFAPEGGTGNEEI |
| | | VLLYYLKDILYAMRNENFHFSTASINDGSWDTDLIGRMFAYDCTR |
| | | AGVGQKNKFYSNNLPMFYKSEDLERALHILYDHYSERASQVPAFN |
| | | TVFVRKNFSEILKGQNLPMPTSAEESLKYQNAIYYLYKEIYYNVF |
| | | LSSSESRDYFIKAVKSLRWENSNEENAVKDFQNRINELTGKYSLS |
| | | QICQLIMTEYNQQNSGSRKKKTAKDEQNKPDIFKHYKMLLYKSIR |
| | | EAMLKYVDDKSEDFGFIKSPVFGKDDNCIALEEFLPDYESTQNAK |
| | | LIERVKSDFRLQKWYILGRLLNPKQVNQLAGSIRSYIQYSDDVKR |
| | | RAKENGNKIHVSTESYPYQTVLRVIDLCAKLSGLTTNNIDDYFDG |
| | | SGDYLSYLARFVEYDPNDIPKIYHDEANPILNRNIIMAKLYGAGD |
| | | VITNAVEHVNTSMIRDLESYEKKTLGYRSSGVCKDKDEQETLKKY |
| | | QELKNRVELREIVECSEIINELQGQLINWCYLRERDLMYFQLGFH |
| | | YTCLKNSSDKPEMYVKAKTVDGTIDGFILHQIAALYTNGLKLYSC |
| | | GKAVRDDNRKIIHYDLSSGKELKGNDKSAAGKKITDFMGYTSLAL |
| | | NRTENDILPISGDFYYAGLELFENVNEHENIISLRNYIDHFHYYA |
| | | KHDRSMIDIYSEVFDRFFSYDMKYRKNVPNMMYNILLSHFVKAQF |
| | | VFGSGMKESGEKTKSQARFDLKDKAGLEPEQLTYKVANSEKPVQL |
| | | SAKDKQFLKTVALLLYYPEKKTFPEGMYADTRFVEGTSSNKRNNN |
| | | SSGNRHGNGNHNVGGHNKGYNQGRKNGNWSKDKSGDRNAGKKQTN |
| | | KNRKDSTSVYKDEGFSNRINIPSEYYSQKPGKK |
| 17 | Ca2 | MKLSKTGKNGWHHRNGVKVNNSKQEGFVYSIPHNDGESTDKFVED |
| | ContigID: | RKKDFKRLYKVFPSVEKARNISEEIAAVIDKTIRNKRTEIWTGKN |
| | k127_4200118 | DYSEMACRFRNLLQRESMFRQPVEVKTAEYMVYGLLRSSLRSEKT |
| | | EKDLIDFLCHVNDKSSSAGAIFMQELTRDYMGEKINYKSIINQNL |
| | | VIQPVKTESLKDNIDQEDVLLTVSERKNPEDSAKNYKSIENKALR |
| | | SFLLEYASLDDNKRKDLRKKLRRIVVLYFYGKSEADGLGENFDEW |
| | | NDHESRRACEEKFIEFDESTKDNFRSKTSKLIRNANITAYRTSKE |
| | | IIENNHDGLYFANPDYNFLWLRHLSREVERLTSNINADKTYKLNK |
| | | GYLSEKSWKGIINYLSIKYIAIGKSVFNFASSGVDSDGSDIQIGE |
| | | VNREFQNGISSFEYERIKAEETLQRESAVKVAFAARHLASATMNL |
| | | TPEDSDMLLFDKDKMSQNLKDTGRVLADVLQFFGGQSVWKDYIIK |
| | | ETEKYSSEEEFGTDLLYNLKKCVYALRNDSFHFKTLNNKADWDTD |
| | | LVAGLFEKDCENMVGLDKDKFYDNNLYKFFKQEDLKKVLDKLYDK |
| | | IHDRASQIPAFNTVFVRNNFSRFLLSKGITRSFASEEQGRQFASA |
| | | VYYLFKEIYYNDFIQSGNAKTLFLNYVNSIKIEKAINRYGKEETK |
| | | RECKPAEDFKTYITLCRNMSFSEICQAVMTEYNQQNNQSRKKKSA |
| | | FDTAKNKDKFRHYVDILHEGIREAFAAYIGLNDQKNYDGIYGFVK |
| | | SFNSSDVFTVEKDKFIEGYRSERFQNLISKVRQNPELQKWYIVAR |
| | | LLNPKQVNELSGSIRSYKQYIEDVCRRSIAEKCPVRKNDGKEVSK |
| | | ASFDNDVKELMSIDYMGVVAILEICIRLNGRFSTVCDDYFKDGAD |
| | | GYAEYLEQYLDYQDEKTKDAGVSPSTMLSMFSEEVSADNTDKNQG |
| | | IIYHDGTNPIMNRNILLSKLYGGANSVIHSVKKVDNRLIADFIKS |
| | | GKLIQEYNKRGYCINEEEQKNLKRYQALKNRVEFRDIVEYGEILD |
| | | ELQGQLINWSYLRERDLMYFQLGFHYTSLHNSERKFEGYRYITKE |
| | | DGSVIENAVLHQILSLYINGIPFYYSYADVEGRDRFICCALKKKE |
| | | PVDGTSNKFEDTGTKMRYVGYYCKEGDNYLGEGIYLAGLELFENI |
| | | AEHDNIIKLRNYIDHFHYYIEDDRSMLDVYSEVFDRYFSYDIKYQ |
| | | KNVVNMLYNVLLSHFAKAGFEFGEGIKQIGSSKKNEMPLTKKMAR |
| | | ILLKSLESDDFTYKIGNTSEAKNQETWVLPARDDLFLDALGKVLN |
| | | WDGSITDENALQKTEIITGRSGNYLKKSRNSDKKNDGNKRSDIKK |
| | | SFNKDKNIPEKKEALTSTPFANLFNNLHMDFD |
| 18 | Ca3 | MKISKVNHTKSAVSVSEGSPKGILYEDPTKSGTKDLETRILERNE |
| | ContigID: | AAKLLYNPINTSRSRKKTHKIINRSLRAFFNRVKKKTGGSFSWDE |
| | k127_751200 | LKRVSYDSSLDAERDKITDSDIDSWEACLKKSLSTPECIEAVKQI |
| | | TRVLCGKNTSHDLDDKLIGKLSSKLHDDYSKERLLGNIKKSIENQ |
| | | NMVVQPGQVDGESIFKLTGDDSLKENPEKVSFERFLISYANLDKK |
| | | FRDCELRKLRRLIVLYFYGETEVDTTDDFDVWADHKKQRNFKWFI |
| | | SDIEFIATYEKYLKELQFEDRRNHHTKISEPEFREKIRQENINRY |
| | | RNSIAVINKSNEVYFDDPVLNKFWIHHIENSVEKLLKRVNPADSF |
| | | KLNVAYIGEKVWKEVINYLSIKYIAVGKAVYRFAVDDMTYGVIPD |
| | | LYKSGISSFDYELIKADESLQRDIAVSVAFAANNMARATVVLDEK |
| | | SSDFLVESFDLEKSIRTDVALDMAILQFFGGKSSWKQCDALKDCK |
| | | YIDLLYDMKKMLYSIRNMSFHFISSEEGDNGYKTNGIIPAMFNQE |
| | | ITTYTTILKSKFYSNNLPAFYNDSDLEGEFKLLYKNYVERASQVP |
| | | SFNSVVVRKSLPDFVKRDLKIKTALSGDDLTKWHSALYYLLKEIY |
| | | YNLFLASDDAKILFLKAVENNKNSNNSSVSDKNDHRREAGIDFAE |
| | | RIESIKDHSLSEICQIIMTEYNQQNQSRKVKTAQDEKNKKSLFIH |
| | | YKMLLNLCLRNAFKMFLDRNEFSFLKSIHNREVKSSSDEWTAAFC |
| | | ADWTSNAYSMIQDEINKNPSLQSWYILSRFITTKQLNHLSGDIRH |
| | | FIQYVEDVKRRAKETGNACKYDLDNKVCIYRKVLQVLDFCNKTSG |
| | | IVSSEIGDYFKDDDEYAKFVSNYLDFGGTTKLELIAFTNQTVGDD |
| | | QINIYCNDSKPILNRNIVMAKLFAPTDTISKAIAANGNRVTVDDI |
| | | EEFYSIKPIAQKFLSDGDSVAKKEKKQLIEELKKTKRYQEIVNRI |
| | | EFRNIVDYAEMINDLLGQLVSWSYLRERDLLYFQLGFHYLCLIND |
| | | SYKPDKYRVLRDGERIINNAALYQIVSLYSFNVDTFRDDNDKDKG |
| | | KKYNNICEYSLNIGLDEEWQFYTAGLELFETITEHDSIKKFRDYI |
| | | DHFHYYTNQDRSILDMYSEVFDRFFSYDMKFRKNTVVILQNILKS |
| | | YLVIMPVKFNSKYKSTDNGSSKMRANVDMGEKGLKSEVFTYKYSD |
| | | SCKVILPARSINYLKDVASILYYPHKTPRDAVDMEDFKKNYEVAQ |
| | | TLNKAKDNHHKSKNDYKNNDRPKDNYSPFKSQFDKLKKKGITFED |
| | | N |
| 19 | Ca4 | MKISKVDHTRTAVGVNENGPLGIVYSDPSQNAVQNPEIRVRTRIK |
| | ContigID: | KANMLYTVFGPTNDEMDSQRENGIAKEFNKIIKRYNNKIDPKRGE |
| | k127_5935133 | KETDKKIYKMNSDELIKDIKSVFGNYSLNESTRKEIDEALNVLIK |
| | | RSLRKKETIESLNLLFEKTIKGEEFKAEEKDKIQKYWVDRIVADY |
| | | SKNTLSKNTIKSIKNQNLVVQPQNKNGEFVFTQAKNRMNGKVNQG |
| | | SIRISKAQEKDALNDFLDGFAVLDKQMRDKQLMKIRRLVDLYFYG |
| | | IDEVVKEDFSVWERHEKTKGNDKKIIPFSRTDISTLQIKRGDSED |
| | | EKKRKNREKKIIKKSDSAKLDDMIRRWNIDRFRESFSAIDKSDNS |
| | | LFFDDKNISKFFIHHIENEVERLFNSERLDDYKMHIGYVSEKVWK |
| | | GIINYLSIKYISIGKSVYNYAMEELNNSSGDVNLGVIDSRYLTGI |
| | | SSFDYEKISAEETLQRETAVYVSFASSNLSRAVFKDGVDCDLMST |
| | | KIIDNHDKFDESKVKKRVLQFFGGESSWDGFGKTFLSEEYNEFDF |
| | | LEDLKTLIYQMRNESFHFNTEKKNVDIKNPKLFSDMFAYECSKAC |
| | | VSEKDKFYSNNLPLFYSEKPLEKVLNKLYTKYNDRKSQVPSFEKV |
| | | MKRSEFGKYLIKSGVATNFNKEDTDKLESGLYYLYKQIYYNDFLV |
| | | NDMIAKGIFVDNINNKKLRRNENNKVIKADKGLEDFKKRLNEIKN |
| | | YSLSEICQIIMTEYNQQNNQKKKSQKNEEIFQHYKLGLYSYLREA |
| | | LIIYINNNSDIYGFIKQPTIKSEGKMPNINEFLPDYSSSQYDDLI |
| | | AKVSDSFELKKWYVMTRFLNPKQTNHLVGALRNYIQYVESIKRRA |
| | | EETGNKIYIDCQILESVKDITKVVDMCTRICGNTSNEISDYFDDN |
| | | DDYAGYLERFLDFEYKESLGSKSSMLGAFCMTKINSEEIKIYHDG |
| | | TNPILNRNIVLSKLYGANSIISEAVPKVDQNMIKEYYIVADKIKE |
| | | YRKSGDCKNIDEIKQLKEYQELKNRVEFRDIVEYSEILSELQGQL |
| | | VNWAYLRERDLMYFQLGFHYVCLKNDSQKPEAYKMIEVPCVDGSS |
| | | RMINGAILYQIVAMYTYGMNIYYRGHKKDEEYNDSENRWEAFNGS |
| | | IGERIPRFALYSGYMIKGDNAKYKLSYNIYTSGLELFEVLEEHGN |
| | | IVDFRNDIDHFNYYQKKDRSMLDYYSEAFDRFFTYDMKYRKNVPN |
| | | TLSNILASHFLVPSFVFGTSSKKVGNKNYIEKKCAHIRFNTKNPL |
| | | KPGSFTYVISEDKRVVGPARLKGYVKNVLNILYYPEVPEMELLDS |
| | | SYIFKEEKKRKLLK |
| 20 | Ca5 | MKISKVRGTQGKGSKLTINAKAAVVINPTGQEGILYDDPSRMGES |
| | ContigID: | RKNDKQRESYIKDRIRASQKLYSIFNSNQKIPKNKKTESEKAIDM |
| | k141_14579520 | IIAGFSSEDGASFRLMFKDFAEILDKYAEKSYENRRNHIDESPEL |
| | | SKLGVNISDNQINALSNLLSEESIAIKIKKGTESVKDKVKVSERD |
| | | IDSAISNCLKKCMCRVKTKKALKALLMKVFDIPYTLDGDVNIRRD |
| | | FIDYAIEDYCRIRVKNSVSESIKKNNMPVQPTSSEGVTVFQMPSL |
| | | QETKSTKSKEREAFNHFLSEYADLDENKRKSLRIKLRRLNDLYFY |
| | | GKDATMALADNEDVDVWEDHAKHGDIKELFIKVQKPQITGDGKAD |
| | | KLAMSQYEDNIRTKYREANITCYRKAVEEIDNDKSLFFEDNMLNM |
| | | FVLHRIESGVERIYSHIKANEEYKLQTGYVSEKVWKDLINYISIK |
| | | YIAIGKAVYNYAMDELVSGDKSIEMGKINDNYISGISSFDYELIK |
| | | AEEMLQRETAVYVAFAARHLAHQTVDLDEKNSDFLLFPDKSRKDK |
| | | DGKNINDFIKEGINLRSTILQYFGGASSWSDFSFEKYMTDGRDDV |
| | | DLLTDLQKAIYSMRNDSFHYTSKNHNNDGWNKELIGALFEYEANR |
| | | LTIIQKDKFYSNNLPMFYDESNLKELLSSLYSKSVERASQVPSFN |
| | | SVFVRKSFPKVCTQDLSIDVKTMNEEDKLKFYNALYFMFKEIYYN |
| | | LFLNDSNVLNRFIDISTKTKKNGKGDEGTHYWAEKDFRQRILSII |
| | | ESRKNYTLSQICQLIMTEYNQQNTGNMRHKSADKNGKNPDSYQHY |
| | | KMLLLSYLGEAFVEFVKEKYDFVFTPVKRDLMDKEAFLPDFAKTV |
| | | NPLGDLIERVKESGVLQKWYIVGRFLSPKQANQMLGSLHSYKQYV |
| | | WDIYRRAEETGTKINKRVSEDTISGVAIRDIDSVLDLCVKMSGTI |
| | | TNNLTDYFKDKEEYAAYINDFLDFEYKTGDYNWALKDFCKEITDE |
| | | DDKEGIYYDGENPIINRNIVISKLYGEAEFVSKIFKRVNKEDIKV |
| | | YKDLKKNIEPYQNMGTFETKEQQENVKRFQELKNHIEFRDLVDYS |
| | | EITNELQGQLVNWIYLRERDLMYFQLGFHYLCLNNNSEKPELYKK |
| | | IEFKDEKVIDNAVLYQICAMYTNGLPLYYSSTKNANIKEVSAKAG |
| | | TSTKVDKFYSSGIRANGESYSRDYTTYMAGLELFENTKEHINITM |
| | | FRNDIEHFRYLVSNTRSMLDVYSEIFDRFFTYDMKYRKNIPNILY |
| | | NILLAHFVNVQFDFSTGKKNIGTGENIYEKKCAKINIQNNGGIVS |
| | | EKFTYKLKDEKTIDLPARGRRYMETVARLLYYPETVDEEKMVKDL |
| | | VIKDNKPFGKKRNNKYSNRKEGASDRKKYEENKARKKDNSFMSGM |
| | | DGVDWSKLNFK |
| 21 | Ca6 | MKISKVDHTRMAVAKGNELRRDEISGILYKDPTKAGSINFDERFN |
| | ContigID: | KLNQSAKILYHVFNGVVTGNKHFINTVKRVNDNLDRVLFTGRNDE |
| | k141_10995992 | RKSITDTDVVLRNADRINAFDRISTDERKQIIDELLEIQLRKGLR |
| | | KGKTGLREILLIGAGVKGRTDRKQDIAKFLEILDEDFNKTKQAKN |
| | | IKLSIENQGLVVAPVEKGEDRIFDVSGVQKGKSSKKAQEKEALSA |
| | | FLSDYADLDKSVRTEYLRKIRRLINLYFYVKNDDDLSSAEIPAEV |
| | | NLEKDFDIWRDHEQKKGEKGDFVDYPDILLADRDEKKRNSKQVKI |
| | | AEKQLRESIRENNIKRYRFSIKTIEKDDGTYFFADKQISAFWIHH |
| | | IENAVERILGSINDKKLYRLHLGYLGEKVWKDILNFLSIKYIAVG |
| | | KAVFNFAMDDLQEKDRDIEPGKISEKALNGLTSFDYEQIKADEML |
| | | QREVAVNVAFAANNLARVTVDIPQDENKDKEDILLWNKQDIHKYK |
| | | KKSQKGILKSTLQFFGGASTWDLKMFEKAYPDQKEDYEEEYLYDI |
| | | IRIIYALRNKSFHFKTYDQGDRNWNSKLIGMMIEHDAEKWVSVER |
| | | EKFHSNNLPMFYKDADLEKMLDLLYSDYTGRASQVPAFNTVLVRK |
| | | NFPEFLRKDMGYKVHFSNPEVENQWHSAVYYLYKEIYYNLFLRDK |
| | | DVKNLFYTSLKNIGNEVSDKKQKLASDDFASRCKEIKDRDLSEIC |
| | | QMIMTEYNAQNSGNRKVKSQRMIEKNKDIFRHYKMLLIKTLSGAF |
| | | ALYLKQEKFAFIGNAATIPYETTDVKEFLPEWKSGMYASLVDEIK |
| | | ENLDLQEWYITGRFLNGRMLNQLAGSLRSYIQYAEDIERRAAENR |
| | | NKLFYKSDEKIETCKKAVRVLDLCIKISTRISAEFTDYFDSEDDY |
| | | ADYLENYLSYQDDTIKELSGSSYAALDHFCNKDDLKFDIYVNAGQ |
| | | KPILQRNIVMAKLFGPDSILPEVMEKVTESDIREYYDYLKKVSGY |
| | | RVKGKCSTVKEQDDLLKFQRLKNAVEFRDVTEYAEVINELLGQLI |
| | | SWSYLRERDLLYFQLGFHYMCLKNKSFKPAEYMDIKRKNGTTIHN |
| | | AILYQIVSMYINGLDFYSCEKDNDKLEVAAAGKGVGSKISLFIKY |
| | | SEYLYNDPSYKYEIYNAGLEVFENNDEHDNITDLRKYVDHFKYYA |
| | | SDDSDKKMSLLDLYSEFFDRFFTYDMKYQKNVVNVLENILLRHFV |
| | | IFYPKFGSGTKEVGVKNCKKEKDRAQIEISEQSLTSEDFMFKLDD |
| | | KSEGEPKKFPARDERYLQTIAKLLYYPKKDVDLNKFMTKEESMNK |
| | | KVQFNRKKETNRRQQNNSSSGALSSSMGDLLKNIKL |
| 22 | Ca7 | MKISKVNHVRTGTRIKENNGEGVLYANPSKQTNAVKDLSKHIQDV |
| | ContigID: | NQKAQGLYSPLNPVKSLINPKMPKEKKDEINGSYKAFKSVVIGIV |
| | k141_12677984 | KENETGIPDSASVIRTLYEKAKKIDLKVSDASYLSSKLIDKCLRK |
| | | SLESKSEIAKEILKAIISTDKSAVNSLNAEEVKAFFELVHKDYYK |
| | | KEQLKAIEKSIENKDVKVQVKTGQNGENHLVLSNADSAKKHYYFD |
| | | FVKEFATKDKAEREEMIIRFRQLIILFYSGSESYKLSIGSDVGAW |
| | | TFGSSLPEVTANVDDEIASLIAEYNENIARKNDIQKSIDLKSNQM |
| | | KNYKFNSPEYKKLDDQVSKLKDEQGDCKHAISDAKRKIKALVENL |
| | | ICTKYRDAVKAEGLTDSDIFWIGYIQQVAQKQFSKKDAYNNYRIS |
| | | TKYLYEVTFNEWISFMASKYIDLGKAVYHFAMPDFSDIKSGKEVH |
| | | AGKVQPAFEDGITSFDYERIKAKETLARDFSVYATYSSGIFSNAV |
| | | TDSEYRLKDEKEDALFYKQEDWEQALLPNAKKKLLMYFGGQTKWE |
| | | DSEIEKLSDLEMTKAFQDMINVIRNSNYHYAGSVLEPGEQSVNIA |
| | | KMLFEKEFSQLGRIIREKYLSNNVPVYYNVEDINKMMTYLYQGES |
| | | KREAQIPSFGNVLKKKEMPGFVSKYIPGNLLAKFDSEGMDKFRAS |
| | | LYFVLKEAYYYGFLNETNLKDRFIMAFKNSEKDAKNPEAIENFKA |
| | | RIADMDDSCSFGEICQILMTDYNQQNQGEYKVKSQIKQNQDEKDN |
| | | KGHKYSHFKMLLYVTLQKAFIDYIFEKQDIYGYIKAPIFKSNFFD |
| | | GDEPQKFVESWEANLFGDVKKTTETDSYYLAWYVLSHMLPAKQVN |
| | | QLQGGIKSYIQFVTDINRREKSVLGTEKDNSLVNNIDYYQNILKV |
| | | LEFVMCFVGKTSNVLTDYFADEDDYALHLYSYVGFAGKKEEKTNS |
| | | TLSGFCSKSITKAGKVLTDRIGIYHDGTNPIVNNNVVKALMYGNE |
| | | NVLSEAVTRVSADLINGEITKYYEVKNKLEKVFEKGECSNIEEQK |
| | | ELREFQNLKNRIELQDISIFTEIINDYMSELVNMAYLRERDLMYY |
| | | QLGYNYIRLEYGNVEDKYKELQGDNINIKSGALLYQIVALYTHEL |
| | | PIVYKDKDSYKYTNNGKIGRFVKSYCEEEFNDLDNTYLKGLELFE |
| | | DIKLHDDLHMFRNEIDHLKYFIRADKSILQMYSRIYNGFFSYDLK |
| | | LKKSVSYIFANILAKYFIIADTEMKSSVENGKRVAMLSVKGLESD |
| | | VFTYKGKKRDKEGKERDSKYTLPVRSDEFLKEVKKLLGYKSM |
| 23 | Cb8 | MPNVKFTLVPVDYSKPYDEQPDCKRHVIGAYANLARHNMTLTINT |
| | ContigID: | IMQAIGMPLFNENEIENAFNKSHRKKLEALDNIQKVKLQKRLYRH |
| | k127_4804511 | FPFFKRMKLEDEEKKTVQLKSLMTVMSLFTSLMADIRNNYTHYRP |
| | | YNNKEEQNRQLELKKEVGKKLQYLYENSSQTFKSMEELDHSSNEV |
| | | LSALRIPEDVVERFSPDDPDYKKLLNTLHDSNIPKWKKSGLKLDM |
| | | KTQIITKKSVRYVRNPNYQAYMMDEEKGLSDIGIIYFLCLFLDKQ |
| | | VSFSLMDEVGFNQQIKFTGEHAEQQLMYVKEIMCMNRIRMVKARI |
| | | DSEMSDTALALDMLSELRKCPRPLYDVFCKEARNEFKDDATVVWE |
| | | NTHGEEAVITEEQGDIGEETDAIAANTTGKNTPRSTFVRWEDRFP |
| | | QLALKYIDLTGMFDHLRFQLNLGKYRFAFYQHDKAYSVDNAERLR |
| | | ILQKELHGFGRIQEVNEMMKEKWQDVMEIKNVEDGQIYKEPDVAG |
| | | QKPYVTQQNAQYDFDTKSHSIGIRWEGWHNNHSDNHYGDLDRRDM |
| | | FIPRLPANPASPEGDKRQTNQAEELLPPQCMLSLYELPAILFYHY |
| | | LLKKYQKNTGLVEKKISDFYTNMKNFLTEVSEGNILPADETTLIR |
| | | ELQTRGLKFSDIPVKLKKLLKGEVTDNAKRMEESALLRLHERKDK |
| | | KRRALESFIAKCKMIGTKENKFNKIRAVVKTGSLGQLLARDIMEW |
| | | LTTDTKKRMNLTGQNYVAMQTALSMMGQSFELAPEAKVTCEKMRN |
| | | IFVKANILPMNDDDFDADFHHPFLLDVFDEEPVSIEDFYKIYLEK |
| | | EIFYIDYLTEHFKKYKAKGAALYIPFLHCERLRWKNTEQNGLKEL |
| | | AARYLQRPLQLPNGLFTDDIFHLLEDIATKNADFAKVLEKQKKDN |
| | | HQLQQNVAYMIRIYMKTVEYDQPQNFYNTMPVGDTNSPYRHIYRI |
| | | FKKFFGESIPKTNKTTSPAYTIEEIRAILNNKQLLKDKIDFFVKE |
| | | EKEKLKKQQIRDFRNYEKKQWKLLKAKNEAAPKGQHFNVKAEVQK |
| | | RLNEKREEQRKALDLLVMDVKQKLEGKLRKVNDNERAIRRYKTQD |
| | | ILLLFMAREILKAKSQDEDFTKGFCLKYVMSDSLLDKPIDFQWTV |
| | | NFQNKEKKTIAKTIEQKDMKMKNYGQFYKFASDHQRLSSLLSRLP |
| | | ADIFERAEIENEFAYYDTNRSEVFRQAYIIESKAYQLKPELTDDA |
| | | NANEEWFTYLDKKTKKLRAKRNNFGELLKILAAGGDGVLDDVEKS |
| | | LLQSTRNAFGHNTYDVDMPVIFSGKLDKMNIPEVANGIKDKIIEQ |
| | | TEQLKKNV |
| 24 | Cb9 | MAIFVIKVEIQRQMTLAFKNETEQKPVLGAYAAMARNNAFLTVMD |
| | ContigID: | IMDQLHIPRTVLTDNSGKEVDPESHIWRLNLFPKNYRLLPEQEAR |
| | k127_1483864 | ASRLLRNHFPFLDLADDLKDVENDHGQAARKNVSYEDLCNSFLTM |
| | | MEVLTHLRDVNLHYKIKDERIADFYFRRAEKETGHILREVLKAAP |
| | | RKIKDRYKGTALMDETSLSFFTDGNYIQKGRKYSFNSRWAFNPQR |
| | | QPQPSEVKVLKNNAPVIDRNTGIPRMFERLSTFGEILFIALFTEK |
| | | RYIPDLLRDSGLDNNFMASGDNGKMSQQRIIREIISAYSIRLPER |
| | | KLDIETGATQIMLDMLNELARCPSELFDVLPESERRSFEITGSDG |
| | | SQVLMKRYSDRYVPLVLRYLDVTEEFKRLRFQVNTGLLRYEHHGP |
| | | KEYMDGVARFRIVQSSINGFGRIQEMEAARTAGATYLGFPLLKTD |
| | | DDGNMTEMPYITDSAARYVLNGDLIGLSFGDAAPKIDTLPNGAGF |
| | | KYKVSCPQPDCWLSRYELPALAFYTFLSRKYHISRSTEDIVEDAL |
| | | NEYRAFFAGIADGSITSMDGVGIPRKNIPEKLLDYLEGRGKRTDF |
| | | KKYKEGLVAKMLLNTEALLSRLKEDLKVIGTKDNRIGKKSYVRIR |
| | | PGKLAEFLAEDIVRFQGHPAGMPEKKLTGQQYSILQGMIATFHEG |
| | | LADACRNAGLLDGDSAHPFLSLVFTRHAQGMTSTVDFYRAYLEER |
| | | RTYLKGVVPDEAPFLHQERRKWSANKDSAYYKSLALRYIKDEKTG |
| | | DKVGVFLPRGLFDNAVHAIIKEHCPNTSKVINASERANMAFIILT |
| | | YLEKELDDQNQGFYFNEERLKEYGFSKAIRKELEESGMKRLSHVL |
| | | RLERNTNPSGLYYEALREESGWKDDRRKGGQLDRKTEEFAEKLRH |
| | | SYKRMCDNEKTIRRYMVQDITLFLLARSLVRIAGNSVNLWSVGPE |
| | | GNGILDQWVDVMTPYKKYIIRQKGIKIKDYGEIYKILKDRRIDSL |
| | | LLNQKKRVPEAIDLEEIKEELVTYNRKRASMVSAIQVYEKGVFEE |
| | | NREHFDSMTSRFGFKEILEADVRSSSLTKEAVKKVRNAVSHNQYP |
| | | DRMVIKDGRSMVLYSPDLPDMAKGIAETTDRLTKYGTDISKQTDE |
| 25 | Cb10 | MLQTEKNDRGAFWAAYFNTATNNVQAILQFAGKNVQLEELSNQEF |
| | ContigID: | KLSANGIGDEETKELEWSAESYPAIQTLKMTGDEVNIPEQIRIMK |
| | Cas13/ | TLSKHLPFMKRISTRVQNGVRKNGKTEKAGEEMTPQMFAEILIGY |
| | 21_contig | VNYLYDLRNYFTHYKHVPVSRKNMQSEYLGILFDANVGTAKERFY |
| | -81_616 | SEDKIAKDDKRFNNFRMHKGAESVPDKKGGMKKQPKLNKDFLFYL |
| | | WETDPITPKGKDNPYLELTAQGLAFFLCLFLEKKSANMLLDSVGI |
| | | EEDAESLIDGFGFENNSGDNRTLLKRIFTITCARLPRTRLESENL |
| | | ISNQVLGLDIMNYLHKCPKEFYNLLSPQDQHKFRTLSDDHGTETL |
| | | LKRFDDRFPYLALNVMDRLECFDSLRFCIDMGTFYFRCHSRVQID |
| | | GSRLENRRLKKKLTCYTRRQDAIEYFQTERAAENTFYQTDNLAPA |
| | | PKAYRTDMLPQYDLGRGRKQENRIGIALKSLDDSRPMFNQPTLDP |
| | | AGQIKPKTYKPDAWLSTYELAPALFLSLHGKGSDVEKRIEEFIRS |
| | | WKGFADWMSKASKEQLKELRYNSAREEFEVFKSRFEKQFGLSVND |
| | | IPDEFRYYLVNGKIKPIYMRFQRGSAVPITMDEAAQIWLYNECDK |
| | | TRSIIRKFNNEQSYDFKLGKGRQRRYTSGNIALWLVRDFMRFQKA |
| | | NDNPREGFGKLKSSPDFNVLQSSLALFNSRKDSLKEILKNARLIY |
| | | NPSGNHPFLENVINRSGALLSIEGFFNAYLDERLEWLSNASGREV |
| | | YQLRKLYERGEKKRSAAKGLPPANVYLGEMAKQFLDESVCLPRGL |
| | | FDDLVRSALKEIYPEQYAKDVPDGQRANFTWLMQKHLKWSGDDHQ |
| | | WFYKELKQSMTAEEFKKLFELLGTTDVRYDNAGKKTLNDKLEETY |
| | | SQRWYDLEKSLKNDKEFKSLSPNDKQSAIDDRMGQENQMYRSLLN |
| | | RLRDVWKRIRMSSVQDVVLRDAVWQLLGLPEKSVRLCNVKPEYDL |
| | | KTQRMIRDDAGDGSISNDAILNETHSLENIIDLPKGLGTIVLKGE |
| | | MKLKNFGNFYRMLSDPKLPSFLCLYKQFGFNAVDYSYLELQEFEF |
| | | YDRKFRPAVFNWVHQLEETVLEKYPDLPMKYGKFVDFWTIAGKAN |
| | | GGYIFTQLLLTSIRNAVSHQYYPEFCVPSDWTNQKDIDTYSPQFK |
| | | DQLLSVKAEFLKRRSEDKDALLSETIYDYAQSLFENAIAFVEQQP |
| 26 | Cb11 | MANFQTPQRHIFGTYLNIARANFYKTILHVFSASGIDCYTKRGDL |
| | ContigID: | FVREDTVDKVISALYLIVNGENAGYHAIKEIVSKSYDKRWKEDKA |
| | k141_16137484 | LQGNLSGSELKARKEEFKSPLKDEGPDGEDARICKSFTLGSEQEE |
| | | RMRKLLFRHIPLLSPIMADVVAMQFKETTNEHQEANKTLHDATLA |
| | | DCFKELSNIARCLSESRNFYTHKNPYNSIEAQRTQLQLQKVIANN |
| | | LDKAFIGSRRIAKKRNSYSEKDLAFLTGHDNDCRMEEIFVLDENG |
| | | NKIWKVEKDKNGKDKLDKDKNPIYVYKKVKAKDGKGREKLDEKGK |
| | | PVYETLRENGEPVHEYEKKFVERKDFYFRIRGKREVLAPDLTPTG |
| | | EECDGLSAFGMLYFCSLFLSKEQTAQLCTESRVFVTSPYQPAGNL |
| | | KNNIILNMMFVYAIHIPRGKRLDSETDSQALGMDMLNELRRCPIE |
| | | LYDVLPSIGKRDFEDNVKHENNRTPELSKRIRLKDRFPYLAMRYI |
| | | DQQQLFKRIRFHVRLGSFRFCFYDKTCIDGKSHPRQLHKDINGFG |
| | | RLQDMEKERKEQYGHFFQQSREQSIWQKDENAYVNLKQLEPIKAG |
| | | DQPHITDMFAQYNIHQNRIGLFWNTDEECKLVNKTNAQGEIIYNG |
| | | YYLPPLNYVDSPTENNKHKRKAPVDMPAPLCSLSVFELPAMLFYN |
| | | FLRNTDSLGGEEFPDVEEIVIKQYDNIRKFFLEVKDIQPTDNIEN |
| | | LATILNAYGLSKQSVPKKIYDYLSNKNTLISKDIRKSTEKEVKNR |
| | | LRRAIIRKQRFEQDQEHIENTKDNKIGKDSFVSIRYAKIAEELAK |
| | | SMMEWQSGNTKMTGLNFRVLTAALAKFGDGVIKRDTIISMLQKAE |
| | | IMGGDNPHCFIEQAIEQEQYDIEEFYLDYISAEIEYLKRFLMIDG |
| | | KTIILKDEQLLDALRKDKEVHDDVRIRLKNDVDFGQLPFIHKSRL |
| | | RWQQSKIEELANRYLYVKEEGEETLGRATLLLPDGMFFPYIMKGF |
| | | QKCHQELTNSIEALSDEQKKGIENNVAYLINLYFESKGEKSQSFY |
| | | DSTEPSHYNDNIRQLAPYKYARSYEFFKIIKGWQIHLSCDEMKKR |
| | | LTGKKTIIDNKINALKEKGNYISLEEAKNALRRKLHNTFRDMQDN |
| | | ERVIRRYKTQDRILFLMARDMMGEIVNKKADLFKLENVCKDDFLS |
| | | QKVKASIAVHLSMGEVFKIQKDEMAIKDYGKLYRLLRDDRITKLL |
| | | SYALYETGETIDYDDITDELKEYESCRSAAFEAVQMIEDTRYQQD |
| | | KEVLSNPNENNFYCGNIRYKNGKDNGRENEAKRNNFRTLLEDLQK |
| | | FTPEQMEMFSQEDRELIISIRNAFGHNSYPKQVDFERLINQERKN |
| | | NPNFKIELKQVASFILDKLEEYVNQVNPQT |
| 27 | Cb12 | MSYTPSSRPPRRPQIVEGSRNNALRILKITPDEQTAFVTYLNYAV |
| | ContigID: | NNLSEVAGVAFSDERKVRADVFRGEPADIQRRISRLADFLWSFRE |
| | k127_333529 | KDPSADESGYKAKLGGGHDDMAVWLTEKIVSLRNFFSHVNRQDCT |
| | | PLVISHDEYVFVEGILGGAARDAAMGPGLNPAKAQKLKLATHHVK |
| | | EAHTYEFTRKGLVFLTCLGLYKDEAEEFCHLFHEMKVPDRIEDAD |
| | | LDEELPDGRHRAVLGLEDLDKFAGLKGKGRALIELFSFYSYRRGR |
| | | QSLDAANLDFMQFADVVGCLNKVPAPAFEYLPLEKEHAELEALKA |
| | | ASTESEENKRTKYVLRRRDRNRFLSFAAAYCEDFGVLPSVRFKRL |
| | | DVRRTTGRHQYVFGPGADAETDEEESNRVRLNRHYAIRRDAIPFE |
| | | FEPDRHYGPVRIGALRSAVSATEMRRLLYLHAQGADIDAVLRGYF |
| | | EAYHRVLERMVNAGSLAEISLDDETFLADFATICGTSPEAFKADP |
| | | EAFRKFVPESLRRYFSKDAGPRSEQRLHALLCSKLLAAVNRTCDI |
| | | LVRHDALEAWREASRPWLDARDDWRGRIDVWRKANPKKDLPDELK |
| | | TFEAYLETLPADERPARPEEPRCRVGEGEGEITNPPTWCRFSDAD |
| | | YVSFVFDWFNLFLPNNRKFRQLPISEQHREGVEDHLFQMVHAAIG |
| | | KFSLDQKGLWSLLEKNRAELKPYADLLQYRTDIRVLVAPFVRELN |
| | | HQLPENRRLPEFDNGVDKDEKEIASPILNIFRHFRQNELWQFLQN |
| | | FRPELKSWCRSMKSKLQDAMEKKPSRKDGKPHFPKVEDLFEVLNL |
| | | RRPSPTLEDLAVEAVKLSNDMFLEETNRWGAANPMSVSRDDLLAA |
| | | CRRFGVRPGMPVEYKSLLKTVLGIDYDAWRHAFDYGTGRPFADRR |
| | | LEDEEHVAAQIPLPNGFADRVAASLPRKVRERFQAPGSAAFDFNA |
| | | AFRAWSPDPAVSLRDFYDVKPLVASQIARKHPPEGGAATADPFAA |
| | | LSAKTLDSAVREIKDAENQDKVLLFVAMKYWERFRGGDTYSTGKL |
| | | KMPFSEKTTLREFFDTPVQIEKDGLTVSFRPNDVNRPAFATLFGN |
| | | SRAAKDYRAKIAKILSPDGSRTAFDFYEMVVAFREQKARDRHERL |
| | | AFTPYAVHFDALCEIPASAYEKAAEGRFGEAKTEAIRAMEFERYK |
| | | AVLPGLTREDYDAVADARNAVFHTGFKFDCSKAIAVCKRLGVLGM |
| | | MPGEAAPAGRRTSSHSGPKWSGPRRNGGSRW |
| 28 | Cd13 | VSKNDNIKSKAKALGLKSTFQVGDEVVMTSFGKGNKAIVEKIVRG |
| | ContigID: | TDVQSVPTEPNFSAEIEGKKFDLVGRAHIQTKSDNPQYSKKRTGD |
| | k127_2411982 | DMIGAKAALEKRFFGGTFDDNIHIQLAYNVLDIEKILSVHINNIV |
| | | YTLNNIRRKDNAEDDDFIGYMSTRNDYDTFIEPRKHNISEDAAKS |
| | | IDKSRASFEEYLQPPVKDCLHYFGNTFFAPREIEKTYTDNRGFER |
| | | KKKVTVNSLIDEKEIYYIFALLGGLRQFCTHDNKSGRNWLYSLEE |
| | | NGINSDAKAVLDKYYNSAVARIDESFVDNASKTNFKLIFNAMDVS |
| | | DEALQDNIAKAFYKFTVCKSFKNMGFSIKKLREQLLELPEYEGLK |
| | | DKHYDSVRSKLYQIMDFVIYLSFKDEKFKKDNEEKINNIVNELRA |
| | | TLNEEDKGRVYASYAERMKSELKPAIARLKSDIDKIKDSRVKEFE |
| | | LDASVKYRLSKVVESVRLKDRATYFTKLIYLTTLFLDGKEINDLL |
| | | TTLIHQFENIASFIDVMNDRGIDCRFSDGYKLFESSKQIAFELRN |
| | | VNSFARMTRTSKDDENATHMMYIDAAEILGTDYTEEQIEEHLNLE |
| | | KKRMIPGTKKADMNFRNFIINNVIKSSRFNYLVRYSNPKKIRALA |
| | | DNEGVIRFVLGELPDAQIDRYTLLCGFNPDADRQEKTDKLAKAIT |
| | | GLRFNDFENVKQGANTEGESQESIDKAQKQGLISLYLTVLYLLTK |
| | | NLVYVNSRYFLAFHCLERDAQLLGSGAGHHEPYVALTQRFINEDK |
| | | LNEHACEYLKTNIANSDEYTIRIFRNNAAHLSAVRNANLYIDKLK |
| | | EFKSYYEIYHFLSQENIYGKYCVDKKYVTADENGSKTISVKISKD |
| | | YCPQVYIDKSLEYFDKLNKYGTYCKDFTKALNSPFGYNLARYKNL |
| | | SIEGLFDRNRPGDKGENTFED |
| 29 | Cd14 | MAKKLSPKEIREAAKAEKMKSIKAAEAEREKAAEEAKLKAEAEKK |
| | ContigID: | EKAEKNEREKALKRFRLDEKSRMALPKSERKSLAKAAGVKSAFAV |
| | k141_15335538 | GNDIYLTSFDRGNDAIVEKKITDTVVTNLRSDESFEVNENTITEM |
| | | SVPIKSKRISDLYAIADNPLYRKDSATKVQPDKLLLKDTLEKLYF |
| | | GKTFDDTLHIQIIYNILDIEKILTVYSINTIYCLNNLFGKESGEK |
| | | EDLISKLTYQITYDEFKESKAHNEFIDFYNLNTLGYYGNIFFKEK |
| | | KKRSQKEIYDIIALIATIRQWCVHCEEDKRTWLFNTETVLSKEFL |
| | | DILDDVYESLVEKVNRNFLKDNKVNLQILEDVLEIKDSESREKLI |
| | | RQYYRFIVTKEQKLLGFSIKKLREAMLEETEFKTDKKYDTVRSKL |
| | | YKLIDFLLFTGYTTDEAEKEKALFLIKSLRESLTEETKDRIYKSE |
| | | AARLWLKYENTITNKIREALNEKSISELKKDKSFDDKSITSIITD |
| | | EVSGKKATYFSKTIYLLAQFIDGKEVNDLTTSLINKFDNIRSLID |
| | | TAGQIGLDCKFTEEYKFFENSDQIRTELHVIKNLTNMEYYDTTVK |
| | | KQMYKDAVHILGIQDDVSDAELEKIINSILLLNENGKPLPGTKGK |
| | | KGFRNFIISNVLKSRRFIYLIKYCNPKKIRKIAGNRKIIKFVLSR |
| | | ITDSQLERYYYSCNPELKSGIYPGRDDAVNDLSILIADMKFEDFK |
| | | NVDQSANVHDNNNAAREKMKYQTIISLYLTVCYHLVKNLVNINAR |
| | | YAMAFHALERDARLYQIFSSEENYVDNLNADYAILTKTLLKDNYE |
| | | NAGNLYLRNKKWNKLTRENLDNYIPQAAANFRNAVAHLNPIRNAD |
| | | MLLEDIEDVSSYYAIYHYIMQKSVTNRTIRVSNTTEDEKRILTDY |
| | | QNKIKRHHGYNKDFVKALCVPFAYNIVRFKSLSIYEMFDRNYHEK |
| | | TSPETDDSQSP |
| 30 | Cd15 | MDKEKTTVEGKNTNQKSDVLKSLAKANGLKSSFVIGNEVVMTSFG |
| | Cas13/23_ | RGNSAILKSKVFFDKIKKGNKLGYFGQAFYYATGTGKNLIFTKKS |
| | contig | SETIYELLALVGSLREKKITGSRIENLNPNVAFYVKKHISDSCNP |
| | ContigID: | DSGKYDVKSKRMKEKAVVDDPVYVSPEKASNVHAGQDLIGCKNVL |
| | -81_4932 | EERYFGKTFDDNIHIQLIYNILDIEKILAVHVSNATFAINNILGI |
| | | EGKENEDFIGNLSVLNTFDEFENYETHPKFANKSAIKENLRQFCV |
| | | HDEVMVDNKVKSRSWLYNAQKELKPDFLKALDELYSKEVEKIDSD |
| | | FIVNNTVDLHIIHDAIDVIDGSADWQKITNEYYDFIIRKCFKNIG |
| | | FSIKRLRETMIEEQMKVLCGKCDRENCKGYGKCFKNKKYDSVRSR |
| | | LNRIVDFIIFRHYNDEAILKNVSLLRTCMSEEEKQKRFYLPEAKA |
| | | LWKKYNYVFRNYVLKKLNGKSISGLKEKAIENSIDINSVKISLGD |
| | | PDYFCKFIYLLTFFLDGKEINDLLTTLINKFDNIASFISVMKNDK |
| | | LSIDCEFVPEYSFFANSAQITSDLRVINSFARMQAPAEPSKDDMY |
| | | RDALDILGMDDLSEDGKKQLEDTVLCRDENGKYMKKEDNNPKRDT |
| | | NFRNFLGNNVLASTRFKYLIRYNNAKKTRALANNKAVIIFMLNKI |
| | | NKQNPEQIVSYYKACRDDSDPVASDAEAKIEFLAEKIMNVSCTQF |
| | | RYVKNGTKVRPDEAKEKERFKAIIGLYLTVMYLITKNMVYINSRY |
| | | VTAFHCLERDSELHGVKFDQKKLQPNLTKKFIDPKTCGDYGLRNN |
| | | KRARTYIEQNMDKMSNCTSYWNEYRNAVAHLSVIRNMNQYIKNVK |
| | | NIGSCFELYHYIMQRFLLDKENIAESLREYDDFIKKKGCYRKDFV |
| | | KALNTPFGYNLARYKNLSIAELFDRNDTELERTNKLRNEAIKADI |
| | | EEI |

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

### General and Definitions

For purposes of interpreting this specification, the following definitions will apply and whenever appropriate, terms used in the singular will also include the plural and vice versa. In the event that any definition set forth conflicts with any document incorporated herein by reference, the definition set forth below shall prevail.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

In the following, the invention will be described in greater detail. The examples and preferred embodiments described throughout the specification should not be construed to limit the present invention to only the explicitly described embodiments. This description should be understood to support and encompass embodiments which combine the explicitly described embodiments with any number of the disclosed and/or preferred elements.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

As used herein, except where the context requires otherwise, the term “comprise” and variations of the term, such as “comprising”, “comprises” and “comprised”, are not intended to exclude further additives, components, integers or steps.

The term “and/or”, e.g., “X and/or Y” shall be understood to mean either “X and Y” or “X or Y” and shall be taken to provide explicit support for both meanings or for either meaning.

Unless otherwise indicated, the term “at least” preceding a series of elements is to be understood to refer to every element in the series. The term “at least one” refers to a minimum of one, but also encompasses two, three or more such as four, five, six, seven, eight, nine, ten and more.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, “protein,” “amino acid chain,” or any other term used to refer to a chain or chains of two or more amino acids, are included within the definition of “polypeptide,” and the term “polypeptide” may be used instead of, or interchangeably with any of these terms.

“Cas13 polypeptide”, and the specific subtypes Cas13a, 13b and 13d, may be used interchangeably with Cas13 protein, Cas13 peptide, Cas13 effector enzyme and Cas13 enzyme. At times throughout the specification, the phrase “Cas13 polypeptide” may also be abbreviated to “Cas13”.

As used herein, “Cas13” is a CRISPR-Cas effector enzyme that displays at least collateral cleavage activity upon binding to a cognate target RNA complementary to the spacer sequences in the guide sequence. The collateral activity of the Cas13 enzyme enables it to cleave RNase or endonuclease activity against a non-target RNA. This property of the Cas13 polypeptides of the invention is also referred to as trans-collateral activity or trans-cleavage activity and is used interchangeable in the specification.

The term “cis cleavage”, or “cis cleavage activity” or “cis activity” will be understood to mean specific, targeted degradation of target RNAs (referred to as cis-cleavage activity) both in vitro and in vivo.

The Cas13 polypeptides of the invention, while naturally occurring in bacteria, are all isolated from bacteria or are expressed from a nucleic acid molecule that is itself engineered or recombinant. Unless the Cas13 polypeptide and/or nucleic acid molecule encoding it are being discussed in the context of the bacteria in which they are naturally found, all references in this specification are to the non-natural versions of both and may be described as non-natural or engineered.

More specifically, the term “isolated” in relation to a nucleic acid sequence, or a polypeptide, is used herein to define that by virtue of its origin or source of derivation, it is not associated with naturally-associated components that accompany it in its native state; it is substantially free of other proteins from the same source. A nucleic acid sequence or polypeptide may be rendered substantially free of naturally associated components or substantially purified by isolation, using techniques known in the art. The term “isolated” is also used herein to recombinant nucleic acids and polypeptides.

The term “engineered”, which can be used synonymously with terms such as “non-naturally occurring” is used herein with respect to CRISPR/Cas systems that are not found to occur naturally in eukaryotic cells. That is, the components of the system do not exist together naturally in eukaryotic cells.

A “functional variant”, as used herein in respect of the Cas13 polypeptides of the invention, refers to a sequence variant of the Cas13 polypeptide which retains at least 95% of the cleavage of the reference Cas13 polypeptide, when both are subjected to the same methodology of assessing cleavage activity. The amino acid sequence of a functional variant has at least 80%, 85%, 90%, 91%, 92%, 93%, 94%, 95%, 96%, 97%, 98%, or 99% identity amino acid sequence. The cleavage function may be cis activity, trans activity or preferably both.

A “target RNA” or “RNA of interest” as used herein refers to an RNA polynucleotide being, or comprising, the target sequence. The target RNA may be an RNA that is endogenous in a target host cell (derived from mammalian, yeast, insect or bacterial cells) or it may be a foreign source of RNA, such as viral RNA. The target RNA may be RNA that has been isolated and purified; or may be an RNA that has been synthesised.

A “CRISPR RNA”, or “crRNA”, also referred to in the literature as “guide RNA” or “gRNA”, includes one or more spacers and one or more Cas13-specific direct repeats. The spacers are capable of specifically hybridising with one or more target RNAs. Hybridisation promotes the formation of a CRISPR complex the Cas13 polypeptide at the site of potential cleavage of the RNA.

The Cas13 proteins and crRNAs are assembled in to a ribonucleoprotein (RNP) complex.

As used herein, “direct repeat” or “direct repeat sequence” refers to the RNA sequence in the crRNA that folds to form a secondary hairpin RNA structure, to which the Cas13 polypeptide binds.

The “spacer” within a crRNA can include a nucleotide sequence that is fully or partially complementary to a specific sequence within a target RNA.

The terms “hybridising” or “hybridise” can refer to the pairing of substantially complementary or complementary nucleic acid sequences within two different molecules. Pairing can be achieved by any process in which a nucleic acid sequence joins with a substantially or fully complementary sequence through base pairing to form a hybridization complex.

“Homology”, “identity” or “similarity” refers to sequence similarity between two polypeptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

As used herein, “contacting” a cell with a nucleic acid molecule, can be allowing the nucleic acid molecule to be in sufficient proximity with the cell such that the nucleic acid molecule can be introduced into the cell. Similarly, “contacting” a nucleic acid with an RNP complex, or the Cas13 proteins and crRNAs components of an RNP complex, means bringing them in to sufficient proximity for the RNP complex to bind to a target sequence in the nucleic acid if the target is present. Conducting a binding and cleavage reaction in a single vessel is sufficient proximity.

As used herein, “detector RNA” is used synonymously with the term probes, detection molecules and reporter sequence. It is any RNA sequence that is cleaved by the trans cleavage activity of the Cas13 polypeptides of the invention, and which is in turn indicative of the presence of the target sequence in a sample with a nucleic acid.

As used herein, the Cas13 polyprotein of the invention is “activated” upon the presence of a target sequence to which the spacers of the crRNA of the RNP complex binds.

As used herein, the term “operably linked to” means positioning a promoter or other regulatory element relative to a nucleic acid such that expression of the nucleic acid is controlled by the promoter or other regulatory element.

As used herein, the term “promoter” is to be taken in its broadest context as a naturally occurring sequence, or a recombinant, synthetic or fusion nucleic acid, or derivative which confers, activates or enhances the expression of a nucleic acid to which it is operably linked that initiates transcription of a gene. Suitable promoters include but are not limited to tissue-specific promoters, inducible promoters, and constitutive promoters.

A used herein, a “vector” is DNA molecule (often plasmid or virus) that is used as a vehicle to carry a particular DNA segment into a host cell. The vector typically assists in replicating and/or expressing the inserted DNA sequence inside the host cell.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

### i) Cas13 Polypeptide

One aspect of the invention provides a Cas13 polypeptide, or a nucleotide sequence encoding the Cas13 polypeptide. There is also provided a composition comprising a Cas13 polypeptide, or a nucleotide sequence encoding the Cas13 polypeptide.

The Cas13 polypeptides of the invention may be isolated from bacterial species in which they naturally occur, or may be expressed in vivo or in vitro from non-naturally occurring nucleic acid molecules.

In a preferred embodiment of the invention, the Cas13 polypeptide is a Cas13a polypeptide, a Cas13b polypeptide, or a Cas13d polypeptide. Alternatively, in a preferred embodiment of the invention, the nucleic acid molecule comprising a sequence encoding a Cas13 polypeptide is a nucleic acid molecule encoding a Cas13a polypeptide, a Cas13b polypeptide, or a Cas13d polypeptide. The Cas13a, Cas13b, and Cas13d polypeptide have at least trans cleavage activity and preferably both trans cleavage and cis cleavage activity. The Cas13 polypeptide of the invention is preferably a Cas13a or Cas13d polypeptide, and more preferably, is Cas13a7, Cas13d13, Cas13d14 and Cas13d15.

In embodiments wherein the Cas13 polypeptide is a Cas13a polypeptide, the Cas13a polypeptide has an amino acid sequence of SEQ ID NO: 16, or SEQ ID NO:17, or SEQ ID NO: 18, or SEQ ID NO:19, or SEQ ID NO: 20, or SEQ ID NO:21, or SEQ ID NO:22, or is selected from a sequence being at least 80%, 85%, 90%, 91%, 92%, 93%, 94%, 95%, 96%, 97%, 98%, or 99% identical to SEQ ID NO: 16, or SEQ ID NO:17, or SEQ ID NO: 18, or SEQ ID NO:19, or SEQ ID NO: 20, or SEQ ID NO:21, or SEQ ID NO:22.

In embodiments wherein the Cas13 polypeptide is a Cas13a polypeptide, preferably the nucleic acid molecule encoding the Cas13a polypeptide comprises a sequence selected from SEQ ID NO: 1, or SEQ ID NO:2, or SEQ ID NO: 3, or SEQ ID NO:4, or SEQ ID NO: 5, or SEQ ID NO:6, or SEQ ID NO:7, or is selected from a sequence being at least 80%, 85%, 90%, 91%, 92%, 93%, 94%, 95%, 96%, 97%, 98%, or 99% identical to SEQ ID NO: 1, or SEQ ID NO:2, or SEQ ID NO: 3, or SEQ ID NO:4, or SEQ ID NO: 5, or SEQ ID NO:6, or SEQ ID NO:7.

In embodiments wherein the Cas13 polypeptide is a Cas13b polypeptide, the Cas13b polypeptide has an amino acid sequence of SEQ ID NO: 23, or SEQ ID NO:24, or SEQ ID NO: 25, or SEQ ID NO:26, or SEQ ID NO: 27, or is selected from a sequence being at least 80%, 85%, 90%, 91%, 92%, 93%, 94%, 95%, 96%, 97%, 98%, or 99% identical to SEQ ID NO: 13, or SEQ ID NO:14, or SEQ ID NO: 15.

In embodiments wherein the Cas13 polypeptide is a Cas13b polypeptide, preferably the nucleic acid molecule encoding the Cas13b polypeptide comprises a sequence selected from SEQ ID NO: 8, or SEQ ID NO:9, or SEQ ID NO: 10, or SEQ ID NO:11, or SEQ ID NO: 12, or is selected from a sequence being at least 80%, 85%, 90%, 91%, 92%, 93%, 94%, 95%, 96%, 97%, 98%, or 99% identical to SEQ ID NO: 8, or SEQ ID NO:9, or SEQ ID NO: 10, or SEQ ID NO:11, or SEQ ID NO: 12.

In embodiments wherein the Cas13 polypeptide is a Cas13d polypeptide, the Cas13d polypeptide has an amino acid sequence of SEQ ID NO: 28, or SEQ ID NO:29, or SEQ ID NO: 30, or is selected from a sequence being at least 80%, 85%, 90%, 91%, 92%, 93%, 94%, 95%, 96%, 97%, 98%, or 99% identical to SEQ ID NO: 28, or SEQ ID NO:29, or SEQ ID NO: 30.

In embodiments wherein the Cas13 polypeptide is a Cas13d polypeptide, preferably the nucleic acid molecule encoding the Cas13d polypeptide comprises a sequence selected SEQ ID NO: 13, or SEQ ID NO:14, or SEQ ID NO: 15, or is selected from a sequence being at least 80%, 85%, 90%, 91%, 92%, 93%, 94%, 95%, 96%, 97%, 98%, or 99% identical to SEQ ID NO: 13, or SEQ ID NO:14, or SEQ ID NO: 15.

In the abovementioned embodiments, wherein the Cas13 polypeptide is described as being a sequence with at least 80%, 85%, 90%, 91%, 92%, 93%, 94%, 95%, 96%, 97%, 98%, or 99% identity (ie a functional variant), it is intended that the function of the Cas13 polypeptide be substantially the same as a polypeptide of the defined, reference SEQ ID NO despite the variation in the sequence. By “substantially the same”, in this context, it is meant that the Cas13 functional variant has cleavage activity within +/−5% compared to the reference sequence, when both are subjected to the same methodology of assessing cleavage activity.

In that regard, the polypeptide sequence may vary in preferred embodiments only by way of a “conservative amino acid substitution”. As would be understood by the skilled person, that means the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Similarly, as a result of the redundancy in the genetic code, whereby most amino acids are specified by more than one mRNA codon, a nucleotide sequence can be altered while maintaining the same amino acid sequence of the encoded protein. The sequence encoding a Cas13 polypeptide of the invention may be at least 80%, 85%, 90%, 91%, 92%, 93%, 94%, 95%, 96%, 97%, 98%, or 99% identical to the reference sequence encoding the polypeptide of the defined SEQ ID NO. Accordingly, in embodiments of the invention, a sequence having at least 80%, 85%, 90%, 91%, 92%, 93%, 94%, 95%, 96%, 97%, 98%, or 99% identity to:
SEQ ID NO: 1 encodes a Cas13 polypeptide of SEQ ID NO:16
SEQ ID NO: 2 encodes a Cas13 polypeptide of SEQ ID NO:17
SEQ ID NO: 3 encodes a Cas13 polypeptide of SEQ ID NO:18
SEQ ID NO: 4 encodes a Cas13 polypeptide of SEQ ID NO:19
SEQ ID NO: 5 encodes a Cas13 polypeptide of SEQ ID NO:20
SEQ ID NO: 6 encodes a Cas13 polypeptide of SEQ ID NO:21
SEQ ID NO: 7 encodes a Cas13 polypeptide of SEQ ID NO:22
SEQ ID NO: 8 encodes a Cas13 polypeptide of SEQ ID NO:23
SEQ ID NO: 9 encodes a Cas13 polypeptide of SEQ ID NO:24
SEQ ID NO: 10 encodes a Cas13 polypeptide of SEQ ID NO:25
SEQ ID NO: 11 encodes a Cas13 polypeptide of SEQ ID NO:26
SEQ ID NO: 12 encodes a Cas13 polypeptide of SEQ ID NO:27
SEQ ID NO: 13 encodes a Cas13 polypeptide of SEQ ID NO:28
SEQ ID NO: 14 encodes a Cas13 polypeptide of SEQ ID NO:29
SEQ ID NO: 15 encodes a Cas13 polypeptide of SEQ ID NO:30

Two Higher Eukaryotes and Prokaryotes Nucleotide-binding domains (HEPN) provide the RNAse activity of Cas13.

The Cas13 polypeptides of the invention have at least trans-collateral cleavage activity, and preferably also retain their cis cleavage activity. The Cas13 polypeptide of the invention is preferably a Cas13a or Cas13d polypeptide, and more preferably, is Cas13a3 of SEQ ID NO:18, Cas13a7 of SEQ ID NO:22, Cas13d13 of SEQ ID NO:28, Cas13d14 of SEQ ID NO: 29 and Cas13d15 of SEQ ID NO:30.

The Cas13 polypeptides of the invention may be isolated from, or expressed from nucleic acid molecules engineered on the basis of the sequence of nucleic acid molecules within a bacterial organism. To assist with expression in cells, the coding sequence may be may be codon-optimised for expression in a eukaryotic cell.

The protospacer flanking sequence (PFS) for Cas13, which is analogous to the PAM sequence for Cas9, consists of a single base pair. In some embodiments, the Cas13 polypeptides of the invention are sensitive to, and require the presence of a PFS in the target sequence. In turn, the Cas13 polypeptides may have a preference for, and preferentially cleave, targets with a particular PFS. This characteristic of the Cas13 polypeptide can be exploited in, for example, nucleic acid detection assays, particularly where the sample contains multiple targets.

In alternative embodiments, the Cas13 polypeptides of the invention are capable of binding to target RNAs in a protospacer flanking sequence (PFS)-independent manner. This is particularly advantageous for use of the Cas13 in a CRISPR system as the crRNA can be designed without having to ensure the presence of a PFS in the target sequence. Preferably, the Cas13 of the invention that is PFS-independent is a Cas13d, most preferably Cas13d13, Cas13d14 and Cas13d15.

ii) CRISPR RNA (crRNA)

Cas13 only binds and cuts ssRNA. Cas13 finds its target with the help of a CRISPR-RNA (crRNA) also known as a CRISPR-RNA (crRNA). The crRNA consists of:
a sequence which, via the presence of at least 2 direct repeat sequences of at least 16 nucleotides and up to 50 nucleotides in length, forms a double stranded, hairpin-like structure that is bound by Cas13; and
a variable “spacer” sequence of approximately 15 to 40 nucleotides that is complementary to, and capable of hybridising, the target RNA. The spacer need not be 100% complementary, but must be sufficiently complementary to permit hybridisation. In each embodiment of the invention, the spacer sequence is designed for the specific target/s of interest.

In some embodiments, the Cas13-specific direct repeats are at least 16 nucleotides in length, but preferably no longer than 50 nucleotides in length. In some embodiments, the Cas13-specific direct repeats are 30 to 40 nucleotides in length. In some embodiments, the Cas13-specific direct repeats are about 35-37 nucleotides in length.

In some embodiments, the spacers are 15 to 40 nucleotides in length. In some embodiments, the spacers are 25 to 35 nucleotides in length. In some embodiments, the spacers are about 30 nucleotides in length. As noted above, if the Cas13 is PFS-sensitive, the spacer sequence is designed to include a PFS.

The direct repeat sequences may be located upstream of the spacer sequence, or the direct repeat sequence may be located downstream from spacer sequence.

The ability of a crRNA sequence to direct sequence-specific binding of a CRISPR/Cas13 complex to a target nucleic acid sequence may be assessed by any suitable assay.

The crRNA can include multiple spacer sequences to target multiple RNAs or to target multiple sequences within the same target RNA. The crRNA preferably has at least 1 spacer sequence, but may include 2 spacer sequences or 3 spacer sequences or more. The crRNA may be a precursor crRNA, that is processed in to the individual crRNAs. Alternatively, when being used for targeting multiple RNAs, multiple crRNAs can be used.

iii) CRISPR Systems

The at least one Cas13 polypeptide of the invention forms a complex with the at least one crRNA via binding to the direct repeats that have formed a double stranded, hairpin-like structure, and wherein the at least one crRNA directs the complex to the one or more target RNA molecules by way of the engineered spacer sequences, thereby targeting the one or more target RNA molecules.

In one aspect, there is provided a CRISPR/Cas13 system for targeting RNA molecules, the system comprising
i) at least one Cas13 polypeptide wherein the Cas13 polypeptide has an amino acid sequence selected from the group consisting of SEQ ID NOS: 16-30, or a sequence being at least 80%, 85%, 90%, 91%, 92%, 93%, 94%, 95%, 96%, 97%, 98%, or 99% identical to SEQ ID NOS: 16-30; or a nucleic acid molecule comprising a sequence encoding the Cas13 polypeptide and
ii) at least one CRISPR RNA (crRNA) or a nucleic acid molecule encoding the crRNA, the crRNA comprising one or more spacers and one or more Cas13-specific direct repeats, wherein the crRNA is capable of hybridising with one or more target RNA molecules.

Preferably the nucleic acid molecule comprising a sequence encoding the Cas13 polypeptide is selected from the group consisting of SEQ ID NOS: 1-15, or is selected from a sequence being at least 80%, 85%, 90%, 91%, 92%, 93%, 94%, 95%, 96%, 97%, 98%, or 99% identical to SEQ ID NOS: 1-15.

As defined above, the Cas13 polypeptide in the CRISPR system of the invention may be a protein, but alternatively may in the form of a nucleic acid molecule that encodes the protein. It will be appreciated that the nucleic acid molecule encodes the Cas13 polypeptide in expressible form such that expression results in a functional Cas13 polypeptide. Similarly, the crRNA component of the CRISPR system may be in the form of RNA itself, or a nucleic acid molecule that encodes the crRNA.

In one embodiment, the CRISPR system for targeting RNA molecules comprises (i) at least one Cas13 polypeptide and ii) at least one CRISPR RNA (crRNA), the crRNA comprising one or more spacers and one or more Cas13-specific direct repeats, wherein said crRNA is capable of hybridising with one or more target RNA molecules,
wherein the at least one Cas13 polypeptide has an amino acid sequence selected from the group consisting of SEQ ID NOS: 16-30, or a sequence being at least 80%, 85%, 90%, 91%, 92%, 93%, 94%, 95%, 96%, 97%, 98%, or 99% identical to SEQ ID NOS: 16-30 (ie functional variants). Specifically:
   when the Cas13 polypeptide is a Cas13a polypeptide, the Cas13a has an amino acid sequence selected from SEQ ID NO: 16, or SEQ ID NO:17, or SEQ ID NO: 18, or SEQ ID NO:19, or SEQ ID NO: 20, or SEQ ID NO:21, or SEQ ID NO:22, or is a functional variant thereof having 80-99% sequence identity to SEQ ID NOS: 16-22;
   when the Cas13 polypeptide is a Cas13b polypeptide, the Cas13b has an amino acid sequence selected from SEQ ID NO: 23, or SEQ ID NO:24, or SEQ ID NO: 25, or SEQ ID NO:26, or SEQ ID NO: 27, or is a functional variant thereof having 80-99% sequence identity to SEQ ID NOS: 16-22; or
   when the Cas13 polypeptide is a Cas13d polypeptide, the Cas13d has an amino acid sequence selected from SEQ ID NO: 28, or SEQ ID NO:29, or SEQ ID NO: 30 or is a functional variant thereof having 80-99% sequence identity to SEQ ID NOS: 28-30.

The Cas13 proteins and crRNAs are assembled in to ribonucleoprotein (RNP) complexes by standard methods known to the skilled person, by typically mixing the purified Cas13 with crRNA, together with an RNase inhibitor in a cleavage buffer. The RNP complexes are then mixed with the target RNA, wherein if the target it present, the RNP complex recognises and binds the target via the spacer sequences.

In an alternative embodiment, the CRISPR system comprises a nucleic acid molecule comprising a sequence encoding the Cas13 and ii) a nucleic acid molecule encoding said crRNA, the crRNA comprising one or more spacers and one or more Cas13-specific direct repeats, wherein said crRNA is capable of hybridising with one or more target RNA molecules. In this embodiment, the system further comprises one or more vectors for delivering and/or expressing the nucleic acid molecules. The vectors preferably comprise:
i) a first regulatory element operably linked to a nucleic acid molecule comprising a sequence encoding a Cas13 polypeptide wherein the Cas13 polypeptide has an amino acid sequence selected from the group consisting of SEQ ID NOS: 16-30, or a sequence being at least 80%, 85%, 90%, 91%, 92%, 93%, 94%, 95%, 96%, 97%, 98%, or 99% identical to SEQ ID NOS: 16-30; and
ii) a second regulatory element operably linked to a nucleic acid molecule encoding a CRISPR RNA (crRNA), the crRNA comprising one or more spacers and one or more Cas13-specific direct repeats, wherein the crRNA is capable of hybridising with one or more target RNA molecules.

In these embodiments of the invention, the at least one nucleic acid molecule comprising a sequence encoding said Cas13 (element (i)) is preferably selected from the group consisting of SEQ ID NOS: 1-15, or is selected from a sequence being at least 80%, 85%, 90%, 91%, 92%, 93%, 94%, 95%, 96%, 97%, 98%, or 99% identical to SEQ ID NOS: 1-15. (i.e. a sequence encoding functional variants). Specifically:
when the Cas13 polypeptide is a Cas13a polypeptide, the nucleic acid molecule encoding the Cas13a polypeptide comprises a sequence selected from SEQ ID NO: 1, or SEQ ID NO:2, or SEQ ID NO: 3, or SEQ ID NO:4, or SEQ ID NO: 5, or SEQ ID NO:6, or SEQ ID NO:7, or is a sequence that encodes a functional variant thereof;
when the Cas13 polypeptide is a Cas13b polypeptide, the nucleic acid molecule encoding the Cas13b polypeptide comprises a sequence selected from SEQ ID NO: 8, or SEQ ID NO:9, or SEQ ID NO: 10, or SEQ ID NO:11, or SEQ ID NO: 12, or is a sequence that encodes a functional variant thereof; or
when the Cas13 polypeptide is a Cas13d polypeptide, the nucleic acid molecule encoding the Cas13d polypeptide comprises a sequence selected from SEQ ID NO: 13, or SEQ ID NO:14, or SEQ ID NO: 15 or is a sequence that encodes a functional variant thereof.

Any of the above-mentioned methods can utilise two or more Cas13 polypeptides of the invention and two or more Cas13 CRISPR RNA in order to target different target sites of the same target RNA and/or can target different target RNA molecules e.g., based on variation such as single-nucleotide polymorphisms etc., and such could be used for example to target multiple strains of a virus such as Coronavirus variants, influenza virus variants, HIV variants, and the like.

The crRNA can include multiple spacer sequences to target multiple RNAs or to target multiple sequences within the same target RNA and/or the crRNA may be a precursor crRNA, that is processed in to the individual crRNAs. Or in a further alternative, when being used for targeting multiple RNAs, multiple crRNAs can be used.

### iv) Methods

There is also provided use of a CRISPR/Cas13 system in an in vitro method of modifying a target RNA, the method comprising contacting the target RNA with a ribonucleoprotein (RNP) complex of a CRISPR/Cas13 system, the system comprising
i) at least one Cas13 polypeptide wherein the Cas13 polypeptide has an amino acid sequence selected from the group consisting of SEQ ID NOS: 16-30, or a sequence being at least 80%, 85%, 90%, 91%, 92%, 93%, 94%, 95%, 96%, 97%, 98%, or 99% identical to SEQ ID NOS: 16-30; and
ii) at least one CRISPR RNA (crRNA), the crRNA comprising one or more spacers and one or more Cas13-specific direct repeats, wherein the crRNA is capable of hybridising with one or more target RNA molecules,
wherein the Cas13 polypeptide and the crRNA form a ribonucleoprotein (RNP) complex, and upon binding of the RNP complex to the target RNA through the one or more spacers, the Cas13 polypeptide modifies the target RNA.

In an alternative embodiment of this aspect of the invention, the in vitro method of modifying a target RNA the CRISPR/Cas13 system includes a vector system, and the method includes the preliminary step of:
a) expressing from the vector system at least one Cas13 polypeptide and at least one CRISPR RNA (crRNA), the vector system comprising one or more vectors comprising:
i) a first regulatory element operably linked to a nucleic acid molecule comprising a sequence encoding a Cas13 polypeptide wherein the Cas13 polypeptide has an amino acid sequence selected from the group consisting of SEQ ID NOS: 16-30, or a sequence being at least 80%, 85%, 90%, 91%, 92%, 93%, 94%, 95%, 96%, 97%, 98%, or 99% identical to SEQ ID NOS: 16-30; and
ii) a second regulatory element operably linked to a nucleic acid molecule encoding a CRISPR RNA (crRNA), the crRNA comprising one or more spacers and one or more Cas13-specific direct repeats, wherein the crRNA is capable of hybridising with one or more target RNA molecules;
wherein components (i) and (ii) are located on the same or different vectors of the system;
b) isolating the expression products of step (a).

The isolated expression products of step (b) can first be assembled in to ribonucleoprotein (RNP) complex as described above, and the RNP complex contacted with the target RNA, or the target RNA can be contacted with the isolated expression products of step (b), wherein the Cas13 polypeptide and the crRNA form the RNP complex, and the complex binds to the target RNA. In either embodiment, binding occurs through the one or more spacers, and once bound, the Cas13 polypeptide modifies the target RNA.

In these embodiments of the invention, the at least one nucleic acid molecule comprising a sequence encoding said Cas13 (element (i) is preferably selected from the group consisting of SEQ ID NOS: 1-15, or is selected from a sequence being at least 80%, 85%, 90%, 91%, 92%, 93%, 94%, 95%, 96%, 97%, 98%, or 99% identical to SEQ ID NOS: 1-15. (i.e. a sequence encoding functional variants). Specifically:
when the Cas13 polypeptide is a Cas13a polypeptide, the nucleic acid molecule encoding the Cas13a polypeptide comprises a sequence selected from SEQ ID NO: 1, or SEQ ID NO:2, or SEQ ID NO: 3, or SEQ ID NO:4, or SEQ ID NO: 5, or SEQ ID NO:6, or SEQ ID NO:7, or is a sequence that encodes a functional variant thereof;
when the Cas13 polypeptide is a Cas13b polypeptide, the nucleic acid molecule encoding the Cas13b polypeptide comprises a sequence selected from SEQ ID NO: 8, or SEQ ID NO:9, or SEQ ID NO: 10, or SEQ ID NO:11, or SEQ ID NO: 12, or is a sequence that encodes a functional variant thereof; or
when the Cas13 polypeptide is a Cas13d polypeptide, the nucleic acid molecule encoding the Cas13d polypeptide comprises a sequence selected from SEQ ID NO: 13, or SEQ ID NO:14, or SEQ ID NO: 15 or is a sequence that encodes a functional variant thereof.

In the above-mentioned methods of the invention, the trans-cleavage activity of the Cas13 polypeptides of the invention can be exploited to detect the cleavage of the target RNA. Because the Cas13 polyprotein of the invention cleaves non-targeted RNA once activated, which occurs when a Cas13 CRISPR RNA hybridizes with a target RNA in the presence of a Cas13 protein, a detectable signal can be any signal that is produced when the non-target RNA is cleaved.

Detection methods include a step of measuring a detectable signal produced by Cas13 trans cleavage of non-target RNA. The step of measuring can include one or more of: nanoparticle based detection, fluorescence or chemiluminescent detection, lateral-flow immunochromatography colloid phase transition/dispersion, electrochemical detection, semiconductor-based sensing, and detection of a RNA reporter (RNA molecules carrying a fluorophore and a quencher. Trans-cleavage activity on target detection causes the spatial separation of the fluorophore and the quencher, and the resulting fluorescence signal).

The readout of such detection methods can be any convenient readout. Examples of possible readouts include but are not limited to: a measured amount of detectable fluorescent signal; a visual analysis of bands on a gel (e.g., bands that represent cleaved product versus uncleaved substrate), a visual or sensor based detection of the presence or absence of a color (i.e., color detection method), and the presence or absence of (or a particular amount of) an electrical signal.

In embodiments of the methods of the invention that include a further detection step, the method can utilise two or more Cas13 polypeptides of the invention and two or more Cas13 CRISPR RNA in order to target and then detect, different target sites of the same target RNA (e.g., which can increase sensitivity of detection) and/or can target different target RNA molecules e.g., based on variation such as single-nucleotide polymorphisms etc., and such could be used for example to detect multiple strains of a virus such as Coronavirus variants, influenza virus variants, HIV variants, and the like.

In some embodiments for detecting multiple RNAs, two or more Cas13 crRNAs can be provided in the method. A precursor Cas13 crRNA can also be provided which can be processed into individual Cas13 crRNAs.

In alternative embodiments for detecting multiple RNAs, two or more Cas13 polypeptides cleaving different RNA probe sequences may be used. For example, one Cas13 polyprotein cleaves polyA RNA probe sequences but not polyU. The other polyprotein can cleave polyU RNA probe but not polyA probes. You can therefore tell which target is present by assessing which probe is cleaved.

### v) Vectors

In certain aspects the invention involves vectors for delivering or as already described, expressing the Cas13 polypeptide and the CRISPR RNA. The vectors may therefore be a part of a CRISPR/Cas13 system.

Vectors include, but are not limited to, nucleic acid molecules that are single-stranded, double-stranded, or partially double-stranded. The vectors can be nucleic acid molecules, plasmids, or viral vectors (e.g., AAV, adenovirus, lentivirus). Such vectors are also referred to herein as “expression vectors”, and may be gene expression vectors, or protein expression vectors.

The gene and protein expression vectors can comprise a nucleic acid molecule of the invention for expression in a host cell. The vectors therefore contain one or more regulatory elements, which may be selected on the basis of the host cells to be used for expression, that is operatively-linked to the nucleic acid sequence to be expressed.

In embodiments wherein the Cas13 polypeptide is encoded by a nucleic acid molecule, and the nucleic acid molecule is part of a vector, the nucleic acid molecule will be operably linked to a promoter. Suitable promoters include but are not limited to ubiquitous promoters (e.g., ubiquitin promoter), tissue-specific promoters, inducible promoters, and constitutive promoters.

### vi) Utility in Nucleic Acid Detection Assays/Kits

The trans-cleavage activity of the Cas13 polypeptides of the invention can be exploited and used in nucleic acid detection assays. Upon complex formation between the Cas13 polypeptide and the crRNA, and binding of the complex to the target RNA, the Cas13 polypeptide is “activated”. It is this activated form of Cas13 polypeptide that then binds and cleaves non-target RNA. Ie without any reliance on the spacer sequences in the crRNA. Detection of cleavage of this non-target RNA is therefore indicative of the presence of the target and can be both qualitative and quantitative.

The CRISPR/Cas13 polypeptide system of the invention can be utilised in any assay platform that requires detection of RNA. These include, but are not limited to:
Assays for detecting the presence of microbial agents in a biological sample from an animal, or in environmental samples. Eg to screen for microbial contamination in water, or contamination in food samples, or agricultural pathogens
Screening for mutations or single nucleotide polymorphisms, which more specifically, may be a diagnostic assay
Cancer screening and diagnosis, via early detection and monitoring of cancer markers
Screening for drug resistance including chemotherapy treatment resistance
Research tools

In a preferred embodiment, the CRISPR/Cas13 polypeptide system of the invention can be utilised in an assay to detect genes and mutations associated with cancer, or mutations associated with cancer drug resistance. Thus, the CRISPR/Cas13 polypeptide system of the invention provides low-cost, rapid, multiplexed cancer detection panels for circulating DNA, such as tumour DNA, particularly for monitoring disease recurrence or the development of common resistance mutations.

In an alternative embodiment, the CRISPR/Cas13 polypeptide system of the invention can be utilised in an assay to detect the presence of Coronavirus variants in an assay for diagnosing Covid infection.

The target RNA can therefore be from any biological, environmental or agricultural source including but not limited to water, soil, blood, human and plant tissue, or can be artificially created. In some embodiments, the target RNAs in the sample may be isolated and/or purified and/or amplified prior to contact with either ribonucleoprotein (RNP) complex or the Cas13 polypeptide and the crRNA. Any suitable RNA amplification technique may be used. Similarly, the target RNA in the sample may first be enriched prior to detection or amplification. This enrichment may be achieved by binding of the target nucleic acids by a CRISPR effector system.

The sample may also contain a target DNA. In these circumstances, the DNA can first be reverse transcribed using any suitable technique to produce RNA, and the transcribed RNA detected. If necessary, the DNA can also be isolated and/or purified and/or amplified and/or enriched just using any suitable technique known to the skilled person, prior to reverse transcription.

Multiple target sequences may be contained within the one sample.

In the nucleic acid detection method provided by the present invention, the method may further include determining binding and cleavage of the target RNA by means of a probe or detection molecule (“detector RNA”) that is itself cleaved via the trans cleavage activity of the Cas13 polypeptide of the invention.

SHERLOCK and DETECTR are two assays that already exploit the trans cleavage activity of different Cas13 polypeptides, and Cas12a polypeptides respectively. The concept behind them both is similar: reporters sequences/probes are mixed with the sample, such that when the Cas polypeptide within the Cas/crRNA complex is activated after binding to the specific target sequence, the reporter sequences are then indiscriminately cleaved. By using reporter sequences bound to a fluorophore at one end, and a quencher on the other, degradation of the reporter sequence releases fluorophores and results in stable and strong fluorescent signal detected by a fluorimeter. The presence and intensity of the fluorescent signal thus indicates the amount of the target in the biological sample.

In another aspect of the invention there is provided a nucleic acid detection system for detecting a target RNA in a sample, which may come in a kit form, the system comprising:
i) at least one Cas13 polypeptide wherein the Cas13 polypeptide has an amino acid sequence selected from the group consisting of SEQ ID NOS: 16-30, or a sequence being at least 80%, 85%, 90%, 91%, 92%, 93%, 94%, 95%, 96%, 97%, 98%, or 99% identical to SEQ ID NOS: 16-30; or a nucleic acid molecule comprising a sequence encoding the Cas13 polypeptide and
ii) at least one CRISPR RNA (crRNA) or a nucleic acid molecule encoding the crRNA, the crRNA comprising one or more spacers and one or more Cas13-specific direct repeats, wherein the crRNA is capable of hybridising with one or more target RNA molecules; and preferably
iii) a detector RNA.

Preferably the nucleic acid molecule comprising a sequence encoding the Cas13 polypeptide is selected from the group consisting of SEQ ID NOS: 1-15, or is selected from a sequence being at least 80%, 85%, 90%, 91%, 92%, 93%, 94%, 95%, 96%, 97%, 98%, or 99% identical to SEQ ID NOS: 1-15.

When the detection assay comprises a nucleic acid molecule comprising a sequence encoding the Cas13 polypeptide and a nucleic acid molecule encoding the crRNA, both nucleic acid molecules are preferably expressed from vectors, the vectors comprising:
i) a first regulatory element operably linked to a nucleic acid molecule comprising a sequence encoding a Cas13 polypeptide wherein the Cas13 polypeptide has an amino acid sequence selected from the group consisting of SEQ ID NOS: 16-30, or a sequence being at least 80%, 85%, 90%, 91%, 92%, 93%, 94%, 95%, 96%, 97%, 98%, or 99% identical to SEQ ID NOS: 16-30; and
ii) a second regulatory element operably linked to a nucleic acid molecule encoding a CRISPR RNA (crRNA), the crRNA comprising one or more spacers and one or more Cas13-specific direct repeats, wherein the crRNA is capable of hybridising with one or more target RNA molecules.

Preferably the nucleic acid molecule comprising a sequence encoding the Cas13 polypeptide is selected from the group consisting of SEQ ID NOS: 1-15, or is selected from a sequence being at least 80%, 85%, 90%, 91%, 92%, 93%, 94%, 95%, 96%, 97%, 98%, or 99% identical to SEQ ID NOS: 1-15.

In some embodiments, when there are two or more Cas13 crRNAs, the crRNAs can be provided via a nucleic acid molecule encoding a precursor Cas13 crRNA which can be processed into individual Cas13 crRNAs each of which has a different spacer sequence within it designed against different targets.

The detector RNA can be, for example, a labeled detector RNA such as a fluorescence-emitting dye pair, i.e., a FRET pair and/or a quencher/fluor pair or RNA molecule generating any other detectable signal after collateral cleavage.

The step of measuring can include one or more of: nanoparticle based detection, fluorescence or chemiluminescent detection, lateral-flow immunochromatography colloid phase transition/dispersion, electrochemical detection, semiconductor-based sensing, and detection of a RNA reporter (RNA molecules carrying a fluorophore and a quencher. Trans-cleavage activity on target detection causes the spatial separation of the fluorophore and the quencher, and the resulting fluorescence signal).

The readout of such detection methods can be any convenient readout. Examples of possible readouts include but are not limited to: a measured amount of detectable fluorescent signal; a visual analysis of bands on a gel (e.g., bands that represent cleaved product versus uncleaved substrate), a visual or sensor based detection of the presence or absence of a color (i.e., color detection method), and the presence or absence of (or a particular amount of) an electrical signal.

The systems, assays/kits and methods disclosed herein may also be adapted for detection of polypeptides (or other molecules) in addition to detection of nucleic acids, via incorporation of a specifically configured polypeptide detection aptamer. Accordingly, in certain example embodiments, the systems, assays/kits and methods may further comprise one or more detection aptamers.

Embodiments disclosed herein can detect both RNA and DNA with comparable levels of sensitivity and can differentiate targets from non-targets based on single base pair differences. Moreover, the embodiments disclosed herein can be prepared in freeze-dried format for convenient distribution and point-of-care (POC) applications.

The nucleic acid detection system of the invention for detecting a target RNA in a sample, and as already mentioned above in the context of the methods of the invention, can utilise two or more Cas13 polypeptides of the invention and two or more Cas13 crRNA in order to target and detect different target sites of the same target RNA (e.g., which can increase sensitivity of detection) by designing the spacer of the crRNA appropriately, and/or can target different target RNA molecules e.g., based on variation such as single-nucleotide polymorphisms etc., and such could be used for example to detect multiple strains of a virus such as Coronavirus variants, influenza virus variants, HIV variants, and the like.

In some embodiments, two or more Cas13 CRISPR RNAs can be present on an array. For example, a precursor Cas13 crRNA array which can be cleaved into individual Cas13 crRNAs with different spacer sequences in order to bind different targets. In alternative embodiments, two or more Cas13 polypeptides cleaving different RNA probe sequences may be used. For example, one Cas13 polypeptides cleaves polyA RNA probe sequences but not polyU. The other polyprotein can cleave polyU RNA probe but not polyA probes and two or more probes (e.g., with different RNA sequences).

The nucleic acid detection systems and kits of the invention can be applied to any sample being assessed for the presence of a target RNA. Or as already noted, a sample containing DNA that has been reverse transcribed to RNA. The sample can therefore be from any biological, environmental or agricultural source. In some embodiments, the target RNAs in the sample may be isolated and/or purified and/or amplified prior to contact with either ribonucleoprotein (RNP) complex or the Cas13 polypeptide and the crRNA. Any suitable RNA amplification technique may be used. Similarly, the target RNA in the sample may first be enriched prior to detection or amplification. This enrichment may be achieved by binding of the target nucleic acids by a CRISPR effector system.

Multiple target sequences may be contained within the one sample.

The invention also seeks to provide a method of using the nucleic acid detection systems and kits of the invention as described herein for detecting a target RNA in a sample, the method including one or more of the following steps:
Obtaining a sample if one has not already been supplied
Processing the sample if need be to isolate any nucleic acids present in the sample
Purifying and/or enriching and/or amplifying the nucleic acids if need be
Reverse transcribing DNA from the sample to RNA if need be
Contacting the sample/nucleic acid with either (i) a pre-formed RNP complex as described herein; or (ii) a Cas13 polypeptide and a crRNA of the invention; or (iii) vectors from which a Cas13 polypeptide and a crRNA of the invention can be expressed
Adding one or more detector RNAs to the reaction
Detecting cleavage of the one or more probe/reporter RNAs when the target RNA is present in the sample.

As will be demonstrated in the examples, the Cas13 polypeptides of the invention represent important new sensitive and specific enzymes that will be particularly useful a CRISPR/Cas system in nucleic acid detection assays.

### EXAMPLES

### 1. Metagenomics Data Analysis and Computational Identification of Novel Cas13a, Cas13b, and Cas13d

### Method

Metagenome sequencing of camel, cattle, and sheep rumen was performed using Illumina HiSeq 2500 system with 150 bp paired-end sequencing. All metagenome sequences of each species were co-assembled using MEGAHIT v1.2.9 with the following options: --k-min 31, --k-max 141, --k-step 10, --min-count 2, and --min-contig-len 200. The FASTAsplitter software (Version 0.2.6) was used to split initial camel, cattle, and sheep rumen metagenomic contigs into files with <50M size. CRISPRone and CRISPRCasFinder 1.1.2 was then used for CRISPR/Cas prediction and region analysis. CRISPRDetect was then used to predict the orientation of the direct repeat in the Cas13 CRISPR array. In order to detect the transcription direction of the CRISPR region, the sequence alignment and secondary structure prediction were performed for CRISPR repeats from all 15 selected Cas13 proteins.

Protein sequence alignment of Cas13s was performed using ClustalW in MEGA11 with default settings. Rumen metagenomics derived Cas13 were compared with previously known (characterized) Cas13a, Cas13b, and Cas13d. The proteins similarity search was performed by BLASTp search against the nonredundant protein database curated by the National Centre for Biotechnology Information (NCBI)(http//blast.ncbi.nlm.nih.gov/blast.cgi).

References for contigs used to discover Cas13 polyproteins:
Gharechahi J, et al. NPJ Biofilms Microbiomes. 2022 Jun. 8; 8(1):46.
Gharechahi J, et al. ISME J. 2021 April; 15(4):1108-1120.

### Results

### a) Identification

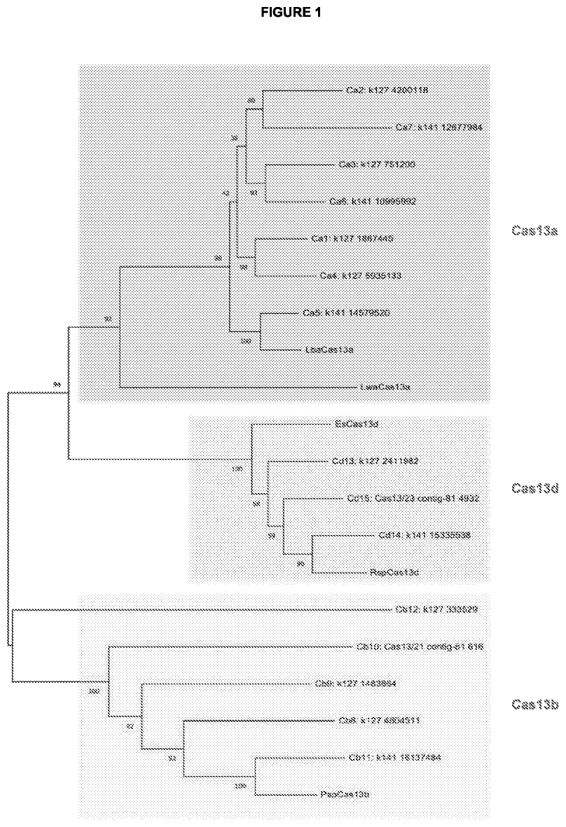

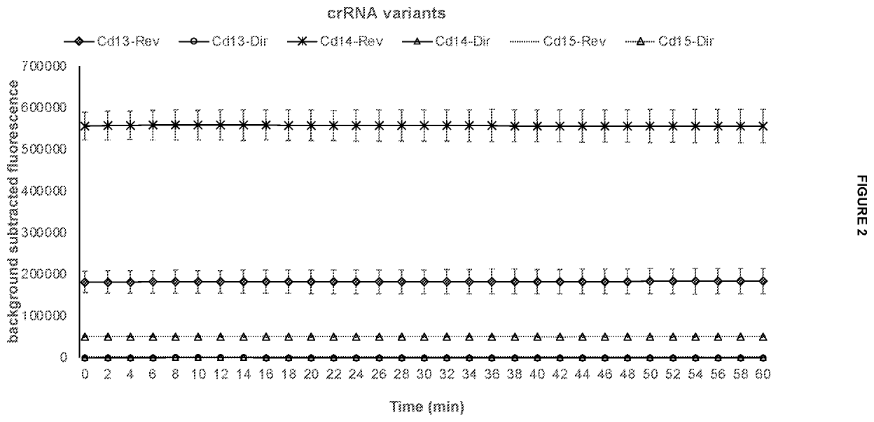

Fifteen (15) candidate Cas13 proteins encoding intact CRISPR-Cas13 systems and containing active site domains and residues were selected (Table 2 and phylogenic analysis FIG. 1):

TABLE 2
Novel Cas13 enzymes subtyping and nomenclature

| Subtype | Enzyme of the invention |
|---|---|
| Cas 13a | Ca1, Ca2, Ca3, Ca4, Ca5, Ca6 and Ca7 |
| Cas13b | Cb8, Cb9, Cb10, Cb11 and Cb12 |
| Cas13d | Cd13, Cd14 and Cd15. |

A Blast search against the non-redundant protein database curated by NCBI showed that all of the Cas13a enzymes of the invention showed less than 60 percent identity to other Cas13a proteins deposited in the data, most of the Cas13b enzymes of the invention showed less than 40-50 percent identity to proteins deposited in this database, and all 3 of the Cas13d enzymes of the invention showed less than 50 percent identity to proteins deposited in this database (Table 3).

This demonstrates how different the Cas13 polypeptides of the invention are from those already known in the art.

TABLE 3
The % identity of novel Cas13 proteins of the invention to proteins
deposited in GenBank. The Expect value (E) is a parameter that
describes the number of hits one can “expect” to see
by chance when searching a database of a particular size.

| | SEQ | | | | | |
|---|---|---|---|---|---|---|
| | ID | | | Protein | % | E |
| Subtype | NO: | ID | Contig ID | length | Identity | value |
| A | 16 | Ca1 | k127_1867445 | 1338 | <56% | 2e⁻¹¹ |
| | 17 | Ca2 | k127_4200118 | 1382 | <40% | 0.0 |
| | 18 | Ca3 | k127_751200 | 1307 | <39% | 0.0 |
| | 19 | Ca4 | k127_5935133 | 1319 | <59% | 0.0 |
| | 20 | Ca5 | k141_14579520 | 1406 | <53% | 0.0 |
| | 21 | Ca6 | k141_10995992 | 1341 | <49% | 0.0 |
| | 22 | Ca7 | k141_12677984 | 1302 | <44% | 7e⁻⁰⁵ |
| B | 23 | Cb8 | k127_4804511 | 1313 | <68% | 7e⁻¹⁴⁸ |
| | 24 | Cb9 | k127_1483864 | 1125 | <32% | 1e⁻¹⁴⁴ |
| | 25 | Cb10 | Cas13/21_contig- | 1215 | <79% | 0.0 |
| | | | 18_616 | | | |
| | 26 | Cb11 | k141_16137484 | 1380 | <35% | 1e⁻¹⁷⁶ |
| | 27 | Cb12 | k127_333529 | 1246 | <45% | 3e⁻⁰⁷ |
| D | 28 | Cd13 | K127_2411982 | 921 | <44% | 0.0 |
| | 29 | Cd14 | K141_15335538 | 956 | <44% | 2e⁻⁶³ |
| | 30 | Cd15 | Cas13/23_contig- | 948 | <43% | 1e⁻⁶⁰ |
| | | | 81_4932 | | | |

b) crRNA Modelling

Sequence alignment and secondary structure modelling was performed for the CRISPR repeats (ie the direct repeats) of the crRNA for all 15 selected Cas13 polypeptides to identify the transcription direction of CRISPR region. All the repeats in the crRNA for the Cas13a and Cas13d proteins formed the hairpin at the 5′ end just before the spacer and all of the crRNA for the Cas13b proteins formed the hairpin at the 3′ end.

In the following tables, a number of crRNAs (SEQ ID NOS: 32-54) with direct repeats of 35-37 nucleotides (underlined), are provided for each Cas13 polyprotein of the invention. The consensus direct repeat of the crRNA for each is provided in Table 4A; exemplary variants of the direct repeat is provided in Tables 4B to 4L. All are provided as the coding DNA sequence. As would be understood, the spacer will need to be designed for the target RNA; in Table 4A, an exemplary spacer sequence is used which is designed against an artificial target sequence.

TABLE 4A
List of crRNAs with consensus direct repeat; LWA = Leptotrichia wadei
(LwaCas13a) as reference sequence. All sequences given as the coding DNA sequence.

| SEQ | | | |
|---|---|---|---|
| ID | | | |
| NO: | ID | Direct repeat + exemplary spacer (listed as the coding DNA sequence) | Length |
| | Cas13a | | |
| 31 | LWA | GATTTAGACTACCCCAAAAACGAAGGGGACTAAAACTAGATTGCTGTTCTACCAAGTAATCCAT | 64 |
| 32 | Ca1 | GTGGCATGAAAAAAGCCCGACATAGCGGGCAATCACTAGATTGCTGTTCTACCAAGTAATCCAT | 64 |
| 33 | Ca2 | GTAGAAAAGAAGATAGTCCAACATAGTGGATAATCATAGATTGCTGTTCTACCAAGTAATCCAT | 64 |
| 34 | Ca3 | GGAGATGAAAAAAGCCCGACATAGCGGGCAATCGAATAGATTGCTGTTCTACCAAGTAATCCAT | 64 |
| 35 | Ca4 | GTTAAAAGAAAACAGCCCGACATAGCGGGCGATAACTAGATTGCTGTTCTACCAAGTAATCCAT | 64 |
| 36 | Ca5 | GAATTGGAGAAGATCCCGAGAAAGTGGGAAATAACTAGATTGCTGTTCTACCAAGTAATCCAT | 63 |
| 37 | Ca6 | GTTTGGAAAACAGCCCGACATAGAGGGCAATAGACTAGATTGCTGTTCTACCAAGTAATCCAT | 63 |
| 38 | Ca7 | GTTAGATGAGAACACTCCGAGATAACGGAGAATAACTAGATTGCTGTTCTACCAAGTAATCCAT | 64 |
| | Cas13b | | |
| 39 | Cb8 dir | TAGATTGCTGTTCTACCAAGTAATCCATATGTTGTCATACCCATCCAAACGATAGGCTTCTACAAC | 66 |
| 40 | Cb8 rev | TAGATTGCTGTTCTACCAAGTAATCCATATGTTGTAGAAGCCTATCGTTTGGATGGGTATGACAAC | 66 |
| 41 | Cb9 dir | TAGATTGCTGTTCTACCAAGTAATCCATATGTTGTAAAGACCTTCATTTCGGAAGGAAGAGACAAC | 66 |
| 42 | Cb9 rev | TAGATTGCTGTTCTACCAAGTAATCCATATGTTGTCTCTTCCTTCCGAAATGAAGGTCTTTACAAC | 66 |
| 43 | Cb10 dir | TAGATTGCTGTTCTACCAAGTAATCCATATGTTGTAGAAGCCCCTTCTTCGTGGGGTAAGTGCAAC | 66 |
| 44 | Cb10 rev | TAGATTGCTGTTCTACCAAGTAATCCATATGTTGCACTTACCCCACGAAGAAGGGGCTTCTACAAC | 66 |
| 45 | Cb11 dir | TAGATTGCTGTTCTACCAAGTAATCCATATGTTGTAGAAGCCTATCGTTTGGATAGGTATGACAAC | 66 |
| 46 | Cb11 rev | TAGATTGCTGTTCTACCAAGTAATCCATATGTTGTCATACCTATCCAAACGATAGGCTTCTACAAC | 66 |
| 47 | Cb12 dir | TAGATTGCTGTTCTACCAAGTAATCCATATGCTGTGAACTCCTGCCGAAATGGCAGGCCGAACAGC | 66 |
| 48 | Cb12 rev | TAGATTGCTGTTCTACCAAGTAATCCATATGCTGTTCGGCCTGCCATTTCGGCAGGAGTTCACAGC | 66 |
| | Cas13d | | |
| 49 | Cd13 dir | GGTTTCAGACCCTTACAAAAAGGGTGTAGTACGTTTCTAGATTGCTGTTCTACCAAGTAATCCAT | 65 |
| 50 | Cd13 rev | GAAACGTACTACACCCTTTTTGTAAGGGTCTGAAACCTAGATTGCTGTTCTACCAAGTAATCCAT | 65 |
| 51 | Cd14 dir | GTTTCAGAACCCTGTAATTTGACAGGGTTGTAGTTGTAGATTGCTGTTCTACCAAGTAATCCAT | 64 |
| 52 | Cd14 rev | CAACTACAACCCTGTCAAATTACAGGGTTCTGAAACTAGATTGCTGTTCTACCAAGTAATCCAT | 64 |
| 53 | Cd15 dir | GATCTATAACCCTGCATTTATGTAGGGCTCTAAAACTAGATTGCTGTTCTACCAAGTAATCCAT | 64 |
| 54 | Cd15 rev | GTTTTAGAGCCCTACATAAATGCAGGGTTATAGATCTAGATTGCTGTTCTACCAAGTAATCCAT | 64 |

TABLE 4B
Cas13 Ca2

DR consensus: GTAGAAAAGAAGATAGTCCAACATAGTGGATAATCA
(SEQ ID NO: 86)

| SEQ ID NO: | Direct Repeat variants |
|---|---|
| 55 | GTAGAAAATAAGATAGTCCAACATAGTGAATAATCA |
| 56 | GTAGAAAAGAAGATAGTCCAACATAGCGGATAATCA |
| 57 | GTAGAAAAGAAGATAGTCCAACACAGTGGATAATCA |
| 58 | GTATTAACGAAGACAGTCCAACATAGTGGACACTCA |
| 59 | GAAGAAATGAAGACAGTCCAACATGGTGGATAATCA |
| 60 | GTAGAAATGAGGATAGTCCAACATAGCAACTAATTA |

TABLE 4C
Cas13 Ca3

DR consensus: GGAGATGAAAAAAGCCCGACATAGCGGGCAATCGAA
(SEQ ID NO: 94)

| SEQ ID NO | Direct Repeat variants |
|---|---|
| 61 | GGAGATGAAAAAAGCCCGACATAGCGGGCAATCGAAAT |
| 62 | GGGGATGAAAAAAGCCCGACATAGCGGGCAATCGAACT |
| 63 | GGAGATGAAAAAAGCCCGACATAGCGGGCAATCGAACT |
| 64 | GGAGATGAAAAAAGTCCAACATTATGAAGACTATAGAG |

TABLE 4D
Cas13 Ca4

DR consensus: GTTAAAAGAAAACAGCCCGACATAGCGGGCGATAAC
(SEQ ID NO: 87)

| SEQ ID NO | Direct Repeat variants |
|---|---|
| 65 | GTTAAAAGAAAACAGCCCGACATAGTGGGCGATAAC |
| 66 | GTTAAAAGAAAATAGCCCGACATAGCGGGCGATAAC |
| 67 | GTTAAAAGAAAACAGCCCGACATAACGAGCGATAAC |

TABLE 4E
Cas13 Ca5

DR consensus: GAATTGGAGAAGATCCCGAGAAAGTGGGAAATAAC
(SEQ ID NO: 95)

| SEQ ID NO | Direct Repeat variants |
|---|---|
| 68 | GACTTGGAGAAGATCCCGAGAAAGTGGGAAATAAC |
| 69 | GAATTGGAGAAGATCCCGAGATTAAACACAGAAAA |

TABLE 4F
Cas13 Ca6

DR consensus: GTTTGGAAAACAGCCCGACATAGAGGGCAATAGAC
(SEQ ID NO: 96)

| SEQ ID NO | Direct Repeat variants |
|---|---|
| 70 | GTTTGGAAAACAGCCCGACAAAGAGGGCAATAGAC |

TABLE 4G
Cas13 Cb9

DR consensus: GTTGTAAAGACCTTCATTTCGGAAGGAAGAGACAAC
(SEQ ID NO: 97)

| SEQ ID NO | Direct Repeat variants |
|---|---|
| 71 | GTTGTAAAGACCTTCATTTTGGAAGGAAGAGACAAC |
| 72 | GTTGTAAAGACCTTCATTTTGGAAGGAGGAGACATC |

TABLE 4H
Cas13 Cb10

DR consensus: GTTGTAGAAGCCCCTTCTTCGTGGGGTAAGTGCAAC
(SEQ ID NO: 98)

| SEQ ID NO | Direct Repeat variants |
|---|---|
| 73 | GTTGTAGAAGCCCCTTCTTTGTGGGGTAGGTGCAAC |
| 74 | GTTGTAGAAGCCCCTTCTTTGTGGGGTAAGTGCAAC |
| 75 | GTTGTAGAAGCCCCTTCTTTGTGGGGTATGTGCAAC |

TABLE 4I
Cas13 Cb11

DR consensus: GTTGTAGAAGCCTATCGTTTGGATAGGTATGACAAC
(SEQ ID NO: 99)

| SEQ ID NO | Direct Repeat variants |
|---|---|
| 76 | GTTGTAGAAGCCTATCGTGTGGATAGGTATGACAAC |
| 77 | GTTGTAGAAGCCTATCGTTTGGGTAGGTACGACAAA |

TABLE 4J
Cas13 Cd13

DR consensus: GGTTTCAGACCCTTACAAAAAGGGTGTAGTACGTTTC
(SEQ ID NO: 100)

| SEQ ID NO | Direct Repeat variants |
|---|---|
| 78 | GTTTCAGACCCTTACAAAAAGGGTGTAGTACGTTTCT |
| 79 | GTTTCAGACCCTTATTAAAAGGGTGTAGTACGTTTCG |
| 80 | GTTTCAGACCCTTACTAAAAGGGTGTAGTACAAAACA |
| 81 | GTTTTAGACCCATGCAAAATGGGTGTAGTACAAAACC |
| 82 | TTGTCTTTTCCCAACAAAAAAGGGTGTAGTACGTTTC |

TABLE 4K
Cas13 Cd14

DR consensus: GTTTCAGAACCCTGTAATTTGACAGGGTTGTAGTTG
(SEQ ID NO: 101)

| SEQ ID NO | Direct Repeat variants |
|---|---|
| 83 | CAACTACATCTCTGTAATCTAACAGGGTTGTAGTTG |
| 84 | GTTTCAGAATCCTGTAATTTGACAGGGTTGTAGTTG |

TABLE 4L
Cas13 Cd15

DR consensus: GATCTATAACCCTGCATTTATGTAGGGCTCTAAAAC
(SEQ ID NO: 102)

| SEQ ID NO | Direct Repeat variants |
|---|---|
| 85 | GATCTATAACCCTGCATTTATGCAGGGCTCTAAAAT |

### 2. Expression and Purification of Cas13 Enzymes

Cas13 sequences were N-terminally tagged with a His6-MBP-TEV (His6, six-histidine affinity tag; MBP, maltose binding protein to enhance solubility; TEV, TEV protease recognition site) and C-terminally tagged with enterokinase (EK) cleavage site (EK-His6). His6-MBP-TEV-Cas13-EK-His6 were synthesised and cloned between Ndel and NotI restriction endonuclease site of pET21a(+) by Genscript (USA). After dilution of lyophilized synthesis vector and agarose gel analysis, 50 ng of His6-MBP-TEV-Cas13-EK-His6 pET21a(+) plasmid was transformed into E. coli host strain BL21 (DE3).

First, the expression was performed at a small (20 ml Terrific broth (TB)) scale in 100 cc flask to screen optimum conditions for soluble protein expression.

Cells were grown aerobically in a shaker at 37° C. at 180 rpm up to an optical density of 0.6-0.8, before inducing Cas13 production with inductor concentrations (400 μM of IPTG) in an overnight culture at (16 and 18° C.). Then, the optimum conditions determined previous step were used to grow a large amount of biomass in 1-4 L of Terrific broth (TB) media. After induction, cells were pelleted and re-suspended in lysis buffer (50 mM Tris-HCl pH 7, 500 mM NaCl, 5% glycerol and 1 mM TCEP, 0.5 mM PMSF, EDTA-free protease inhibitor (Roche)). 0.1-1 mg/ml lysozyme and 500 mg-1G/Lit protamine sulphate were added, then incubated on ice for 30 min and mixed 2-3 times by gently swirling the cell suspension. Cells were then sonicated (60% Amplitude, 0.5 Cycle, Sonication Pulse Rate: 360 seconds ON) on ice. Cell debris was removed by centrifugation at 14,000 g for 30 min at 4° C.

Cell debris was removed by 0.45 micron filter. Then the filtered cell lysate was loaded into the column including 1 cc Ni-NTA resin (Sigma) per 10 cc filtered cell (equivalent to 1/10 of the volume of cell supernatant), and subsequently washed with 4 ml wash buffer (50 mM sodium phosphate pH 8, 300 mM NaCl, and 10 mM Imidazole). The protein was then eluted with 2 mL elution buffer each (50 mM sodium phosphate pH 8, 300 mM NaCl, 300 mM Imidazole and 1 mM TCEP). Lysate, flow-through, wash and elution fractions were collected individually and analysed on a 10% SDS gel.

Proteins in 2-14 ml elute fraction were concentrated with 3 or 30 k Amicon filter (Merck Millipore). Concentrated proteins were incubated with TEV and EK proteases at 4° C. overnight while dialyzing into ion exchange buffer (50 mM Tris-HCl pH 7.0, 250 mM KCl, 5% glycerol, 1 mM TCEP) in order to cleave off the N-terminal His6-MBP and C-terminal His6 tags, respectively. Cleaved Cas13d enzymes were loaded onto Amicon Ultra-0.5 ml centrifugal Filter Ultracel-50k (50000NMWL)(Merck Millipore) and cleaved Cas13a and Cas13b enzymes were loaded onto micron Ultra-15 centrifugal Filter Ultracel-100k (100000NMWL)(Merck Millipore) centrifugal filters. Concentrated proteins were diluted with storage buffer (20 mM Tris-HCl pH 7.0, 200 mM KCl, 5% glycerol, 1 mM TCEP) for subsequent enzymatic assays.

The concentrated factions were analysed on a 10% SDS gel.

### 3. Nucleic Acid Preparation

Artificial target sequence with either a C, G, A or U protospacer flanking sequence (PFS) were designed. DNA oligo templates for T7 transcription were synthesised and cloned in pUC57 by GenScript (Table 5).

TABLE 5
DNA fragments for production of RNA substrates (ie target sequences)

| Name | Sequence |
|---|---|
| ssRNA 1 | GGCCAGTGAATTCGAGCTCGGTACCCGGGGATCCTCTAGAAATATGGATTACTTGGTAGAAC |
| (C PFS) | AGCAATCTACTCGACCTGCAGGCATGCAAGCTTGGCGTAATCATGGTCATAGCTGTTTCCTGT |
| | GTTTATCCGCTCACAATTCCACACAACATACGAGCCGGAAGCATAAAG (SEQ ID NO: 88) |
| ssRNA 1 | GGCCAGTGAATTCGAGCTCGGTACCCGGGGATCCTCTAGAAATATGGATTACTTGGTAGAAC |
| (G PFS) | AGCAATCTAGTCGACCTGCAGGCATGCAAGCTTGGCGTAATCATGGTCATAGCTGTTTCCTGT |
| | GTTTATCCGCTCACAATTCCACACAACATACGAGCCGGAAGCATAAAG (SEQ ID NO: 89) |
| ssRNA 1 | GGCCAGTGAATTCGAGCTCGGTACCCGGGGATCCTCTAGAAATATGGATTACTTGGTAGAAC |
| (A PFS) | AGCAATCTAATCGACCTGCAGGCATGCAAGCTTGGCGTAATCATGGTCATAGCTGTTTCCTGT |
| | GTTTATCCGCTCACAATTCCACACAACATACGAGCCGGAAGCATAAAG (SEQ ID NO: 90) |
| ssRNA 1 | GGCCAGTGAATTCGAGCTCGGTACCCGGGGATCCTCTAGAAATATGGATTACTTGGTAGAAC |
| (U PFS) | AGCAATCTATTCGACCTGCAGGCATGCAAGCTTGGCGTAATCATGGTCATAGCTGTTTCCTGT |
| | GTTTATCCGCTCACAATTCCACACAACATACGAGCCGGAAGCATAAAG (SEQ ID NO: 91) |

Phusion™ High-Fidelity PCR Master Mix (2X) (Thermo Fisher Scientific) with forward (TAATACGACTCACTATAG (SEQ ID NO:92)) and reverse (CTTTATGCTTCCGGCTCG (SEQ ID NO:93)) primers were used for amplification of template DNA. PCR assembly reaction in a 25-μL volume was set up according to manufacturer's instructions and using the cycling parameters according to manufacturer's recommendations, with an annealing temperature of 55° C. and 35 cycles. The integrity of amplified sequence was checked by Sanger sequencing. PCR product was run on 2% agarose gel and template band was recovered from gel by GeneJET™ Gel Extraction Kit (Thermo Fisher Scientific). 3 μl of gel recovered DNA was run on 2% agarose gel. The concentration of purified DNA was determined by Picodrop and Qubit Fluorometer.

TranscriptAid T7 High Yield Transcription Kit (Thermo Fisher Scientific) was used for ssRNA template synthesis using the T7 RNA polymerase forward primer.

The ssRNA reaction components were combined at room temperature according to manufacturer's instructions using 1 μg of template, mixed thoroughly, and then centrifuged briefly to collect all drops and incubated at 37° C. for 4 h in water bath. DNase I digestion directly after the IVT reaction was performed to prevent the template DNA from interfering with downstream applications of the RNA transcript. 1 μL of DNase I (at 1 U/μL; included in the kit) was added into the reaction mix immediately after the IVT reaction and incubate at 37° C. for 15 minutes. 0.5 μL of the IVT product was diluted in 10 μL of DEPC-treated water and 10 μL of the diluted sample was mixed with 10 μL of 2×RNA Loading Dye Solution of the TranscriptAid T7 High Yield Transcription Kit and incubated at 70° C. for 10 minutes and then was chilled on ice prior to loading. Samples were run on a 2% Agarose Gel against an RNA Ladder.

RNA purification from IVT reaction was performed using GeneJET RNA Cleanup and Concentration kit (Thermo Fisher Scientific) as instructed by manufacturer. The purified RNA was stored at −80° C. until use. 0.5 μL of the purified IVT product was diluted in 10 μL of DEPC-treated water and 10 μL of the diluted sample was mixed with 10 μL of 2×RNA Loading Dye Solution and was heated the sample at 70° C. for 10 minutes and was chilled on ice prior to loading on a 2% Agarose Gel against an RNA Ladder.

### 4. In Vitro Cis Cleavage Assays and Activity of CRISPR-Cas13

The ability of the Cas13 proteins of the invention to exhibit cis ssRNAse activity was assessed.

### Method

All crRNAs (Table 4A) used in this study were synthesised by GenScript. The cleavage reactions of Cas13 enzymes were performed at 37° C. with the in vitro-transcribed RNA targets shown in Table 5.

Briefly, cleavage reactions were carried out in 20 μL reaction volume with 100 nM Cas13 protein of the invention, 50 nM crRNAs (unless otherwise indicated), and 1000 nM in vitro-transcribed target RNA in a 1× cleavage buffer (Buffer 1, Table 7); the reactions were then incubated at 37° C. for 1 h (unless otherwise indicated). The samples were then boiled at 70° C. for 3 min in a 2×RNA Loading Dye (NEB) and cooled down on the ice for 3 min before loading onto a 10% polyacrylamide-urea denaturing gel. The gel was stained with the SYBR Gold Nucleic Acid Gel Stain (Thermo Fisher Scientific) and visualized using a Bio-Rad Molecular Imager Gel Doc system.

### Results

The corresponding gene sequence of the Cas13 proteins was synthesised and the proteins expressed in Escherichia coli. The proteins were purified and their in vitro cis catalytic activities tested. The in vitro cleavage activity of the Cas13 proteins of the invention with the respective crRNAs listed in Table 4 targeting the single-stranded RNA substrates of Table 5 harbouring target sequences complementary to the crRNA spacers was evaluated. The activity of Cas13 proteins was evaluated by denaturing gel showing the targeted in vitro RNase cleavage activity of the Cas13 proteins of the invention when incubated with the ssRNA target and different crRNAs. The ssRNA cleavage activity of 15 Cas13 enzymes, all of them showed at least some ssRNA cleavage activity.

5. In Vitro Trans Cleavage Assays and Collateral ssRNAse Activity of Cas13 Polypeptides

The ability of the Cas13 proteins of the invention to induce collateral (ie trans) ssRNAse activity was assessed.

### Methods

Collateral cleavage assays of Cas13 enzymes were performed in a 20 μL final reaction volume. Cas13 proteins and crRNAs according to Table 4 were first assembled in to ribonucleoprotein (RNP) complexes by mixing 50 nM purified Cas13 with 25 nM crRNA in the 1× cleavage buffer (Buffer 1, Table 7);) followed by incubation at 37° C. for 60 min (unless otherwise indicated).

Next, the assembled RNP was combined on ice with 2 μL of 250 nM in vitro-transcribed target RNA (Table 5) and 250 nM RNA reporter (UUUUU) (Integrated DNA Technologies) (unless otherwise indicated), and reactions were incubated for 1 h at 37° C. (unless otherwise indicated). Real-time or end-point fluorescence measurements were collected on a microplate reader (Synergy HTX Multi-Mode Reader) or ABI Real-time PCR (Applied Biosystems, CA, USA) at 2 min intervals for real-time measurements. To allow comparisons between different conditions, fluorescence for background conditions (no target ssRNA) were subtracted from samples to generate background subtracted fluorescence.

### Results

Reactions were performed in the presence of fluorescently labelled ssRNA probe (UUUUU) and equally mixed ssRNA targets (ie a mixture of the 4 ssRNAs of Table 5). It is possible to predict the direct repeat direction only for Cas13a proteins; for Cas13b and Cas13d it should be determined experimentally. Accordingly, two crRNA variants (direct (dir) or reverse (rev)) were designed and tested. Trans cleavage (collateral activity) was detected by all proteins; the measurement of real-time background subtracted fluorescence output for a subset of them (Cas13d13, Cas13d14 and Cas13d15) are presented in FIG. 2.

The results showed that Cas13d13 and Cas13d14 were functional using reverse crRNA whereas Cas13d15 showed collateral cleavage activity using direct crRNA.

Optimisation of Collateral ssRNAse Activity of Cas13 Polypeptides

In order to develop a nucleic acid detection platform based on the CRISPR/Cas13 system of the invention, particular elements of the platform and/or assay can be optimised. The following examples demonstrate the elements of a platform that can optionally be optimised for each of the Cas13 polypeptides of the invention, and how to do so.

### 6. Probe Types

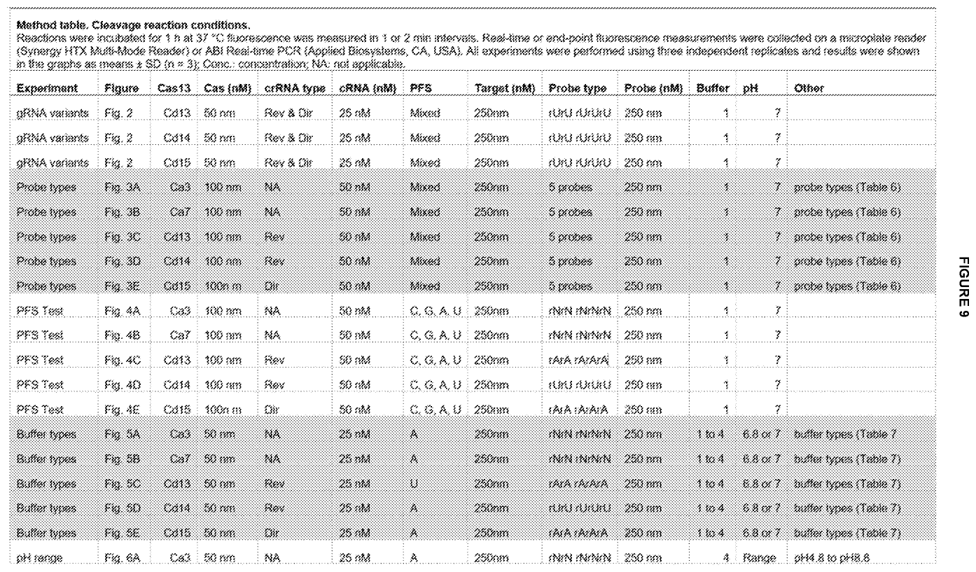

The length and sequence of the ssRNA probes/reporter sequences may also influence Cas13 trans-cleavage activities. Various ssRNA probes containing FAM at 5′ end and 3IABkFQ quencher at the 3′ end (Table 6) were tested. When Cas13 cleaves ssRNA, an increase of fluorescence signal is observed. The experimental conditions are is described in FIG. 9. The fluorescence was measured in 2 min intervals. The measurement of real-time background subtracted fluorescence output are shown in the graphs as means±SD (n=3).

TABLE 6
Probes/reporters tested for their effect on Cas13 collateral activity

| Reporter RNAs | Probe type |
|---|---|
| FAM------3IABkFQ | /56-FAM/rUrUrUrUrU/3IABkFQ/ |
| FAM------3IABkFQ | /56-FAM/TArUrUGC/3IABkFQ/ |
| FAM------3IABkFQ | /56-FAM/rUrG rArCrG rU/3IABkFQ/ |
| FAM------3IABkFQ | /56-FAM/rArA rArArA/3IABkFQ/ |
| FAM------3IABkFQ | /56-FAM/rNrN rNrNrN/3IABkFQ/ |

### Results

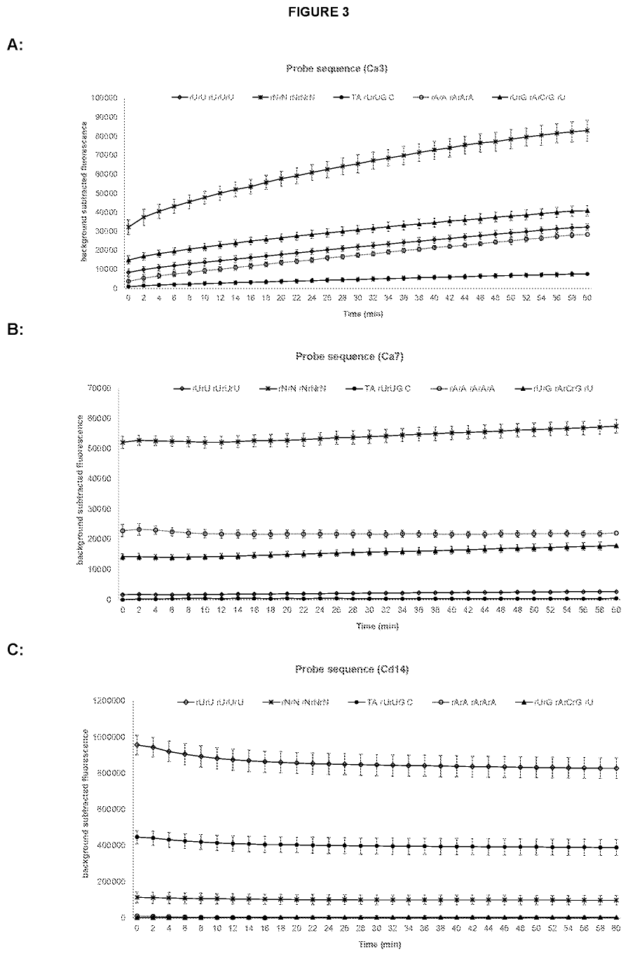

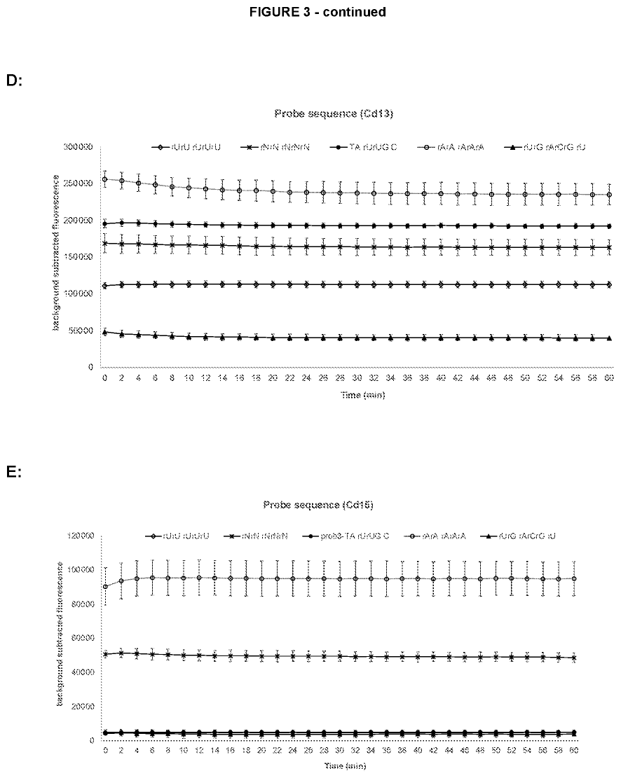

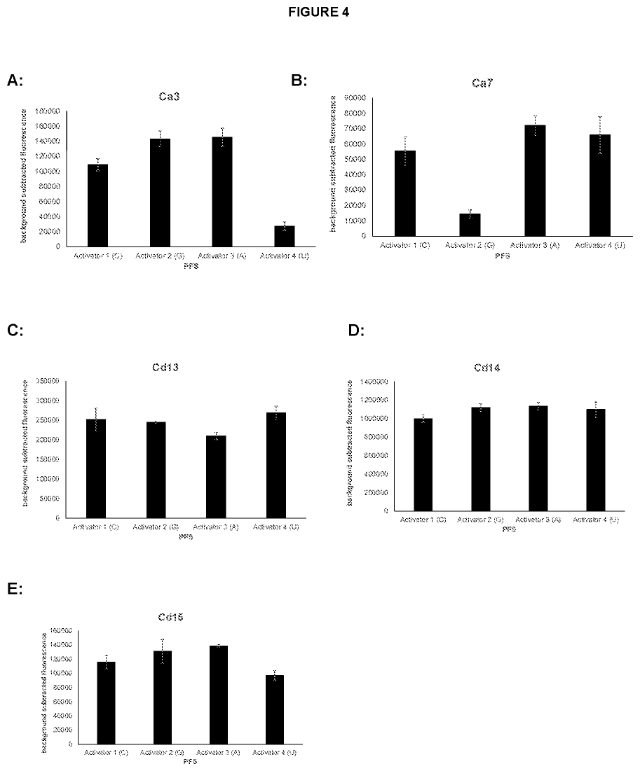

Cas13 polypeptides of the invention showed different activities with various probes (FIG. 4). Cas13a3, Cas13a7, Cas13d13, Cas13d14 and Cas13d15 preferred probes rUrUrUrUrU, rNrNrNrNrN, rArArArArA, rUrUrUrUrU, and rArA rArArA, respectively. The results for a subset of them (Cas13a3, Cas13a7, Cas13d13, Cas13d14 and Cas13d15) are presented in FIG. 3.

### 7. PFS Analysis of Cas13 Polypeptides

Since the activity of Cas13 enzymes may depend on the presence or absence of a PFS in the target sequence, and they may be more or less sensitive to one PFS over another, experiment 6 was repeated but using the single RNA substrates of Table 5 rather than the initial experiment that tested all 4 RNA substrates at once.

The ability of Cas13a3, Cas13a7, Cas13d13, Cas13d14 and Cas13d15 to cleave a target ssRNA with each possible protospacer flanking site (PFS) nucleotide (A, U, C or G) was assayed.

### Method

Test conditions as per FIG. 9, comprising 50 nM crRNA, 100 nM Cas13, 500 250 nM target ssRNA, 250 nM reporter and 1× buffer (20 mM HEPES-Na pH 6-8, 50 mM NaCl, 10 mM MgCl2, and 1 mM TCEP Buffer 1, Table 7). The fluorescence was measured in 2 min intervals. End-point activity of enzymes Values are shown in the graphs as means±SD (n=3).

### Results

Cas13a3 can also robustly cleave a target with A, C, or G PFS, with less activity on the ssRNA with a U PFS. Cas13a7 can also robustly cleave a target with A, U, or C PFS, with less activity on the ssRNA with a G PFS. Cas13d13, Cas13d14 and Cas13d15 can robustly cleave all four targets with slightly less activity on the ssRNA with a A, C, and U PFS, respectively. The results for a subset of them (Cas13a3, Cas13a7, Cas13d13, Cas13d14 and Cas13d15) are presented in FIG. 4.

### 8. Buffers and pH

For the optimisation of buffer compositions, a variety of buffers (Table 7) and pH ranges were tested. The procedure was as described in FIG. 9.

### a) Buffer

First, the buffers in Table 9 were tested:

TABLE 7
Components of buffers used for optimisation

| Buffer 1 | Buffer 2 | Buffer 3 | Buffer 4 |
|---|---|---|---|
| 20 mM HEPES | 20 mM HEPES | 20 mM HEPES | 20 mM HEPES |
| pH 7.0 | pH 7.0 | pH 6.8 | pH 6.8 |
| 50 mM NaCl | 50 mM NaCl | 60 mM NaCl | 50 mM KCl |
| 10 mM MgCl2 | 10 mM MgCl2 | 6 mM MgCl2 | 5 mM MgCl2 |
| 5% glycerol | 5% glycerol | 5% glycerol | 5% glycerol |
| — | 1 μg/ml BSA | — | — |

### Results

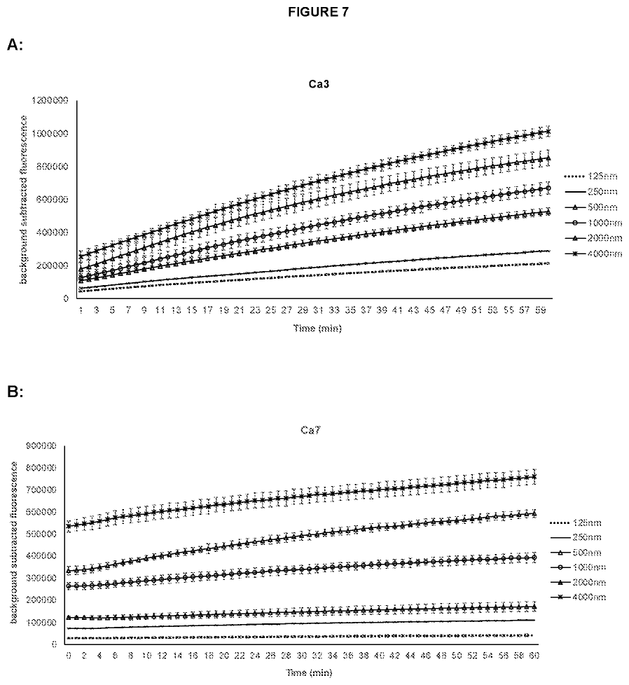

The results from the comparison of the Cas13 reaction buffers are presented in FIG. 6 for a representative sample of cleavage reactions using Cas13a3, Cas13a7, Cas13d13, Cas13d14 and Cas13d15. End-point activity of enzymes are shown in the graphs as means±SD (n=3).

Cas13d13, Cas13d14 and Cas13d15 generally preferred buffer 2 and exhibited the highest collateral activities in the presence of Buffer 2, whereas Cas13a3 and Cas13a7 showed the highest activity in the presence of Buffer 4.

b) pH

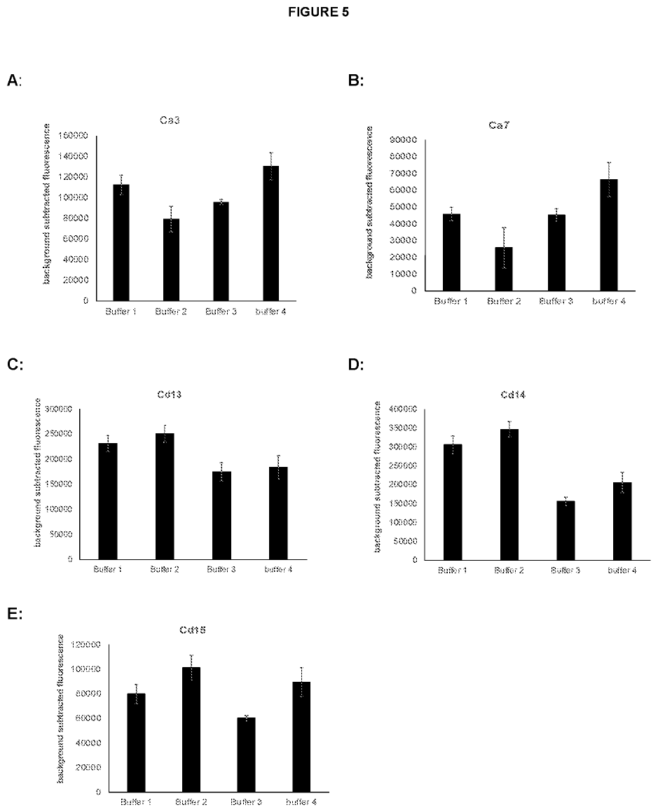

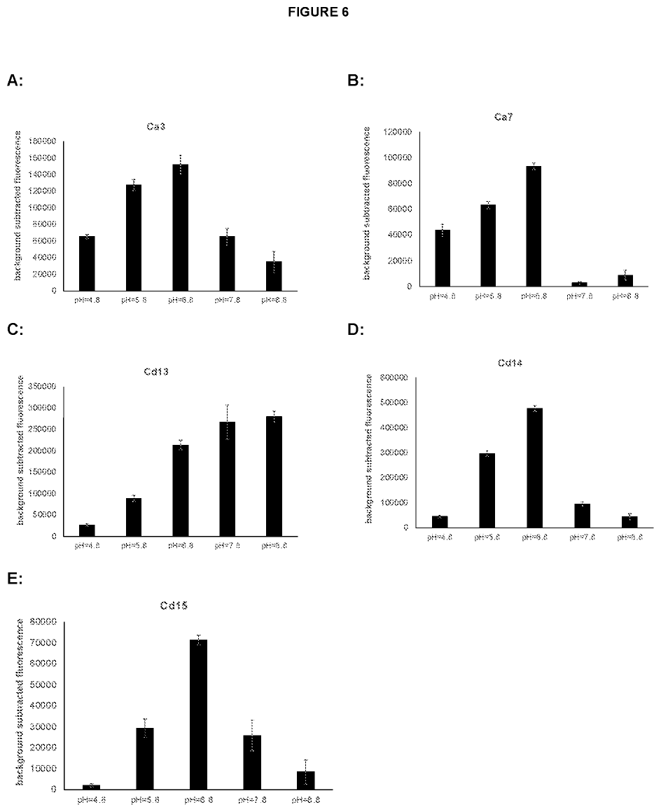

As the pH values may influence the Cas13 trans-cleavage activities, the cleavage assay was repeated in the buffer determined to be the best for each of Cas13a3, Cas13a7, Cas13d13, Cas13d14 and Cas13d15 (see FIG. 5) but with pH values ranging from 4.8 to 8.8 in increments of 1.0.

### Results

The results from the comparison of the pH ranges are presented in FIG. 6 for a representative sample of cleavage reactions using Cas13a3, Cas13a7, Cas13d13, Cas13d14 and Cas13d15. End-point activity of enzymes are shown in the graphs as means±SD (n=3).

Cas13a3, Cas13a7, Cas13d14 and Cas13d15 had the highest collateral activity at the pH 6.8, whereas Cas13d13 demonstrated the highest activity at alkaline pH 8.8.

### 9. Probe Concentration

Since the fluorescent probe is another key component that influences the reaction, we optimized the assay by incubating increasing amounts of probes with constant concentrations of Cas13-crRNA and target ssRNA with the optimum buffer and pH for each Cas13 enzyme.

### Method

The procedure was as described in FIG. 9. The cleavage assay was performed in the buffer and pH using probe sequences determined to be the best for each of Cas13a3, Cas13a7, Cas13d13, Cas13d14 and Cas13d15 (see FIGS. 5 and 6) but with probe concentration of 125, 250, 500, 100, 2000 and 4000 nM.

### Results

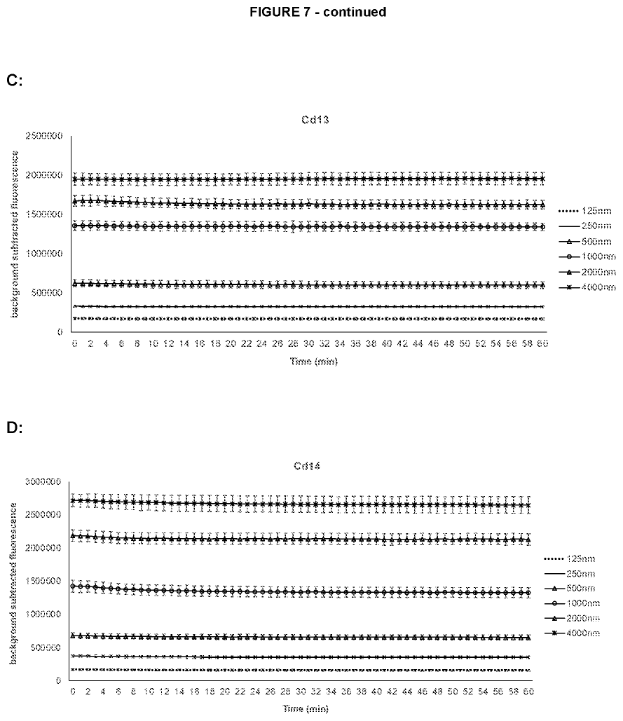

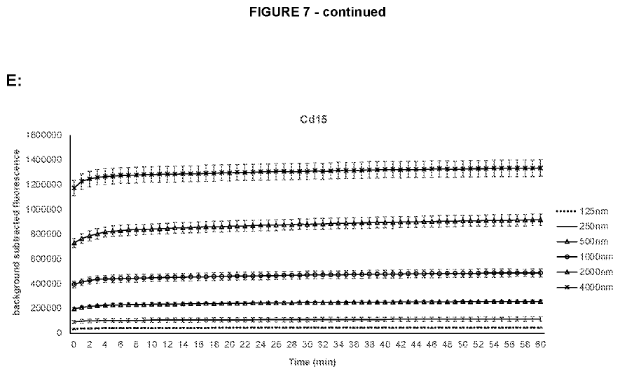

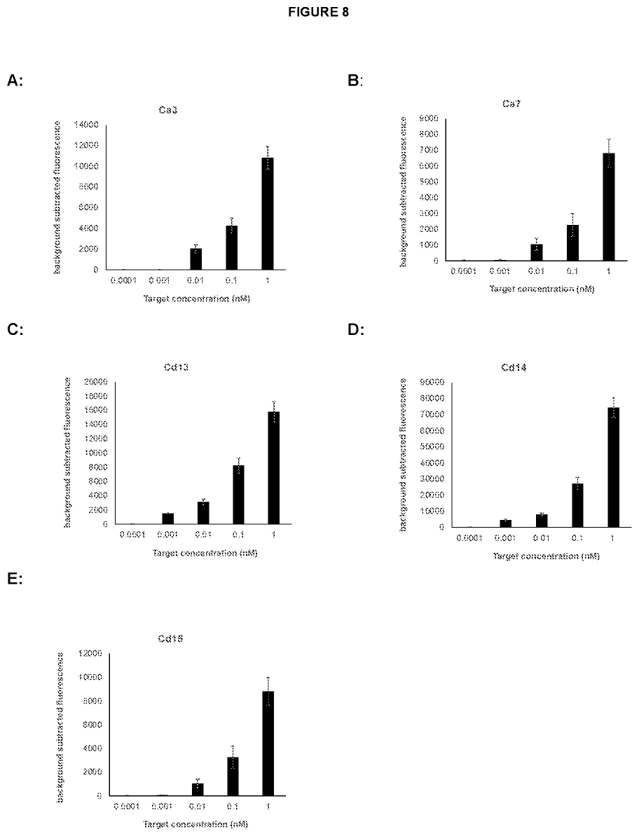

The results from the comparison of the probe concentrations are presented in FIG. 7 for a representative sample of cleavage reactions using Cas13a3, Cas13a7, Cas13d13, Cas13d14 and Cas13d15. The measurement of real-time background subtracted fluorescence output are shown in the graphs as means±SD (n=3). The results demonstrated that CRISPR-mediated fluorescence signal intensities increased with increasing amounts of probes (from 125 nM to 4000 nM).

### Summary of Optimisation Experiments

FIG. 9 presents the results from examples 6 to 9 for a representative sample of cleavage reactions using Cas13a3, Cas13a7, Cas13d13, Cas13d14 and Cas13d15.

Initial test: this is example 5 which confirms trans cleavage (collateral activity), and the direct repeat direction for the Cas13d polypeptides tested (see column labelled crRNA type). This information is taken in to the next example: probe types.

Probe types: this is example 6 and confirms the preference for probe type of each of Cas13a3, Cas13a7, Cas13d13, Cas13d14 and Cas13d15 (see column labelled probe type). The preferred probe is used in the next example: PFS test.

PFS test: This is example 7 and assesses whether each of the activity of Cas13a3, Cas13a7, Cas13d13, Cas13d14 and Cas13d15 depend on the presence or absence of a PFS in the target sequence, and if so, if they may be more or less sensitive to one PFS over another (see column labelled PFS). If there is a dependence on a particular PFS that is used in the next experiment; if there is no dependence on, or preference for a PFS, any PFS or mixtures thereof can be used in subsequent examples: Buffer types.

Buffer types: This is example 8a and assesses which buffer gives the best cleavage activity for each of Cas13a3, Cas13a7, Cas13d13, Cas13d14 and Cas13d15 (see column labelled buffer). The next example is conducted using the preferred buffer: pH test.

pH: this is example 8b and assesses the best buffer from the previous example at a range of pHs (see column labelled pH). The next experiment is conducted in the preferred buffer at the preferred pH: probe concentration.

Probe concentration: this is example 9, to optimise how much probe to use in each reaction (see column labelled Probe).

By the end of this process, which can be applied to any Cas13 polyprotein, the optimal conditions for a representative subset of Cas13 polypeptides of the invention have been determined as set out in Table 8:

TABLE 8
The optimal conditions for a representative
subset of Cas13 polypeptides

| Cas13 | | | | | |
|---|---|---|---|---|---|
| protein | Direction | PFS | Probe type | Buffer | pH |
| Cas13a3 | NA | G = A > C > U | rNrN rNrNrN | 4 | 6.8 |
| Cas13a7 | NA | A = U > C > G | rNrN rNrNrN | 4 | 6.8 |
| Cas13a13 | Rev | U = C = G > A | rArA rArArA | 2 | 8.8 |
| Cas13a14 | Rev | G = A = U > C | rUrU rUrUrU | 2 | 6.8 |
| Cas13a15 | Dir | A = G = C > U | rArA rArArA | 2 | 6.8 |

### 10. Limit of Detection (LoD) Experiments

An important feature of a nucleic acid detection platform is the detection sensitivity. The detection sensitivities of the Cas13 polypeptides were therefore investigated. Having determined the optimal reaction conditions for the Cas13 polypeptides of the invention as per Table 8, different concentrations of target RNA were tested to evaluate the sensitivity of the detection system.

### Methods

To determine the LoD for each Cas13 enzyme, a 20 μL detection system was prepared that consisted of 100 nM Cas13, 50 nM crRNA, 1000 nM ssRNA reporter, and different concentrations of target RNA (between 1 nM and 0.0001 nM in 10-fold dilutions), for each enzyme in the corresponding reaction buffer. Fluorescence readouts were taken in 2 min intervals for a total of 60 min on the ABI Real-time PCR (Applied Biosystems, CA, USA).

### Results

As shown in FIG. 7, the fluorescence signal intensities of samples composed of 1 nM, 100 pM, 10 pM, 1 pM, and 100 fM target ssRNA were analysed. Cd13 and Cd14 could specifically and stably detect 1 pM target RNA; Ca3, Ca7 and Cd15 could detect up to 10 pM.

Altogether, these data indicated that the identified Cas13 polypeptides are catalytically active with a robust trans cleavage activity and good detection sensitivity, and thus are suitable for developing nucleic acid detection platforms.

### 11. Covid Detection

The ability of Cas13 polypeptides to rapidly detect nucleic acids with high sensitivity aids in disease diagnosis and monitoring, epidemiology, and general laboratory tasks. To address this, the cis and trans activities in vitro of the Cas13 polypeptides of the invention were evaluated for use in a Cas13 SARS-CoV-2-based detection assays. Furthermore, two different crRNAs for Cas13d14 were designed, targeting two different regions in the SARS-CoV-2 nucleocapsid (N) gene.

### Methods

Fragment of DNA template of SARS-CoV-2 N gene (Target) (Broughton J P, et al. CRISPR-Cas12-based detection of SARS-CoV-2. Nat Biotechnol. 2020 July; 38(7):870-874) for T7 in-vitro transcription (IVT) was synthesised and cloned in pUC57 by GenScript (Table 9). The amplification of N gene DNA oligo template were performed using forward and reverse primers as described in Example 3. TranscriptAid T7 High Yield Transcription Kit (Thermo Fisher Scientific) was used for ssRNA template (Target) synthesis. After DNaseI treatment of IVT reaction, RNA purification from IVT reaction were performed as described in Example 3.

TABLE 9
Sense DNA template of SARS-CoV-2 N gene for production of RNA substrates
(ie target sequences)

| Name | Sequence |
|---|---|
| Fragment of SARS- | 5′CCAAATTGGCTACTACCGAAGAGCTACCAGACGAATTCGTGGTGGTGACGGTAAA |
| CoV-2 N gene | ATGAAAGATCTCAGTCCAAGATGGTATTTCTACTACCTAGGAACTGGGCCAGAAGCT |
| | GGACTTCCCTATGGTGCTAACAAAGACGGCATCATATGGGTTGCAACTGAGGGAGC |
| | CTTGAATACACCAAAAGATCACATTGGCACCCGCAATCCTGCTAACAATGCTGCAAT |
| | CGTGCTACAACTTCCTCAAGGAACAACATTGCCAAAAGGCTTCTACGCAGAAGGGAG |
| | CAGAGGCGGCAGTCAAGCCTCTTCTCGTTCCTCATCACGTAGTCGCAACAGTTCAAG |
| | AAATTCAACTCCAGGCAGCAGTAGGGGAACTTCTCCTGCTAGAATGGCTGGCAATG |
| | GCGGTGATGCTGCTCTTGCTTTGCTGCTGCTTGACAGATTGAACCAGCTTGAGAGCA |
| | AAATGTCTGGTAAAGGCCAACAACAACAAGGCCAAACTGTCACTAAGAAATCTGCTG |
| | CTGAGGCTTCTAAGAAGCCTCGGCAAAAACGTACTGCCACTAAAGCATACAATGTAA |
| | CACAAGCTTTCGGCAGACGTGGTCCAGAACAAACCCAAGGAAATTTTGGGGACCAG |
| | GAACTAATCAGACAAGGAACTGATTACAAACATTGGCCGCAAATTGCACAATTTGCC |
| | CCCAGCGCTTCAGCGTTCTTCGGAATGTCGCGCATTGGCATGGAAGTCACACCTTC |
| | GGGAACGTGGTTGACCTACACAGGTGCCATCAAATTGGATGACAAAGATCCAAATTT |
| | CAAAGATCAAGTCATTTTGCTGAATAAGCATATTGACGCATACAAAACATTCCCACCA |
| | ACAGAGCCTAAAAAGGACAAAAAGAAGAAGGCTGATGAAACTCAAGCCTTACCGCA |
| | GAGACAGAAGAAACAGCAAACTGTG-3′ (SEQ ID NO: 103) |

Short 81 or 82-bp dsDNA made by annealing of complementary oligos were used to generate crRNAs dsDNA coding sequence (Fozouni P, et al. Amplification-free detection of SARS-CoV-2 with CRISPR-Cas13a and mobile phone microscopy. Cell. 2021 Jan. 21; 184(2):323-333). First, concentrated complementary oligonucleotides (Table 10) were mixed together at a 1:1 molar ratio in a microcentrifuge tube and were diluted to a final concentration of 1 pmol/μl with a Tris or phosphate buffer (10 mM Tris, 1 mM EDTA, 50 mM NaCl (pH 8.0) or 100 mM sodium phosphate, 150 mM NaCl, 1 mM EDTA (pH 7.5)). To generate crRNA dsDNA, complementary oligonucleotides were annealed using thermocycler with a denaturation step of 95° C. for 5 min; then, the temperature gradually decreased by 1 degree per minute to 25° C. The resultant 81 or 82-bp crRNA dsDNA was used to synthesize crRNA (Table 11) using T7 RNA polymerase primer as described in Example 3.

TABLE 10
Complementary oligonucleotides for synthesis of crRNA dsDNA for SARS-CoV-2.
Complementary oligonucleotides are shown as a and b for each crRNA.

| Name | Sequence |
|---|---|
| Ca3-CoV crRNAa | TAATACGACTCACTATAGGAGATGAAAAAAGCCCGACATAGCGGGCAATCGAATTGGT |
| | GTATTCAAGGCTCCCTCAGTTGC (SEQ ID NO: 104) |
| Ca7-CoV crRNAb | GCAACTGAGGGAGCCTTGAATACACCAATTCGATTGCCCGCTATGTCGGGCTTTTTTC |
| | ATCTCCTATAGTGAGTCGTATTA (SEQ ID NO: 105) |
| Ca7-CoV crRNAa | TAATACGACTCACTATAGTTAGATGAGAACACTCCGAGATAACGGAGAATAACTTGGT |
| | GTATTCAAGGCTCCCTCAGTTGC (SEQ ID NO: 106) |
| Ca7-CoV crRNAb | GCAACTGAGGGAGCCTTGAATACACCAAGTTATTCTCCGTTATCTCGGAGTGTTCTCA |
| | TCTAACTATAGTGAGTCGTATTA (SEQ ID NO: 107) |
| Cd13-CoV crRNAa | TAATACGACTCACTATAGAAACGTACTACACCCTTTTTGTAAGGGTCTGAAACCTTGTG |
| | CAATTTGCGGCCAATGTTTGTAA (SEQ ID NO: 108) |
| Cd13-CoV crRNAb | TTACAAACATTGGCCGCAAATTGCACAAGGTTTCAGACCCTTACAAAAAGGGTGTAGT |
| | ACGTTTCTATAGTGAGTCGTATTA (SEQ ID NO: 109) |
| Cd14-CoV crRNA | TAATACGACTCACTATAGCAACTACAACCCTGTCAAATTACAGGGTTCTGAAACTTGTG |
| 1a | CAATTTGCGGCCAATGTTTGTAA (SEQ ID NO: 110) |
| Cd14-CoV crRNA | TTACAAACATTGGCCGCAAATTGCACAAGTTTCAGAACCCTGTAATTTGACAGGGTTGT |
| 1b | AGTTGCTATAGTGAGTCGTATTA (SEQ ID NO: 111) |
| Cd14-CoV crRNA | TAATACGACTCACTATAGCAACTACAACCCTGTCAAATTACAGGGTTCTGAAACTTGGT |
| 2a | GTATTCAAGGCTCCCTCAGTTGC (SEQ ID NO: 112) |
| Cd14-CoV crRNA | GCAACTGAGGGAGCCTTGAATACACCAAGTTTCAGAACCCTGTAATTTGACAGGGTTG |
| 2b | TAGTTGCTATAGTGAGTCGTATTA (SEQ ID NO: 113) |
| Cd15-CoV crRNAa | TAATACGACTCACTATAGATCTATAACCCTGCATTTATGTAGGGCTCTAAAACTTGGTG |
| | TATTCAAGGCTCCCTCAGTTGC (SEQ ID NO: 114) |
| Cd15-CoV crRNAb | GCAACTGAGGGAGCCTTGAATACACCAAGTTTTAGAGCCCTACATAAATGCAGGGTTA |
| | TAGATCTATAGTGAGTCGTATTA (SEQ ID NO: 115) |

TABLE 11
crRNA sequences (5′ to 3′) for for SARS-CoV-2 detection.

| Name | Sequence |
|---|---|
| Ca3-CoV crRNA | GGAGAUGAAAAAAGCCCGACAUAGCGGGCAAUCGAAUUGGUGUAUUCAAGGCUCCC |
| | UCAGUUGC (SEQ ID NO: 116) |
| Ca7-CoV crRNA | GUUAGAUGAGAACACUCCGAGAUAACGGAGAAUAACUUGGUGUAUUCAAGGCUCCC |
| | UCAGUUGC (SEQ ID NO: 117) |
| Cd13-CoV crRNA | GAAACGUACUACACCCUUUUUGUAAGGGUCUGAAACCUUGUGCAAUUUGCGGCCAA |
| | UGUUUGUAA (SEQ ID NO: 118) |
| Cd14-CoV crRNA 1 | GCAACUACAACCCUGUCAAAUUACAGGGUUCUGAAACUUGUGCAAUUUGCGGCCAA |
| | UGUUUGUAA (SEQ ID NO: 119) |
| Cd14-CoV crRNA 2 | GCAACUACAACCCUGUCAAAUUACAGGGUUCUGAAACUUGGUGUAUUCAAGGCUCC |
| | CUCAGUUGC (SEQ ID NO: 120) |
| Cd15-CoV crRNA | GAUCUAUAACCCUGCAUUUAUGUAGGGCUCUAAAACUUGGUGUAUUCAAGGCUCCC |
| | UCAGUUGC (SEQ ID NO: 121) |

A 20 μL detection system was prepared that consisted of 100 nM Cas13, 50 nM crRNA, 250 nM ssRNA reporter, and 250 nM target ssRNA, for each enzyme in the corresponding reaction buffer. Fluorescence readouts were taken in 2 min intervals for a total of 60 min on the ABI Real-time PCR (Applied Biosystems, CA, USA).

### Results

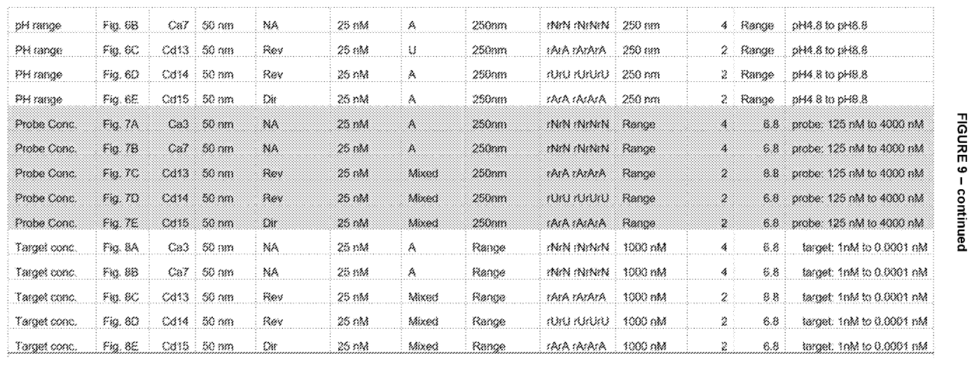

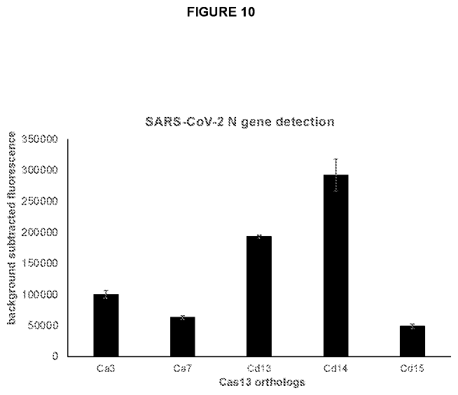

In vitro cis- and trans-cleavage activities of Cas13 polypeptides tested revealed the that all 5 could detect SARS-CoV-2 N gene, with Cas13d14 exhibiting the highest efficiency (FIG. 10). These results confirm the ability for the Cas13 polypeptides to detect different targets and their use in detection assays, such as a Cas13 SARS-CoV-2-based detection assay.

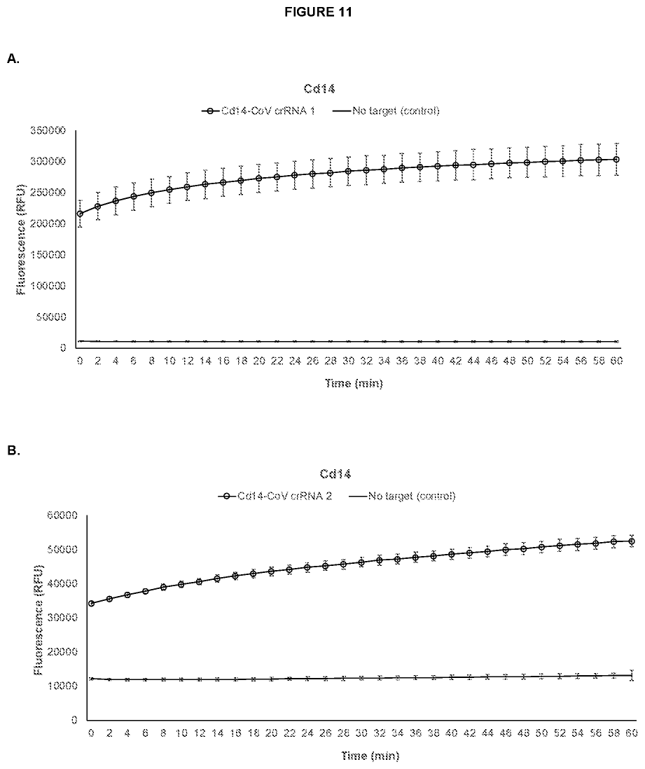

In addition, Cas13d14 showed different collateral (trans) cleavage activity with two crRNAs, with crRNA 1 (labelled as crRNA 1) mediating the highest efficiency relative to crRNA 2 (labelled as crRNA 2) and controls. This result demonstrated the efficiency of Cas13 detection can be impacted by the crRNAs (FIG. 11A and B).

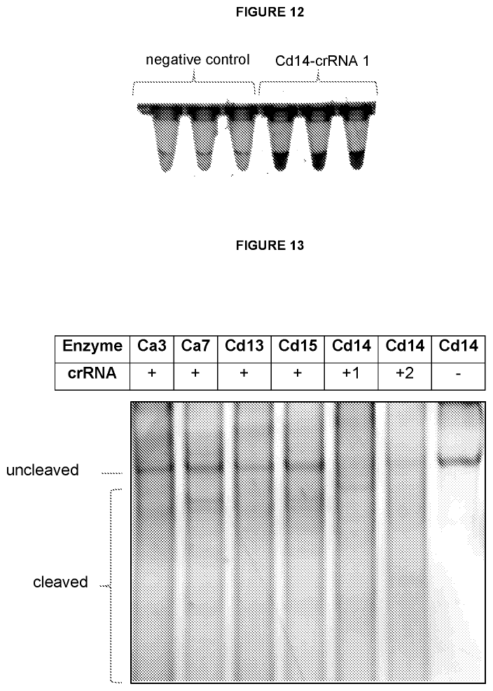

Furthermore, the results from the visual-based detection assay were compared to the results in FIG. 11A. The image of strip of reactions were captured using SYNGENE transilluminator (420 nm wavelength), 15 minutes after beginning of collateral activity (FIG. 12). The results showed concordance between the two assays, indicating that the developed Cas13 visual detection assay is reliable. Visual-based detection assays can therefore be used for home detection kits.

The results of in vitro cis-cleavage activity of Cas13 polypeptides showed different efficiencies, with Cas13d14 exhibiting the highest relative efficiency enzymes (FIG. 13). The presence and absence of crRNAs has been designated as ‘+’ and ‘−’, respectively. Cd14-CoV crRNA 1 and Cd14-CoV crRNA 2 were shown by ‘+1’ and ‘+2’, respectively. The reaction containing Cas13d14 without crRNA was used as control.

1-19. (canceled)

20. An engineered Cas13d polypeptide, wherein the Cas13d polypeptide has an amino acid sequence selected from the group consisting of SEQ ID NO: 29, SEQ ID NO: 28 or SEQ ID NO: 30, or a sequence being at least 80%, 85%, 90%, 91%, 92%, 93%, 94%, 95%, 96%, 97%, 98%, or 99% identical to SEQ ID NO: 29, SEQ ID NO: 28 and SEQ ID NO: 30.

21. An engineered Cas13d polypeptide according to claim 20, wherein the Cas13d polypeptide is encoded by a nucleic acid molecule selected from by SEQ ID NO: 14, SEQ ID NO: 13 or SEQ ID NO: 15, or is encoded by a sequence being at least 80%, 85%, 90%, 91%, 92%, 93%, 94%, 95%, 96%, 97%, 98%, or 99% identical to SEQ ID NO: 14, SEQ ID NO:13 or SEQ ID NO: 15.

22. A composition comprising the engineered Cas13d polypeptide of claim 20.

23. A vector comprising the nucleic acid molecule described in claim 21.

24. A CRISPR/Cas13d system for targeting RNA molecules, the system comprising a) at least one Cas13d polypeptide wherein the Cas13d polypeptide has an amino acid sequence selected from the group consisting of SEQ ID NO: 29, SEQ ID NO: 28 or SEQ ID NO: 30, or a sequence being at least 80%, 85%, 90%, 91%, 92%, 93%, 94%, 95%, 96%, 97%, 98%, or 99% identical to SEQ ID NO: 29, SEQ ID NO: 28 or SEQ ID NO: 30; or a nucleic acid molecule comprising a sequence encoding the Cas13d polypeptide; and b) at least one CRISPR RNA (crRNA) or at least one nucleic acid molecule encoding the at least one crRNA, the crRNA comprising one or more spacers and one or more Cas13-specific direct repeats, wherein the crRNA is capable of hybridising with one or more target RNA molecules.

25. A CRISPR/Cas13d system for targeting RNA molecules according to claim 24, the system comprising:
a) a nucleic acid molecule comprising a sequence encoding the Cas13d polypeptide; and
b) at least one nucleic acid molecule encoding the at least one crRNA, the crRNA comprising one or more spacers and one or more Cas13-specific direct repeats, wherein the crRNA is capable of hybridising with one or more target RNA molecules.

26. A CRISPR/Cas13d system for targeting RNA molecules according to claim 25, wherein the system further comprises a vector system of one or more vectors comprising: i) a first regulatory element operably linked to the nucleic acid molecule of element (a); and ii) a second regulatory element operably linked to the nucleic acid molecule of element (b); wherein components (i) and (ii) are located on the same or different vectors of the system.

27. A CRISPR/Cas13d system according to claim 24, wherein the nucleic acid molecule encoding the Cas13d polypeptide is selected from the group consisting of SEQ ID NO: 14, SEQ ID NO: 13 or SEQ ID NO: 15, or is selected from a sequence being at least 80%, 85%, 90%, 91%, 92%, 93%, 94%, 95%, 96%, 97%, 98%, or 99% identical to SEQ ID NO: 14, SEQ ID NO: 13 or SEQ ID NO: 15.

28. An in vitro method of modifying a target RNA, the method comprising contacting the target RNA with a ribonucleoprotein (RNP) complex of a CRISPR/Cas13d system, the system comprising:
i) at least one Cas13d polypeptide wherein the Cas13d polypeptide has an amino acid sequence selected from the group consisting of SEQ ID NO: 29, SEQ ID NO: 28 or SEQ ID NO: 30, or a sequence being at least 80%, 85%, 90%, 91%, 92%, 93%, 94%, 95%, 96%, 97%, 98%, or 99% identical to SEQ ID NO: 29, SEQ ID NO: 28 or SEQ ID NO: 30; and
ii) at least one CRISPR RNA (crRNA), the crRNA comprising one or more spacers and one or more Cas13-specific direct repeats, wherein the crRNA is capable of hybridising with one or more target RNA molecules, wherein the Cas13d polypeptide and the crRNA form a ribonucleoprotein (RNP) complex, and upon binding of the complex to the target RNA through the one or more spacers, the Cas13d polypeptide modifies the target RNA.

29. The method of modifying a target RNA according to claim 28, wherein prior to contacting the target RNA with the RNP complex, the method comprises:
a) expressing from a vector system at least one Cas13d polypeptide and at least one CRISPR RNA (crRNA), the vector system comprising one or more vectors comprising:
i) a first regulatory element operably linked to a nucleic acid molecule comprising a sequence encoding a Cas13d polypeptide wherein the Cas13d polypeptide has an amino acid sequence selected from the group consisting of SEQ ID NO: 29, SEQ ID NO: 28 or SEQ ID NO: 30, or a sequence being at least 80%, 85%, 90%, 91%, 92%, 93%, 94%, 95%, 96%, 97%, 98%, or 99% identical to SEQ ID NO: 29, SEQ ID NO: 28 or SEQ ID NO: 30; and
ii) a second regulatory element operably linked to a nucleic acid molecule encoding a CRISPR RNA (crRNA), the crRNA comprising one or more spacers and one or more Cas13-specific direct repeats, wherein the crRNA is capable of hybridising with one or more target RNA molecules; wherein components (i) and (ii) are located on the same or different vectors of the system; and
b) isolating the expression products of step (a); and then
c) contacting the target RNA with the isolated expression products of step (b), wherein the Cas13d polypeptide and the crRNA form an RNP complex, and upon binding of the RNP complex to the target RNA through the one or more spacers, the Cas13d polypeptide modifies the target RN A.

30. The method according to claim 29, wherein the isolated expression products of step (b) are assembled into the RNP complex prior to contact with the target RNA in step (c).

31. The method according to claim 28, wherein the nucleic acid molecule encoding the Cas13d polypeptide is selected from the group consisting of SEQ ID NO: 14, SEQ ID NO: 13 or SEQ ID NO: 15, or is selected from a sequence being at least 80%, 85%, 90%, 91%, 92%, 93%, 94%, 95%, 96%, 97%, 98%, or 99% identical to SEQ ID NO: 14, SEQ ID NO: 13 or SEQ ID NO: 15.

32. A nucleic acid detection system, the system comprising:
i) at least one Cas13d polypeptide wherein the Cas13d polypeptide has an amino acid sequence selected from the group consisting of SEQ ID NO: 29, SEQ ID NO: 28 or SEQ ID NO: 30, or a sequence being at least 80%, 85%, 90%, 91%, 92%, 93%, 94%, 95%, 96%, 97%, 98%, or 99% identical to SEQ ID NO: 29, SEQ ID NO: 28 or SEQ ID NO: 30, or a nucleic acid molecule comprising a sequence encoding the Cas13d polypeptide and
ii) at least one CRISPR RNA (crRNA) or a nucleic acid molecule encoding the crRNA, the crRNA comprising one or more spacers and one or more Cas13-specific direct repeats, and
iii) a detector RNA wherein the crRNA is capable of hybridising with one or more target RNA molecules, and the Cas13d polypeptide has at least trans cleavage activity.
